(19) United States
(12) Patent Application Publication
(10) Pub. No.: US 2024/0229055 A9
(43) Pub. Date: Jul. 11, 2024

(54) MOGROSIDE COMPOSITIONS AND METHODS OF PRODUCING SAME

(71) Applicant: Elo Life Systems, Durham, NC (US)

(72) Inventors: Tengfang Huang, Cary, NC (US); Jack Q. Wilkinson, Cary, NC (US); Anna Matthiadis, Durham, NC (US); Devarshi Selote, Apex, NC (US); Matthew Kilgore, Cary, NC (US); Matthew DiLeo, Durham, NC (US); Fayaz Khazi, Chapel Hill, NC (US); Kevin Potter, Cary, NC (US); Daniel Stauffer, Raleigh, NC (US); Todd Rands, Naples, FL (US)

(21) Appl. No.: 18/470,037

(22) Filed: Sep. 19, 2023

Related U.S. Application Data
(60) Provisional application No. 63/491,721, filed on Mar. 22, 2023.
(60) Provisional application No. 63/376,194, filed on Sep. 19, 2022.

(51) Int. Cl. C12N 15/82; A23L 27/30; C12N 9/02; C12N 9/04; C12N 9/10; C12N 9/90; C12N 15/52

(52) U.S. Cl. CPC C12N 15/8243; A23L 27/36; C12N 9/0004; C12N 9/0006; C12N 9/0042; C12N 9/0071; C12N 9/1051; C12N 9/90; C12N 15/52; C12Y 101/01088; C12Y 106/02004; C12Y 504/99003

(57) ABSTRACT

The present disclosure provides compositions and methods for producing transgenic plants and other organisms that exhibit increased production of mogroside compounds, in particular mogroside V, and the mogroside compounds, plants and plant parts so produced.

### CROSS-REFERENCE TO RELATED APPLICATIONS

This application claims the benefit under 35 U.S.C. § 119(e) of U.S. provisional application Ser. No. 63/376,194, filed on Sep. 19, 2022, and 63/491,721, filed on Mar. 22, 2023, each of which is herein incorporated by reference in its entirety.

### INCORPORATION OF SEQUENCE LISTING

A sequence listing containing the file named “ELSS002US_ST26.xml” which is 540 kilobytes (measured in MS-Windows®) and created on Sep. 14, 2023, and comprises 305 sequences, is incorporated herein by reference in its entirety.

### FIELD OF THE INVENTION

The present disclosure relates to the field of genetically engineering plants and other organisms, and more specifically to methods and compositions for producing plants and other organisms exhibiting increased production of mogroside compounds, in particular mogroside V. The present disclosure also relates to the use of such plants and other organisms to produce novel ingredients (e.g., plant extracts, purified and partially purified fractions containing mogrosides) for foods and beverages, and novel foods and beverages (and other compositions of matter) resulting therefrom.

### BACKGROUND OF THE INVENTION

Low or non-caloric sweeteners, in particular natural low or non-caloric sweeteners, as an alternative to traditional high calorie sweeteners and artificial sweeteners are becoming increasingly important to the food and beverage industry, in addition to other industries. These alternative sweeteners are used as a substitute for artificial sweeteners or high calorie sweeteners comprising sucrose, fructose, and glucose. Like some artificial sweeteners, some of these alternative sweeteners provide a greater sweetening effect than comparable amounts of caloric sweeteners, and therefore smaller amounts of these alternative sweeteners are required to achieve sweetness comparable to that of sugar. However, some low calorie sweeteners can be expensive to produce and/or possess unfavorable taste characteristics and/or off-tastes, including but not limited to sweetness linger, delayed sweetness onset, negative mouth feel, and bitter, metallic, cooling, astringent, and licorice-like tastes.

A few natural plants produce low or non-calorie sweeteners. For example, mogrosides, an important class of natural sweeteners, are chemically a class of triterpene glycosides or mogrol glycosides naturally produced by monkfruit (also known as luohan guo; scientific name: Siraitia grosvenorii). Mogrosides contain “zero” calories (less than 5 calories per 8 oz. serving), and are 100400 times sweeter than sucrose. Mogrosides have also been reported to have a variety of important pharmacological effects. However, although plants like Siraitia grosvenorii make mogrosides, production of mogrosides from these plants is limited and expensive due to the limited natural or agricultural production of these plants. Also, Siraitia grosvenorii prefers to grow in subtropical mountainous regions and requires laborious pollination to set fruits. In addition, production of mogrosides in vitro or in microorganisms has been attempted, but due to extensive processing and other issues has not proven to be economically feasible.

Therefore there is a need for new compositions and methods for the efficient production of mogrosides.

### SUMMARY

The present disclosure solves these and other problems in the art by providing novel compositions and methods for the efficient and cost-effective production of mogrosides in plants and other organisms, in particular sweet mogrosides, such as those including more than three glucose residues in the molecule, including, but not limited to, mogroside V, isomogroside V, siamenoside I, α-siamenoside I, mogroside IV, mogroside IV A, mogroside III, mogroside III E, mogroside III A1 and 11-oxo-mogroside V.

The present disclosure provides a transgenic plant, plant part or seed, comprising: a) a first polynucleotide sequence that encodes a cytochrome P450 polypeptide having at least 91% sequence identity to SEQ ID NO:2 or at least 95% sequence identity to SEQ ID NO:86; b) a second polynucleotide sequence that encodes a cucurbitadienol synthase polypeptide having at least 90% sequence identity to SEQ ID NO:5; c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33; d) a fourth polynucleotide sequence that encodes a uridine phosphorylase dependent glycosyltransferase polypeptide having at least 90% sequence identity to SEQ ID NO:9; e) a fifth polynucleotide sequence that encodes a uridine phosphorylase dependent glycosyltransferase polypeptide having at least 90% sequence identity to SEQ ID NO:11; f) a sixth polynucleotide sequence that encodes a squalene epoxidase polypeptide having at least 90% sequence identity to SEQ ID NO:13; g) a seventh polynucleotide sequence that encodes an epoxy hydrolase polypeptide having at least 90% sequence identity to SEQ ID NO:15; or h) an eighth polynucleotide sequence that encodes a truncated 3-hydroxy-3-methylglutaryl-CoA reductase polypeptide having at least 90% sequence identity to SEQ ID NO:17, SEQ ID NO:274, SEQ ID NO:276 or SEQ ID NO:278; wherein the first, second, third, fourth, fifth, sixth, seventh or eighth polynucleotide sequence is operably linked to a heterologous promoter, and wherein the transgenic plant, plant part or seed produces at least a first mogroside compound. In certain embodiments the first, second, third, fourth, fifth, sixth, seventh or eighth polynucleotide sequences are operably linked to different heterologous promoters. In some embodiments at least two of the first, second, third, fourth, fifth, sixth, seventh or eighth polynucleotide sequences are operably linked to a single heterologous promoter. In other embodiments one or more of the first, second, third, fourth, fifth, sixth, seventh or eighth polynucleotide sequences are present in multiple copies, and are operably linked to the same or different endogenous or heterologous promoters.

In various embodiments the plant is a Cucurbitaceae, Solanaceae or Asteraceae plant. In some embodiments the plant is a Cucurbita, Citrullus, Cucumis, Momordica, Solanum or Lactuca plant. In further embodiments the plant is a watermelon, cantaloupe, honeydew, winter melon, casaba melon, Persian melon, citron melon, muskmelon, bailan melon, crenshaw melon, Christmas melon, sprite melon, caravelle melon, hami melon, rocky melon, golden Langkawi melon, Korean melon, saticoy melon, galia melon, jade dew melon, golden prize melon, ten me melon, new century melon, banana melon, yubari king melon, sugar melon, tiger melon, vert grimpant melon, horned melon, cucamelon, casabanana melon, pepino melon, ananas melon, camouflage melon, canary melon, bitter melon, charentais melon, crane melon, SkyRocket melon, honey globe melon, gac melon, autumn sweet melon, snap melon, lettuce, spinach, rice, oat, maize, sorghum, wheat, alfalfa, bitter apple (Citrullus colocynthis), pumpkin, chard, tobacco, switch grass, tomato, cucumber, potato, amaranth, sunflower, canola, dry bean, field pea, flax, safflower, buckwheat, cotton, soybean, sugar beet or Nicotiana benthamiana plant. In particular embodiments the plant is a watermelon, tomato, lettuce or cucumber plant, or other leafy bulky biomass green plant or other food crop plant that can be used as foundational ingredients for processed foods. In certain embodiments the plant is a monocot plant or a dicot plant. In some embodiments the plant part is a fruit, leaf, root, flower, shoot, cell, endosperm, ovule, or pollen. In other embodiments the plant is a species useful for producing mogrosides for processing into foods where the mogrosides can replace or partially replace the need for added sugars.

In certain embodiments the heterologous promoter is an inducible, plant, bacterial, viral, synthetic, constitutive, tissue specific, developmentally regulated, cell cycle regulated, temporally regulated, spatially regulated, and/or spatio-temporally regulated promoter. In other embodiments the heterologous promoter is a FSgt/PFLt (SEQ ID NO:62), FMVSgt (SEQ ID NO:69), CsVMV (SEQ ID NO:68), dMMV (SEQ ID NO:63), HLVH12 (SEQ ID NO:60), NOS (SEQ ID NO:66), ScBV (SEQ ID NO:67), DCMV (SEQ ID NO:61), CmYLCV (SEQ ID NO:64), FS1_1 (SEQ ID NO:70), FE_3 (SEQ ID NO:71), e35S (SEQ ID NO:65), AtUBQ10 (SEQ ID NO:259), PCLSV (SEQ ID NO:260), FS4 (SEQ ID NO:261), AtACT2 (SEQ ID NO:262), enhanced AtEf-1A (SEQ ID NO:263), FuasFScp (SEQ ID NO:264), FE4 (SEQ ID NO:269), cucumisin (SEQ ID NO:270) or SgCDS (SEQ ID NO:271) promoter. In further embodiments the first, second, third, fourth, fifth, sixth, seventh or eighth polynucleotide sequence is operably linked to a heterologous terminator. In yet further embodiments the first, second, third, fourth, fifth, sixth, seventh or eighth heterologous terminator sequence is a GmaxMYB2 (SEQ ID NO:74), 35S T (SEQ ID NO:), ATHSP18.2 (SEQ ID NO:77), AtRBCS2b (SEQ ID NO:75), AtUBQ3 (SEQ ID NO:73), Pea E9 (SEQ ID NO:76), Pea3A (SEQ ID NO:72), potato Ubi3 (SEQ ID NO:78), AtTubB9 (SEQ ID NO:79), AtFAD2 (SEQ ID NO:265), AtNDUFA8 (SEQ ID NO:266), CsHSP17.3 (SEQ ID NO:267) or CsHSP22 (SEQ ID NO:268) terminator sequence. In additional embodiments the transgenic plant, plant part or seed further comprises a selectable marker sequence. In some embodiments the selectable marker sequence is a β-glucuronidase, green fluorescent protein, or antibiotic resistance sequence. In particular embodiments the selectable marker sequence is a hygromycin B phosphotransferase (HygR) or neomycin phosphotransferase II (nptII) selectable marker sequence.

In some embodiments the transgenic plant, plant part or cell further comprises i) a ninth polynucleotide sequence that encodes an NADPH: cytochrome P450 reductase polypeptide having at least 90% sequence identity to SEQ ID NO:19; or j) a tenth polynucleotide sequence that encodes a uridine phosphorylase dependent glycosyltransferase polypeptide having at least 90% sequence identity to SEQ ID NO:21, SEQ ID NO:23, SEQ ID NO:120 or SEQ ID NO:126; k) an eleventh polynucleotide sequence that encodes a 3-hydroxy-3-methylglutaryl-CoA synthase polypeptide having at least 90% sequence identity to SEQ ID NO:227; or 1) a twelfth polynucleotide sequence that encodes a geranyl diphosphate synthase polypeptide having at least 90% sequence identity to SEQ ID NO:256; wherein the ninth, tenth, eleventh or twelfth polynucleotide sequence is operably linked to a heterologous promoter. In other embodiments the transgenic plant, plant part or cell further comprises a 2A linker, insulator, selectable marker or stuffer sequence.

In certain embodiments the at least a first mogroside compound is a non-native mogrol precursor, mogrol, mogroside, or a metabolite or derivative thereof. In various embodiments the mogroside is mogroside II A, mogroside II A1, mogroside II A2, mogroside II E, 11-oxo-mogroside II, mogroside III, mogroside III A1, mogroside III A2, mogroside III E 11-oxo-mogroside III, mogroside IV, mogroside IV A, 11-oxo-mogroside IV, siamenoside I, mogroside V, 11-oxo-mogroside V or mogroside VI, or an isomer thereof. In additional embodiments the transgenic plant, plant part or seed produces at least 10 ng/g to 30 mg/g dry weight of the at least a first mogroside compound. In some embodiments the transgenic plant, plant part or seed produces at least 10 ng/g, at least 25 ng/g, at least 50 ng/g, at least 75 ng/g, at least 100 ng/g, at least 250 ng/g, at least 500 ng/g, at least 750 ng/g, at least 1 mg/g, at least 2.5 mg/g, at least 5 mg/g, at least 7.5 mg/g, at least 10 mg/g, at least 12.5 mg/g, at least 15 mg/g, at least 17.5 mg/g, at least 20 mg/g, at least 22.5 mg/g, at least 25 mg/g, at least 27.5 mg/g, or at least 30 mg/g of the at least a first mogroside compound. In further embodiments the amount of at least a first mogroside compound is greater than the level in a non-transgenic plant, plant part or seed of the same species. In other embodiments the ratio of individual mogroside compounds is different from the ratio of mogroside compounds in native plant tissues or fruit or other plants or organisms that produce mogroside compounds. For example, the ratio of a mogroside compound produced by the plants or organisms of the present disclosure to a mogroside compound produced by a native plant tissues or fruit or other plants or organisms can be 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 7.5:1, 8:1, 9:1, 10:1, 12.5:1, 15:1, 20:1, 25:1, 30:1, 40:1, 50:1, 60:1, 70:1, 75:1, 80:1, 90:1, 100:1, 250:1, 500:1, 750:1, 1000:1, or more, or 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:7.5, 1:8, 1:9, 1:10, 1:12.5, 1:15, 1:20, 1:25, 1:30, 1:40, 1:50, 1:60, 1:70, 1:75, 1:80, 1:90, 1:100, 1:250, 1:500, 1:750, 1:1000, or lower. These ratios can apply to one or more of the following mogroside compounds: mogroside II A, mogroside II A1, mogroside II A2, mogroside II E, 11-oxo-mogroside II, mogroside III, mogroside III A1, mogroside III A2, mogroside III E 11-oxo-mogroside III, mogroside IV, mogroside IV A, 11-oxo-mogroside IV, siamenoside I, mogroside V, 11-oxo-mogroside V or mogroside VI, or an isomer thereof. Generally higher ratios of sweet mogroside compounds, such as those including more than three glucose residues in the molecule, including, but not limited to, mogroside V, isomogroside V, siamenoside I, α-siamenoside I, mogroside IV, mogroside IV A, mogroside III, mogroside III E, mogroside III A1 and 11-oxo-mogroside V, to other mogroside compound(s) are used. In other words this applies to the ratio of one or more mogroside compound, for example mogroside V, to one or more other mogroside compound.

The present disclosure additionally provides a recombinant host cell comprising: a) a first polynucleotide sequence that encodes a cytochrome P450 polypeptide having at least 91% sequence identity to SEQ ID NO:2 or at least 95% sequence identity to SEQ ID NO:86; b) a second polynucleotide sequence that encodes a cucurbitadienol synthase polypeptide having at least 90% sequence identity to SEQ ID NO:5; c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33; d) a fourth polynucleotide sequence that encodes a uridine phosphorylase dependent glycosyltransferase polypeptide having at least 90% sequence identity to SEQ ID NO:9; e) a fifth polynucleotide sequence that encodes a uridine phosphorylase dependent glycosyltransferase polypeptide having at least 90% sequence identity to SEQ ID NO:11; f) a sixth polynucleotide sequence that encodes a squalene epoxidase polypeptide having at least 90% sequence identity to SEQ ID NO:13; g) a seventh polynucleotide sequence that encodes an epoxy hydrolase polypeptide having at least 90% sequence identity to SEQ ID NO:15; or h) an eighth polynucleotide sequence that encodes a truncated 3-hydroxy-3-methylglutaryl-CoA reductase polypeptide having at least 90% sequence identity to SEQ ID NO:17, SEQ ID NO:274, SEQ ID NO:276 or SEQ ID NO:278; wherein the first, second, third, fourth, fifth, sixth, seventh or eighth polynucleotide sequence is operably linked to a heterologous promoter. In certain embodiments the recombinant host cell produces at least a first mogroside compound.

The present disclosure also provides a processed lower calorie food or beverage product produced from a transgenic plant, plant part or seed comprising: a) a first polynucleotide sequence that encodes a cytochrome P450 polypeptide having at least 91% sequence identity to SEQ ID NO:2 or at least 95% sequence identity to SEQ ID NO:86; b) a second polynucleotide sequence that encodes a cucurbitadienol synthase polypeptide having at least 90% sequence identity to SEQ ID NO:5; c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33; d) a fourth polynucleotide sequence that encodes a uridine phosphorylase dependent glycosyltransferase polypeptide having at least 90% sequence identity to SEQ ID NO:9; e) a fifth polynucleotide sequence that encodes a uridine phosphorylase dependent glycosyltransferase polypeptide having at least 90% sequence identity to SEQ ID NO:11; f) a sixth polynucleotide sequence that encodes a squalene epoxidase polypeptide having at least 90% sequence identity to SEQ ID NO:13; g) a seventh polynucleotide sequence that encodes an epoxy hydrolase polypeptide having at least 90% sequence identity to SEQ ID NO:15; or h) an eighth polynucleotide sequence that encodes a truncated 3-hydroxy-3-methylglutaryl-CoA reductase polypeptide having at least 90% sequence identity to SEQ ID NO:17, SEQ ID NO:274, SEQ ID NO:276 or SEQ ID NO:278; wherein the first, second, third, fourth, fifth, sixth, seventh or eighth polynucleotide sequence is operably linked to a heterologous promoter, and wherein the transgenic plant, plant part or seed produces at least a first mogroside compound. In certain embodiments the product is produced from a juice, extract, powder, fruit or rind of a fruit, vegetable, legume, tuber or grain of the transgenic plant, plant part or seed.

The present disclosure additionally provides a juice, powder or extract produced from a transgenic plant, plant part or seed comprising: a) a first polynucleotide sequence that encodes a cytochrome P450 polypeptide having at least 91% sequence identity to SEQ ID NO:2 or at least 95% sequence identity to SEQ ID NO:86; b) a second polynucleotide sequence that encodes a cucurbitadienol synthase polypeptide having at least 90% sequence identity to SEQ ID NO:5; c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33; d) a fourth polynucleotide sequence that encodes a uridine phosphorylase dependent glycosyltransferase polypeptide having at least 90% sequence identity to SEQ ID NO:9; e) a fifth polynucleotide sequence that encodes a uridine phosphorylase dependent glycosyltransferase polypeptide having at least 90% sequence identity to SEQ ID NO:11; f) a sixth polynucleotide sequence that encodes a squalene epoxidase polypeptide having at least 90% sequence identity to SEQ ID NO:13; g) a seventh polynucleotide sequence that encodes an epoxy hydrolase polypeptide having at least 90% sequence identity to SEQ ID NO:15; or h) an eighth polynucleotide sequence that encodes a truncated 3-hydroxy-3-methylglutaryl-CoA reductase polypeptide having at least 90% sequence identity to SEQ ID NO:17, SEQ ID NO:274, SEQ ID NO:276 or SEQ ID NO:278; wherein the first, second, third, fourth, fifth, sixth, seventh or eighth polynucleotide sequence is operably linked to a heterologous promoter, and wherein the transgenic plant, plant part or seed produces at least a first mogroside compound.

The present disclosure further provides a recombinant DNA molecule comprising: a) a first polynucleotide sequence that encodes a cytochrome P450 polypeptide having at least 91% sequence identity to SEQ ID NO:2 or at least 95% sequence identity to SEQ ID NO:86; b) a second polynucleotide sequence that encodes a cucurbitadienol synthase polypeptide having at least 90% sequence identity to SEQ ID NO:5; c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33; d) a fourth polynucleotide sequence that encodes a uridine phosphorylase dependent glycosyltransferase polypeptide having at least 90% sequence identity to SEQ ID NO:9; e) a fifth polynucleotide sequence that encodes a uridine phosphorylase dependent glycosyltransferase polypeptide having at least 90% sequence identity to SEQ ID NO:11; f) a sixth polynucleotide sequence that encodes a squalene epoxidase polypeptide having at least 90% sequence identity to SEQ ID NO:13; g) a seventh polynucleotide sequence that encodes an epoxy hydrolase polypeptide having at least 90% sequence identity to SEQ ID NO:15 or h) an eighth polynucleotide sequence that encodes a truncated 3-hydroxy-3-methylglutaryl-CoA reductase polypeptide having at least 90% sequence identity to SEQ ID NO:17, SEQ ID NO:274, SEQ ID NO:276 or SEQ ID NO:278; wherein the first, second, third, fourth, fifth, sixth, seventh or eighth polynucleotide sequence is operably linked to a heterologous promoter. In certain embodiments the first, second, third, fourth, fifth, sixth, seventh or eighth polynucleotide sequences are operably linked to different heterologous promoters. In other embodiments at least two of the first, second, third, fourth, fifth, sixth, seventh or eighth polynucleotide sequences are operably linked to a single heterologous promoter. In some embodiments one or more of the first, second, third, fourth, fifth, sixth, seventh or eighth polynucleotide sequences are present in multiple copies, and are operably linked to the same or different endogenous or heterologous promoters.

The present disclosure also provides a DNA molecule comprising polynucleotide sequence that encodes a cytochrome P450 polypeptide having at least 91% sequence identity to SEQ ID NO:2 or at least 95% sequence identity to SEQ ID NO:86. In certain embodiments the DNA molecule is operably linked to a heterologous promoter. The present disclosure additionally provides a DNA molecule exhibiting a gene regulatory functional activity comprising a polynucleotide sequence selected from the group consisting of: a) a sequence with at least 90 or 95 percent sequence identity to SEQ ID NOs:70 or 71 and exhibiting promoter activity; b) a sequence comprising SEQ ID NOs:70 or 71; and c) a fragment of SEQ ID NOs:70 or 71, wherein said fragment exhibits promoter activity; wherein said DNA molecule is operably linked to a heterologous transcribable polynucleotide molecule. In certain embodiments SEQ ID NO:70 and SEQ ID NO:71 can lead to increased expression in the fruit of a plant.

The present disclosure further provides a method of producing at least a first mogroside compound, comprising growing a transgenic plant or organism comprising: a) a first polynucleotide sequence that encodes a cytochrome P450 polypeptide having at least 91% sequence identity to SEQ ID NO:2 or at least 95% sequence identity to SEQ ID NO:86; b) a second polynucleotide sequence that encodes a cucurbitadienol synthase polypeptide having at least 90% sequence identity to SEQ ID NO:5; c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33; d) a fourth polynucleotide sequence that encodes a uridine phosphorylase dependent glycosyltransferase polypeptide having at least 90% sequence identity to SEQ ID NO:9; e) a fifth polynucleotide sequence that encodes a uridine phosphorylase dependent glycosyltransferase polypeptide having at least 90% sequence identity to SEQ ID NO:11; f) a sixth polynucleotide sequence that encodes a squalene epoxidase polypeptide having at least 90% sequence identity to SEQ ID NO:13; g) a seventh polynucleotide sequence that encodes an epoxy hydrolase polypeptide having at least 90% sequence identity to SEQ ID NO:15; or h) an eighth polynucleotide sequence that encodes a truncated 3-hydroxy-3-methylglutaryl-CoA reductase polypeptide having at least 90% sequence identity to SEQ ID NO:17, SEQ ID NO:274, SEQ ID NO:276 or SEQ ID NO:278; wherein the first, second, third, fourth, fifth, sixth, seventh or eighth polynucleotide sequence is operably linked to a heterologous promoter, and wherein the transgenic plant, plant part or seed produces at least a first mogroside compound. In certain embodiments the transgenic plant produces the at least a first mogroside compound in a plant part or seed of the plant. In some embodiments the transgenic plant produces the at least a first mogroside compound in a fruit or leaf of the plant. In additional embodiments the method further comprises the step of isolating the at least a first mogroside compound from the transgenic plant. In other embodiments the at least a first mogroside compound is isolated, purified or partially purified from a plant part or seed of the transgenic plant. In yet other embodiments the at least a first mogroside compound is isolated from a fruit, leaf, vegetable, legume, tuber or grain of the transgenic plant. In further embodiments a combination of mogroside compounds are purified or partially purified from the transgenic plant, plant part or seed.

The present disclosure also provides a composition comprising: a) about 80% mogroside V, about 15% 11-oxo-mogroside V and about 5% mogroside III-A1; or b) about 40% siamenoside I, about 40% mogroside V and about 20% 11-oxo-mogroside V. In certain embodiments the composition comprises about 75%, about 76%, about 77%, about 78%, about 79%, about 80%, about 81%, about 82%, about 83%, about 84% or about 85% of mogroside V, about 10%, about 11%, about 12%, about 13%, about 14%, about 15%, about 16%, about 17%, about 18%, about 19% or about 20% of 11-oxo-mogroside V, and about 1%, about 2%, about 3%, about 4%, about 5%, about 6%, about 7%, about 8%, about 9% or about 10% of mogroside III A1. In some embodiments, the composition comprises about 35%, about 36%, about 37%, about 38%, about 39%, about 40%, about 41%, about 42%, about 43%, about 44% or about 45% of siamenoside I, about 35%, about 36%, about 37%, about 38%, about 39%, about 40%, about 41%, about 42%, about 43%, about 44% or about 45% of mogroside V, and about 15%, about 16%, about 17%, about 18%, about 19%, about 20%, about 21%, about 22%, about 23%, about 24% or about 25% of 11-oxo-mogroside V. In other embodiments the composition is a liquid. In yet other embodiments the composition is a dry powder.

### BRIEF DESCRIPTION OF THE DRAWINGS

The following drawings form part of the present specification and are included to further demonstrate certain aspects of the present disclosure. The present disclosure may be better understood by reference to one or more of these drawings in combination with the detailed description of specific embodiments presented herein.

FIG. 1. A mogroside biosynthetic pathway from Siraitia grosvenorii.

FIG. 2. Mogroside V production in transient expression of watermelon fruit with a number of different expression constructs.

FIG. 3. Map of expression construct SP3139.

FIG. 4. Mogroside and siamenoside production in transient expression in watermelon fruit using various constructs.

FIG. 5. Mogroside V production in transient expression in watermelon fruit using various constructs.

FIG. 6. Siamenoside production in transient expression in watermelon fruit using various constructs.

FIG. 7. Mogroside and siamenoside production in transient expression in watermelon fruit using a combination of SP2015, SP4332, SP5029, SP5030, SP5031, SP5032, and SP5033 constructs (control), and the combination excluding one of the constructs.

FIG. 8. Mogroside V production in transient expression in watermelon fruit using a combination of SP2015, SP4332, SP5029, SP5030, SP5031, SP5032, and SP5033 constructs (control), and the combination excluding one of the constructs.

FIG. 9. Mogroside V production in transient expression in lettuce leaves infiltrated with the SP1463 construct compared to negative lettuce control and mogroside standard mix.

FIG. 10. Mass spectrum fingerprinting of mogroside V production in transient expression in lettuce leaves infiltrated with the SP1463 construct compared to negative lettuce control and mogroside V reference standard.

FIG. 11. Map of expression construct SP1463.

FIG. 12. Mogroside and siamenoside production in transient expression in lettuce leaves using various constructs.

FIG. 13. Mogroside V production in transient expression in lettuce leaves using various constructs.

FIG. 14. Siamenoside production in transient expression in lettuce leaves using various constructs.

FIG. 15. Mogroside and siamenoside production in transient expression in lettuce leaves using a combination of SP2015, SP4332, SP5029, SP5030, SP5031, SP5032, and SP5033 constructs (control), and the combination excluding one of the constructs.

FIG. 16. Mogroside V production in transient expression in lettuce leaves using a combination of SP2015, SP4332, SP5029, SP5030, SP5031, SP5032, and SP5033 constructs (control), and the combination excluding one of the constructs.

FIG. 17. Production of mogroside V in transgenic watermelon with expression construct SP1463 compared to mogroside V reference standard.

FIG. 18. Mass spectrum fingerprinting of mogroside V in transgenic watermelon with expression construct SP1463 compared to mogroside V reference standard.

FIG. 19. Mogroside production in various transgenic watermelon lines with expression constructs SP0336, SP1463, SP1908, SP3488 and SP3190.

FIG. 20. Map of expression construct SP0336.

FIG. 21. Map of expression construct SP1908.

FIG. 22. Production of various mogrosides and siamenoside in transgenic watermelon with expression constructs SP0565, SP0641, SP1463, SP1908, SP3015, SP3016, SP3029, SP3190, SP3308 and SP3488.

FIG. 23. Production of mogrosides and siamenoside in transgenic watermelon cell suspension.

FIG. 24. Concentration of mogrosides and siamenoside in transgenic watermelon cell suspension.

FIG. 25. Production of various mogrosides and siamenoside in transgenic tomato with expression constructs SP0641, SP1463, SP1908, SP3016, SP3029, SP3190 and SP3684.

FIG. 26. Production of various mogrosides and siamenoside in transgenic potato with expression constructs SP0641, SP1463, SP3016, SP3029, SP3190 and SP3684.

FIG. 27. Production of mogroside V and unknown mogroside V isomer in transient watermelon fruit co-infiltrated with the construct SP3684 and either t72143 or green fluorescent protein (GFP).

FIG. 28. Mass spectrum fingerprinting of mogroside V and the unknown mogroside V isomer.

FIG. 29. Production of mogroside IV and unknown mogroside IV isomers in transient watermelon fruit co-infiltrated with the construct SP3684 and either t72143 or green fluorescent protein (GFP).

FIG. 30. Mass spectrum fingerprinting of mogroside IV and the unknown mogroside IV isomers.

FIG. 31. Production of mogroside III and unknown mogroside III isomer in transient watermelon fruit co-infiltrated with the construct SP3684 and either t72143 or green fluorescent protein (GFP).

FIG. 32. Mass spectrum fingerprinting of mogroside III and the unknown mogroside III isomer.

FIG. 33. Production of mogroside II and unknown mogroside II isomer in transient watermelon fruit co-infiltrated with the construct SP3684 and either t72143 or green fluorescent protein (GFP).

FIG. 34. Mass spectrum fingerprinting of mogroside II and the unknown mogroside II isomer.

FIG. 35. Production of 11-oxo-mogroside III and unknown 11-oxo-mogroside III isomer in transient watermelon fruit co-infiltrated with the construct SP3684 and either t72143 or green fluorescent protein (GFP).

FIG. 36. Mass spectrum fingerprinting of 11-oxo-mogroside III and the unknown 11-oxo-mogroside III isomer.

FIG. 37. Production of 11-oxo-mogroside IV and unknown 11-oxo-mogroside IV isomer in transient watermelon fruit co-infiltrated with the construct SP3684 and either t72143 or green fluorescent protein (GFP).

FIG. 38. Mass spectrum fingerprinting of 11-oxo-mogroside IV and the unknown 11-oxo-mogroside IV isomer.

FIG. 39. Production of 11-oxo-mogroside V and unknown 11-oxo-mogroside V isomer in transient watermelon fruit co-infiltrated with the construct SP3684 and either t72143 or green fluorescent protein (GFP).

FIG. 40. Mass spectrum fingerprinting of 11-oxo-mogroside V and the unknown 11 oxo-mogroside V isomer.

FIG. 41. Concentration response curve for Mogroside Blend 80/15/5.

FIG. 42. Concentration response curves of Mogroside-V and Mogroside Blend 80/15/5.

FIG. 43. Onset and time to reach maximum sweetness of Mogroside-V, Mogroside Blend 80/15/5, Rebaudioside M, Rebaudioside A and monk fruit 50 (MF50).

FIG. 44. Graph of sweetness decay over time of Mogroside-V, Mogroside Blend 80/15/5, Rebaudioside M, Rebaudioside A and MF50.

FIG. 45. Descriptive analysis scores of sweetener attributes for Mogroside-V, Mogroside Blend 80/15/5, Rebaudioside M, Rebaudioside A and MF50.

FIG. 46. Descriptive analysis of sweetener attributes for Mogroside-V, Mogroside Blend 80/15/5, Rebaudioside M, Rebaudioside A and MF50.

FIG. 47. Concentration response curve for V90.

FIG. 48. Concentration response curves of Mogroside V, Blend 80/15/5, and V90.

FIG. 49. Onset and time to maximum sweetness for Mogroside V and V90.

FIG. 50. Sweetness decay of Mogroside V and V90.

FIG. 51. Descriptive analysis scores of Mogroside V and V90.

FIG. 52. Concentration response curve for 11-oxo Mogroside-V.

FIG. 53. Concentration response curve for Mogroside IIIA-1.

FIG. 54. Onset and time to maximum sweetness of Mogroside V, 11-oxo Mogroside-V and Mogroside IIIA-1.

FIG. 55. Lingering aftertaste decay over time of Mogroside V, 11-oxo Mogroside-V and Mogroside IIIA-1.

FIG. 56. Descriptive analysis scores of sweetener attributes for Mogroside-V, 11-oxo-mogroside V and mogroside IIIA-1.

FIG. 57. Descriptive analysis of sweetener attributes for Mogroside-V, 11-oxo-mogroside V and mogroside IIIA-1.

FIG. 58. Concentration of mogrosides and siamenoside in transgenic potato transformed with vector SP1463 and then re-transformed that transgenic event with S1UGT (SP5027).

FIG. 59. Concentration of mogrosides and siamenoside in transgenic sugar beet callus transformed with vector SP3684.

### BRIEF DESCRIPTION OF THE SEQUENCES

SEQ ID NO:1: cytochrome P450-72 (CYP72 Zm) nucleic acid sequence based on Zea mays codon usage.
SEQ ID NO:2: cytochrome P450-72 (CYP72) amino acid sequence.
SEQ ID NO:3: cytochrome P450-72 (CYP72 GC) nucleic acid sequence with higher GC content than SEQ ID NO:1.
SEQ ID NO:4: cucurbitadienol synthase (CDS) nucleic acid sequence.
SEQ ID NO:5: cucurbitadienol synthase (CDS) amino acid sequence.
SEQ ID NO:6: cytochrome P450-87 (CYP87) nucleic acid sequence.
SEQ ID NO:7: cytochrome P450-87 (CYP87) amino acid sequence.
SEQ ID NO:8: uridine phosphorylase dependent glycosyltransferase-720 (UGT720) nucleic acid sequence.
SEQ ID NO:9: uridine phosphorylase dependent glycosyltransferase-720 (UGT720) amino acid sequence.
SEQ ID NO:10: uridine phosphorylase dependent glycosyltransferase-94 (UGT94) nucleic acid sequence.
SEQ ID NO:11: uridine phosphorylase dependent glycosyltransferase-94 (UGT94) amino acid sequence.
SEQ ID NO:12: squalene epoxidase (SQE) nucleic acid sequence.
SEQ ID NO:13: squalene epoxidase (SQE) amino acid sequence.
SEQ ID NO:14: epoxy hydrolase (EPH) nucleic acid sequence.
SEQ ID NO:15: epoxy hydrolase (EPH) amino acid sequence.
SEQ ID NO:16: truncated 3-hydroxy-3-methylglutaryl-CoA reductase (tHMGR) nucleic acid sequence.
SEQ ID NO:17: truncated 3-hydroxy-3-methylglutaryl-CoA reductase (tHMGR) amino acid sequence.
SEQ ID NO:18: NADPH:cytochrome P450 reductase (SgCPR2) nucleic acid sequence.
SEQ ID NO:19: NADPH:cytochrome P450 reductase (SgCPR2) amino acid sequence.
SEQ ID NO:20: uridine phosphorylase dependent glycosyltransferase 74 (UGT74_3) nucleic acid sequence.
SEQ ID NO:21: uridine phosphorylase dependent glycosyltransferase 74 (UGT74_3) amino acid sequence.
SEQ ID NO:22: uridine phosphorylase dependent glycosyltransferase 74 (UGT74_4) nucleic acid sequence.
SEQ ID NO:23: uridine phosphorylase dependent glycosyltransferase 74 (UGT74_4) amino acid sequence.
SEQ ID NO:24: cytochrome P450-87 (C1CYP87D18_B m3) nucleic acid sequence from Citrullus lanatus.
SEQ ID NO:25: cytochrome P450-87 (C1CYP87D18_B m3) amino acid sequence from Citrullus lanatus.
SEQ ID NO:26: mutated cytochrome P450-87 (CYP87D17 m2) nucleic acid sequence.
SEQ ID NO:27: mutated cytochrome P450-87 (CYP87D17 m2) amino acid sequence.
SEQ ID NO:28: mutated cytochrome P450-87 (CYP87D17 m3) nucleic acid sequence.
SEQ ID NO:29: mutated cytochrome P450-87 (CYP87D17 m3) amino acid sequence.
SEQ ID NO:30: mutated cytochrome P450-87 (CYP87D20 m2) nucleic acid sequence.
SEQ ID NO:31: mutated cytochrome P450-87 (CYP87D20 m2) amino acid sequence.
SEQ ID NO:32: mutated cytochrome P450-87 (CYP87D20 m3) nucleic acid sequence.
SEQ ID NO:33: mutated cytochrome P450-87 (CYP87D20 m3) amino acid sequence.
SEQ ID NO:34: cytochrome P450-72 (CYP72)-2A-cytochrome P450-72 (CYP72) bicistronic nucleic acid sequence.
SEQ ID NO:35: first CYP72 amino acid sequence that can be produced from cytochrome P450-72 (CYP72)-2A-cytochrome P450-72 (CYP72) bicistronic nucleic acid sequence after cleavage.
SEQ ID NO:36: second CYP72 amino acid sequence that can be produced from cytochrome P450-72 (CYP72)-2A-cytochrome P450-72 (CYP72) bicistronic nucleic acid sequence after cleavage.
SEQ ID NO:37: full length CYP72-2A-CYP72 amino acid sequence from cytochrome P450-72 (CYP72)-2A-cytochrome P450-72 (CYP72) bicistronic nucleic acid sequence.
SEQ ID NO:38: uridine phosphorylase dependent glycosyltransferase-720 (UGT720)-2A-uridine phosphorylase dependent glycosyltransferase-720 (UGT720) bicistronic nucleic acid sequence.
SEQ ID NO:39: first UGT720 amino acid sequence that can be produced from uridine phosphorylase dependent glycosyltransferase-720 (UGT720)-2A-uridine phosphorylase dependent glycosyltransferase-720 (UGT720) bicistronic nucleic acid sequence after cleavage.
SEQ ID NO:40: second UGT720 amino acid sequence that can be produced from uridine phosphorylase dependent glycosyltransferase-720 (UGT720)-2A-uridine phosphorylase dependent glycosyltransferase-720 (UGT720) bicistronic nucleic acid sequence after cleavage.
SEQ ID NO:41: full length UGT720 amino acid sequence from uridine phosphorylase dependent glycosyltransferase-720 (UGT720)-2A-uridine phosphorylase dependent glycosyltransferase-720 (UGT720) bicistronic nucleic acid sequence.
SEQ ID NO:42: uridine phosphorylase dependent glycosyltransferase-94 (UGT94)-2A-uridine phosphorylase dependent glycosyltransferase-94 (UGT94) bicistronic nucleic acid sequence.
SEQ ID NO:43: first UGT94 amino acid sequence that can be produced from uridine phosphorylase dependent glycosyltransferase-94 (UGT94)-2A-uridine phosphorylase dependent glycosyltransferase-94 (UGT94) bicistronic nucleic acid sequence after cleavage.
SEQ ID NO:44: second UGT94 amino acid sequence that can be produced from uridine phosphorylase dependent glycosyltransferase-94 (UGT94)-2A-uridine phosphorylase dependent glycosyltransferase-94 (UGT94) bicistronic nucleic acid sequence after cleavage.
SEQ ID NO:45: full length UGT94-2A-UGT94 amino acid sequence that can be produced from uridine phosphorylase dependent glycosyltransferase-94 (UGT94)-2A-uridine phosphorylase dependent glycosyltransferase-94 (UGT94) bicistronic nucleic acid sequence after cleavage.
SEQ ID NO:46: truncated 3-hydroxy-3-methylglutaryl-CoA reductase (tHMGR)-2A-truncated 3-hydroxy-3-methylglutaryl-CoA reductase (tHMGR) bicistronic nucleic acid sequence.
SEQ ID NO:47: first tHMGR amino acid sequence that can be produced from truncated 3-hydroxy-3-methylglutaryl-CoA reductase (tHMGR)-2A-truncated 3-hydroxy-3-methylglutaryl-CoA reductase (tHMGR) bicistronic nucleic acid sequence after cleavage.
SEQ ID NO:48: second tHMGR amino acid sequence that can be produced from truncated 3-hydroxy-3-methylglutaryl-CoA reductase (tHMGR)-2A-truncated 3-hydroxy-3-methylglutaryl-CoA reductase (tHMGR) bicistronic nucleic acid sequence after cleavage.
SEQ ID NO:49: full length tHMGR-2A-tHMGR amino acid sequence that can be produced from truncated 3-hydroxy-3-methylglutaryl-CoA reductase (tHMGR)-2A-truncated 3-hydroxy-3-methylglutaryl-CoA reductase (tHMGR) bicistronic nucleic acid sequence after cleavage.
SEQ ID NO:50: uridine phosphorylase dependent glycosyltransferase-720 (UGT720)-2A-cytochrome P450-72 (CYP72) bicistronic nucleic acid sequence.
SEQ ID NO:51: UGT720 amino acid sequence that can be produced from uridine phosphorylase dependent glycosyltransferase-720 (UGT720)-2A-cytochrome P450-72 (CYP72) bicistronic nucleic acid sequence after cleavage.
SEQ ID NO:52: CYP72 amino acid sequence that can be produced from uridine phosphorylase dependent glycosyltransferase-720 (UGT720)-2A-cytochrome P450-72 (CYP72) bicistronic nucleic acid sequence after cleavage.
SEQ ID NO:53: full length UGT720-2A-CYP72 amino acid sequence from uridine phosphorylase dependent glycosyltransferase-720 (UGT720)-2A-cytochrome P450-72 (CYP72) bicistronic nucleic acid sequence.
SEQ ID NO:54: uridine phosphorylase dependent glycosyltransferase-94 (UGT94)-2A-truncated 3-hydroxy-3-methylglutaryl-CoA reductase (tHMGR) bicistronic nucleic acid sequence.
SEQ ID NO:55: UGT94 amino acid sequence that can be produced from uridine phosphorylase dependent glycosyltransferase-94 (UGT94)-2A-truncated 3-hydroxy-3-methylglutaryl-CoA reductase (tHMGR) bicistronic nucleic acid sequence after cleavage.
SEQ ID NO:56: tHMGR amino acid sequence that can be produced from uridine phosphorylase dependent glycosyltransferase-94 (UGT94)-2A-truncated 3-hydroxy-3-methylglutaryl-CoA reductase (tHMGR) bicistronic nucleic acid sequence after cleavage.
SEQ ID NO:57: UGT94-2A-tHMGR amino acid sequence from uridine phosphorylase dependent glycosyltransferase-94 (UGT94)-2A-truncated 3-hydroxy-3-methylglutaryl-CoA reductase (tHMGR) bicistronic nucleic acid sequence.
SEQ ID NO:58: Hygromycin resistance (HygR) gene nucleic acid sequence.
SEQ ID NO:59: Hygromycin resistance (HygR) gene amino acid sequence.
SEQ ID NO:60: HLVH12 promoter nucleic acid sequence.
SEQ ID NO:61: DCMV promoter nucleic acid sequence.
SEQ ID NO:62: FMVSgt:PCLSVFlt (also referred to as FSgt/PFLt) chimeric promoter nucleic acid sequence.
SEQ ID NO:63: Duplicated MMV (dMMV) promoter nucleic acid sequence.
SEQ ID NO:64: CmYLCV promoter nucleic acid sequence.
SEQ ID NO:65: CaMV e35S (e35S) promoter nucleic acid sequence.
SEQ ID NO:66: NOS promoter nucleic acid sequence.
SEQ ID NO:67: ScBV promoter nucleic acid sequence.
SEQ ID NO:68: CsVMV promoter nucleic acid sequence.
SEQ ID NO:69: FMVSgt promoter nucleic acid sequence.
SEQ ID NO:70: FS1_1 promoter nucleic acid sequence.
SEQ ID NO:71: FE_3 promoter nucleic acid sequence.
SEQ ID NO:72: Pea3A terminator nucleic acid sequence.
SEQ ID NO:73: At UBQ3 terminator nucleic acid sequence.
SEQ ID NO:74: Gmax MYB2 terminator nucleic acid sequence.
SEQ ID NO:75: AtRBCS2B terminator nucleic acid sequence.
SEQ ID NO:76: Pea E9 terminator nucleic acid sequence.
SEQ ID NO:77: AtHSP18.2 terminator nucleic acid sequence.
SEQ ID NO:78: Potato Ubi3 terminator nucleic acid sequence.
SEQ ID NO:79: At Tubulin B9 (AtTub) terminator nucleic acid sequence.
SEQ ID NO:80: 35S terminator nucleic acid sequence.
SEQ ID NO:81: SynJ 5UTR nucleic acid sequence.
SEQ ID NO:82: TM6 MAR insulator nucleic acid sequence.
SEQ ID NO:83: 2A self-cleaving peptide nucleic acid sequence.
SEQ ID NO:84: 2A self-cleaving peptide amino acid sequence.
SEQ ID NO:85: cytochrome P450-72 (CYP72 V1) nucleic acid sequence.
SEQ ID NO:86: cytochrome P450-72 (CYP72 V1) amino acid sequence.
SEQ ID NO:87: alternative squalene epoxidase (SQE) nucleic acid sequence.
SEQ ID NO:88: alternative cytochrome P450-87 (CYP87) nucleic acid sequence.
SEQ ID NO:89: alternative cucurbitadienol synthase (CDS) nucleic acid sequence.
SEQ ID NO:90: alternative epoxy hydrolase (EPH) nucleic acid sequence.
SEQ ID NO:91: alternative uridine phosphorylase dependent glycosyltransferase-720 (UGT720) nucleic acid sequence.
SEQ ID NO:92: alternative uridine phosphorylase dependent glycosyltransferase-94 (UGT94) nucleic acid sequence.
SEQ ID NO:93: upstream terpenoid biosynthetic enzyme (FPS; SP0231), Citrullus lanatus cDNA, nucleic acid sequence.
SEQ ID NO:94: upstream terpenoid biosynthetic enzyme (FPS; SP0231), Citrullus lanatus, amino acid sequence.
SEQ ID NO:95: cytochrome P450 biosynthetic enzyme, high GC version (CYP87D20 m2 (V2-I46L-A49L-C343Y) GC63; SP0577), Siraitia grosvenorii cDNA, nucleic acid sequence.
SEQ ID NO:96: cytochrome P450 biosynthetic enzyme, high GC version (CYP87D20 m2 (V2-I46L-A49L-C343Y) GC63; SP0577), Siraitia grosvenorii, nucleic acid sequence.
SEQ ID NO:97: upstream squalene biosynthetic enzyme (SQS; SP0951), Citrullus lanatus cDNA, nucleic acid sequence.
SEQ ID NO:98: upstream squalene biosynthetic enzyme (SQS; SP0951), Citrullus lanatus, amino acid sequence.
SEQ ID NO:99: cytochrome P450 biosynthetic enzyme, mutant, high GC version (CYP87D17 m3 GC63; SP1333), Siraitia grosvenorii cDNA, nucleic acid sequence.
SEQ ID NO:100: cytochrome P450 biosynthetic enzyme, mutant, high GC version (CYP87D17 m3 GC63; SP1333), Siraitia grosvenorii, amino acid sequence.
SEQ ID NO:101: cytochrome P450 biosynthetic enzyme, mutant, high GC version (CYP87D17 m2 GC63; SP2503), Siraitia grosvenorii cDNA, nucleic acid sequence.
SEQ ID NO:102: cytochrome P450 biosynthetic enzyme, mutant high GC version (CYP87D17 m2 GC63; SP2503), Siraitia grosvenorii, protein.
SEQ ID NO:103: cytochrome p450 reductase biosynthetic enzyme, high GC version (SgCPR2 GC66; SP2571), Siraitia grosvenorii cDNA, nucleic acid sequence.
SEQ ID NO:104: cytochrome p450 reductase biosynthetic enzyme, high GC version (SgCPR2 GC66; SP2571), Siraitia grosvenorii, amino acid sequence.
SEQ ID NO:105: uridine phosphorylase dependent glycosyltransferase 74, high GC version (SgUGT74_406_2 GC64; SP2666), Siraitia grosvenorii cDNA, nucleic acid sequence.
SEQ ID NO:106: uridine phosphorylase dependent glycosyltransferase 74, high GC version (SgUGT74_406_2 GC64; SP2666), Siraitia grosvenorii, amino acid sequence.
SEQ ID NO:107: uridine phosphorylase dependent glycosyltransferase 74, high GC version (SgUGT74_345_2 GC65; SP3201), Siraitia grosvenorii cDNA, nucleic acid sequence.
SEQ ID NO:108: uridine phosphorylase dependent glycosyltransferase 74, high GC version (SgUGT74_345_2 GC65; SP3201), Siraitia grosvenorii, amino acid sequence.
SEQ ID NO:109: cytochrome P450 biosynthetic enzyme, mutant, high GC version (C1CYP87D18_B m3 GC62; SP4900), Citrullus lanatus cDNA, nucleic acid sequence.
SEQ ID NO:110: cytochrome P450 biosynthetic enzyme, mutant, high GC version (C1CYP87D18_B m3 GC62; SP4900), Citrullus lanatus, amino acid sequence.
SEQ ID NO:111: cytochrome P450 biosynthetic enzyme (CYP87D20 m3 (V2-I46L-A49L-C343Y L193KO) GC63; SP4910), Siraitia grosvenorii cDNA, nucleic acid sequence.
SEQ ID NO:112: cytochrome P450 biosynthetic enzyme (CYP87D20 m3 (V2-I46L-A49L-C343Y L193KO) GC63; SP4910), Siraitia grosvenorii, amino acid sequence.
SEQ ID NO:113: uridine phosphorylase dependent glycosyltransferase (Solyc01g107825.1 UGT, SP5024), Solanum lycopersicum cDNA, nucleic acid sequence.
SEQ ID NO:114: uridine phosphorylase dependent glycosyltransferase (Solyc01g107825.1 UGT, SP5024), Solanum lycopersicum, amino acid sequence.
SEQ ID NO:115: uridine phosphorylase dependent glycosyltransferase (Solyc02g070020.1 UGT; SP5025), Solanum lycopersicum cDNA, nucleic acid sequence.
SEQ ID NO:116: uridine phosphorylase dependent glycosyltransferase (Solyc02g070020.1 UGT; SP5025), Solanum lycopersicum, amino acid sequence.
SEQ ID NO:117: uridine phosphorylase dependent glycosyltransferase (Solyc09g092500.1 UGT; SP5026), Solanum lycopersicum cDNA, nucleic acid sequence.
SEQ ID NO:118: uridine phosphorylase dependent glycosyltransferase (Solyc09g092500.1 UGT; SP5026), Solanum lycopersicum, amino acid sequence.
SEQ ID NO:119: uridine phosphorylase dependent glycosyltransferase (Solyc10g085230.2 UGT; SP5027), Solanum lycopersicum cDNA, nucleic acid sequence.
SEQ ID NO:120: uridine phosphorylase dependent glycosyltransferase (Solyc10g085230.2 UGT; SP5027), Solanum lycopersicum, amino acid sequence.
SEQ ID NO:121: uridine phosphorylase dependent glycosyltransferase (Solyc10g085880.1 UGT; SP5028), Solanum lycopersicum cDNA, nucleic acid sequence.
SEQ ID NO:122: uridine phosphorylase dependent glycosyltransferase (Solyc10g085880.1 UGT; SP5028), Solanum lycopersicum, amino acid sequence.
SEQ ID NO:123: uridine phosphorylase dependent glycosyltransferase 94 (Sg UGT94-289-3-MS1; SP5034), Siraitia grosvenorii cDNA, nucleic acid sequence.
SEQ ID NO:124:-uridine phosphorylase dependent glycosyltransferase 94 (Sg UGT94-289-3-MS1; SP5034), Siraitia grosvenorii, amino acid sequence.
SEQ ID NO:125: upstream terpenoid biosynthetic enzyme (GPS1; SP5035), Citrullus lanatus cDNA, nucleic acid sequence.
SEQ ID NO:126: upstream terpenoid biosynthetic enzyme (GPS1; SP5035), Citrullus lanatus, amino acid sequence.
SEQ ID NO:127: uridine phosphorylase dependent glycosyltransferase, high GC version (SgUGT75-281-2 GC65; SP5036), Siraitia grosvenorii cDNA, nucleic acid sequence.
SEQ ID NO:128: uridine phosphorylase dependent glycosyltransferase, high GC version (SgUGT75-281-2 GC65; SP5036), Siraitia grosvenorii, amino acid sequence.
SEQ ID NO:129: uridine phosphorylase dependent glycosyltransferase 74 (SgUGT74AC1 (no stop); SP5037), Siraitia grosvenorii cDNA, nucleic acid sequence.
SEQ ID NO:130: uridine phosphorylase dependent glycosyltransferase 74 (SgUGT74AC1 (no stop); SP5037), Siraitia grosvenorii, amino acid sequence.
SEQ ID NO:131: uridine phosphorylase dependent glycosyltransferase (Sg MG1 GC63; SP5038), Siraitia grosvenorii cDNA, nucleic acid sequence.
SEQ ID NO:132: uridine phosphorylase dependent glycosyltransferase (Sg MG1 GC63; SP5038), Siraitia grosvenorii, amino acid sequence.
SEQ ID NO:133: uridine phosphorylase dependent glycosyltransferase (t134248; SP5039), Stevia rebaudiana cDNA, nucleic acid sequence.
SEQ ID NO:134: uridine phosphorylase dependent glycosyltransferase (t134248; SP5039), Stevia rebaudiana, amino acid sequence.
SEQ ID NO:135: uridine phosphorylase dependent glycosyltransferase (t72140; SP5040), Stevia rebaudiana cDNA, nucleic acid sequence.
SEQ ID NO:136:uridine phosphorylase dependent glycosyltransferase (t72140; SP5040), Stevia rebaudiana, amino acid sequence.
SEQ ID NO:137: uridine phosphorylase dependent glycosyltransferase (t72143; SP5041), Stevia rebaudiana cDNA, nucleic acid sequence.
SEQ ID NO:138: uridine phosphorylase dependent glycosyltransferase (t72143; SP5041), Stevia rebaudiana, amino acid sequence.
SEQ ID NO:139: uridine phosphorylase dependent glycosyltransferase (t74692; SP5042), Stevia rebaudiana cDNA, nucleic acid sequence.
SEQ ID NO:140: uridine phosphorylase dependent glycosyltransferase (t74692; SP5042), Stevia rebaudiana, amino acid sequence.
SEQ ID NO:141: uridine phosphorylase dependent glycosyltransferase (t85004; SP5043), Stevia rebaudiana cDNA, nucleic acid sequence.
SEQ ID NO:142: uridine phosphorylase dependent glycosyltransferase (t85004; SP5043), Stevia rebaudiana, amino acid sequence.
SEQ ID NO:143: upstream terpenoid biosynthetic enzyme (Sg 00000109.2_FPS1; SP5044), Siraitia grosvenorii cDNA, nucleic acid sequence.
SEQ ID NO:144: upstream terpenoid biosynthetic enzyme (Sg 00000109.2_FPS1; SP5044), Siraitia grosvenorii, amino acid sequence.
SEQ ID NO:145: upstream squalene biosynthetic enzyme (Sg 00000892.729_SQS1; SP5045), Siraitia grosvenorii cDNA, nucleic acid sequence.
SEQ ID NO:146: upstream squalene biosynthetic enzyme (Sg 00000892.729_SQS1; SP5045), Siraitia grosvenorii, amino acid sequence.
SEQ ID NO:147: upstream terpenoid biosynthetic enzyme (Bj HMGS mutant; SP5046), Brassica junea cDNA, nucleic acid sequence.
SEQ ID NO:148: upstream terpenoid biosynthetic enzyme (Bj HMGS mutant; SP5046), Brassica junea, amino acid sequence.
SEQ ID NO:149: UDPG pyrophosphorylase (Ta UDP-Glucose Pyrophosphorylase; SP5047), Thermocrispum agreste cDNA, nucleic acid sequence.
SEQ ID NO:150: UDPG pyrophosphorylase (Ta UDP-Glucose Pyrophosphorylase; SP5047), Thermocrispum agreste, amino acid sequence.
SEQ ID NO:151: upstream terpenoid biosynthetic enzyme (C1GPS_2; SP5048), Citrullus lanatus cDNA, nucleic acid sequence.
SEQ ID NO:152: upstream terpenoid biosynthetic enzyme (C1GPS_2; SP5048), Citrullus lanatus, amino acid sequence.
SEQ ID NO:153: upstream terpenoid biosynthetic enzyme (MpGPS-SSU; SP5049), Peppermint (Mentha x piperita) cDNA, nucleic acid sequence.
SEQ ID NO:154: upstream terpenoid biosynthetic enzyme (MpGPS-SSU; SP5049), Peppermint (Mentha x piperita), amino acid sequence.
SEQ ID NO:155: upstream terpenoid biosynthetic enzyme (LcGPS-SSU; SP5050), Litsea cubeba cDNA, nucleic acid sequence.
SEQ ID NO:156: upstream terpenoid biosynthetic enzyme (LcGPS-SSU; SP5050), Litsea cubeba, amino acid sequence.
SEQ ID NO:157: hydrolase biosynthetic enzyme (DbExg1; SP5051), Dekkera bruxellensis cDNA, nucleic acid sequence.
SEQ ID NO:158: hydrolase biosynthetic enzyme (DbExg1; SP5051), Dekkera bruxellensis, amino acid sequence.
SEQ ID NO:159: upstream terpenoid biosynthetic enzyme (C1CG09G011560.1_PMK; SP5072), Citrullus lanatus cDNA, nucleic acid sequence.
SEQ ID NO:160: upstream terpenoid biosynthetic enzyme (C1CG09G011560.1_PMK; SP5072), Citrullus lanatus, amino acid sequence.
SEQ ID NO:161: upstream terpenoid biosynthetic enzyme (C1CG10G001230.1_PMK; SP5073), Citrullus lanatus cDNA, nucleic acid sequence.
SEQ ID NO:162: upstream terpenoid biosynthetic enzyme (C1CG10G001230.1_PMK; SP5073), Citrullus lanatus, amino acid sequence.
SEQ ID NO:163: upstream terpenoid biosynthetic enzyme (C1CG09G022040.1_IPK; SP5074), Citrullus lanatus cDNA, nucleic acid sequence.
SEQ ID NO:164: upstream terpenoid biosynthetic enzyme (C1CG09G022040.1_IPK; SP5074), Citrullus lanatus, amino acid sequence.
SEQ ID NO:165: upstream terpenoid biosynthetic enzyme (C1CG05G021710.1_MVD; SP5075), Citrullus lanatus cDNA, nucleic acid sequence.
SEQ ID NO:166: upstream terpenoid biosynthetic enzyme (C1CG05G021710.1_MVD; SP5075), Citrullus lanatus, amino acid sequence.
SEQ ID NO:167: upstream terpenoid biosynthetic enzyme (Sg 00153574.101_FPS1; SP5076), Siraitia grosvenorii cDNA, nucleic acid sequence.
SEQ ID NO:168: upstream terpenoid biosynthetic enzyme (Sg 00153574.101_FPS1; SP5076), Siraitia grosvenorii, amino acid sequence.
SEQ ID NO:169: upstream terpenoid biosynthetic enzyme (Sg 00010190.2_SQS1; SP5077), Siraitia grosvenorii cDNA, nucleic acid sequence.
SEQ ID NO:170: upstream terpenoid biosynthetic enzyme (Sg 00010190.2_SQS1; SP5077), Siraitia grosvenorii, amino acid sequence.
SEQ ID NO:171: upstream terpenoid biosynthetic enzyme (Sg 00153449.125_PMK; SP5078), Siraitia grosvenorii cDNA, nucleic acid sequence.
SEQ ID NO:172: upstream terpenoid biosynthetic enzyme (Sg 00153449.125_PMK; SP5078), Siraitia grosvenorii, amino acid sequence.
SEQ ID NO:173: upstream terpenoid biosynthetic enzyme, high GC version (tHMGR GC69; SP5079), Avena strigose cDNA, nucleic acid sequence.
SEQ ID NO:174: upstream terpenoid biosynthetic enzyme, high GC version (tHMGR GC69; SP5079), Avena strigose, amino acid sequence.
SEQ ID NO:175: uridine phosphorylase dependent glycosyltransferase, high GC version (Sg UGT720-269-1 Itkin GC65; SP5080), Siraitia grosvenorii cDNA, nucleic acid sequence.
SEQ ID NO:176: uridine phosphorylase dependent glycosyltransferase, high GC version (Sg UGT720-269-1 Itkin GC65; SP5080), Siraitia grosvenorii, amino acid sequence.
SEQ ID NO:177: uridine phosphorylase dependent glycosyltransferase, high GC version (Sg UGT720-269-4 Itkin GC65; SP5081), Siraitia grosvenorii cDNA, nucleic acid sequence.
SEQ ID NO:178: uridine phosphorylase dependent glycosyltransferase, high GC version (Sg UGT720-269-4 Itkin GC65; SP5081), Siraitia grosvenorii, amino acid sequence.
SEQ ID NO:179: uridine phosphorylase dependent glycosyltransferase (Sg UGT94-289-2; SP5082), Siraitia grosvenorii cDNA, nucleic acid sequence.
SEQ ID NO:180: uridine phosphorylase dependent glycosyltransferase (Sg UGT94-289-2; SP5082), Siraitia grosvenorii, amino acid sequence.
SEQ ID NO:181: uridine phosphorylase dependent glycosyltransferase (Sg UGT94-289-2 Itkin; SP5083), Siraitia grosvenorii cDNA, nucleic acid sequence.
SEQ ID NO:182: uridine phosphorylase dependent glycosyltransferase (Sg UGT94-289-2 Itkin; SP5083), Siraitia grosvenorii, amino acid sequence.
SEQ ID NO:183: uridine phosphorylase dependent glycosyltransferase (Sg UGT94-289-3 Itkin; SP5084), Siraitia grosvenorii cDNA, nucleic acid sequence.
SEQ ID NO:184: uridine phosphorylase dependent glycosyltransferase (Sg UGT94-289-3 Itkin; SP5084), Siraitia grosvenorii, amino acid sequence.
SEQ ID NO:185: uridine phosphorylase dependent glycosyltransferase (t134583; SP5085), Siraitia grosvenorii cDNA, nucleic acid sequence.
SEQ ID NO:186: uridine phosphorylase dependent glycosyltransferase (t134583; SP5085), Siraitia grosvenorii, amino acid sequence.
SEQ ID NO:187: uridine phosphorylase dependent glycosyltransferase (t74645; SP5086), Siraitia grosvenorii cDNA, nucleic acid sequence.
SEQ ID NO:188: uridine phosphorylase dependent glycosyltransferase (t74645; SP5086), Siraitia grosvenorii, amino acid sequence.
SEQ ID NO:189: uridine phosphorylase dependent glycosyltransferase (t74693; SP5087), Siraitia grosvenorii cDNA, nucleic acid sequence.
SEQ ID NO:190: uridine phosphorylase dependent glycosyltransferase (t74693; SP5087), Siraitia grosvenorii, amino acid sequence.
SEQ ID NO:191: upstream terpenoid biosynthetic enzyme (Sg 00001291.26_PMK; SP5088), Siraitia grosvenorii cDNA, nucleic acid sequence.
SEQ ID NO:192: upstream terpenoid biosynthetic enzyme (Sg 00001291.26_PMK; SP5088), Siraitia grosvenorii, amino acid sequence.
SEQ ID NO:193: upstream terpenoid biosynthetic enzyme (Sg 00154122.1_IPK; SP5089), Siraitia grosvenorii cDNA, nucleic acid sequence.
SEQ ID NO:194: upstream terpenoid biosynthetic enzyme (Sg 00154122.1_IPK; SP5089), Siraitia grosvenorii, amino acid sequence.
SEQ ID NO:195: upstream terpenoid biosynthetic enzyme (Sg 27366_MVD; SP5090), Siraitia grosvenorii cDNA, nucleic acid sequence.
SEQ ID NO:196: upstream terpenoid biosynthetic enzyme (Sg 27366_MVD; SP5090), Siraitia grosvenorii, amino acid sequence.
SEQ ID NO:197: hydrolase biosynthetic enzyme (E142A-RRK67; SP5091), Aspergillus oryzae cDNA, nucleic acid sequence.
SEQ ID NO:198: hydrolase biosynthetic enzyme (E142A-RRK67; SP5091), Aspergillus oryzae, amino acid sequence.
SEQ ID NO:199: upstream terpenoid biosynthetic enzyme (Mc CDS; SP5092), Momordica charantia cDNA, nucleic acid sequence.
SEQ ID NO:200: upstream terpenoid biosynthetic enzyme (Mc CDS; SP5092), Momordica charantia, amino acid sequence.
SEQ ID NO:201: upstream terpenoid biosynthetic enzyme (Cp CDS; SP5093), Cucurbita pepo cDNA, nucleic acid sequence.
SEQ ID NO:202: upstream terpenoid biosynthetic enzyme (Cp CDS; SP5093), Cucurbita pepo, amino acid sequence.
SEQ ID NO:203: upstream terpenoid biosynthetic enzyme (Tc CDS; SP5094), Trichosanthes cucumerina cDNA, nucleic acid sequence.
SEQ ID NO:204: upstream terpenoid biosynthetic enzyme (Tc CDS; SP5094), Trichosanthes cucumerina, amino acid sequence.
SEQ ID NO:205: upstream terpenoid biosynthetic enzyme (Cc CDS; SP5095), Citrullus colocynthis cDNA, nucleic acid sequence.
SEQ ID NO:206: upstream terpenoid biosynthetic enzyme (Cc CDS; SP5095), Citrullus colocynthis, amino acid sequence.
SEQ ID NO:207: upstream terpenoid biosynthetic enzyme (Ac SQS with intron 6; SP5096), Amaranthus cruentus cDNA, nucleic acid sequence.
SEQ ID NO:208: upstream terpenoid biosynthetic enzyme (Ac SQS with intron 6; SP5096), Amaranthus cruentus, amino acid sequence.
SEQ ID NO:209: upstream terpenoid biosynthetic enzyme (Oe SQS; SP5097), Olea europaea cDNA, nucleic acid sequence.
SEQ ID NO:210: upstream terpenoid biosynthetic enzyme (Oe SQS; SP5097), Olea europaea, amino acid sequence.
SEQ ID NO:211: upstream terpenoid biosynthetic enzyme (Cr SQS; SP5098), Chlamydomonas reinhardtii cDNA, nucleic acid sequence.
SEQ ID NO:212: upstream terpenoid biosynthetic enzyme (Cr SQS; SP5098), Chlamydomonas reinhardtii, amino acid sequence.
SEQ ID NO:213: epoxidase biosynthetic enzyme (Cp SQE1; SP5099), Cucurbita pepo cDNA, nucleic acid sequence.
SEQ ID NO:214: epoxidase biosynthetic enzyme (Cp SQE1; SP5099), Cucurbita pepo, amino acid sequence.
SEQ ID NO:215: epoxidase biosynthetic enzyme (Mc SQE1; SP5100), Momordica charantia cDNA, nucleic acid sequence.
SEQ ID NO:216: epoxidase biosynthetic enzyme (Mc SQE1; SP5100), Momordica charantia, amino acid sequence.
SEQ ID NO:217: epoxidase biosynthetic enzyme (Pg SQE1; SP5101), Panax ginseng cDNA, nucleic acid sequence.
SEQ ID NO:218: epoxidase biosynthetic enzyme (Pg SQE1; SP5101), Panax ginseng, amino acid sequence.
SEQ ID NO:219: upstream terpenoid biosynthetic enzyme (Ab tHMGR; SP5102), Achyranthes bidentata cDNA, nucleic acid sequence.
SEQ ID NO:220: upstream terpenoid biosynthetic enzyme (Ab tHMGR; SP5102), Achyranthes bidentata, amino acid sequence.
SEQ ID NO:221: upstream terpenoid biosynthetic enzyme (Hb tHMGR; SP5103), Hevea brasiliensis cDNA, nucleic acid sequence.
SEQ ID NO:222: upstream terpenoid biosynthetic enzyme (Hb tHMGR; SP5103), Hevea brasiliensis, amino acid sequence.
SEQ ID NO:223: upstream terpenoid biosynthetic enzyme (Pg tHMGR; SP5104), Panax ginseng cDNA, nucleic acid sequence.
SEQ ID NO:224: upstream terpenoid biosynthetic enzyme (Pg tHMGR; SP5104), Panax ginseng, amino acid sequence.
SEQ ID NO:225: transcription factor (At MYC2D105N mutant; SP5105), Arabidopsis thaliana cDNA, nucleic acid sequence.
SEQ ID NO:226: transcription factor (At MYC2D105N mutant; SP5105), Arabidopsis thaliana, amino acid sequence.
SEQ ID NO:227: transcription Factor (Cs bHLH; SP5106), Cucumis sativus cDNA, nucleic acid sequence.
SEQ ID NO:228: transcription Factor (Cs bHLH; SP5106), Cucumis sativus, amino acid sequence.
SEQ ID NO:229: transcription Factor (Bh bHLH; Sp5107), Benincasa hispida cDNA, nucleic acid sequence.
SEQ ID NO:230: transcription Factor (Bh bHLH; Sp5107), Benincasa hispida, amino acid sequence.
SEQ ID NO:231: transcription Factor 2 (Cs bHLH; SP5108), Cucumis sativus cDNA, nucleic acid sequence.
SEQ ID NO:232: transcription Factor 2 (Cs bHLH; SP5108), Cucumis sativus, amino acid sequence.
SEQ ID NO:233: transcription Factor 2 (Bh bHLH; SP5109), Benincasa hispida cDNA, nucleic acid sequence.
SEQ ID NO:234: transcription Factor 2 (Bh bHLH; SP5109), Benincasa hispida, amino acid sequence.
SEQ ID NO:235: poplar sucrose synthase (SP5110) Populus alba cDNA, nucleic acid sequence.
SEQ ID NO:236: poplar sucrose synthase (SP5110) Populus alba, amino acid sequence.
SEQ ID NO:237: UDPG pyrophosphorylase (Lg UDP-Glucose Pyrophosphorylase; SP5111), Larix gmelinii cDNA, nucleic acid sequence.
SEQ ID NO:238: UDPG pyrophosphorylase (Lg UDP-Glucose Pyrophosphorylase; SP5111), Larix gmelinii, amino acid sequence.
SEQ ID NO:239: transcription factor (At PAP2; SP5112), Arabidopsis thaliana cDNA, nucleic acid sequence.
SEQ ID NO:240: transcription factor (At PAP2; SP5112), Arabidopsis thaliana, amino acid sequence.
SEQ ID NO:241: heat shock protein (Ms HSP17.6; SP5113), Medicago sativa cDNA, nucleic acid sequence.
SEQ ID NO:242: heat shock protein (Ms HSP17.6; SP5113), Medicago sativa, amino acid sequence.
SEQ ID NO:243: transcription factor (At JUNGBRUNNEN1 Nac factor; SP5114), Arabidopsis thaliana cDNA, nucleic acid sequence.
SEQ ID NO:244: transcription factor (At JUNGBRUNNEN1 Nac factor; SP5114), Arabidopsis thaliana, amino acid sequence.
SEQ ID NO:245: upstream terpenoid biosynthetic enzyme (AmGPS-SSU (no stop); SP5115), Antirrhinum majus cDNA, nucleic acid sequence.
SEQ ID NO:246: upstream terpenoid biosynthetic enzyme (AmGPS-SSU (no stop); SP5115), Antirrhinum majus, amino acid sequence.
SEQ ID NO:247: upstream terpenoid biosynthetic enzyme (PgFPS; SP5116), Panax ginseng cDNA, nucleic acid sequence.
SEQ ID NO:248: upstream terpenoid biosynthetic enzyme (PgFPS; SP5116), Panax ginseng, amino acid sequence.
SEQ ID NO:249: upstream terpenoid biosynthetic enzyme (PgSQS; SP5117), Panax ginseng cDNA, nucleic acid sequence.
SEQ ID NO:250: upstream terpenoid biosynthetic enzyme (PgSQS; SP5117), Panax ginseng, amino acid sequence.
SEQ ID NO:251: upstream terpenoid biosynthetic enzyme (gbHMGS1; SP5118), Ginkgo biloba cDNA, nucleic acid sequence.
SEQ ID NO:252: upstream terpenoid biosynthetic enzyme (gbHMGS1; SP5118), Ginkgo biloba, amino acid sequence.
SEQ ID NO:253: uridine phosphorylase dependent glycosyltransferase (UGT73AM3; SP5263), Cucumis sativus cDNA, nucleic acid sequence.
SEQ ID NO:254: uridine phosphorylase dependent glycosyltransferase (UGT73AM3; SP5263), Cucumis sativus, amino acid sequence.
SEQ ID NO:255: uridine phosphorylase dependent glycosyltransferase, high GC version (Solyc10g085230.2 UGT GC61; SP5265), Solanum lycopersicum cDNA, nucleic acid sequence.
SEQ ID NO:256: uridine phosphorylase dependent glycosyltransferase, high GC version (Solyc10g085230.2 UGT GC61; SP5265), Solanum lycopersicum, amino acid sequence.
SEQ ID NO:257: uridine phosphorylase dependent glycosyltransferase, high GC version (UGT73AM3 GC64; SP5350), Cucumis sativus cDNA, nucleic acid sequence.
SEQ ID NO:258: uridine phosphorylase dependent glycosyltransferase, high GC version (UGT73AM3 GC64; SP5350), Cucumis sativus, amino acid sequence.
SEQ ID NO:259: AtUBQ10 promoter, Arabidopsis thaliana, nucleic acid sequence.
SEQ ID NO:260: PCLSV promoter, Peanut chlorotic streak virus, nucleic acid sequence.
SEQ ID NO:261: FS4 promoter, Citrullus lanatus, nucleic acid sequence.
SEQ ID NO:262: AtACT2 promoter, Arabidopsis thaliana, nucleic acid sequence.
SEQ ID NO:263: Enhanced AtEf-1A promoter, Cauliflower mosaic virus/Arabidopsis thaliana, nucleic acid sequence.
SEQ ID NO:264: FuasFScp promoter, Figwort mosaic virus hybrid P, nucleic acid sequence.
SEQ ID NO:265: AtFAD2 terminator, Arabidopsis thaliana, nucleic acid sequence.
SEQ ID NO:266: AtNDUFA8 terminator, Arabidopsis thaliana, nucleic acid sequence.
SEQ ID NO:267: CsHSP17.3 terminator, Cucumis sativus, nucleic acid sequence.
SEQ ID NO:268: CsHSP22 terminator, Cucumis sativus, nucleic acid sequence.
SEQ ID NO:269: FE4 promoter, Citrullus lanatus, nucleic acid sequence.
SEQ ID NO:270: Cucumisin promoter, Cucumis melo L., nucleic acid sequence.
SEQ ID NO:271: SgCDS promoter, Siraitia grosvenorii, nucleic acid sequence.
SEQ ID NO:272: upstream terpenoid biosynthetic enzyme, high GC version (C1tHMGR GC; SP2040), Citrullus lanatus cDNA, nucleic acid sequence.
SEQ ID NO:273: upstream terpenoid biosynthetic enzyme, high GC version (C1tHMGR GC 2A; SP2040), Citrullus lanatus, amino acid sequence.\
SEQ ID NO:274: upstream terpenoid biosynthetic enzyme (C1tHMGR GC 2A; SP3351), Citrullus lanatus cDNA, nucleic acid sequence.
SEQ ID NO:275: upstream terpenoid biosynthetic enzyme (C1tHMGR GC 2A; SP3351), Citrullus lanatus, amino acid sequence.
SEQ ID NO:276: upstream terpenoid biosynthetic enzyme (C1tHMGR Zm (for 2A); SP4989), Citrullus lanatus cDNA, nucleic acid sequence.
SEQ ID NO:277: upstream terpenoid biosynthetic enzyme (C1tHMGR Zm (for 2A); SP4989), Citrullus lanatus, amino acid sequence.
SEQ ID NO:278: cytochrome P450 biosynthetic enzyme, optimized to corn (mf CYP72A459v1a Zm (GC48); SP2015), Siraitia grosvenorii cDNA, nucleic acid sequence.
SEQ ID NO:279: cytochrome P450 biosynthetic enzyme, optimized to corn (mf CYP72A459v1a Zm (GC48); SP2015), Siraitia grosvenorii, amino acid sequence.
SEQ ID NO:280: uridine phosphorylase dependent glycosyltransferase (mf UGT720; SP4263), Siraitia grosvenorii cDNA, nucleic acid sequence.
SEQ ID NO:281: uridine phosphorylase dependent glycosyltransferase (mf UGT720; SP4263), Siraitia grosvenorii, amino acid sequence.
SEQ ID NO:282: uridine phosphorylase dependent glycosyltransferase, high GC version (Sg UGT94-289-1 GC65; SP4332), Siraitia grosvenorii cDNA, nucleic acid sequence.
SEQ ID NO:283: uridine phosphorylase dependent glycosyltransferase, high GC version (Sg UGT94-289-1 GC65; SP4332), Siraitia grosvenorii, amino acid sequence.
SEQ ID NO:284: upstream terpenoid biosynthetic enzyme, optimized for corn (Sg CDS Z mays GC51; SP5029), Siraitia grosvenorii cDNA, nucleic acid sequence.
SEQ ID NO:285: upstream terpenoid biosynthetic enzyme, optimized for corn (Sg CDS Z mays GC51; SP5029), Siraitia grosvenorii, amino acid sequence.
SEQ ID NO:286: uridine phosphorylase dependent glycosyltransferase, high GC version (mf UGT720 GC; SP5030), Siraitia grosvenorii cDNA, nucleic acid sequence.
SEQ ID NO:287: uridine phosphorylase dependent glycosyltransferase, high GC version (mf UGT720 GC; SP5030), Siraitia grosvenorii, amino acid sequence.
SEQ ID NO:288: cytochrome P450 biosynthetic enzyme, high GC version (Sg CYP87D18 GC62; SP5031), Siraitia grosvenorii cDNA, nucleic acid sequence.
SEQ ID NO:289: cytochrome P450 biosynthetic enzyme, high GC version (Sg CYP87D18 GC62; SP5031), Siraitia grosvenorii, amino acid sequence.
SEQ ID NO:290: upstream squalene biosynthetic enzyme, high GC version (Sg SQE1 GC66; SP5032), Siraitia grosvenorii cDNA, nucleic acid sequence.
SEQ ID NO:291: upstream squalene biosynthetic enzyme, high GC version (Sg SQE1 GC66; SP5032), Siraitia grosvenorii, amino acid sequence.
SEQ ID NO:292: epoxide hydrolase biosynthetic enzyme, high GC version (Sg EPH3 GC64; SP5033), Siraitia grosvenorii cDNA, nucleic acid sequence.
SEQ ID NO:293: epoxide hydrolase biosynthetic enzyme, high GC version (Sg EPH3 GC64; SP5033), Siraitia grosvenorii, amino acid sequence.
SEQ ID NO:294: uridine phosphorylase dependent glycosyltransferase, high GC version (SgUGT720 GC 2A; SP3186), Siraitia grosvenorii cDNA, nucleic acid sequence.
SEQ ID NO:295: uridine phosphorylase dependent glycosyltransferase, high GC version (SgUGT720 GC 2A; SP3186), Siraitia grosvenorii, amino acid sequence.
SEQ ID NO:296: cytochrome P450 biosynthetic enzyme (Sg SgCYP72v1a Zm (for 2A, GC47); SP3204), Siraitia grosvenorii cDNA, nucleic acid sequence.
SEQ ID NO:297: cytochrome P450 biosynthetic enzyme (Sg SgCYP72v1a Zm (for 2A, GC47); SP3204), Siraitia grosvenorii, amino acid sequence.
SEQ ID NO:298: uridine phosphorylase dependent glycosyltransferase (SgUGT94-1 GC 2A; SP3807), Siraitia grosvenorii cDNA, nucleic acid sequence.
SEQ ID NO:299: uridine phosphorylase dependent glycosyltransferase (SgUGT94-1 GC 2A; SP3807), Siraitia grosvenorii, amino acid sequence.
SEQ ID NO:300: uridine phosphorylase dependent glycosyltransferase (SgUGT720 Zm (for 2A); SP3897), Siraitia grosvenorii cDNA, nucleic acid sequence.
SEQ ID NO:301: uridine phosphorylase dependent glycosyltransferase (SgUGT720 Zm (for 2A); SP3897), Siraitia grosvenorii, amino acid sequence.
SEQ ID NO:302: uridine phosphorylase dependent glycosyltransferase (mf UGT720; SP4263), Siraitia grosvenorii cDNA, nucleic acid sequence.
SEQ ID NO:303: uridine phosphorylase dependent glycosyltransferase (mf UGT720; SP4263), Siraitia grosvenorii, amino acid sequence.
SEQ ID NO:304: uridine phosphorylase dependent glycosyltransferase, high GC version (Sg UGT94-289-1 GC65; SP4332), Siraitia grosvenorii cDNA, nucleic acid sequence.
SEQ ID NO:305: uridine phosphorylase dependent glycosyltransferase, high GC version (Sg UGT94-289-1 GC65; SP4332), Siraitia grosvenorii, amino acid sequence.

### DETAILED DESCRIPTION

The present disclosure generally describes transgenic plants and biosynthetic systems thereof for making mogrol/mogroside pathway enzymes and mogrosides, and methods for making such transgenic plants. The following sections provide embodiments that describe the subject matter in greater detail.

### I. Mogroside Pathway Enzymes and Mogrosides

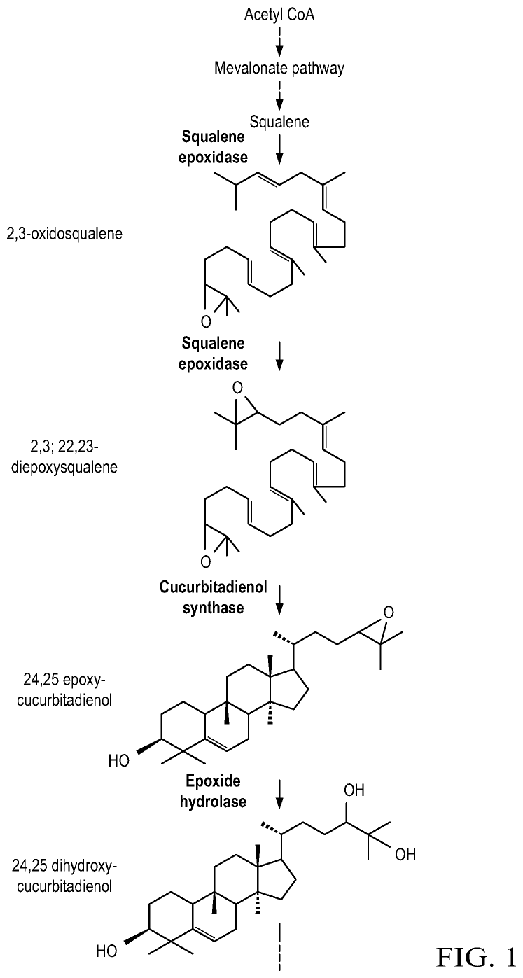

Mogrosides are highly stable molecules based on a triterpene skeleton, formed of varying numbers of glucose units, from 1 to 6 attached to the triterpene backbone. Mogrosides may also comprise a non-glucose moiety, such as grosmomoside I. FIG. 1 shows a mogroside biosynthetic pathway from Siraitia grosvenorii (Itkin et al., Proc Nat Acad Sci USA 113:E7619-E7628, 2016; Seki et al., Biosci. Biotechnol. Biochem. 82:927-934, 2018), and certain enzymes capable of catalyzing the reactions in the pathway. Importantly, the enzymatic pathway used for production of mogrosides according to the present disclosure is not limited by the mechanisms shown in FIG. 1. Other terpene structures, mogrol precursors, enzyme-catalyzed reactions or conversion mechanisms are also possible. Additionally, certain enzyme may catalyze more than one type of reaction, and one or more additional genes can be used to produce any of the mogrol intermediates, mogrol, or any of the mogroside compounds.

Provided herein are exemplary nucleic acid and protein sequences for certain mogroside pathway (also referred to as mogroside biosynthetic pathway) enzymes for conversion of mogrol precursors, such as squalene, to mogrol and eventually various mogroside compounds, including, but not limited to, mogroside V. The enzymes of the mogroside pathway include, but are not limited to, squalene epoxidase (SQE), cucurbitadienol synthase (CDS), epoxy or epoxide hydrolase (EPH), various cytochrome P450 enzymes (CYP), including, but not limited to, CYP72 and CYP87, uridine phosphorylase dependent glycosyltransferase enzymes (UGT), including, but not limited to, UGT720, UGT94 and UGT74, and can additionally include 3 hydroxy-3-methylglutaryl-CoA reductase (HMGR), or truncated versions thereof, and NADPH:cytochrome P450 reductase (CPR2).

SQE, CDS, CYP and EPH are involved in the successive steps of producing and converting mogrol precursors, such as squalene, into mogrol. Intermediate products of the enzymatic pathway include, but are not limited to, 2,3-oxidosqualene, 2,3;22.23-dioxidosqualene, 24,25 epoxycucurbitadienol, and 24,25-dihydroxycucurbitadienol. CDS, which is an oxidosqualene cyclase, uses 2,3;22,23-diepoxysqualene as its substrate to produce 24,25-epoxycucurbitadienol. Genomic analysis has revealed that S. grosvenorii has five genes that may encode SQEs. Of these, two are strongly expressed during the initial stages of fruit development, as are CDS, CYP enzymes, and EPHs, which catalyze subsequent steps. The S. grosvenorii genome contains eight genes encoding EPHs, which catalyze conversion of 24,25-epoxycucurbitadienol to 24,25-dihydroxycucurbitadienol. Certain enzyme may catalyze more than one type of reaction.

After the formation of mogrol, a series of glycosylations occurs to add glucose molecules, at position C-3 and position C-24, to produce Mogrosides I-VI with various degrees of glycosylation. The Roman numeral I, II, III, IV, V, and VI respectively stand for the number of glucose unit(s) in the corresponding glycosylated mogroside, isomogroside, or oxomogroside. Two UGTs contribute to these steps. One is UGT720, which is strongly expressed in the initial stages of fruit development and transfers one glucose molecule each to the hydroxyl groups at positions C-24 and C 3 of mogrol. The second is UGT94, which is strongly expressed in the latter stages of fruit development and adds sugars to the other sugars already present on the acceptor molecule.

Although the mogroside pathway was initially described in S. grosvenorii (monkfruit), certain non-monkfruit plants can make tetracyclic triterpenoid compounds similar to mogrol, because at least one of the intermediates, such as triterpenes, exist in the cellular pathway. Moreover, given that the related pathways for modifying tetracyclic triterpenoid compounds require associated enzymes such as reductases, these plants already express certain associated network enzymes. Although such non-monkfruit plants may express enzymes that produce mogrol precursors, these plants do not naturally produce all enzymes in a coordinated fashion required to produce mogrosides. For instance, plants such as cucumber, melon, and watermelon naturally express cucurbitadienol synthases, which are capable of producing cucurbitadienol. However, other enzymes like the CYP enzymes, which are capable of altering the cucurbitadienol scaffold, redirect this intermediate to other terpene derivatives. Therefore, changes to the genomes of these non-monkfruit plants, either by recombinant, gene editing, or other modern plant breeding technologies, may allow for these non-monkfruit plants to produce mogrol and mogrosides. Therefore, in certain embodiments one or more additional genes could be introduced into non-monkfruit plants, or enzymes native to such non-monkfruit plants could be upregulated, to allow for intermediate metabolite production in order to yield mogrol, mogrosides, and mogroside-based sweeteners.

The term “mogrol precursor” broadly encompasses all possible terpene derivatives and intermediate products towards the production of mogrol and mogroside compounds, including, but not limited to, 2,3-oxidosqualene, 2,3;22.23-dioxidosqualene, 24,25-epoxycucurbitadienol, and 24,25-dihydroxycucurbitadienol, cucurbitadienol, 11-hydroxy-cucurbitadienol, and 11-oxo-cucurbitadienol. Mogrosides refer to any possible glycosylation products of mogrol, including, but not limited to, Siamenoside I, Siratose (a stereoisomer of Siamenoside I), Mogroside VI, Mogroside V, Isomogroside V, Mogroside IV, Mogroside III, Mogroside IIIE, Mogroside IIE, Mogroside IIA, Mogroside IE, and Mogroside IA. Other examples of mogrosides include, but are not limited to, Mogroside IIB, 7-Oxomogroside IIE, 11 Oxomogroside A1, Mogroside III A2, 11-Deoxymogroside III, 11-Oxomogroside IVA, 7 Oxomogroside V, and 11-Oxo-mogroside V. Metabolites and derivatives of mogrosides refer to any close variation of mogrosides through a metabolic reaction, naturally occurring reaction, or non-naturally occurring reaction. Derivatives of mogrosides may comprise deletions, alterations, or additions of atom(s) or functional groups compared with standard mogrosides. However, metabolites and derivatives of mogrosides retain substantially the same function and characteristics of standard mogrosides. In certain instances the mogroside compounds produced by the presently disclosed transgenic plants and organisms can also undergo non-enzymatic (spontaneous) conversion to other mogroside compounds.

One or more of these mogroside compounds can be isolated, purified or partially purified from the disclosed transgenic plants, or transgenic organisms grown via fermentation techniques. For example the one or more mogroside compounds can be harvested via wet extraction or an aqueous layer containing the mogroside compounds in bulk processing steps that do not necessarily result in the isolation of a single mogroside compound. Alternatively, the whole transgenic plant (or part thereof) or transgenic organism producing the mogroside compounds may be dried and pulverized into powder or extracted, or the whole transgenic plant (or part thereof) or transgenic organism producing the mogroside compounds can be minimally processed and used as a food ingredient.

In certain embodiments the presently disclosed transgenic plants and organisms will produce a unique ratio of mogroside compounds. In some embodiments the presently disclosed transgenic plants and organisms can produce more of one or more of the mogroside compounds and less of other mogroside compounds, for example higher amounts of mogroside V compared to other mogroside compounds.

In further embodiments the complete mogroside biosynthetic pathway can be established in a selected plant or organism using a combination approach, for example by endogenously activating one or more mogroside biosynthetic pathway nucleic acid sequences that are naturally present in the plant or organism and providing any mogroside biosynthetic pathway nucleic acid sequences that are not naturally present in the plant or organism via one or more expression vector(s) comprising the non-endogenous mogroside biosynthetic pathway nucleic acid sequences.

In further embodiments the transgenic plant or organism can also be engineered to express one or more nucleic acids involved in the biosynthesis of other sweeteners, for example genes involved in the production of siamenoside I, α-siamenoside, steviol glycosides (stevia), Rebaudioside M or glycyrrhizin. Additionally, the transgenic plants or organisms can be engineered to produce mogrosides and other sweeteners together in the same plant or organism, for example to produce mogrosides and Rebaudioside M.

### II. Recombinant Host Cells and Species

Although transgenic plants are generally described throughout the present disclosure, other host cells and organisms are also envisioned for use in certain embodiments of the present disclosure.

As used herein, the term “recombinant host cell” is intended to refer to any host cell whose genome has been engineered to include at least one of the presently disclosed mogroside biosynthetic pathway nucleic acid sequences, which in certain embodiments encode one or more polypeptides. These sequences include, but are not limited to, nucleic acid or amino acid sequences that are not naturally present in the host cell or organism, DNA sequences that are not normally transcribed into RNA or translated into a protein (“expressed”), and other sequences that have been altered from those normally present in the host cell, for example by increasing the copy number of the DNA sequence or altering the expression patterns or expression levels.

In addition to the plants species disclosed herein, a number of prokaryotes and additional eukaryotes are suitable for use as recombinant hosts in different aspects of the present disclosure. In addition to any plant species, the recombinant host cells may be a bacteria, yeast or fungi. A host cell or species selected for mogroside compound production can be analyzed to determine if any mogroside biosynthetic pathway genes are endogenous to the host cell or species, and which mogroside biosynthetic pathway genes are not present. Genes for which an endogenous counterpart is not present in the host cell or organism are generally assembled in one or more recombinant constructs, which are then transformed into the host cell or organism in order to supply the missing function(s).

Exemplary prokaryotic and eukaryotic species useful in certain aspects of the present disclosure include, but are not limited to, Agaricus, Aspergillus, Bacillus, Candida, corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Sphaceloma, Schizosaccharomyces, Xanthophyllomyces and Yarrowia. In some embodiments, a recombinant host may be a microorganism, for example Pichia pastoris, Schizosaccharomyces pombe, Aspergillus niger, or Saccharomyces cerevisiae. In some embodiments, a recombinant host may be a microorganism such as Escherichia coli or Agrobacterium tumefaciens. It will be appreciated that certain microorganisms can be used to screen and test genes of interest in a high throughput manner, while other microorganisms with desired productivity or growth characteristics can be used for large-scale production of mogroside compounds. In certain embodiments food grade microorganisms may be useful for large-scale production purposes.

### III. Nucleic Acid and Polypeptide Sequences

Certain embodiments of the current disclosure concern nucleic acid sequences (polynucleotides) and the corresponding amino acid sequences (proteins or polypeptides) for mogroside biosynthesis pathway genes. Complements to any nucleic acid or protein sequences described herein are also provided.

“Identity,” as is well understood in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. Methods to determine “identity” are designed to give the largest match between the sequences tested. Moreover, methods to determine identity are codified in publicly available programs. “Identity” can be readily calculated by any of the many methods known to those of skill in the art. Computer programs can be used to determine “identity” between two sequences these programs include but are not limited to, GCG; suite of five BLAST programs, three designed for nucleotide sequences queries (BLASTN, BLASTX, and TBLASTX) and two designed for protein sequence queries (BLASTP and TBLASTN). The BLASTX program is publicly available from NCBI and other sources (BLAST Manual, NCBI NLM NIH, Bethesda, Md. 20894). The well-known Smith Waterman algorithm can also be used to determine identity.

In accordance with the present disclosure, a polynucleotide or polypeptide sequence as described herein may exhibit at least from about 34%, 40%, 50%, 60%, 62% or 70% to about 100% sequence identity to at least one of the sequences set forth herein. For example, in one embodiment, a mogroside biosynthesis pathway gene as described herein may comprise, for example, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from SEQ ID NOs:1, 3, 4, 6, 8, 10, 12, 14, 16, 18, 20, 22, 24, 26, 28, 30, 32, 34, 38, 42, 46, 50, 54, 85 or 87-92, or a complement thereof. In other embodiments, a mogroside biosynthesis pathway protein as described herein may comprise for example, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a sequence selected from SEQ ID NOs:2, 5, 7, 9, 11, 13, 15, 17, 19, 21, 23, 25, 27, 29, 31, 33,35-37, 39-41, 43-45, 47-49, 51-53, 55-57 or 86.

Parameters for polypeptide sequence comparison include the following: Algorithm: Needleman and Wunsch (J. Mol. Biol. 48:443-453, 1970); Comparison matrix: BLOSUM62 from Hentikoff and Hentikoff, (Proc. Natl. Acad. Sci. USA 89:10915-10919, 1992); Gap Penalty: 12; and Gap Length Penalty: 4. A program that can be used with these parameters is publicly available as the “gap” program from Genetics Computer Group, Madison WI. The above parameters along with no penalty for end gap may serve as default parameters for peptide comparisons.

Parameters for nucleic acid sequence comparison include the following: Algorithm: Needleman and Wunsch (supra); Comparison matrix: matches=+10; mismatches=0; Gap Penalty: 50; and Gap Length Penalty: 3. A program that can be used with these parameters is publicly available as the “gap” program from Genetics Computer Group, Madison Wis. The above parameters may serve as the default parameters for nucleic acid comparisons.

As used herein, “hybridization,” “hybridizes,” or “capable of hybridizing” is understood to mean the forming of a double- or triple-stranded molecule or a molecule with partial double- or triple-stranded nature. Such hybridization may take place under relatively high-stringency conditions, including low salt and/or high temperature conditions, such as provided by a wash in about 0.02 M to about 0.15 M NaCl at temperatures of about 50° C. to about 70° C. for 10 min. In one embodiment of the present disclosure, the conditions are 0.15 M NaCl and 70° C. Stringent conditions tolerate little mismatch between a nucleic acid and a target strand. Such conditions are well-known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like. Also included may be a protein or polypeptide, or fragment thereof, such as any of those set forth herein.

“Fragment”, with respect to the nucleic acid sequences disclosed herein, refers to any part of a polynucleotide molecule that retains a usable, functional characteristic. Useful fragments include oligonucleotides and polynucleotides that may be used as probes or primers in hybridization or amplification technologies or in the regulation of replication, transcription or translation. A polynucleotide fragment refers to any subsequence of a polynucleotide, typically, of at least about 15 consecutive nucleotides, at least about 16 consecutive nucleotides, at least about 17 consecutive nucleotides, at least about 18 consecutive nucleotides, at least about 19 consecutive nucleotides, at least about 20 consecutive nucleotides, at least about 21 consecutive nucleotides, at least about 22 consecutive nucleotides, at least about 23 consecutive nucleotides, at least about 24 consecutive nucleotides, at least about 25 consecutive nucleotides, at least about 30 consecutive nucleotides, at least about 35 nucleotides, at least about 40 consecutive nucleotides, at least about 45 consecutive nucleotides, or at least about 50 nucleotides or more, of any of the nucleic acid sequences provided herein.

Fragments may also include subsequences of polypeptides and protein molecules, or a subsequence of the polypeptide, as disclosed herein. Fragments may have antigenic potential, or may be a subsequence of the polypeptide that performs at least one biological function of the intact polypeptide in substantially the same manner, or to a similar extent, as does the intact polypeptide. Fragments can vary in size from as few as 5 amino acids to the full length of the intact polypeptide, but are preferably at least about 10 amino acids in length, at least about 15 amino acids in length, at least about 20 amino acids in length, at least about 25 amino acids in length, at least about 30 amino acids in length, at least about 35 amino acids in length, at least about 40 amino acids in length, at least about 45 amino acids in length, at least about 50 amino acids in length, at least about 55 amino acids in length, or at least about 60 amino acids in length or more, of any of the amino acid sequences provided herein.

The nucleic acids provided herein as SEQ ID NOs:1, 3, 4, 6, 8, 10, 12, 14, 16, 18, 20, 22, 24, 26, 28, 30, 32, 34, 38, 42, 46, 50, 54, 58, 85 or 87-92, and amino acids provided herein as SEQ ID NOs:2, 5, 7, 9, 11, 13, 15, 17, 19, 21, 23, 25, 27, 29, 31, 33, 35-37, 39-41, 43-45, 47-49, 51-53, 55-57, 59 or 86, may be from any source, e.g., identified as naturally occurring in a plant, or synthesized, e.g., by mutagenesis of SEQ ID NOs: 1, 3, 4, 6, 8, 10, 12, 14, 16, 18, 20, 22, 24, 26, 28, 30, 32, 34, 38, 42, 46, 50, 54, 58, 85 or 87-92, for example to create a coding sequence with a G/C content more like the G/C content of naturally occurring genes from a particular plant. The naturally occurring sequence may be from any plant or algal species, as described herein.

### IV. Transformation Constructs

Vectors used for plant transformation, or transformation of other host cells or organisms, may include, for example, plasmids, cosmids, YACs (yeast artificial chromosomes), BACs (bacterial artificial chromosomes) or any other suitable cloning system, as well as fragments of DNA therefrom. Thus when the term “vector” or “expression vector” is used, all of the foregoing types of vectors, as well as nucleic acid sequences isolated therefrom, are included. It is contemplated that utilization of cloning systems with large insert capacities will allow introduction of large DNA sequences comprising more than one selected gene. In accordance with the present disclosure, this could be used to introduce genes corresponding to an entire biosynthetic pathway into a plant. Introduction of such sequences may be facilitated by use of bacterial or yeast artificial chromosomes (BACs or YACs, respectively), or even plant artificial chromosomes. For example, the use of BACs for Agrobacterium-mediated transformation was disclosed by Hamilton et al. (Proc. Natl. Acad. Sci. USA 93:9975-9979, 1996).

Particularly useful for transformation are expression cassettes that have been isolated from such vectors. DNA segments used for transforming plant cells will, of course, generally comprise the cDNA, gene or genes that one desires to introduce into and have expressed in the host cells. These DNA segments can further include structures such as promoters, enhancers, polylinkers, terminators or even regulatory genes as desired. The DNA segment or gene chosen for cellular introduction will often encode a protein that will be expressed in the resultant recombinant cells resulting in a screenable or selectable trait and/or that will impart an improved phenotype to the resulting transgenic plant. However, this may not always be the case, and the present disclosure also encompasses transgenic plants incorporating non-expressed transgenes. As previously discussed, in addition to plant cells the host cells in certain aspects of the present disclosure may be a bacterial cell, such as Escherichia colt or Agrobacterium tumefaciens, yeast cells, fungal, algal or cyanobacterial cells. The skilled artisan is aware of the genetic elements that must be present on a vector in order to successfully transform, select and propagate host cells containing a sequence of interest. Components that may be included with vectors used in the current disclosure are as follows.

### A. Promoters and Other Regulatory Elements

In certain embodiments, the presently disclosed expression cassettes further comprise one or more promoters, for example one or more of the nucleotide sequences set forth in SEQ ID NOs:60-71, or a nucleotide sequence having a sequence identity of at least 70%, at least 75%, at least 80%, at least 85%, at least 90%, at least 95%, at least 96%, at least 97%, at least 98%, or at least 99% to one or more of the nucleotide sequences set forth in SEQ ID NOs:60-71.

In addition to the promoter sequences disclosed in the Sequence Listing, other exemplary promoters for expression of a nucleic acid sequence include a plant promoter such as the CaMV 35S promoter (Odell et al., Nature 313:810-812, 1985), or others such as CaMV 19S (Lawton et al., Plant Mol. Biol. 9:315-324, 1987), nos (Ebert et al., Proc. Natl. Acad. Sci. USA 84:5745-5749, 1987), Adh (Walker et al., Proc. Natl. Acad. Sci. USA 84:6624-6628, 1987), sucrose synthase (Yang and Russell, Proc. Natl. Acad. Sci. USA 87:4144-4148, 1990), α-tubulin, actin (Wang et al., Mol. Cell Biol. 12:3399-3406, 1992), cab (Sullivan et al., Mol. Gen. Genet. 215:431-440, 1989), PEPCase (Hudspeth and Grula, Plant Mol. Biol. 12:579-589, 1989) or those associated with the R gene complex (Chandler et al., Plant Cell 1:1175-1183, 1989). Tissue specific promoters such as root cell promoters (Conkling et al., Plant Physiol. 93:1203-1211, 1990) and tissue specific enhancers are also contemplated to be useful, as are inducible promoters such as ABA- and turgor-inducible promoters. The PAL2 promoter may in particular be useful with the disclosure (U.S. Patent Application Publication No. 2004/0049802, the entire disclosure of which is specifically incorporated herein by reference). In one embodiment of the present disclosure, the native promoter of one or more of the mogroside pathway genes is used. In some embodiments, the promoter is a strong promoter or a weak promoter.

The DNA sequence between the transcription initiation site and the start of the coding sequence, i.e., the untranslated leader sequence, can also influence gene expression. One may thus wish to employ a particular leader sequence with a transformation construct of the present disclosure. Leader sequences are contemplated to include those that comprise sequences predicted to direct optimum expression of the attached gene, i.e., to include a consensus leader sequence that may increase or maintain mRNA stability and prevent inappropriate initiation of translation. The choice of such sequences will be known to those of skill in the art in light of the present disclosure. Sequences that are derived from genes that are highly expressed in plants may be desirable.

It is contemplated that vectors for use in accordance with the present disclosure may be constructed to include an ocs enhancer element. This element was first identified as a 16 bp palindromic enhancer from the octopine synthase (ocs) gene of Agrobacterium (Ellis et al., EMBO J. 6:3203-3208, 1987), and is present in at least 10 other promoters (Bouchez et al., EMBO J. 8:4197-4204, 1989). The use of an enhancer element, such as the ocs element and particularly multiple copies of the element, may act to increase the level of transcription from adjacent promoters when applied in the context of plant transformation.

It is envisioned that mogroside biosynthesis pathway coding sequences may be introduced under the control of novel promoters or enhancers, etc., or homologous or tissue specific promoters or control elements. Vectors for use in tissue-specific targeting of genes in transgenic plants will typically include tissue-specific promoters and may also include other tissue-specific control elements such as enhancer sequences. Promoters that direct specific or enhanced expression in certain plant tissues will be known to those of skill in the art in light of the present disclosure. These include, for example, the rbcS promoter, specific for green tissue; the ocs, nos and mas promoters that have higher activity in roots or wounded leaf tissue.

### B. Terminators

In certain embodiments, the presently disclosed expression cassettes further comprise one or more terminators, for example one or more of the nucleotide sequences set forth in SEQ ID NOs:72-80, or a nucleotide sequence having a sequence identity of at least 70%, at least 75%, at least 80%, at least 85%, at least 90%, at least 95%, at least 96%, at least 97%, at least 98%, or at least 99% to one or more of the nucleotide sequences set forth in SEQ ID NOs:72-80.

Transformation constructs prepared in accordance with the present disclosure will typically include a 3′ end DNA sequence that acts as a signal to terminate transcription and allow for the polyadenylation of the mRNA produced by coding sequences operably linked to a promoter. In one embodiment of the present disclosure, the native terminator of a mogroside biosynthesis pathway coding sequence is used. Alternatively, a heterologous 3′ end may enhance the expression of sense or antisense mogroside biosynthesis pathway coding sequences. In addition to the terminator sequences disclosed in the Sequence Listing, further examples of terminators that are deemed to be useful in this context include those from the nopaline synthase gene of Agrobacterium tumefaciens (nos 3′ end) (Bevan et al., Nucl. Acids Res. 11:369-385, 1983), the terminator for the T7 transcript from the octopine synthase gene of Agrobacterium tumefaciens, and the 3′ end of the protease inhibitor I or II genes from potato or tomato. Regulatory elements such as an Adh intron (Callis et al., Genes Dev. 1:1183-1200, 1987), sucrose synthase intron (Vasil et al., Plant Physiol. 91:1575-1579, 1989) or TMV omega element (Gallie and Kado, Proc. Natl. Acad. Sci. USA 86:129-132, 1989), may further be included where desired.

### C. Transit or Signal Peptides

In certain embodiments of the present disclosure transit or signal sequences may be incorporated into the mogroside biosynthesis pathway coding sequences. Sequences that are joined to the coding sequence of an expressed gene, which are removed post-translationally from the initial translation product and that facilitate the transport of the protein into or through intracellular or extracellular membranes, are termed transit (usually into vacuoles, vesicles, plastids and other intracellular organelles) and signal sequences (usually to the endoplasmic reticulum, golgi apparatus and outside of the cellular membrane). By facilitating the transport of the protein into compartments inside and outside the cell, these sequences may increase the accumulation of gene product protecting them from proteolytic degradation. These sequences also allow for additional mRNA sequences from highly expressed genes to be attached to the coding sequence of the genes. Since mRNA being translated by ribosomes is more stable than naked mRNA, the presence of translatable mRNA in front of the gene may increase the overall stability of the mRNA transcript from the gene and thereby increase synthesis of the gene product. Since transit and signal sequences are usually post-translationally removed from the initial translation product, the use of these sequences allows for the addition of extra translated sequences that may not appear on the final polypeptide. It further is contemplated that targeting of certain proteins may be desirable in order to enhance the stability of the protein (U.S. Pat. No. 5,545,818, incorporated herein by reference in its entirety).

Additionally, vectors may be constructed and employed in the intracellular targeting of a specific gene product within the cells of a transgenic plant or in directing a protein to the extracellular environment. This generally will be achieved by joining a DNA sequence encoding a transit or signal peptide sequence to the coding sequence of a particular gene. The resultant transit, or signal, peptide will transport the protein to a particular intracellular, or extracellular destination, respectively, and will then be post-translationally removed.

### D. Marker Genes

By employing a selectable or screenable marker protein, one can provide or enhance the ability to identify transformants. “Marker genes” are genes that impart a distinct phenotype to cells expressing the marker protein and thus allow such transformed cells to be distinguished from cells that do not have the marker. Such genes may encode either a selectable or screenable marker, depending on whether the marker confers a trait that one can “select” for by chemical means, i.e., through the use of a selective agent (e.g., a herbicide, antibiotic, or the like), or whether it is simply a trait that one can identify through observation or testing, i.e., by “screening” (e.g., the green fluorescent protein). In addition to the marker genes disclosed in the Sequence Listing, many additional examples of suitable marker proteins are known to the art and can be employed in the practice of the present disclosure.

Included within the terms “selectable” or “screenable” markers also are genes that encode a “secretable marker” whose secretion can be detected as a means of identifying or selecting for transformed cells. Examples include markers that are secretable antigens that can be identified by antibody interaction, or even secretable enzymes that can be detected by their catalytic activity. Secretable proteins fall into a number of classes, including small, diffusible proteins detectable, e.g., by ELISA; small active enzymes detectable in extracellular solution (e.g., α-amylase, β-lactamase, phosphinothricin acetyltransferase); and proteins that are inserted or trapped in the cell wall (e.g., proteins that include a leader sequence such as that found in the expression unit of extensin or tobacco PR S).

Many selectable marker coding regions are known and could be used with the present disclosure including, but not limited to, neo (Potrykus et al., Mol. Gen. Genet. 199:169-177, 1985), which provides kanamycin resistance and can be selected for using kanamycin, G418, paromomycin, etc.; bar, which confers bialaphos or phosphinothricin resistance; a mutant EPSP synthase protein conferring glyphosate resistance; a nitrilase such as bxn from Klebsiella ozaenae, which confers resistance to bromoxynil (Stalker et al., J. Biol. Chem. 263:6310-6314, 1988); a mutant acetolactate synthase (ALS), which confers resistance to imidazolinone, sulfonylurea or other ALS inhibiting chemicals (European Patent Application 154,204, 1985); a methotrexate resistant DHFR (Thillet et al., J. Biol. Chem. 263:12500-12508, 1988), a dalapon dehalogenase that confers resistance to the herbicide dalapon; or a mutated anthranilate synthase that confers resistance to 5-methyl tryptophan, or sequences that confer resistance to dicamba.

An illustrative embodiment of selectable marker capable of being used in systems to select transformants are those that encode the enzyme phosphinothricin acetyltransferase, such as the bar gene from Streptomyces hygroscopicus or the pat gene from Streptomyces viridochromogenes. The enzyme phosphinothricin acetyl transferase (PAT) inactivates the active ingredient in the herbicide bialaphos, phosphinothricin (PPT). PPT inhibits glutamine synthetase, causing rapid accumulation of ammonia and cell death.

Screenable markers that may be employed include a β glucuronidase (GUS) or uidA gene, which encodes an enzyme for which various chromogenic substrates are known; an R-locus gene, which encodes a product that regulates the production of anthocyanin pigments (red color) in plant tissues; a β lactamase gene (Sutcliffe, Proc. Natl. Acad. Sci. USA 75:3737-3741, 1978), which encodes an enzyme for which various chromogenic substrates are known (e.g., PADAC, a chromogenic cephalosporin); a xylE gene (Zukowsky et al., Proc. Natl. Acad. Sci. USA 80:1101-1105, 1983), which encodes a catechol dioxygenase that can convert chromogenic catechols; an α-amylase gene (Ikuta et al., Biotechnology 8:241-242, 1990); a tyrosinase gene (Katz et al., J. Gen. Microbiol. 129:2703-2714, 1983), which encodes an enzyme capable of oxidizing tyrosine to DOPA and dopaquinone, which in turn condenses to form the easily-detectable compound melanin; a β galactosidase gene, which encodes an enzyme for which there are chromogenic substrates; a luciferase (lux) gene (Ow et al., Science 234:856-859, 1986), which allows for bioluminescence detection; an aequorin gene (Prasher et al., Biochem. Biophys. Res. Commun. 126:1259-1268, 1985), which may be employed in calcium-sensitive bioluminescence detection; or a gene encoding for green fluorescent protein (GFP; Sheen et al., Plant J. 8:777-784, 1995; Haseloff et al., Proc. Natl. Acad. Sci. USA 94:2122-2127, 1997; Reichel et al., Proc. Natl. Acad. Sci. USA 93:5888-5893, 1996; WO 97/41228) is also contemplated as a useful reporter gene. Expression of green fluorescent protein may be visualized in a cell or plant as fluorescence following illumination by particular wavelengths of light.

### E. Additional Agronomic Traits

In certain embodiments one or more additional agronomically beneficial trait(s) are engineered into the presently disclosed transgenic plants or organisms. A “trait” refers to a physiological, morphological, biochemical, or physical characteristic of a plant or organism or particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g., by measuring uptake of carbon dioxide, or by the observation of the expression level of a gene or genes, e.g., by employing northern analysis, RT-PCR, microarray gene expression assays, or reporter gene expression systems, or by agricultural observations such as stress tolerance, yield, or pathogen tolerance. Any technique can be used to measure the amount of, comparative level of, or difference in any selected chemical compound or macromolecule in the transgenic plants, however.

“Trait modification” refers to a detectable difference in a characteristic in a plant or organism expressing or ectopically expressing a polynucleotide or polypeptide relative to a plant or organism not doing so, such as a wild-type or other control plant or organism. In some cases, the trait modification can be evaluated quantitatively. For example, the trait modification can entail at least about a 2% increase or decrease in an observed trait (difference), at least a 5% difference, at least about a 10% difference, at least about a 20% difference, at least about a 30%, at least about a 50%, at least about a 70%, or at least about a 100%, or an even greater difference compared with a wild-type or other control plant or organism. It is known that there can be a natural variation in the modified trait. Therefore, the trait modification observed entails a change of the normal distribution of the trait in the plant or organism compared with the distribution observed in wild-type plant or organism.

Trait modifications of particular interest in the presently disclosed plants include those to seed (such as embryo or endosperm), fruit, root, flower, leaf, stem, shoot, seedling or the like, including: enhanced tolerance to environmental conditions, including freezing, chilling, heat, drought, water saturation, radiation and ozone; improved tolerance to microbial, fungal or viral diseases; improved tolerance to pest infestations, including insects, nematodes, mollicutes, parasitic higher plants or the like; decreased herbicide sensitivity or increased herbicide tolerance, for example increased glyphosate or dicamba tolerance; improved tolerance of heavy metals or enhanced ability to take up heavy metals; improved growth under poor photoconditions (e.g., low light and/or short day length), or changes in expression levels of genes of interest. Other phenotypes that can be modified relate to the production of plant metabolites, such as variations in the production of taxol, tocopherol, tocotrienol, sterols, phytosterols, vitamins, wax monomers, anti-oxidants, amino acids, lignins, cellulose, tannins, prenyllipids (such as chlorophylls and carotenoids), glucosinolates, and terpenoids, enhanced or compositionally altered protein or oil production (especially in seeds), or modified sugar (insoluble or soluble) and/or starch composition. Physical plant characteristics that can be modified include cell development (such as the number of trichomes), fruit and seed size and number, yields of plant parts such as stems, leaves, inflorescences, and roots, the stability of the seeds during storage, characteristics of the seed pod (e.g., susceptibility to shattering), root hair length and quantity, internode distances, or the quality of seed coat. Plant growth characteristics that can be modified include growth rate, germination rate of seeds, vigor of plants and seedlings, leaf and flower senescence, male sterility, apomixis, flowering time, flower abscission, rate of nitrogen uptake, osmotic sensitivity to soluble sugar concentrations, biomass or transpiration characteristics, as well as plant architecture characteristics such as apical dominance, branching patterns, number of organs, organ identity, organ shape or size. Additionally, the amount of natural sugar(s) can be reduced, and color can be reduced or removed.

### V. Gene Editing

One method for producing the transgenic plants of the present disclosure is through genome modification using site-specific integration or genome editing. Targeted modification of plant genomes through the use of genome editing methods can be used to create improved plant lines through modification of plant genomic DNA. As used herein “site-directed integration” refers to genome editing methods that enable targeted insertion of one or more nucleic acids of interest into a plant genome. Suitable methods for altering a wild-type DNA sequence or a preexisting transgenic sequence or for inserting DNA into a plant genome at a pre-determined chromosomal site include any method known in the art. Exemplary methods include the use of sequence specific nucleases, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, or a CRISPR/Cascade system). Several embodiments relate to methods of genome editing by using single-stranded oligonucleotides to introduce precise base pair modifications in a plant genome. Methods of genome editing to modify, delete, or insert nucleic acid sequences into genomic DNA are known in the art.

In certain embodiments, the present disclosure provides modification or replacement of an existing coding sequence, such as an existing transgenic insert, within a plant genome with a sequence encoding a different protein, or an expression cassette comprising such a protein. Several embodiments relate to the use of a known genome editing methods, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, or a CRISPR/Cascade system).

Several embodiments may therefore relate to a recombinant DNA construct comprising an expression cassette(s) encoding a site-specific nuclease and, optionally, any associated protein(s) to carry out genome modification. These nuclease-expressing cassette(s) may be present in the same molecule or vector as a donor template for templated editing. Several methods for site-directed integration are known in the art involving different sequence-specific nucleases (or complexes of proteins or guide RNA or both) that cut the genomic DNA to produce a double strand break (DSB) or nick at a desired genomic site or locus. As understood in the art, during the process of repairing the DSB or nick introduced by the nuclease enzyme, the donor template DNA, transgene, or expression cassette may become integrated into the genome at the site of the DSB or nick. The presence of the homology arm(s) in the DNA to be integrated may promote the adoption and targeting of the insertion sequence into the plant genome during the repair process through homologous recombination, although an insertion event may occur through non-homologous end joining (NHEJ). As used herein, the term “double-strand break inducing agent” refers to any agent that can induce a double-strand break (DSB) in a DNA molecule. In some embodiments, the double-strand break inducing agent is a site-specific genome modification enzyme.

As used herein, the term “site-specific genome modification enzyme” refers to any enzyme that can modify a nucleotide sequence in a sequence-specific manner. In some embodiments, a site-specific genome modification enzyme modifies the genome by inducing a single-strand break. In some embodiments, a site-specific genome modification enzyme modifies the genome by inducing a double-strand break. In some embodiments, a site-specific genome modification enzyme comprises a cytidine deaminase. In some embodiments, a site specific genome modification enzyme comprises an adenine deaminase. Site-specific genome modification enzymes include endonucleases, recombinases, transposases, deaminases, helicases and any combination thereof. In some embodiments, the site-specific genome modification enzyme is a sequence-specific nuclease.

In one aspect, the endonuclease is selected from a meganuclease, a zinc-finger nuclease (ZFN), a transcription activator-like effector nucleases (TALEN), an Argonaute (non-limiting examples of Argonaute proteins include Thermus thermophilus Argonaute (TtAgo), Pyrococcus furiosus Argonaute (PfAgo), and Natronobacterium gregoryi Argonaute (NgAgo)), an RNA-guided nuclease, such as a CRISPR associated nuclease (non-limiting examples of CRISPR associated nucleases include, but are not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, homologs thereof, or modified versions thereof).

In some embodiments, the site-specific genome modification enzyme is a recombinase. Non-limiting examples of recombinases include a tyrosine recombinase attached to a DNA recognition motif and is selected from the group consisting of a Cre recombinase, a Gin recombinase, a Flp recombinase, and a Tnp1 recombinase. In one aspect, a Cre recombinase or a Gin recombinase is tethered to a zinc-finger DNA-binding domain, or a TALE DNA binding domain, or a Cas9 nuclease. In another aspect, a serine recombinase attached to a DNA recognition motif is selected from the group consisting of a PhiC31 integrase, an R4 integrase, and a TP-901 integrase. In another aspect, a DNA transposase attached to a DNA binding domain provided herein is selected from the group consisting of a TALE-piggyBac and TALE-Mutator.

Any DNA of interest as provided herein can be integrated into a target site of a chromosome sequence by introducing the DNA of interest and the disclosed site-specific genome modification enzymes. Any method provided herein can utilize any site-specific genome modification enzyme disclosed herein.

### VI. Antisense and RNAi Constructs

Antisense and RNAi treatments represent one way of altering mogroside biosynthesis pathway gene activity in accordance with the present disclosure (e.g., by down regulation of genes or transcription factors that inhibit expression of mogroside biosynthesis pathway genes).

Techniques for RNAi are well known in the art and are described in, for example, Lehner et al., (Brief Funct. Genomic Proteomic 3:68-83, 2004) and Downward (BMJ 328:1245-1248, 2004). The technique is based on the fact that double stranded RNA is capable of directing the degradation of messenger RNA with sequence complementary to one or the other strand (Fire et al., Nature 391:806-811, 1998). Therefore, by expression of a particular coding sequence in sense and antisense orientation, either as a fragment or longer portion of the corresponding coding sequence, the expression of that coding sequence can be down-regulated.

Antisense, and in some aspects RNAi, methodology takes advantage of the fact that nucleic acids tend to pair with “complementary” sequences. By complementary, it is meant that polynucleotides are those that are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense oligonucleotides, when introduced into a target cell, specifically bind to their target polynucleotide and interfere with transcription, RNA processing, transport, translation and/or stability. Antisense and RNAi constructs, or DNA encoding such RNA's, may be employed to inhibit gene transcription or translation or both within a host cell, either in vitro or in vivo, such as within a host plant cell. In certain embodiments of the present disclosure, such an oligonucleotide may comprise any unique portion of a nucleic acid sequence provided herein. In certain embodiments of the present disclosure, such a sequence comprises at least 18, 20, 25, 30, 50, 75 or 100 or more contiguous nucleic acids of a nucleic acid sequence of interest, and/or complements thereof, which may be in sense and/or antisense orientation. By including sequences in both sense and antisense orientation, increased suppression of the corresponding coding sequence may be achieved.

Constructs may be designed that are complementary to all or part of the promoter and other control regions, exons, introns or even exon-intron boundaries of a gene. It is contemplated that the most effective constructs may include regions complementary to intron/exon splice junctions. Thus, it is proposed that one embodiment includes a construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences that are completely complementary will be sequences that are entirely complementary throughout their entire length and have no base mismatches. Other sequences with lower degrees of homology also are contemplated. For example, an RNAi or antisense construct that has limited regions of high homology, but also contains a non-homologous region (e.g., ribozyme; see above) could be designed. Methods for selection and design of sequences that generate RNAi are well known in the art (e.g., Reynolds et al., Nat. Biotechnol. 22:326-330, 2004). These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone may be used. The cDNA or a synthesized polynucleotide may provide more convenient restriction sites for the remaining portion of the construct and, therefore, would be used for the rest of the sequence. Constructs useful for generating RNAi may also comprise concatemers of sub-sequences that display gene regulating activity.

### VII. Transformation

In some embodiments, transgenic plants of the present disclosure are created by transforming the selected natural plants with one or more of the expression cassettes disclosed herein. The natural plants prior to transformation do not naturally produce all mogrol/mogroside pathway enzymes, and do not produce non-native mogrol and mogroside compounds. Although the natural plants may produce one or more enzymes capable of producing mogrol precursors or mogrol, these plants do not produce non-native mogrosides naturally. In certain embodiments, the selected natural plants for transformation include wild-type, or untransformed, or non-transformed watermelons, which do not naturally produce detectable amounts of mogrol or mogroside compounds. Other plants that can be transformed with one or more of the expression cassettes disclosed herein include, but are not limited to, cantaloupe, honeydew, winter melon, casaba melon, Persian melon, citron melon, muskmelon, bailan melon, crenshaw melon, Christmas melon, sprite melon, caravelle melon, hami melon, rocky melon, golden Langkawi melon, Korean melon, saticoy melon, galia melon, jade dew melon, golden prize melon, ten me melon, new century melon, banana melon, yubari king melon, sugar melon, tiger melon, vert grimpant melon, horned melon, cucamelon, casabanana melon, pepino melon, ananas melon, camouflage melon, canary melon, bitter melon, charentais melon, crane melon, SkyRocket melon, honey globe melon, gac melon, autumn sweet melon, snap melon, cucumber, tomato, lettuce, spinach, rice, oat, maize, sorghum, bitter apple (Citrullus colocynthis), pumpkin, chard, tobacco, switch grass, bush cherry, peach, nectarine, apricot, radish, plum, sour cherry, apple, pear, sweet cherry, citrus, zucchini, pea, turnip or Nicotiana benthamiana plants. Mogroside compounds can be isolated from any part of the transformed plant, including, but not limited to, the fruit (juice or rind), leaves, roots, seeds or flowers.

Suitable methods for transformation of plant or other cells for use with the current disclosure are believed to include virtually any method by which DNA can be introduced into a cell, such as by direct delivery of DNA such as by PEG-mediated transformation of protoplasts (Omirulleh et al., Plant. Mol. Biol. 21:414-428, 1993), by desiccation/inhibition-mediated DNA uptake (Potrykus et al., Mol. Gen. Genet. 199:169-177, 1985), by electroporation (U.S. Pat. No. 5,384,253, specifically incorporated herein by reference in its entirety), by agitation with silicon carbide fibers (U.S. Pat. Nos. 5,302,523 and 5,464,765, specifically incorporated herein by reference in their entirety), by Agrobacterium-mediated transformation (U.S. Pat. Nos. 5,591,616 and 5,563,055; both specifically incorporated herein by reference in their entirety) and by acceleration of DNA coated particles (U.S. Pat. Nos. 5,550,318; 5,538,877; and 5,538,880; each specifically incorporated herein by reference in their entirety), etc. Through the application of techniques such as these, the cells of virtually any plant species may be transiently transformed, or stably transformed, and these cells developed into transgenic plants.

### A. Agrobacterium-mediated Transformation

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example, the methods described by Fraley et al., (Proc. Natl. Acad. Sci. USA 80:4803-4807, 1985), and U.S. Pat. No. 5,563,055, specifically incorporated herein by reference in its entirety.

Agrobacterium-mediated transformation is most efficient in dicotyledonous plants and is an efficient method for transformation of dicots, including Arabidopsis, tobacco, tomato, alfalfa and potato. Indeed, while Agrobacterium-mediated transformation has been routinely used with dicotyledonous plants for a number of years, it has only recently become applicable to monocotyledonous plants. Advances in Agrobacterium-mediated transformation techniques have now made the technique applicable to nearly all monocotyledonous plants. For example, Agrobacterium-mediated transformation techniques have now been applied to rice (Hiei et al., Plant Mol. Biol. 35:205-218, 1997; U.S. Pat. No. 5,591,616, specifically incorporated herein by reference in its entirety), wheat and barley (McCormac et al., Mol. Biotechnol. 9:155-159, 1998), alfalfa and maize (Ishida et al., Nat. Biotechnol. 14:745-750, 1996). Similarly, Agrobacterium-mediated transformation has also proven to be effective in switchgrass.

Modern Agrobacterium transformation vectors are capable of replication in E. coli as well as Agrobacterium, allowing for convenient manipulations. Moreover, recent technological advances in vectors for Agrobacterium-mediated gene transfer have improved the arrangement of genes and restriction sites in the vectors to facilitate the construction of vectors capable of expressing various polypeptide coding genes. The vectors have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant strains where Agrobacterium-mediated transformation is efficient, it is the method of choice because of the facile and defined nature of the gene transfer.

### B. Electroporation

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wounding in a controlled manner. Examples of some species that have been transformed by electroporation of intact cells include maize (U.S. Pat. No. 5,384,253, incorporated herein by reference in its entirety; Rhodes et al., Methods Mol. Biol. 55:121-131, 1995; D'Halluin et al., Plant Cell 4:1495-1505, 1992), wheat (Zhou et al., Plant Cell Rep. 12:612-616, 1993), tomato (Tsukada et al., Plant Cell Physiol. 30:599-603, 1989), soybean (Christou et al., Proc. Natl. Acad. Sci. USA 84:3962-3966, 1987) and tobacco (Riggs and Bates, Proc. Natl. Acad. Sci. USA 83:5602-5606, 1986).

One also may employ protoplasts for electroporation transformation of plants (Bates, Mol. Biotechnol. 2:135-145, 1994; Lazzeri, Methods Mol. Biol. 49:95-106, 1995). For example, the generation of transgenic soybean plants by electroporation of cotyledon-derived protoplasts is described in WO 9217598 (specifically incorporated herein by reference). Other examples of species for which protoplast transformation has been described include barley (Lazzeri, supra), sorghum (Battraw et al., Theor. Appl. Genet. 82:161-168, 1991), maize (Rhodes et al., Science 240:204-207, 1988), wheat (He et al., Plant Cell Rep. 14:192-196, 1994) and tomato (Tsukada, supra).

### C. Microprojectile Bombardment

Another method for delivering transforming DNA segments to plant cells in accordance with the present disclosure is microprojectile bombardment (U.S. Pat. Nos. 5,550,318; 5,538,880; 5,610,042; and PCT Application WO 94/09699; each of which is specifically incorporated herein by reference in its entirety). In this method, particles may be coated with nucleic acids and delivered into cells by a propelling force. Exemplary particles include those comprised of tungsten, platinum, and often, gold. It is contemplated that in some instances DNA precipitation onto metal particles would not be necessary for DNA delivery to a recipient cell using microprojectile bombardment. However, it is contemplated that particles may contain DNA rather than be coated with DNA. Hence, it is proposed that DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a filter surface covered with monocot plant cells cultured in suspension. The screen disperses the particles so that they are not delivered to the recipient cells in large aggregates. Microprojectile bombardment techniques are widely applicable, and may be used to transform virtually any plant species. Examples of species that have been transformed by microprojectile bombardment include monocot species such as maize (PCT Application WO 95/06128), barley (Ritala et al., Plant Mol. Biol. 24:317-325, 1994; Hensgens et al., Plant Mol. Biol. 22:1101-1127, 1993), wheat (U.S. Pat. No. 5,563,055, specifically incorporated herein by reference in its entirety), rice (Hensgens et al., supra), oat (Torbet et al., Crop Science 38:226-231, 1998), rye (Hensgens et al., supra), sugarcane (Bower et al., Plant J. 2:409-416, 1992), and sorghum (Casas et al., Proc. Natl. Acad. Sci. USA 90:11212-11216, 1993; Hagio et al., Plant Cell Rep. 10:260-264, 1991); as well as a number of dicots including tobacco (Tomes et al., Plant Mol. Biol. 14:261-268, 1990), soybean (U.S. Pat. No. 5,322,783, specifically incorporated herein by reference in its entirety), sunflower (Knittel et al., Plant Cell Rep. 14:81-86, 1994), peanut (Singsit et al., Transgenic Res. 6:169-176, 1997), cotton (McCabe and Martinell, Nat. Biotechnol. 11:596-598, 1993), tomato (VanEck et al., Plant Cell. Rep. 14:299-304, 1995), switchgrass (Richards et al., Plant Cell Rep. 20:48-54, 2001) and legumes in general (U.S. Pat. No. 5,563,055, specifically incorporated herein by reference in its entirety).

### D. Other Transformation Methods

Transformation of protoplasts can be achieved using methods based on calcium phosphate precipitation, polyethylene glycol treatment, electroporation, and combinations of these treatments (see, e.g., Potrykus et al., supra; Omirulleh et al., supra;). Application of these systems to different plant strains depends upon the ability to regenerate that particular plant strain from protoplasts. Illustrative methods for the regeneration of cereals from protoplasts have been described (Toriyama et al., Nat. Biotechnol. 6:1072-1074, 1988; Abdullah et al., Nat. Biotechnol. 4:1087-1090, 1986; Omirulleh et al., supra, and U.S. Pat. No. 5,508,184; each specifically incorporated herein by reference in its entirety). Examples of the use of direct uptake transformation of cereal protoplasts include transformation of rice (Ghosh-Biswas et al., J. Biotechnol. 32:1-10, 1994), sorghum (Battraw et al., supra), barley (Lazzeri, supra), oat, and maize (Omirulleh et al., supra).

To transform plant strains that cannot be successfully regenerated from protoplasts, other ways to introduce DNA into intact cells or tissues can be utilized. For example, regeneration of cereals from immature embryos or explants can be effected as described (Vasil, supra). Also, silicon carbide fiber-mediated transformation may be used with or without protoplasting (Kaeppler et al., Theor. Appl. Genet. 84:560-566, 1992; U.S. Pat. No. 5,563,055, specifically incorporated herein by reference in its entirety). Transformation with this technique is accomplished by agitating silicon carbide fibers together with cells in a DNA solution. DNA passively enters as the cells are punctured. This technique has been used successfully with, for example, the monocot cereals maize (PCT Application WO 95/06128, specifically incorporated herein by reference in its entirety) and rice (Nagatani et al., Biotechnol. Tech. 11:471-473, 1997).

### E. Tissue Cultures

Tissue cultures may be used in certain transformation techniques for the preparation of cells for transformation and for the regeneration of plants therefrom. Maintenance of tissue cultures requires use of media and controlled environments. “Media” refers to the numerous nutrient mixtures that are used to grow cells in vitro, that is, outside of the intact living organism. The medium usually is a suspension of various categories of ingredients (salts, amino acids, growth regulators, sugars, buffers) that are required for growth of most cell types. However, each specific cell type requires a specific range of ingredient proportions for growth, and an even more specific range of formulas for optimum growth. Rate of cell growth also will vary among cultures initiated with the array of media that permit growth of that cell type.

Nutrient media is prepared as a liquid, but this may be solidified by adding the liquid to materials capable of providing a solid support. Agar is most commonly used for this purpose. BACTO®AGAR, GELRITE®, and GELGRO® are specific types of solid support that are suitable for growth of plant cells in tissue culture.

Some cell types will grow and divide either in liquid suspension or on solid media. As disclosed herein, plant cells will grow in suspension or on solid medium, but regeneration of plants from suspension cultures typically requires transfer from liquid to solid media at some point in development. The type and extent of differentiation of cells in culture will be affected not only by the type of media used and by the environment, for example, pH, but also by whether media is solid or liquid.

Tissue that can be grown in a culture includes meristem cells, Type I, Type II, and Type III callus, immature embryos and gametic cells such as microspores, pollen, sperm and egg cells. Type I, Type II, and Type III callus may be initiated from tissue sources including, but not limited to, immature embryos, seedling apical meristems, root, leaf, microspores and the like. Those cells that are capable of proliferating as callus also are recipient cells for genetic transformation.

Somatic cells are of various types. Embryogenic cells are one example of somatic cells that may be induced to regenerate a plant through embryo formation. Non-embryogenic cells are those that typically will not respond in such a fashion. Certain techniques may be used that enrich recipient cells within a cell population. For example, Type II callus development, followed by manual selection and culture of friable, embryogenic tissue, generally results in an enrichment of cells. Manual selection techniques that can be employed to select target cells may include, e.g., assessing cell morphology and differentiation, or may use various physical or biological means. Cryopreservation also is a possible method of selecting for recipient cells.

Manual selection of recipient cells, e.g., by selecting embryogenic cells from the surface of a Type II callus, is one means that may be used in an attempt to enrich for particular cells prior to culturing (whether cultured on solid media or in suspension).

Where employed, cultured cells may be grown either on solid supports or in the form of liquid suspensions. In either instance, nutrients may be provided to the cells in the form of media, and environmental conditions controlled. There are many types of tissue culture media comprised of various amino acids, salts, sugars, growth regulators and vitamins. Most of the media employed in the practice of the present disclosure will have some similar components, but may differ in the composition and proportions of their ingredients depending on the particular application envisioned. For example, various cell types usually grow in more than one type of media, but will exhibit different growth rates and different morphologies, depending on the growth media. In some media, cells survive but do not divide. Various types of media suitable for culture of plant cells previously have been described. Examples of these media include, but are not limited to, the N6 medium described by Chu et al., (Sci. Sin. [Peking] 18:659-668, 1975) and MS media (Murashige and Skoog, Physiol. Plant 15:473-479, 1962).

### VIII. Production and Characterization of Stably Transformed Plants

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the present disclosure. In this case, one would then generally assay the potentially transformed cell population by exposing the cells to a selective agent or agents, or one would screen the cells for the desired marker gene trait.

### A. Selection

It is believed that DNA is introduced into only a small percentage of target cells in any one study. In order to provide an efficient system for identification of those cells receiving DNA and integrating it into their genomes one may employ a means for selecting those cells that are stably transformed. One exemplary embodiment of such a method is to introduce into the host cell, a marker gene that confers resistance to some normally inhibitory agent, such as an antibiotic or herbicide. Examples of antibiotics that may be used include the aminoglycoside antibiotics neomycin, kanamycin and paromomycin, or the antibiotic hygromycin. Resistance to the aminoglycoside antibiotics is conferred by aminoglycoside phosphotransferase enzymes such as neomycin phosphotransferase II (NPT II) or NPT I, whereas resistance to hygromycin is conferred by hygromycin phosphotransferase.

Potentially transformed cells then are exposed to the selective agent. In the population of surviving cells will be those cells where, generally, the resistance-conferring gene has been integrated and expressed at sufficient levels to permit cell survival. Cells may be tested further to confirm stable integration of the exogenous DNA.

One herbicide that constitutes a desirable selection agent is the broad spectrum herbicide bialaphos. Bialaphos is a tripeptide antibiotic produced by Streptomyces hygroscopicus and is composed of phosphinothricin (PPT), an analogue of L-glutamic acid, and two L-alanine residues. Upon removal of the L-alanine residues by intracellular peptidases, the PPT is released and is a potent inhibitor of glutamine synthetase (GS), a pivotal enzyme involved in ammonia assimilation and nitrogen metabolism (Ogawa et al., Sci. Rep. Meiji Seika 13:42-48, 1973). Synthetic PPT, the active ingredient in the herbicide Liberty™ also is effective as a selection agent. Inhibition of GS in plants by PPT causes the rapid accumulation of ammonia and death of the plant cells.

The organism producing bialaphos and other species of the genus Streptomyces also synthesizes an enzyme phosphinothricin acetyl transferase (PAT), which is encoded by the bar gene in Streptomyces hygroscopicus and the pat gene in Streptomyces viridochromogenes. The use of the herbicide resistance gene encoding phosphinothricin acetyl transferase (PAT) is referred to in DE 3642 829 A, wherein the gene is isolated from Streptomyces viridochromogenes. In the bacterial source organism, this enzyme acetylates the free amino group of PPT preventing auto-toxicity (Thompson et al., EMBO J. 6:2519-2523, 1987). The bar gene has been cloned (Thompson et al., supra) and expressed in transgenic tobacco, tomato, potato (De Block et al., EMBO J. 6:2513-2518, 1987) Brassica (De Block et al., Plant Physiol. 91:694-701, 1989) and maize (U.S. Pat. No. 5,550,318, incorporated herein by reference in its entirety).

Another example of a herbicide that is useful for selection of transformed cell lines in the practice of the present disclosure is the broad spectrum herbicide glyphosate. Glyphosate inhibits the action of the enzyme EPSPS, which is active in the aromatic amino acid biosynthetic pathway. Inhibition of this enzyme leads to starvation for the amino acids phenylalanine, tyrosine, and tryptophan and secondary metabolites derived thereof. U.S. Pat. No. 4,535,060 (incorporated herein by reference in its entirety) describes the isolation of EPSPS mutations that confer glyphosate resistance on the Salmonella typhimurium gene for EPSPS, aroA. The EPSPS gene was cloned from Zea mays and mutations similar to those found in a glyphosate resistant aroA gene were introduced in vitro. Mutant genes encoding glyphosate resistant EPSPS enzymes are described in, for example, International Patent WO 97/4103.

To use the bar-bialaphos or the EPSPS-glyphosate selective system, transformed tissue is cultured for 0-28 days on nonselective medium and subsequently transferred to medium containing from 1-3 mg/l bialaphos or 1-3 mM glyphosate as appropriate. While ranges of 1-3 mg/l bialaphos or 1-3 mM glyphosate may be beneficial, it is proposed that ranges of 0.1-50 mg/l bialaphos or 0.1-50 mM glyphosate will find utility.

An example of a screenable marker trait is the enzyme luciferase. In the presence of the substrate luciferin, cells expressing luciferase emit light that can be detected on photographic or x-ray film, in a luminometer (or liquid scintillation counter), by devices that enhance night vision, or by a highly light sensitive video camera, such as a photon counting camera. These assays are nondestructive and transformed cells may be cultured further following identification. The photon counting camera is especially valuable as it allows one to identify specific cells or groups of cells that are expressing luciferase and manipulate those in real time. Another screenable marker that may be used in a similar fashion is the gene coding for green fluorescent protein.

### B. Regeneration and Seed Production

Cells that survive the exposure to the selective agent, or cells that have been scored positive in a screening assay, may be cultured in media that supports regeneration of plants. In an exemplary embodiment, MS and N6 media may be modified by including further substances such as growth regulators. One such growth regulator is dicamba or 2,4-D. However, other growth regulators may be employed, including NAA, NAA+2,4-D or picloram. Media improvement in these and like ways has been found to facilitate the growth of cells at specific developmental stages. Tissue may be maintained on a basic media with growth regulators until sufficient tissue is available to begin plant regeneration efforts, or following repeated rounds of manual selection, until the morphology of the tissue is suitable for regeneration, at least 2 weeks, then transferred to media conducive to maturation of embryoids. Cultures are transferred every 2 weeks on this medium. Shoot development will signal the time to transfer to medium lacking growth regulators.

The transformed cells, identified by selection or screening and cultured in an appropriate medium that supports regeneration, will then be allowed to mature into plants. Developing plantlets are transferred to soilless plant growth mix, and hardened, e.g., in an environmentally controlled chamber, for example, at about 85% relative humidity, 600 ppm CO₂, and 25-250 microeinsteins m 2 s-1 of light. Plants may be matured in a growth chamber or greenhouse. Plants can be regenerated from about 6 weeks to 10 months after a transformant is identified, depending on the initial tissue. During regeneration, cells are grown on solid media in tissue culture vessels. Illustrative embodiments of such vessels are petri dishes and Plant Cons. Regenerating plants can be grown at about 19 to 28° C. After the regenerating plants have reached the stage of shoot and root development, they may be transferred to a greenhouse for further growth and testing.

Seeds on transformed plants may occasionally require embryo rescue due to cessation of seed development and premature senescence of plants. To rescue developing embryos, they are excised from surface-disinfected seeds 10-20 days post-pollination and cultured. An embodiment of media used for culture at this stage comprises MS salts, 2% sucrose, and 5.5 g/l agarose. In embryo rescue, large embryos (defined as greater than 3 mm in length) are germinated directly on an appropriate media. Embryos smaller than that may be cultured for 1 week on media containing the above ingredients along with 10-5 M abscisic acid and then transferred to growth regulator-free medium for germination.

### C. Characterization

To confirm the presence of the exogenous DNA or “transgene(s)” in the regenerating plants, a variety of assays may be performed. Such assays include, for example, “molecular biological” assays, such as Southern and Northern blotting and PCR™; “biochemical” assays, such as detecting the presence of a protein product, e.g., by immunological means (ELISAs and Western blots) or by enzymatic function; plant part assays, such as leaf or root assays; and also, by analyzing the phenotype of the whole regenerated plant.

### D. DNA Integration, RNA Expression and Inheritance

Genomic DNA may be isolated from cell lines or any plant parts to determine the presence of the exogenous gene through the use of techniques well known to those skilled in the art. Note, that intact sequences will not always be present, presumably due to rearrangement or deletion of sequences in the cell. The presence of DNA elements introduced through the methods of this disclosure may be determined, for example, by polymerase chain reaction (PCR™). Using this technique, discreet fragments of DNA are amplified and detected by gel electrophoresis. This type of analysis permits one to determine whether a gene is present in a stable transformant, but does not prove integration of the introduced gene into the host cell genome. It is typically the case, however, that DNA has been integrated into the genome of all transformants that demonstrate the presence of the gene through PCR™ analysis. In addition, it is not typically possible using PCR™ techniques to determine whether transformants have exogenous genes introduced into different sites in the genome, i.e., whether transformants are of independent origin. It is contemplated that using PCR™ techniques it would be possible to clone fragments of the host genomic DNA adjacent to an introduced gene.

Positive proof of DNA integration into the host genome and the independent identities of transformants may be determined using the technique of Southern hybridization. Using this technique specific DNA sequences that were introduced into the host genome and flanking host DNA sequences can be identified. Hence the Southern hybridization pattern of a given transformant serves as an identifying characteristic of that transformant. In addition it is possible through Southern hybridization to demonstrate the presence of introduced genes in high molecular weight DNA, i.e., confirm that the introduced gene has been integrated into the host cell genome. The technique of Southern hybridization provides information that is obtained using PCR™, e.g., the presence of a gene, but also demonstrates integration into the genome and characterizes each individual transformant.

It is contemplated that using the techniques of dot or slot blot hybridization, which are modifications of Southern hybridization techniques, one could obtain the same information that is derived from PCR™, e.g., the presence of a gene.

Both PCR™ and Southern hybridization techniques can be used to demonstrate transmission of a transgene to progeny. In most instances the characteristic Southern hybridization pattern for a given transformant will segregate in progeny as one or more Mendelian genes (Spencer et al., 1992) indicating stable inheritance of the transgene.

Whereas DNA analysis techniques may be conducted using DNA isolated from any part of a plant, RNA will only be expressed in particular cells or tissue types and hence it will be necessary to prepare RNA for analysis from these tissues. PCR™ techniques also may be used for detection and quantitation of RNA produced from introduced genes. In this application of PCR™ it is first necessary to reverse transcribe RNA into DNA, using enzymes such as reverse transcriptase, and then through the use of conventional PCR™ techniques amplify the DNA. In most instances PCR™ techniques, while useful, will not demonstrate integrity of the RNA product. Further information about the nature of the RNA product may be obtained by Northern blotting. This technique will demonstrate the presence of an RNA species and give information about the integrity of that RNA. The presence or absence of an RNA species also can be determined using dot or slot blot Northern hybridizations. These techniques are modifications of Northern blotting and will only demonstrate the presence or absence of an RNA species.

### E. Gene Expression

While Southern blotting and PCR™ may be used to detect the gene(s) in question, they do not provide information as to whether the corresponding protein is being expressed. Expression may be evaluated by specifically identifying the protein products of the introduced genes or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures also may be used to identify the expression of proteins by their functionality, especially the ability of enzymes to catalyze specific chemical reactions involving specific substrates and products. These reactions may be followed by providing and quantifying the loss of substrates or the generation of products of the reactions by physical or chemical procedures. Examples are as varied as the enzyme to be analyzed and may include assays for PAT enzymatic activity by following production of radiolabeled acetylated phosphinothricin from phosphinothricin and ¹⁴C-acetyl CoA or for anthranilate synthase activity by following loss of fluorescence of anthranilate, to name two.

Very frequently the expression of a gene product is determined by evaluating the phenotypic results of its expression. These assays also may take many forms including, but not limited to, analyzing changes in the chemical composition, morphology, or physiological properties of the plant. Chemical composition may be altered by expression of genes encoding enzymes or storage proteins that change amino acid composition and may be detected by amino acid analysis, or by enzymes that change starch quantity, which may be analyzed by near infrared reflectance spectrometry. Morphological changes may include greater stature or thicker stalks. Most often changes in response of plants or plant parts to imposed treatments are evaluated under carefully controlled conditions termed bioassays.

### IX. Mogroside Containing Sweeteners and Consumables

In some embodiments, the present disclosure relates generally to a sweetener or sweetening composition comprising mogroside and/or metabolites or derivatives thereof, wherein the sweetener or sweetening composition is derived from a transgenic plant producing and comprising non-native mogrol/mogrosides. The term “sweetener”, as used herein, refers to a consumable product, which produces a sweet taste when consumed alone. In certain embodiments, the sweetener or sweetening composition is derived from the mogrol/mogroside pathway transgenic plants made according to the present disclosure. In some embodiments the sweeteners are high intensity or low intensity sweeteners. Mogroside-containing sweeteners can be derived from the mogrol/mogroside pathway transgenic plants of the present disclosure upon appropriate processing. The resulting sweeteners could be used to provide low or non-caloric sweetness for many purposes. Examples of such uses to provide sweetness are in beverages, such as tea, coffee, fruit juice, and fruit beverages, foods, such as jams and jellies, peanut butter, pies, puddings, cereals, candies, ice creams, yogurts, bakery products; health care products, such as toothpastes, mouthwashes, cough drops, cough syrups; chewing gums; and sugar substitutes.

In certain embodiments, the sweetener is in a juice of the fruit from a transgenic plant according to the present disclosure. Applications for juice, for example, watermelon juice, containing one or more mogroside compound include, but are not limited to, as a beverage, including, for example, premixed cocktails and dairy alternatives, as an ingredient, for example to be sprayed onto bars or cereal, or used to sweeten ketchup or other common products. In such embodiments the juice can be devitalized, have the protein removed or concentrated. Additionally concentrated fruit or vegetable syrup, for example watermelon syrup produced from the presently disclosed watermelons, can be used to substitute for high fructose corn syrup in various foods and beverages. In some embodiments of the present disclosure the mogroside compounds are produced in transgenic tomatoes, which can then be used, for example, to produce lower calorie ketchup or other tomato-based sauces or soups.

In some embodiments, the present disclosure also relates to methods of making a sweetener derived from the presently disclosed transgenic plants producing non-native mogrol/mogrosides. The methods generally encompasses steps that can include, but are not limited to, pre-treatment cleaning and crushing of the transgenic plant or the parts thereof, extraction of the transgenic plant or the parts thereof, sedimentation and/or centrifuge, adsorption and/or separation, concentration and recovery to produce the crude sweetener, further purification, optional concentration/drying, and formulation. Means of extraction encompasses water-extraction at room temperatures, or heated temperature, or refrigerated temperature; extraction via organic solvent such as alcohol, etc. Means of separation and purification encompasses centrifuge, steeping, gravity sedimentation, filtration, micro-filtration, nano filtration, ultra-filtration, reverse osmosis, chromatography, absorption chromatogram, exchanged resin purification, etc.

In further embodiments the presently disclosed transgenic plants can be processed to produce mogroside-containing ingredients, for example by whole plant extracts, tissue extraction, fruit processing, aqueous separation of small molecules having a mogroside fraction, removal of residual proteins to yield an aqueous fraction free from any genetically engineered components. The resulting mogroside containing ingredient(s) can be in any form, including, but not limited to, a powder, liquid, syrup, concentrate or extract. Additionally in some embodiments a whole mogroside containing fruit or vegetable is the consumable.

In certain embodiments, the sweetener is obtained from the leaves of the transgenic plant made according to the present disclosure. In other embodiments, the sweetener is obtained from the fruit, a part of a fruit (e.g., the rind), or other part of an organ or tissue of the transgenic plant made according to the present disclosure.

Additionally the mogroside compounds produced by the presently disclosed transgenic plants and organisms can be blended with one or more other naturally occurring or artificial sweeteners, such as steviol glycosides, siamenoside I, α-siamenoside I, sucrose, glucose, fructose, lactose, maltose, sorbitol, galactose, thaumtin, sucrooctate, bernadame, sucrononic acid, carrelame, lugduname, high fructose corn syrup, RealSweet™ Sugarcane RebM, erythritol, xylitol, yacon syrup, allulose, saccharin, aspartame, acesulfame potassium, sucralose, neotame, advantame, cyclamates or glycyrrhizin. The ratio of the mogroside compound(s) to the other sweetener in the final formulation can be, for example, 10/90, 20/80, 30/70, 40/60/50/50, 60/40, 70/30, 80/20 or 90/10, or any other desired ratio. In one embodiment, the ratio is about 80% mogroside V, about 15% 11-oxo-mogroside V and about 5% mogroside III A1. In another embodiment, the ratio is about 40% siamenoside I, about 40% mogroside V and about 20% 11-oxo-mogroside V.

In certain embodiments, the one or more additional sweeteners may be a carbohydrate sweetener. Non-limiting examples of suitable carbohydrate sweeteners include sucrose, fructose, glucose, erythritol, maltitol, lactitol, sorbitol, mannitol, xylitol, tagatose, trehalose, galactose, rhamnose, cyclodextrin (e.g., α-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, fuculose, glucuronic acid, gluconic acid, glucono-lactone, abequose, galactosamine, xylo-oligosaccharides (xylotriose, xylobiose and the like), gentio-oligoscaccharides (gentiobiose, gentiotriose, gentiotetraose and the like), galacto-oligosaccharides, sorbose, ketotriose (dehydroxyacetone), aldotriose (glyceraldehyde), nigero-oligosaccharides, fructooligosaccharides (kestose, nystose and the like), maltotetraose, maltotriol, tetrasaccharides, mannan-oligo saccharides, malto-oligosaccharides (maltotriose, maltotetraose, maltopentaose, maltohexaose, maltoheptaose and the like), dextrins, lactulose, melibiose, raffinose, rhamnose, ribose, isomerized liquid sugars such as high fructose corn/starch syrup (HFCS/HFSS) (e.g., HFCS55, HFCS42, or HFCS90), coupling sugars, soybean oligosaccharides, glucose syrup and combinations thereof. D- or L-configurations can be used when applicable. In other embodiments, the additional sweetener is a carbohydrate sweetener selected from the group consisting of glucose, fructose, sucrose and combinations thereof. In another embodiment, the additional sweetener is a carbohydrate sweetener selected from D-allose, D-psicose, L-ribose, D-tagatose, L-glucose, L-fucose, L-Arabinose, Turanose and combinations thereof.

In yet other embodiments, the one or more additional sweeteners is not directly derived from a natural extraction. Such a sweetener characteristically has a sweetness potency greater than sucrose, fructose, or glucose, yet has less calories. Non-limiting examples of such sweeteners suitable for embodiments of this disclosure include sucralose, potassium acesulfame, acesulfame acid and salts thereof, aspartame, alitame, saccharin and salts thereof, neohesperidin dihydrochalcone, cyclamate, cyclamic acid and salts thereof, neotame, advantame, glucosylated steviol glycosides (GSGs) and combinations thereof. The at least one sweetener not directly derived from natural extraction is present in the sweetener composition in an amount effective to provide a concentration from about 0.3 ppm to about 3,500 ppm when present in a sweetened composition, such as, for example, a food, other consumable or beverage. In one embodiment, the at least one sweetener not directly derived from natural extraction is present in the sweetener composition in an amount effective to provide a concentration from about 0.5 ppm to about 3,000 ppm, from about 1.0 ppm to about 2,500 ppm, from about 5.0 ppm to about 2,000 ppm, from about 10 ppm to about 1,500 ppm, from about 50 ppm to about 1000 ppm, from about 100 ppm to about 800 ppm, or from about 400 ppm to about 600 ppm when present in a sweetened beverage. In another embodiment, the at least one embodiment, the at least one sweetener not directly derived from natural extraction is present in the sweetener composition in an amount effective to provide a concentration greater than about 0.3 ppm, greater than about 0.5 ppm, greater than about 1.0 ppm, greater than about 5.0 ppm, greater than about 10 ppm, greater than about 20 ppm, greater than about 50 ppm, greater than about 100 ppm, greater than about 250 ppm, greater than about 500 ppm or greater than about 1000 ppm when present in a sweetened composition, such as, for example, a food, other consumable or beverage.

In still other embodiments, the additional sweetener can be a natural high potency sweetener. Suitable natural high potency sweeteners include, but are not limited to, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside M, rebaudioside N, rebaudioside O, dulcoside A, dulcoside B, rubusoside, Stevia, stevioside, mogroside IV, mogroside V, Luo Han Guo, miraculin, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, steviolbioside and cyclocarioside I. The natural high potency sweetener can be provided as a pure compound or, alternatively, as part of an extract. For example, rebaudioside A can be provided as a sole compound or as part of a Stevia extract. The natural high potency sweetener is present in the sweetener composition in an amount effective to provide a concentration from about 0.1 ppm to about 3,000 ppm when present in a sweetened composition, such as, for example, a food, other consumable or beverage. In one embodiment, the natural high potency sweetener is present in the sweetener composition in an amount effective to provide a concentration from about 0.5 ppm to about 2500 ppm, from about 1.0 ppm to about 2000 ppm, from about 5 ppm to about 1500 ppm, from about 10 ppm to about 1000 ppm, or about 25 ppm to about 500 ppm when present in a sweetened composition, such as, for example, a food, other consumable or beverage. In one embodiment, the natural high potency sweetener is present in the sweetener composition in an amount effective to provide a concentration of greater than about 0.1 ppm, about 0.5 ppm, about 1.0 ppm, about 2.5 ppm, about 5.0 ppm, about 10 ppm, about 20 ppm, about 25 ppm, about 50 ppm, about 75 ppm, about 100 ppm, about 200 ppm, about 500 ppm, about 1000 ppm, about 2000 ppm, or about 300 ppm when present in a sweetened composition, such as, for example, a food, other consumable or beverage.

In still other embodiments, the additional sweetener can be chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by an enzymatic transglycosylation reaction catalyzed by various enzymes possessing transglycosylating activity.

When the sweetener composition contains more than one sweetener, the sweeteners may exhibit synergy when combined and have improved flavor and temporal profiles compared to each sweetener alone. As used herein, the term “temporal profile” of a composition means the intensity of sweetness perceived over time in tasting of a composition by a human. The term “flavor profile” or “taste profile,” as generally used herein, refers to the intensity of various flavor/taste attributes of a sweetener or sweetened composition. Exemplary flavor/taste attributes are sweetness intensity, bitterness intensity, salty intensity, licorice intensity, cooling intensity, and licorice intensity. Methods of determining the flavor profile of a given sweetener or sweetened composition are known in the art. The term “synergistic” or “synergistic effect” refers to an effect (e.g., flavor, temporal profile) achieved with the combination of two or more sweeteners which is greater than the sum of the effects that effect from using the particular sweeteners alone or separately. Advantageously, such synergy between the two or more sweeteners allows for the use of smaller doses of one or both sweeteners or provides greater effect at the same amounts. The amount or degree of synergism may vary.

The amount of sucrose in a reference solution may be described in degrees Brix (° Bx). One degree Brix is 1 gram of sucrose in 100 grams of solution and represents the strength of the solution as percentage by weight (% w/w) (strictly speaking, by mass). In one embodiment, a sweetener composition contains one or more of the presently disclosed sweetener compounds in an amount effective to provide sweetness equivalent from of at least about 5 degrees Brix of sugar when present in a sweetened composition, such as, for example, from at least about 5, at least about 6, at least about 7, at least about 8, at least about 9, at least about 10, at least about 11, at least about 12, at least about 13, at least about 14 or at least about 15 or more degrees Brix.

The sweetness of a non-sucrose sweetener can also be measured against a sucrose reference by determining the non-sucrose sweetener's sucrose equivalence. Typically, taste panelists are trained to detect sweetness of reference sucrose solutions containing between 1-15% sucrose (w/v). Other non-sucrose sweeteners are then tasted at a series of dilutions to determine the concentration of the non-sucrose sweetener that is as sweet as a given percent sucrose reference. For example, if a 1% solution of a sweetener is as sweet as a 10% sucrose solution, then the sweetener is said to be 10 times as potent as sucrose.

The sweetener compositions can be customized to provide the desired calorie content. For example, sweetener compositions can be “full-calorie”, such that they impart the desired sweetness when added to a sweetenable composition (such as, for example, a food, other consumable or beverage) and have about 120 calories per 8 oz. serving. Alternatively, sweetener compositions can be “mid-calorie”, such that they impart the desired sweetness when added to a sweetenable composition and have less than about 60 calories per 8 oz. serving. In other embodiments, sweetener compositions can be “low-calorie”, such that they impart the desired sweetness when added to a sweetenable composition and have less than 40 calories per 8 oz. serving. In still other embodiments, the sweetener compositions can be “zero-calorie”, such that they impart the desired sweetness when added to a sweetenable composition and have less than 5 calories per 8 oz. serving.

The presently disclosed the sweetener compositions can optionally include one or more additional additives. In some embodiments, the sweetener composition contains additives including, but not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, weighing agents, gums, antioxidants, colorants, flavonoids, alcohols, polymers and combinations thereof. In some embodiments, the additives act to improve the temporal and flavor profile of the sweetener to provide a sweetener composition with a taste similar to sucrose. The sweetened compositions can contain one or more functional ingredients, as detailed above. Functional ingredients include, but are not limited to, vitamins, minerals, antioxidants, preservatives, glucosamine, polyphenols and combinations thereof. Any suitable functional ingredient described herein can be used.

There is a beneficial environmental impact of the presently disclosed mogroside sweeteners compared to existing monkfruit production. The presently disclosed mogroside sweeteners enables local production, resulting in less transportation and fewer food miles as a result, compared to for example harvesting and processing only in China and shipping across the globe to food companies. The presently disclosed mogroside sweeteners also require minimal processing because of the ease of access to the mogroside sweeteners in the presently disclosed transgenic plants or organisms compared to, for example, factory processing of monkfruit sweetener in China.

### X. Sweetened Compositions

The presently disclosed sweetener compositions can be incorporated in any known edible material (referred to herein as a “sweetenable composition”), such as, for example, pharmaceutical compositions, edible gel mixes and compositions, dental compositions, foodstuffs (confections, condiments, chewing gum, cereal compositions baked goods dairy products, and tabletop sweetener compositions) beverages and beverage products.

The sweetened compositions disclosed here include beverages, i.e., ready to drink liquid formulations, beverage concentrates and the like. In certain embodiments, beverage concentrates are prepared with an initial volume of liquid (e.g., water) to which the additional ingredients are added. Full strength beverage compositions can be formed from the beverage concentrate by adding further volumes of liquid (e.g., water) to the concentrate.

In embodiments of sweetenable compositions using mogroside-containing filler juice concentrate (about 80% mogroside V, about 15% 11-oxo-mogroside V and about 5% mogroside III A1), studies have shown the taste to be the sweetest and cleanest tasting natural sweetener. The presently disclosed mogroside-containing filler juice concentrate can be produced from consumer friendly fruits that can be locally grown with sustainable production, is the only sweetener for high sugar reduction while maintaining 100% juice labeling, and is an affordable drop in solution. The filler juice may be used as single strength or concentrated to deliver clean, sweet taste across various inclusion levels. The filler juice mogroside concentrations can deliver the equivalent of −10 sucrose equivalent value (SEV) when used at various formula inclusions.

Besides concentration, other juice parameters are also readily changeable, resulting in different sweetener products. For example, the natural sugars in the fruit (for example watermelon) may be partially or fully removed, the juice color and/or flavor may be minimized or removed, the pulp may be removed as is typical, or remain fully or partially as in purees, and the acidity may be reduced, or combinations of one or more of these parameters can be changed.

Filler juice applications include, but are not limited to, juices, nectars, fruit/flavored still drinks, energy and sports drinks, carbonated soft drinks, flavored waters, nutritional drinks, vitamins and dietary supplements or oral rehydration in the form of liquids or chews/gummies, snacks such as snack bars or fruit snacks, sugar and gum confectionary in the form of jellies and chews, dairy products such as spoonable yogurt, drinking yogurt and flavored drinks, desserts, ice cream, frozen yogurt, water-based ice pops and sorbets, breakfast cereals and other cold cereals, tabletop sweeteners, sweet spreads such as syrups and fruit spreads, sauces and seasonings such as table sauces and cooking sauces, and processed and packaged fruit and vegetables.

In embodiments of sweetenable compositions using mogroside-containing dry powder (about 40% siamenoside I, about 40% mogroside V, and about 20% 11-oxo-mogroside V), studies have shown the taste to be clean, with high levels of sweetness with no off-tastes in demanding applications. The presently disclosed mogroside-containing dry powder can be produced from consumer friendly vegetables that can be locally grown with sustainable production, results in sugar and calorie reduction with strong positive associations to health benefits, and is a fraction of monk fruit/parity with sucrose. The dry powder may be used across a wide range of food and drink applications to deliver the cleanest, sweetest taste at low inclusion levels. The dry powder concentrations can deliver the equivalent of −10 SEV when used at various purity levels.

Dry powder application include, but are not limited to: wellness and functional drinks, such as energy and sports drinks, carbonated soft drinks, flavored waters, juices, nectars, fruit/flavored still drinks, protein and meal replacement drinks, drink mixes, drink concentrates, ready-to-drink tea and ready-to-drink coffee; dietary supplements and over-the-counter products, such as vitamins and dietary supplements, oral hydration, cold relief, digestive treatments, sleep aids, pain relief in capsule, tablet, liquid, powder, chew/gummy, lozenge and other formats; snacks such as snack bars, fruit snacks, nuts, trail mixes, corn rice, potato and wheat snacks; bakery products such as cookies, cakes and sweet goods, baking mixes and ingredients and breads; dairy and desserts such as spoonable and drinking yogurt, flavored drinks, creamers, ice cream and frozen yogurt, water-based ice pops and sorbets, shelf-stable desserts and dessert toppings; hot and cold breakfast cereals; artificial and other natural sweeteners (tabletop sweeteners); sugar and chocolate confectionary such as jellies and chews, mints, gum, toffee and caramels, marshmallows and various chocolate formats; sweet spreads such as syrups, fruit, nut and chocolate spreads; sauces and seasonings such as table, cooking and pasta sauces, vinegar and dressings and pickled condiments; meals and processed meats such as prepared meals, meal kits, sandwiches and wraps and poultry and meat products; and processed and packaged fruit and vegetables.

### A. Beverage and Beverage Products

In one embodiment, the sweetened composition is a beverage or beverage product. “Beverage product”, as used herein, is a ready-to-drink beverage, a beverage concentrate, a beverage syrup, or a powdered beverage. Suitable ready-to-drink beverages include carbonated and non-carbonated beverages. Carbonated beverages include, but are not limited to, frozen carbonated beverages, enhanced sparkling beverages, cola, fruit-flavored sparkling beverages (e.g., lemon-lime, orange, grape, strawberry and pineapple), ginger-ale, soft drinks and root beer. Non-carbonated beverages include, but are not limited to, fruit juice, fruit-flavored juice or water, juice drinks, nectars, fruit/flavored still drinks, energy and sports drinks, vegetable juice, vegetable-flavored juice, sports drinks, energy drinks, nutritional drinks, enhanced water drinks, enhanced water with vitamins, near water drinks (e.g., water with natural or synthetic flavorants), coconut water, tea type drinks (e.g., black tea, green tea, red tea, oolong tea), coffee, cocoa drink, beverage containing milk components (e.g., milk beverages, coffee containing milk components, cafe au lait, milk tea, fruit milk beverages), beverages containing cereal extracts and smoothies.

In certain embodiments, the beverage is a juice beverage that has been modified to remove at least some sucrose. In certain embodiments, such juice may be modified to remove at least 10%, at least 20%, at least 30%, at least 40%, at least 50% or more of the sucrose in the non-modified juice. In certain embodiments, the modification occurs through filtration of such juice to remove sucrose. In certain embodiments, sucrose in the juice is broken down to fructose and glucose, prior to adding the sweetening composition described herein.

Beverages comprise a matrix, i.e., the basic ingredient in which the ingredients—including the compositions of the present disclosure— are dissolved. In one embodiment, a beverage comprises water of beverage quality as the matrix, such as, for example deionized water, distilled water, reverse osmosis water, carbon-treated water, purified water, demineralized water and combinations thereof, can be used. Additional suitable matrices include, but are not limited to phosphoric acid, phosphate buffer, citric acid, citrate buffer and carbon-treated water. Beverage concentrates and beverage syrups are prepared with an initial volume of liquid matrix (e.g., water) and the desired beverage ingredients. Full strength beverages are then prepared by adding further volumes of water. Powdered beverages are prepared by dry-mixing all of the beverage ingredients in the absence of a liquid matrix. Full strength beverages are then prepared by adding the full volume of water.

It is contemplated that the pH of the beverage does not materially or adversely affect the taste of the sweetener. A non-limiting example of the pH range of the beverage may be from about 1.8 to about 10. In one embodiment, the pH of the beverage is about 4. In another embodiment, the pH of the beverage is less than about 4. In a particular embodiment, the pH of the beverage is less than about 3.8, less than about 3.6, less than about 3.4, less than about 3.2, less than about 3.0, less than about 2.8, less than about 2.6, less than about 2.4 or less than about 2.2. In another embodiment, the pH of the beverage is about 3.8, about 3.6, about 3.4, about 3.2, about 3.0, about 2.8, about 2.6, about 2.4 or about 2.2 or less.

### B. Edible Gel Mixes and Edible Gel Compositions

In one embodiment, the sweetened composition is an edible gel or edible gel mix. Edible gels are gels that can be eaten. Non-limiting examples of edible gel compositions for use in particular embodiments include gel desserts, puddings, jellies, pastes, trifles, aspics, marshmallows, gummy candies/chews, or the like. Edible gel mixes generally are powdered or granular solids to which a fluid may be added to form an edible gel composition. Non-limiting examples of fluids for use in particular embodiments include water, dairy fluids, dairy analogue fluids, juices, alcohol, alcoholic beverages, and combinations thereof. Non-limiting examples of dairy fluids which may be used in particular embodiments include milk, cultured milk, cream, fluid whey, and mixtures thereof. Non-limiting examples of dairy analogue fluids which may be used in particular embodiments include, for example, soy milk and non-dairy coffee whitener.

### C. Confections

In one embodiment, the sweetened composition is a confection. As referred to herein, “confection” can mean a sweet, a lollie, a confectionery, or similar term. The confection generally contains a base composition component and a sweetener component. According to particular embodiments of the present disclosure, the confections may be desserts such as yogurt, jellies, drinkable jellies, puddings, Bavarian cream, blancmange, cakes, brownies, mousse and the like, sweetened food products eaten at tea time or following meals; frozen foods; cold confections, e.g., types of ice cream such as ice cream, ice milk, lacto-ice and the like, and ice confections such as sherbets, dessert ices and the like; general confections, e.g., baked confections or steamed confections such as crackers, biscuits, buns with bean-jam filling, halvah, alfajor, and the like; rice cakes and snacks; table top products; general sugar confections such as chewing gum, hard candy, soft candy, mints, nougat candy, jelly beans, fudge, toffee, taffy, Swiss milk tablet, licorice candy, chocolates, gelatin candies, marshmallow, marzipan, divinity, cotton candy, and the like; sauces including fruit flavored sauces, chocolate sauces and the like; edible gels; cremes including butter cremes, flour pastes, whipped cream and the like; jams including strawberry jam, marmalade and the like; and breads including sweet breads and the like or other starch products, and combinations thereof.

### D. Condiment Compositions

In one embodiment, the sweetened composition is a condiment composition. Condiments, as used herein, are compositions used to enhance or improve the flavor of a food or beverage. Non-limiting examples of condiments include ketchup; mustard; barbecue sauce; butter; chili sauce; chutney; cocktail sauce; curry; dips; fish sauce; horseradish; hot sauce; jellies, jams, marmalades, or preserves; mayonnaise; peanut butter; relish; remoulade; salad dressings, salsa; sauerkraut; soy sauce; steak sauce; syrups; tartar sauce; and Worcestershire sauce. Condiment bases generally comprise a mixture of different ingredients, non-limiting examples of which include vehicles (e.g., water and vinegar); spices or seasonings (e.g., salt, pepper, garlic, mustard seed, onion, paprika, turmeric, and combinations thereof); fruits, vegetables, or their products (e.g., tomatoes or tomato-based products (paste, puree), fruit juices, fruit juice peels, and combinations thereof); oils or oil emulsions, particularly vegetable oils; thickeners (e.g., xanthan gum, food starch, other hydrocolloids, and combinations thereof); and emulsifying agents (e.g., egg yolk solids, protein, gum arabic, carob bean gum, guar gum, gum karaya, gum tragacanth, carageenan, pectin, propylene glycol esters of alginic acid, sodium carboxymethyl-cellulose, polysorbates, and combinations thereof). Recipes for condiment bases and methods of making condiment bases are well known to those of ordinary skill in the art.

### E. Chewing Gum Compositions

In one embodiment, the sweetened composition is a chewing gum composition. Chewing gum compositions generally comprise a water-soluble portion and a water-insoluble chewable gum base portion. The water soluble portion dissipates with a portion of the flavoring agent over a period of time during chewing while the insoluble gum base portion is retained in the mouth. The insoluble gum base generally determines whether a gum is considered chewing gum, bubble gum, or a functional gum.

Flavoring agents may be used in either the insoluble gum base or soluble portion of the chewing gum composition. Such flavoring agents may be natural or artificial flavors. In a particular embodiment, the flavoring agent comprises an essential oil, such as an oil derived from a plant or a fruit, peppermint oil, spearmint oil, other mint oils, clove oil, cinnamon oil, oil of wintergreen, bay, thyme, cedar leaf, nutmeg, allspice, sage, mace, and almonds. In another particular embodiment, the flavoring agent comprises a plant extract or a fruit essence such as apple, banana, watermelon, pear, peach, grape, strawberry, raspberry, cherry, plum, pineapple, apricot, and mixtures thereof. In still another particular embodiment, the flavoring agent comprises a citrus flavor, such as an extract, essence, or oil of lemon, lime, orange, tangerine, grapefruit, citron, or kumquat.

### F. Cereal Compositions

In one embodiment, the sweetened composition is a cereal composition. Cereal compositions typically are eaten either as staple foods or as snacks. Non-limiting examples of cereal compositions for use in particular embodiments include ready-to-eat cereals as well as hot cereals. Ready-to-eat cereals are cereals which may be eaten without further processing (i.e., cooking) by the consumer. Examples of ready-to-eat cereals include breakfast cereals and snack bars. Breakfast cereals typically are processed to produce a shredded, flaky, puffy, or extruded form. Breakfast cereals generally are eaten cold and are often mixed with milk and/or fruit. Snack bars include, for example, energy bars, rice cakes, granola bars, and nutritional bars. Hot cereals generally are cooked, usually in either milk or water, before being eaten. Non-limiting examples of hot cereals include grits, porridge, polenta, rice, and rolled oats.

Cereal compositions generally comprise at least one cereal ingredient. As used herein, the term “cereal ingredient” denotes materials such as whole or part grains, whole or part seeds, and whole or part grass. Non-limiting examples of cereal ingredients for use in particular embodiments include maize, wheat, rice, barley, bran, bran endosperm, bulgur, sorghums, millets, oats, rye, triticale, buckwheat, fonio, quinoa, bean, soybean, amaranth, teff, spelt, and kaniwa.

### G. Baked Goods

In one embodiment, the sweetened composition is a baked good. “Baked goods,” as used herein, include ready to eat and all ready to bake products, flours, and mixes requiring preparation before serving. Non-limiting examples of baked goods include cakes, crackers, cookies, brownies, muffins, rolls, bagels, donuts, strudels, pastries, croissants, biscuits, bread, bread products, and buns.

Baked goods in accordance with particular embodiments of this disclosure generally comprise a combination of sweetener, water, fat and leavening agent. Baked goods made in accordance with many embodiments of this disclosure also contain flour in order to make a dough or a batter.

According to particular embodiments of this disclosure, leavening agents may comprise chemical leavening agents or yeast leavening agents. Non-limiting examples of chemical leavening agents suitable for use in particular embodiments of this disclosure include baking soda (e.g., sodium, potassium, or aluminum bicarbonate), baking acid (e.g., sodium aluminum phosphate, monocalcium phosphate, or dicalcium phosphate), and combinations thereof.

### H. Dairy Products

In one embodiment, the sweetened composition is a dairy product. Dairy products and processes for making dairy products suitable for use in this disclosure are well known to those of ordinary skill in the art. Dairy products, as used herein, comprise milk or foodstuffs produced from milk. Non-limiting examples of dairy products suitable for use in embodiments of this disclosure include milk, milk cream, sour cream, creme fraiche, buttermilk, cultured buttermilk, milk powder, condensed milk, evaporated milk, butter, cheese, cottage cheese, cream cheese, yogurt, ice cream, frozen custard, frozen yogurt, gelato, vla, piima, filmjolk, kajmak, kephir, viili, kumiss, airag, ice milk, casein, ayran, lassi, khoa, or combinations thereof. The dairy products can be produced through conventional means or can be filtered or further modified to adjust the taste properties. In certain embodiments, the dairy products can be liquid dairy products from which one or more of the carbohydrate sugars (lactose or its breakdown products galactose or glucose) are reduced as compared to milk prior to such processing, or are substantially removed and which are supplemented with the sweetening composition described herein. The reduction of carbohydrates can be about 5% or about 10% or about 20% or about 50% or about 70% or more as compared to unprocessed milk.

According to particular embodiments of this disclosure, the dairy compositions also may comprise other additives. Non-limiting examples of suitable additives include sweeteners as disclosed herein and flavorants such as chocolate, strawberry, and banana. Particular embodiments of the dairy compositions provided herein also may comprise additional nutritional supplements such as vitamins (e.g., vitamin D) and minerals (e.g., calcium) to improve the nutritional composition of the milk.

### I. Tabletop Sweetener Compositions

In one embodiment, the sweetened composition is a tabletop sweetener. The tabletop sweetener can further include at least one bulking agent, additive, anti-caking agent, functional ingredient or combination thereof.

Suitable “bulking agents” include, but are not limited to, maltodextrin (10 DE, 18 DE, or 5 DE), corn syrup solids (20 or 36 DE), sucrose, fructose, glucose, invert sugar, sorbitol, xylose, ribulose, mannose, xylitol, mannitol, galactitol, erythritol, maltitol, lactitol, isomalt, maltose, tagatose, lactose, inulin, glycerol, propylene glycol, polyols, polydextrose, fructooligosaccharides, cellulose and cellulose derivatives, and the like, and mixtures thereof. Additionally, in accordance with still other embodiments of the present disclosure, granulated sugar (sucrose) or other caloric sweeteners such as crystalline fructose, other carbohydrates, or sugar alcohol can be used as a bulking agent due to their provision of good content uniformity without the addition of significant calories.

As used herein, the phrase “anti-caking agent” and “flow agent” refer to any composition which assists in content uniformity and uniform dissolution. In accordance with particular embodiments, non-limiting examples of anti-caking agents include cream of tartar, calcium silicate, silicon dioxide, microcrystalline cellulose (Avicel, FMC BioPolymer, Philadelphia, PA), and tricalcium phosphate. In one embodiment, the anti-caking agents are present in the tabletop sweetener composition in an amount from about 0.001 to about 3% by weight of the tabletop sweetener composition.

The tabletop sweetener compositions can be packaged in any form known in the art. Non-limiting forms include, but are not limited to, powder form, granular form, packets, tablets, sachets, pellets, cubes, solids, and liquids.

In one embodiment, the tabletop sweetener composition is a single-serving (portion control) packet comprising a dry-blend. Dry-blend formulations generally may comprise powder or granules. Although the tabletop sweetener composition may be in a packet of any size, an illustrative non-limiting example of conventional portion control tabletop sweetener packets are approximately 2.5 by 1.5 inches and hold approximately 1 gram of a sweetener composition having a sweetness equivalent to 2 teaspoons of granulated sugar (.about.8 g). In a particular embodiment, a dry-blend tabletop sweetener formulation may contain a sweetener an amount from about 1% (w/w) to about 10% (w/w).

A tabletop sweetener composition also may be embodied in the form of a liquid, wherein a composition of the present disclosure is combined with a liquid carrier. Suitable non-limiting examples of carrier agents for liquid tabletop sweeteners include water, alcohol, polyol, glycerin base or citric acid base dissolved in water, and mixtures thereof. The sweetness equivalent of a tabletop sweetener composition for any of the forms described herein or known in the art may be varied to obtain a desired sweetness profile. For example, a tabletop sweetener composition may comprise a sweetness comparable to that of an equivalent amount of standard sugar. In another embodiment, the tabletop sweetener composition may comprise a sweetness of up to 100 times that of an equivalent amount of sugar. In another embodiment, the tabletop sweetener composition may comprise a sweetness of up to 90 times, 80 times, 70 times, 60 times, 50 times, 40 times, 30 times, 20 times, 10 times, 9 times, 8 times, 7 times, 6 times, 5 times, 4 times, 3 times, and 2 times that of an equivalent amount of sugar.

### J. Delivery Systems

The presently disclosed sweetener compositions can also be formulated into various delivery systems having improved ease of handling and rate of dissolution. Non-limiting examples of suitable delivery systems comprise sweetener compositions co-crystallized with a sugar or a polyol, agglomerated sweetener compositions, compacted sweetener compositions, dried sweetener compositions, particle sweetener compositions, spheronized sweetener compositions, granular sweetener compositions, and liquid sweetener compositions.

### XI. Breeding Plants

In addition to direct transformation of a particular plant genotype with a construct prepared according to the current disclosure, transgenic plants may be made by crossing a plant having a selected DNA of the present disclosure to a second plant lacking the construct. For example, a selected mogroside biosynthesis pathway coding sequence can be introduced into a particular plant variety by crossing, without the need for ever directly transforming a plant of that given variety. Therefore, the current disclosure not only encompasses a plant directly transformed or regenerated from cells that have been transformed in accordance with the current disclosure, but also the progeny of such plants.

As used herein the term “progeny” denotes the offspring of any generation of a parent plant prepared in accordance with the instant disclosure, wherein the progeny comprises a selected DNA construct. “Crossing” a plant to provide a plant line having one or more added transgenes relative to a starting plant line, as disclosed herein, is defined as the techniques that result in a transgene of the present disclosure being introduced into a plant line by crossing a starting line with a donor plant line that comprises a transgene of the present disclosure. To achieve this one could, for example, perform the following steps:
(a) plant seeds of the first (starting line) and second (donor plant line that comprises a transgene of the present disclosure) parent plants;
(b) grow the seeds of the first and second parent plants into plants that bear flowers;
(c) pollinate a flower from the first parent plant with pollen from the second parent plant; and
(d) harvest seeds produced on the parent plant bearing the fertilized flower.

Backcrossing is herein defined as the process including the steps of:
(a) crossing a plant of a first genotype containing a desired gene, DNA sequence or element to a plant of a second genotype lacking the desired gene, DNA sequence or element;
(b) selecting one or more progeny plant containing the desired gene, DNA sequence or element;
(c) crossing the progeny plant to a plant of the second genotype; and
(d) repeating steps (b) and (c) for the purpose of transferring a desired DNA sequence from a plant of a first genotype to a plant of a second genotype.

Introgression of a DNA element into a plant genotype is defined as the result of the process of backcross conversion. A plant genotype into which a DNA sequence has been introgressed may be referred to as a backcross converted genotype, line, inbred, or hybrid. Similarly a plant genotype lacking the desired DNA sequence may be referred to as an unconverted genotype, line, inbred, or hybrid.

### XII. Additional Definitions

The following definitions or interpretations of technical terms will be used throughout the present disclosure. The technical terms used herein are generally to be given the meaning commonly applied to them in the pertinent art of plant biology, molecular biology, bioinformatics, and plant breeding. All of the following term definitions apply to the complete content of this application.

To facilitate the understanding of this disclosure, a number of terms are defined below. Terms defined herein have meanings as commonly understood by a person of ordinary skill in the areas relevant to the present disclosure. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of.” As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about,” “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

The terms “peptides,” “oligopeptides,” “polypeptide,” “protein”, or “enzyme” are used interchangeably herein and refer to amino acids in a polymeric form of any length, linked together by peptide bonds, unless mentioned herein otherwise. The terms “gene sequence(s),” “polynucleotide(s),” “nucleic acid sequence(s),” “nucleotide sequence(s),” “nucleic acid(s),” “nucleic acid molecule” are used interchangeably herein and refer to nucleotides, either ribonucleotides or deoxyribonucleotides or a combination of both, in a polymeric unbranched form of any length.

Endogenous. An “endogenous” or “native” nucleic acid and/or protein refers to a nucleic acid and/or protein as found in a plant or other organism in its natural form (i.e., without there being any human intervention, such as recombinant DNA engineering technology),

Exogenous. The term “exogenous” (in contrast to “endogenous”) means a nucleic acid or protein that has been introduced in a plant or other organism by means of recombinant DNA technology. An “exogenous” nucleic acid or protein can either not occur in a plant in its natural form, be different from the nucleic acid or protein as found in a plant in its natural form, be present at a higher or lower level than the nucleic acid or protein naturally present in a plant, or in the case of a nucleic acid can be identical to a nucleic acid found in a plant in its natural form, but integrated at a location different that its natural genetic environment.

Expression: The combination of intracellular processes, including transcription and translation undergone by a coding DNA molecule such as a structural gene to produce a polypeptide.

Expression Cassette. A nucleic acid sequence of interest operably linked to one or more control sequences (at least to a promoter) as described herein. An expression cassette can also include additional transcriptional and/or translational enhancers. An expression cassette can also include terminator, silencer and enhancer sequences, intron sequences added to the 5′ untranslated region (UTR) or in the coding sequence of the nucleic acid sequence, and/or other control sequences such as protein and/or RNA stabilizing elements. An expression cassette may be integrated into the genome of a host cell and replicated together with the genome of said host cell, or transiently present in a host cell.

Genetic Transformation: A process of introducing a DNA sequence or construct (e.g., a vector or expression cassette) into a cell or protoplast in which that exogenous DNA is incorporated into a chromosome or is capable of autonomous replication.

Heterologous: A sequence that is not normally present in a given host genome in the genetic context in which the sequence is currently found In this respect, the sequence may be native to the host genome, but be rearranged with respect to other genetic sequences within the host sequence. For example, a regulatory sequence may be heterologous in that it is linked to a different coding sequence relative to the native regulatory sequence.

Modulation. The term modulation refers to when the expression level is changed in comparison to the expression seen in a control plant. Modulation refers to an expression level that is either increased or decreased.

Obtaining: When used in conjunction with a transgenic plant cell or transgenic plant, obtaining means either transforming a non-transgenic plant cell or plant to create the transgenic plant cell or plant, or planting transgenic plant seed to produce the transgenic plant cell or plant. Such a transgenic plant seed may be from an R0 transgenic plant or may be from a progeny of any generation thereof that inherits a given transgenic sequence from a starting transgenic parent plant.

Operably Linked. The term “operably linked” or “functionally linked” is used interchangeably and, as used herein, refers to a functional linkage between, for example, a promoter sequence and a nucleic acid sequence of interest, such that the promoter sequence is able to direct transcription of the nucleic acid sequence of interest, or a functional linkage between a terminator sequence and a nucleic acid sequence of interest, such that the terminator sequence is able to stop or terminate transcription of the nucleic acid sequence of interest.

Plant. The term “plant” as used herein encompasses whole plants, ancestors and progeny of the plants and plant parts, including fruits, seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

Ploidy. Ploidy or chromosomal ploidy refers the number of complete sets of chromosomes occurring in the nucleus of a cell. Somatic cells, tissues, and individual organisms can be described according to the number of sets of chromosomes present (the “ploidy level”): monoploid (1 set), diploid (2 sets), triploid (3 sets), tetraploid (4 sets), pentaploid (5 sets), hexaploid (6 sets), heptaploid or septaploid (7 sets), etc. The generic term polyploidy is used herein to describe cells with three or more chromosome sets.

Promoter: A recognition site on a DNA sequence or group of DNA sequences that provides an expression control element for a structural gene and to which RNA polymerase specifically binds and initiates RNA synthesis (transcription) of that gene.

R0 transgenic plant: A plant that has been genetically transformed or has been regenerated from a plant cell or cells that have been genetically transformed.

Recombinant. A nucleic acid sequence, expression cassette, genetic construct, or vector comprising a nucleic acid sequence as disclosed herein, or an organism transformed with such nucleic acid sequences, expression cassettes or vectors, created by genetic engineering techniques in which either (a) the sequences of the nucleic acids or a part thereof, or (b) genetic control sequence(s) that is operably linked with the nucleic acid sequence, for example a promoter or terminator, or (c) combinations of (a) and (b), are not located in their natural genetic environment or have been modified and/or inserted artificially by genetic engineering methods.

Regeneration: The process of growing a plant from a plant cell (e.g., plant protoplast, callus or explant).

Selected DNA: A DNA segment that one desires to introduce or has introduced into a plant genome by genetic transformation.

Terminator. A DNA control sequence at the end of a transcriptional unit that signals 3′ processing and polyadenylation of a primary transcript and termination of transcription.

Transformation construct: A chimeric DNA molecule that is designed for introduction into a host genome by genetic transformation. Transformation constructs will often comprise all of the genetic elements necessary to direct the expression of one or more exogenous genes. In particular embodiments of the instant disclosure, it may be desirable to introduce a transformation construct into a host cell in the form of an expression cassette.

Transformed cell: A cell the DNA complement of which has been altered by the introduction of an exogenous DNA molecule into that cell.

Transgene: A segment of DNA that has been incorporated into a host genome or is capable of autonomous replication in a host cell and is capable of causing the expression of one or more coding sequences. Exemplary transgenes will provide the host cell, or plants regenerated therefrom, with a novel phenotype relative to the corresponding non-transformed cell or plant. Transgenes may be directly introduced into a plant by genetic transformation, or may be inherited from a plant of any previous generation that was transformed with the DNA segment.

Transgenic plant: A plant or progeny plant of any subsequent generation derived therefrom, wherein the DNA of the plant or progeny thereof contains an introduced exogenous DNA segment not naturally present in a non-transgenic plant of the same strain. The transgenic plant may additionally contain sequences that are native to the plant being transformed, but wherein the “exogenous” gene has been altered in order to alter the level or pattern of expression of the gene, for example, by use of one or more heterologous regulatory or other elements.

Vector: A DNA molecule designed for transformation into a host cell. Some vectors may be capable of replication in a host cell. A plasmid is an exemplary vector, as are expression cassettes isolated therefrom.

### EXAMPLES

The following examples are included to demonstrate illustrative embodiments of the present disclosure. It should be appreciated by those of skill in the art that the techniques disclosed in the examples that follow represent techniques discovered by the inventor to function well in the practice of the present disclosure, and thus can be considered to constitute one embodiment of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Construction of Expression Cassettes

Various expression cassettes having different combinations of nucleotide sequences encoding the mogroside pathway enzymes and regulatory elements were constructed. Construction of these expression cassettes was carried out following standard genetic engineering methods. The following expression cassettes were constructed.

SP1463: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3139: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a e35S promoter operably linked to a HygR nucleic acid sequence, which is operably linked to a 35S terminator, followed by a TM6 MAR insulator sequence.

SP1908: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a e35S promoter operably linked to a HygR nucleic acid sequence, which is operably linked to a 35S terminator, followed by a TM6 MAR insulator sequence.

SP3488: This expression cassette was assembled using tm6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an CmYLCV promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3015: This expression cassette was assembled using a TM6 MAR insulator sequence, followed by a dMMV promoter operably linked to a CYP72 nucleic acid sequence, which is operably linked to an AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, an e35S promoter operably linked to a UGT720 nucleic acid sequence, which is operably linked to an E9 terminator, a CmYLCV promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3432: This expression cassette was assembled using a TM6 MAR insulator sequence, followed by a CmYLCV promoter operably linked to a SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, an e35S promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1160: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator, a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2916: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator (all in reverse orientation), a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4643: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a CmYLCV promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4870: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator, a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1603: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3095: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0265: This expression cassette was assembled using a TM6 MAR insulator sequence, followed by a CmYLCV promoter operably linked to a SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a UGT720:2A:CYP72 bicistronic nucleic acid sequence, which is operably linked to an E9 terminator, a dMMV promoter operably linked to a UGT94:2A:tHMGR bicistronic nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP4406: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to a AtRBCS2B terminator, a CmYLCV promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2152: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to a AtRBCS2B terminator, a CmYLCV promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4311: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator, a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4378: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator (all in reverse orientation), a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3132: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a CmYLCV promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2355: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator, a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4762: This expression cassette was assembled using a TM6 MAR insulator sequence, followed by an e35S promoter operably linked to a CYP72:2A:CYP72 bicistronic nucleic acid sequence, which is operably linked to a AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0892: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to a ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2249: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 nucleic acid sequence, which is operably linked to an AtRBCS2B terminator, an e35S promoter operably linked to a UGT720 nucleic acid sequence, which is operably linked to an E9 terminator, a CmYLCV promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0796: This expression cassette was assembled using a TM6 MAR insulator sequence, followed by a dMMV promoter operably linked to a CYP72 nucleic acid sequence, which is operably linked to an AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2057: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 nucleic acid sequence, which is operably linked to an AtRBCS2B terminator, a NOS promoter operably linked to an EPH nucleic acid sequence, which is operably linked to an Ubi3 terminator, an e35S promoter operably linked to a UGT94 nucleic acid sequence, which is operably linked to an ATHSP18.2 terminator, a CmYLCV promoter operably linked to a UGT720 nucleic acid sequence, which is operably linked to an E9 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3308: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72 nucleic acid sequence, which is operably linked to an AtRBCS2B terminator, a CmYLCV promoter operably linked to a UGT720 nucleic acid sequence, which is operably linked to an E9 terminator, a dMMV promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1379: This expression cassette was assembled using a TM6 MAR insulator sequence, followed by a dMMV promoter operably linked to a CYP72 nucleic acid sequence, which is operably linked to an AtRBCS2B terminator, a CmYLCV promoter operably linked to a UGT720 nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3494: This expression cassette was assembled using a TM6 MAR insulator sequence, followed by a dMMV promoter operably linked to a CYP72 nucleic acid sequence, which is operably linked to an AtRBCS2B terminator (all in reverse orientation), a CmYLCV promoter operably linked to a UGT720 nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2585: This expression cassette was assembled using a TM6 MAR insulator sequence, followed by a CmYLCV promoter operably linked to a SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 nucleic acid sequence, which is operably linked to an AtRBCS2B terminator, an e35S promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, an e35S promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3635: This expression cassette was assembled using a TM6 MAR insulator sequence, followed by a CmYLCV promoter operably linked to a SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a UGT720:2A:CYP72 bicistronic nucleic acid sequence, which is operably linked to an E9 terminator, an e35S promoter operably linked to a UGT94:2A:tHMGR bicistronic nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP3800: This expression cassette was assembled using a TM6 MAR insulator sequence, followed by a CmYLCV promoter operably linked to a SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, an e35S promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0981: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0137: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2154: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1727: This expression cassette was assembled using a TM6 MAR insulator nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0075: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4305: This expression cassette was assembled using a TM6 MAR insulator nucleic acid sequence, which is operably linked to an AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4221: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3488: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an CmYLCV promoter operably linked to a UGT720:2A:UGT720 bicistronic nucleic acid sequence, which is operably linked to an E9 terminator, a dMMV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4094: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2971: This expression cassette was assembled using a TM6 MAR insulator nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2049: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FMVSgt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4063: This expression cassette was assembled using a TM6 MAR insulator nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0121: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FMVSgt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3358: This expression cassette was assembled using a TM6 MAR insulator sequence, followed by a CmYLCV promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, an e35S promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4513: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2221: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3925: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3748: This expression cassette was assembled using a TM6 MAR insulator nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4511: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3547: This expression cassette was assembled using a TM6 MAR insulator nucleic acid sequence, which is operably linked to an AtRBCS2B terminator, a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3481: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2185: This expression cassette was assembled using a TM6 MAR insulator nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2792: This expression cassette was assembled using a TM6 MAR insulator sequence, followed by a CmYLCV promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a UGT720:2A:CYP72 bicistronic nucleic acid sequence, which is operably linked to an E9 terminator, an e35S promoter operably linked to a UGT94:2A:tHMGR bicistronic nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP1000: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3766: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4353: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0255: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4815: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1073: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP4402: This expression cassette was assembled using a TM6 MAR insulator sequence, followed by a dMMV promoter operably linked to a CYP72:2A:CYP72 bicistronic nucleic acid sequence, which is operably linked to an AtRBCS2B terminator (all in reverse orientation), a CmYLCV promoter operably linked to a UGT720:2A:UGT720 bicistronic nucleic acid sequence, which is operably linked to an E9 terminator, a FMVSgt promoter operably linked to a CDS nucleic acid sequence, which is operably linked to a GmaxMYB2 terminator, a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1415: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2353: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, a CmYLCV promoter operably linked to a UGT720:2A:UGT720 bicistronic nucleic acid sequence, which is operably linked to an E9 terminator, an e35S promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP0565: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FMVSgt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP1202: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FMVSgt promoter operably linked to a CDS nucleic acid sequence, which is operably linked to a GmaxMYB2 terminator, an e35S promoter operably linked to a CYP72:2A:CYP72 bicistronic nucleic acid sequence, which is operably linked to an AtRBCS2B terminator, an e35S promoter operably linked to a UGT720:2A:UGT720 bicistronic nucleic acid sequence, which is operably linked to an E9 terminator, a CmYLCV promoter operably linked to a UGT94:2A:UGT94 bicistronic nucleic acid sequence, which is operably linked to an ATHSP18.2 terminator, a NOS promoter operably linked to an EPH nucleic acid sequence, which is operably linked to an Ubi3 terminator, a FE3 promoter operably linked to a tHMGR:2A:tHMGR bicistronic nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP2808: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub89 terminator, a CsVMV promoter operably linked to a HygR nucleic acid sequence, which is operably linked to a 35S terminator, followed by a TM6 MAR insulator sequence.

SP3684: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, an e35S promoter operably linked to a UGT720 nucleic acid sequence, which is operably linked to an ATHSP18.2 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to a 35S terminator, a CsVMV promoter operably linked to a HygR nucleic acid sequence, which is operably linked to a 35S terminator, followed by a TM6 MAR insulator sequence.

SP4522: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, an e35S promoter operably linked to a CYP72 Zm nucleic acid sequence, which is operably linked to an ATHSP18.2 terminator, an e35S promoter operably linked to a UGT720 nucleic acid sequence, which is operably linked to an ATHSP18.2 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to a 35S terminator, a CsVMV promoter operably linked to a HygR nucleic acid sequence, which is operably linked to a 35S terminator, followed by a TM6 MAR insulator sequence.

SP3842: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a CsVMV promoter operably linked to a HygR nucleic acid sequence, which is operably linked to a 35S terminator, followed by a TM6 MAR insulator sequence.

SP3938: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CsVMV promoter operably linked to a HygR nucleic acid sequence, which is operably linked to a 35S terminator, followed by a TM6 MAR insulator sequence.

SP3318: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CmYLCV promoter operably linked to a UGT74_3 nucleic acid sequence, which is operably linked to a 35S terminator, a CsVMV promoter operably linked to a HygR nucleic acid sequence, which is operably linked to a 35S terminator, followed by a TM6 MAR insulator sequence.

SP3493: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CmYLCV promoter operably linked to a UGT74_4 nucleic acid sequence, which is operably linked to a 35S terminator, a CsVMV promoter operably linked to a HygR nucleic acid sequence, which is operably linked to a 35S terminator, followed by a TM6 MAR insulator sequence.

SP2476: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D20 m2 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CsVMV promoter operably linked to a HygR nucleic acid sequence, which is operably linked to a 35S terminator, followed by a TM6 MAR insulator sequence.

SP4983: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D20 m3 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CsVMV promoter operably linked to a HygR nucleic acid sequence, which is operably linked to a 35S terminator, followed by a TM6 MAR insulator sequence.

SP4003: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D17 m2 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CsVMV promoter operably linked to a HygR nucleic acid sequence, which is operably linked to a 35S terminator, followed by a TM6 MAR insulator sequence.

SP4074: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D17 m3 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CsVMV promoter operably linked to a HygR nucleic acid sequence, which is operably linked to a 35S terminator, followed by a TM6 MAR insulator sequence.

SP2649: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D18_B m3 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CsVMV promoter operably linked to a HygR nucleic acid sequence, which is operably linked to a 35S terminator, followed by a TM6 MAR insulator sequence.

SP3397: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D17 m2 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CmYLCV promoter operably linked to a UGT74_3 nucleic acid sequence, which is operably linked to a 35S terminator, a CsVMV promoter operably linked to a HygR nucleic acid sequence, which is operably linked to a 35S terminator, followed by a TM6 MAR insulator sequence.

SP2771: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D17 m3 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CmYLCV promoter operably linked to a UGT74_3 nucleic acid sequence, which is operably linked to a 35S terminator, a CsVMV promoter operably linked to a HygR nucleic acid sequence, which is operably linked to a 35S terminator, followed by a TM6 MAR insulator sequence.

SP0847: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87D17 m3 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a DCMV promoter operably linked to a SgCPR2 nucleic acid sequence, which is operably linked to an AtUBQ3 terminator, a CmYLCV promoter operably linked to a UGT74_4 nucleic acid sequence, which is operably linked to a 35S terminator, a CsVMV promoter operably linked to a HygR nucleic acid sequence, which is operably linked to a 35S terminator, followed by a TM6 MAR insulator sequence.

SP3468: This expression cassette was assembled using a TM6 MAR insulator sequence, followed by a FE_3 promoter operably linked to a CYP87 nucleic acid sequence, which is operably linked to an ATHSP18.2 terminator, a FE_3 promoter operably linked to a CDS nucleic acid sequence, which is operably linked to a 35S terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, a CmYLCV promoter operably linked to a UGT94 nucleic acid sequence, which is operably linked to an 35S terminator, a FS1_1 promoter operably linked to a tHMGR nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP2177: This expression cassette was assembled using a TM6 MAR insulator sequence, followed by a FS1_1 promoter operably linked to an SQE nucleic acid sequence, which is operably linked to a 35S terminator, a FE_3 promoter operably linked to a CYP87 nucleic acid sequence, which is operably linked to a 35S terminator, a FE_3 promoter operably linked to a CDS nucleic acid sequence, which is operably linked to a 35S terminator, a FE_3 promoter operably linked to a CYP72 Zm nucleic acid sequence, which is operably linked to an 35S terminator, a FE_3 promoter operably linked to a UGT720 nucleic acid sequence, which is operably linked to a 35S terminator, a FE_3 promoter operably linked to a UGT94 nucleic acid sequence, which is operably linked to an 35S terminator, a FS1_1 promoter operably linked to an EPH nucleic acid sequence, which is operably linked to a 35S terminator, a FS1_1 promoter operably linked to a tHMGR nucleic acid sequence, which is operably linked to a 35S terminator, a CsVMV promoter operably linked to a HygR nucleic acid sequence, which is operably linked to a 35S terminator, followed by a TM6 MAR insulator sequence.

SP3804: This expression cassette was assembled using a TM6 MAR insulator sequence, followed by a HLVH12 promoter operably linked to an SQE nucleic acid sequence, which is operably linked to a Pea3A terminator, an e35S promoter operably linked to a CYP87 nucleic acid sequence, which is operably linked to an ATHSP18.2 terminator, a FSgt/PFLt promoter operably linked to a CDS nucleic acid sequence, which is operably linked to a ATHSP18.2 terminator, an e35S promoter operably linked to a CYP72 Zm nucleic acid sequence, which is operably linked to an ATHSP18.2 terminator, an e35S promoter operably linked to a UGT720 nucleic acid sequence, which is operably linked to an ATHSP18.2 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a e35S promoter operably linked to a tHMGR nucleic acid sequence, which is operably linked to an ATHSP18.2 terminator, a CsVMV promoter operably linked to a HygR nucleic acid sequence, which is operably linked to a 35S terminator, followed by a TM6 MAR insulator sequence.

SP3016: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, an e35S promoter operably linked to a UGT720 nucleic acid sequence, which is operably linked to an ATHSP18.2 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP0036: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a CDS nucleic acid sequence, which is operably linked to an ATHSP18.2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP1458: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, an e35S promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to a 35S terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP0336: This expression cassette was assembled using a TM6 MAR insulator sequence, followed by a dMMV promoter operably linked to an green fluorescent protein nucleic acid sequence, which is operably linked to a PBI terminator, a PCLSV promoter operably linked to an SQE-2 nucleic acid sequence, which is operably linked to a Pea3A terminator, a DCMV promoter operably linked to a CYP87-2 nucleic acid sequence, which is operably linked to an AtUBQ3 terminator, a Fsgt/PFLT promoter operably linked to a CDS-2 nucleic acid sequence, which is operably linked to a GmaxMYB2 terminator, a CsVMV promoter operably linked to an EPH-2 nucleic acid sequence, which is operably linked to an ATHSP18.2 terminator, a HLVH12 promoter operably linked to a UGT720-2 nucleic acid sequence, which is operably linked to an E9 terminator, a FMVSgt promoter operably linked to an EPH-2 nucleic acid sequence, which is operably linked to an Ubi3 terminator, an e35S promoter operably linked to a HygR nucleic acid sequence, which is operably linked to a 35S terminator, followed by a TM6 MAR insulator sequence.

SP0315: This expression cassette was assembled using a TM6 MAR insulator sequence, followed by a CmYLCV promoter operably linked to an SQE nucleic acid sequence, which is operably linked to a Pea3A terminator, a DCMV promoter operably linked to a CYP87 nucleic acid sequence, which is operably linked to an AtUBQ3 terminator, a Fgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a HLVH12 promoter operably linked to a UGT720 nucleic acid sequence, which is operably linked to an E9 terminator, a CsVMV promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a 35S promoter operably linked to a HygR nucleic acid sequence, which is operably linked to a 35S terminator, followed by a TM6 MAR insulator sequence.

SP3190: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a CsVMV promoter operably linked to a HygR nucleic acid sequence, which is operably linked to a 35S terminator, followed by a TM6 MAR insulator sequence.

SP3029: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a ScBV promoter operably linked to a tHMGR nucleic acid sequence, which is operably linked to an AtTub terminator, a CsVMV promoter operably linked to a HygR nucleic acid sequence, which is operably linked to a 35S terminator, followed by a TM6 MAR insulator sequence.

SP0545: This expression cassette was assembled using a TM6 MAR insulator sequence, followed by a HLVH12 promoter operably linked to an SQE nucleic acid sequence, which is operably linked to a Pea3A terminator, a DCMV promoter operably linked to a CYP87 nucleic acid sequence, which is operably linked to an AtUBQ3 terminator, a FSgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a CmYLCV promoter operably linked to a UGT720 nucleic acid sequence, which is operably linked to an E9 terminator, an e35S promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a FE_3 promoter operably linked to a tHMGR nucleic acid sequence, which is operably linked to an AtTub terminator, a CsVMV promoter operably linked to a HygR nucleic acid sequence, which is operably linked to a 35S terminator, followed by a TM6 MAR insulator sequence.

SP4156: This expression cassette was assembled using a TM6 MAR insulator sequence, followed by a CmYLCV promoter operably linked to an SQE nucleic acid sequence, which is operably linked to a Pea3A terminator, a DCMV promoter operably linked to a CYP87 nucleic acid sequence, which is operably linked to an AtUBQ3 terminator, a Fgt/PFLt promoter operably linked to a CDS nucleic acid sequence, which is operably linked to a GmaxMYB2 terminator, a dMMV promoter operably linked to a CYP72 Zm nucleic acid sequence, which is operably linked to an AtRBCS2B terminator, a HLVH12 promoter operably linked to a UGT720 nucleic acid sequence, which is operably linked to an E9 terminator, a CsVMV promoter operably linked to a UGT94 nucleic acid sequence, which is operably linked to an ATHSP18.2 terminator, a NOS promoter operably linked to an EPH nucleic acid sequence, which is operably linked to an Ubi3 terminator, a 35S promoter operably linked to a nptII nucleic acid sequence, which is operably linked to a 35S terminator, followed by a TM6 MAR insulator sequence.

### Example 2

### Initiation and Maintenance of Watermelon Anther-Derived Cell Suspension Cultures

This example details how to initiate callus from male flowers of watermelon and how to establish and maintain watermelon cell suspension cultures. Watermelon cell suspension cultures can be used for the production of mogrosides or for gene/parts/vector testing in transient assays.

Materials

Sterile containers (e.g., magenta boxes, flip cap tubes, etc., pick a size that is appropriate for the volume of plant tissue and sterilization solution); scalpel (#10); scalpel handle; forceps; full-strength commercial bleach; sterile water; 70% ethanol; sterile paper towels; sterile filter paper (125 mm); 15 mL Falcon™ tubes; 50 mL Falcon™ tubes; sterile 60×15 mm petri plates; sterile 150×15 mm petri plates; 100% ethanol; sterile 10 mL wide-mouth pipettes; sterile 25 mL pipettes; Pipet-Aid®; sterile 125 mL Erlenmeyer flasks; sterile 250 mL Erlenmeyer flasks; 1000 μm pluriStrainer™ (Pluriselect: SKU 43-50750-03).

Media

Callus initiation from anther and maintenance (CIM): full strength MS salts and vitamins, sucrose: 30 g/L, pH: 5.8, agar: 6.0 g/L, autoclave 500 mL on liquid 25 cycle, after autoclaving and cooling add: 2,4-D: 0.5 mg/L, thidiazuron (TDZ): 0.5 mg/L, pour media into tall culture plates.

Cell suspension liquid media (initiation and maintenance): full strength MS salts and vitamins, sucrose: 30 g/L, pH 5.8, autoclave 500 mL on liquid 25 cycle, after autoclaving and cooling add: 2,4-D: 0.5 mg/L, TDZ: 0.5 mg/L, STABA vitamins: 10 mL per liter.

Method

All procedures are performed in a sterile laminar flow hood unless otherwise specified. It takes anywhere from 1 to 2 months for callus to develop from the male flowers. After callus forms, it takes another 1 to 2 months for callus proliferation.

Disinfection of male flowers and callus initiation: 1. Collect the immature male flowers from the vine. The flower should still be closed at time of collection and petals should be green in color. 2. Place the immature male flowers in a sterile tissue culture container big enough for the sampling. Sterile magenta boxes, flip-cap tubes, and one liter containers can be used. 3. Add 70% ethanol to the container. Use enough to cover the flowers. 4. Incubate the flowers in the ethanol for 1 minute while gently swirling the container. 5. Pour off and discard the ethanol solution. 6. Add a solution of 10% commercial bleach (v/v)+0.05% Tween 20 to the container. Use enough to cover the flowers. Place sterile paper towels on top of the solution so that the towels fully immerse the flowers in the bleach. 7. Place the cap or lid on the sterile container and then place the container on a rotary shaker. Incubate the flowers in the bleach solution for 15 minutes on the rotary shaker (˜90 rpm). 8. In a sterile laminar flow hood, pour off and discard the bleach solution. 9. Rinse the tissue three times with sterile water containing 400 mg/L Timentin. 10. Transfer the flowers to a sterile 150 mm×15 mm petri plate containing sterile 125 mm filter paper. 11. Use the scalpel to gently remove the outer layer of petals. Once the outer layer is removed, carefully dissect out the anthers and place them in petri plates containing callus induction media (CIM). 12. Wrap the plates with cling wrap and place them in a chamber with growth conditions of 16/8 hour day/night cycle at 25° C./23° C.±2° C.

Callus selection and propagation: 1. After 1 month, the anther explants will exhibit signs of swelling and watery, pale, loose callus can be observed on the surface of the explants. 2. Remove and transfer the callus to fresh CIM medium (same as used for initiation). 3. Sub-culture the callus every 2 to 3 weeks for differentiation and proliferation.

Cell suspension establishment and subculture (performed weekly). A 1:4 ratio of old media to new media is maintained during the weekly subculture: 1. After 3 to 4 months, different types of calli will be observed on the plates. White/yellow friable callus that is similar in texture and appearance to cotton candy is the best type to use for initiation of the cell suspension. 2. Using a sterile 25 mL pipet, add 25 m1 of liquid cell suspension medium to a sterile 125 m1 Erlenmeyer flask. 3. Add approximately 2 to 3 grams of watermelon callus to the flask and swirl the contents. The callus pieces should start to break apart in the liquid culture media. 4. Place the flask on a rotary shaker and culture at 110 rpm under constant lighting (1500 Lux) at 24±1° C. 5. After 7 days, transfer the culture to a sterile 50 mL conical tube. Allow the cells to settle. 6. Remove and discard 20 mL of the old media. 7. Add 20 mL of fresh media to the tube and transfer the contents (including cells) to a new 125 mL Erlenmeyer flask. 8. After 7 days, place a 1000 μm nylon mesh cell strainer into a 50 m1 conical tube and filter the culture through the cell strainer. This removes large cell aggregates and leads to a finer cell suspension. 9. Allow the cells to settle to the bottom of the tube and then transfer those cells to a 15 mL conical tube. 10. Repeat steps 8 and 9 either one or two more times. 11. After the small cell population has been collected in the 15 m1 conical tube, transfer the cells along with 10 mL of old media to a 250 mL Erlenmeyer flask. 12. Add 40 mL of fresh media to the flask. Place the flask back on the shaker and culture at 110 rpm under constant lighting (1500 Lux) at 24±1° C. 13. Record the settled cell volume every 7 days after sub-culture and replace old media with fresh media weekly. The cell suspension should begin to increase in volume 3-4× after three weeks of routine sub-culture. Use approximately 3 to 5 mL of settled cell volume per sub-culture. More cells than this will result in an unhealthy cell suspension as resources are diminished more quickly. Use 1000 μm nylon mesh cell strainers to filter the cells every third or fourth sub-culture. This helps to maintain a population of smaller cell aggregates.

### Example 3

### Transient Expression in Plants

Various expression cassettes selected from Example 1 were used for transient expression in various tissues of different plants, such as the fruit of watermelon, tomato, zucchini, and cucumber; leaves of Nicotiana benthamiana, lettuce, and sugar beet; and taproot of sugar beet. Agrobacterium tumefaciens strain EHA105 was transformed with one or more expression cassette plasmids (selected from Example 1) using a free-thaw method (Weigel, CSH Protoc. 2006 Dec. 1; 2006(7): pdb.ip29. doi: 10.1101/pdb.ip29). Briefly, chemically competent Agrobacterium was prepared. After addition of the expression cassette plasmids, the mixture was alternately frozen in liquid nitrogen and thawed to liquid in a 37° C. water bath. The cells were then allowed to recover in LB medium for about 1 hour and plated out on LB plates with kanamycin.

Briefly, transformed EHA105 Agrobacterium strains were grown overnight and diluted to combined OD₆₀₀ readings of 0.5-2.5. Cores of watermelon fruit; whole tomato, zucchini, or cucumber fruits; leaves of Nicotiana benthamiana, lettuce, or sugar beet; or thin slices of sugar beet taproot were used for transient expression by infiltration. The diluted transformed Agrobacterium culture was loaded to a 1 or 5 mL syringe with or without needle, and about 1 mL was injected to the targeted plant tissue such as the fruit or the leaves. Sugar beet taproot slices were infiltrated by incubating in diluted Agrobacterium for 30 minutes. One or more Agrobacterium strains with different expression cassettes can be mixed prior to injection. The plants and plant tissues infiltrated with the transformed Agrobacterium cultures were grown for another four to nine days before sampling.

### Mogroside Detection

About 100 mg of plant tissue was extracted in 500 μl extraction buffer (80% methanol). After centrifugation, the supernatant was forced to pass through 0.22 μM filter in order to remove remaining particles. Waters Acquity UPLC coupled by Waters Xevo Quadrupole Time of Flight Tandem Mass Spectrometer was used for metabolite analysis. For UPLC separation, Waters Acquity BEH C18 1.7 μm, 2.1×50 mm column was used with water and acetonitrile as solvents (both with 1% formic acid). For each analysis, 1.5 μl of sample was injected. MS/MS under negative ESI was used for detection of mogroside compounds. The collision energy was set to 30 V for detection of mogroside compounds.

Preliminary studies in transient assays from Nicotiana bentamiana leaves showed production of mogrosides and oxo-mogrosides.

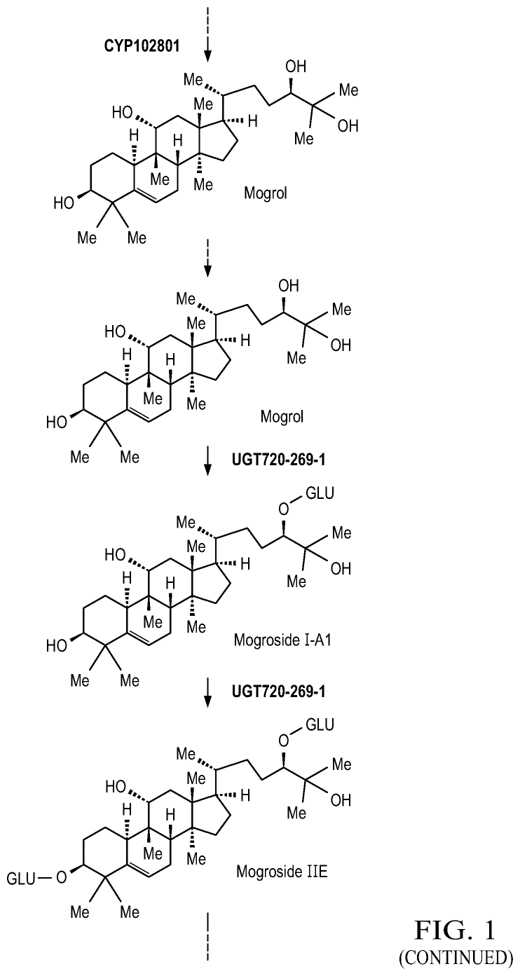

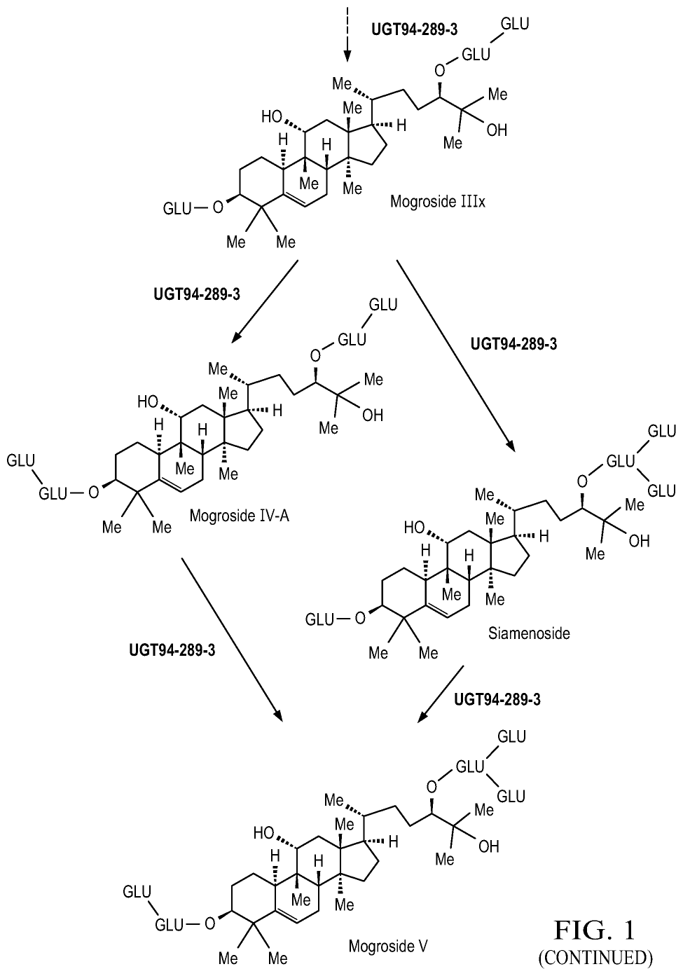

Mogroside V accumulation in transient assays from watermelon fruit infiltrated with a number of different expression constructs described in Example 1 is shown in FIG. 2. A number of the expression constructs produced high levels of mogroside V in these transient assays, but the SP3139 expression construct produced the highest levels of mogroside V. A map of the SP3139 expression construct is shown in FIG. 3.

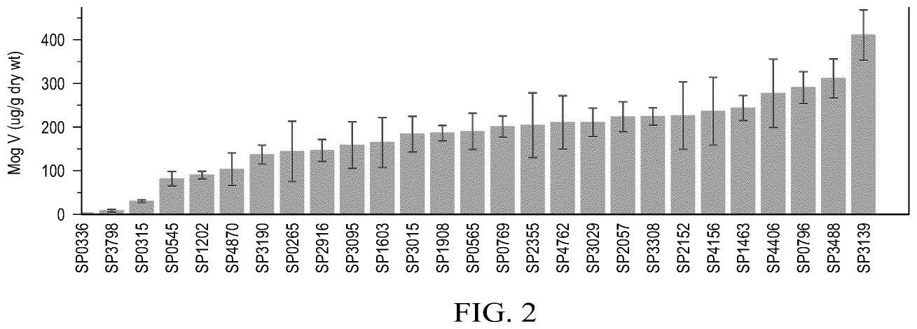

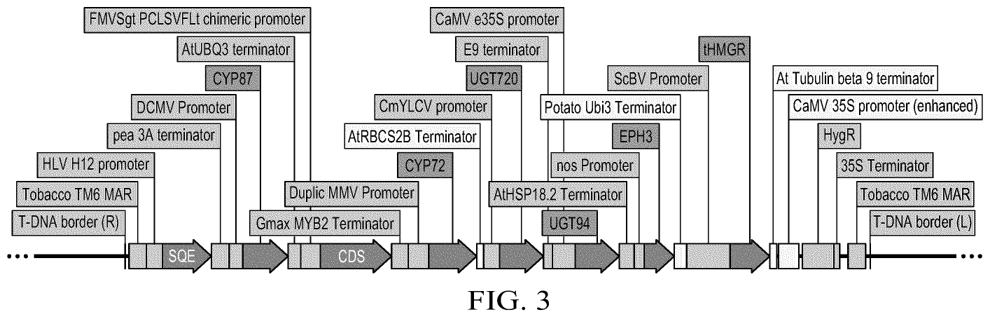

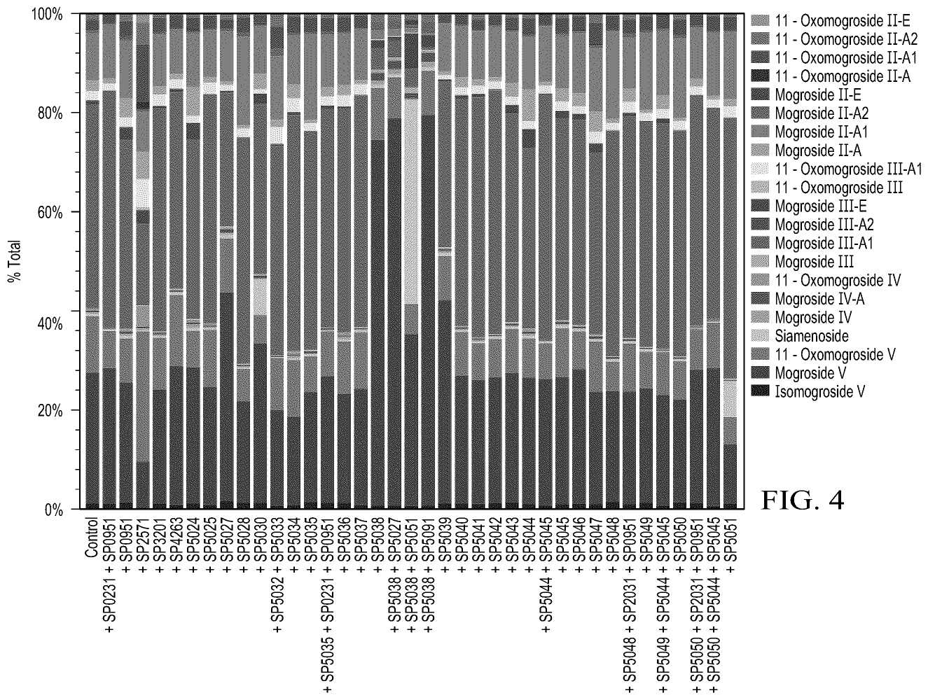

Additional studies in transient assays in watermelon fruit infiltrated with a number of different expression constructs described in Example 1, as well as certain combinations of these constructs, were performed. The production of various mogrosides and siamenoside is shown in FIG. 4. The amount of mogroside V normalized to control is shown in FIG. 5. The SP4263 expression construct produced the highest levels of mogroside V, but the SP5038 construct and the combination of the SP5038 and SP5027 constructs also produced high levels of mogroside V. The amount of siamenoside normalized to control is shown in FIG. 6. The combination of SP5038 and SP5051 produced the highest levels of siamenoside.

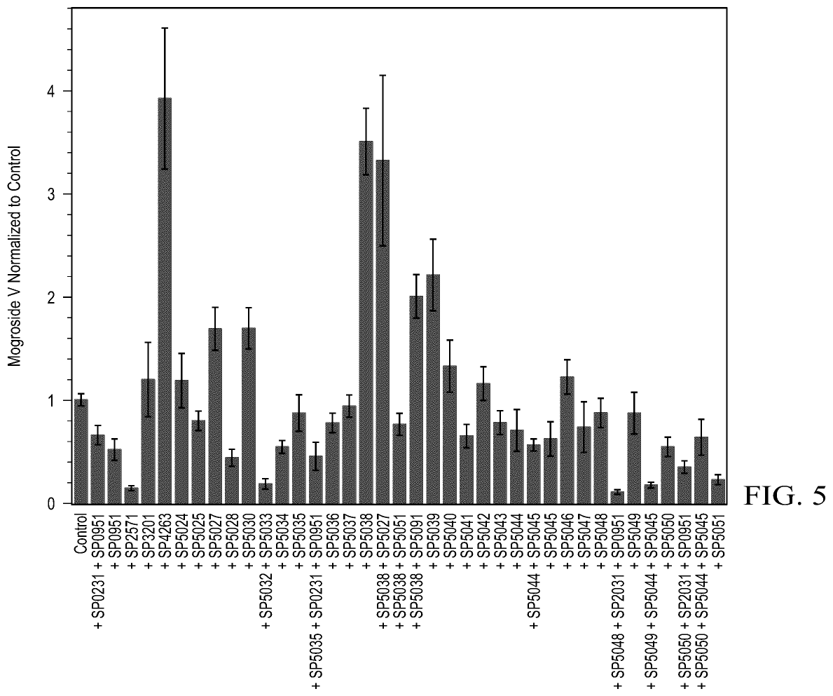

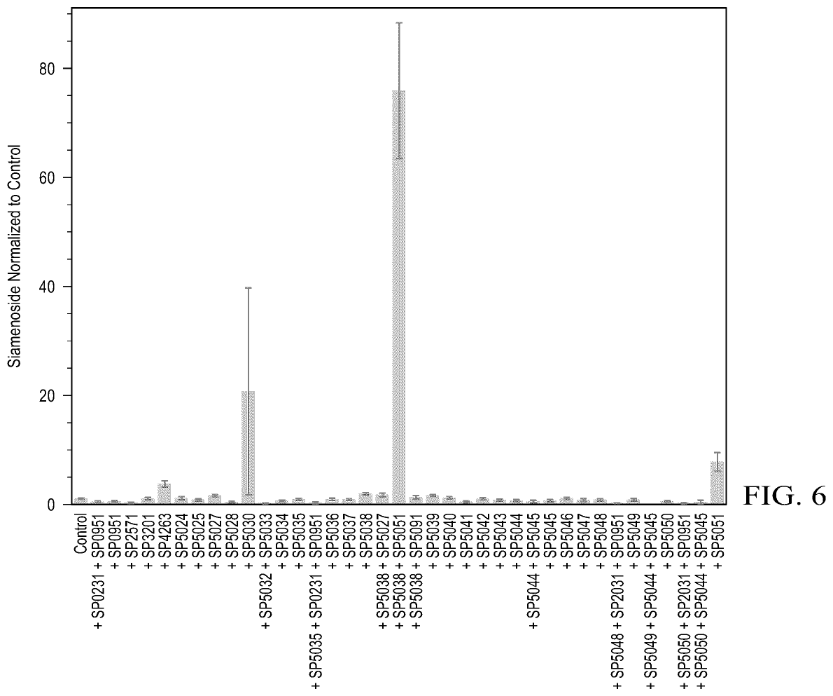

Further studies in transient assays in watermelon fruit infiltrated with a combination of SP2015, SP4332, SP5029, SP5030, SP5031, SP5032, and SP5033 constructs (control), and the combination lacking a single construct, were performed. The production of various mogrosides and siamenoside is shown in FIG. 7. The amount of mogroside V normalized to control is shown in FIG. 8.

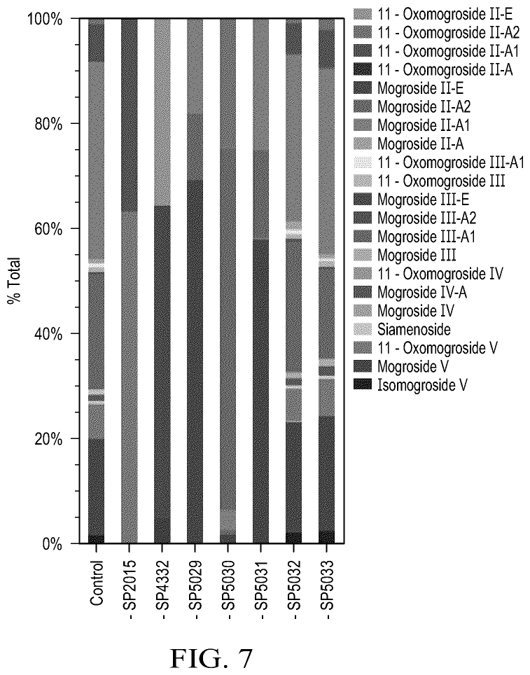

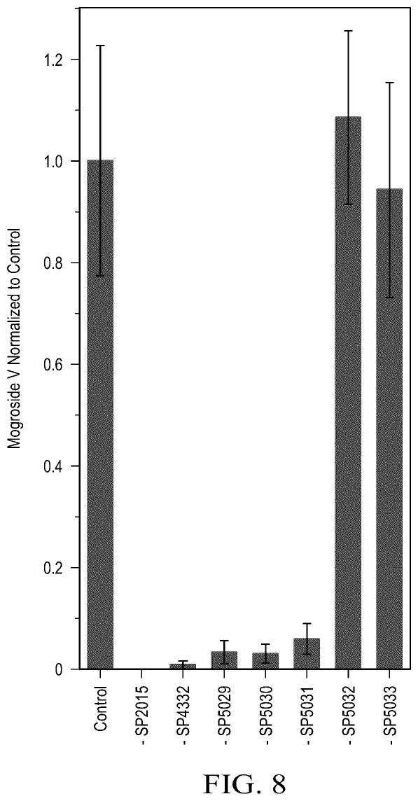

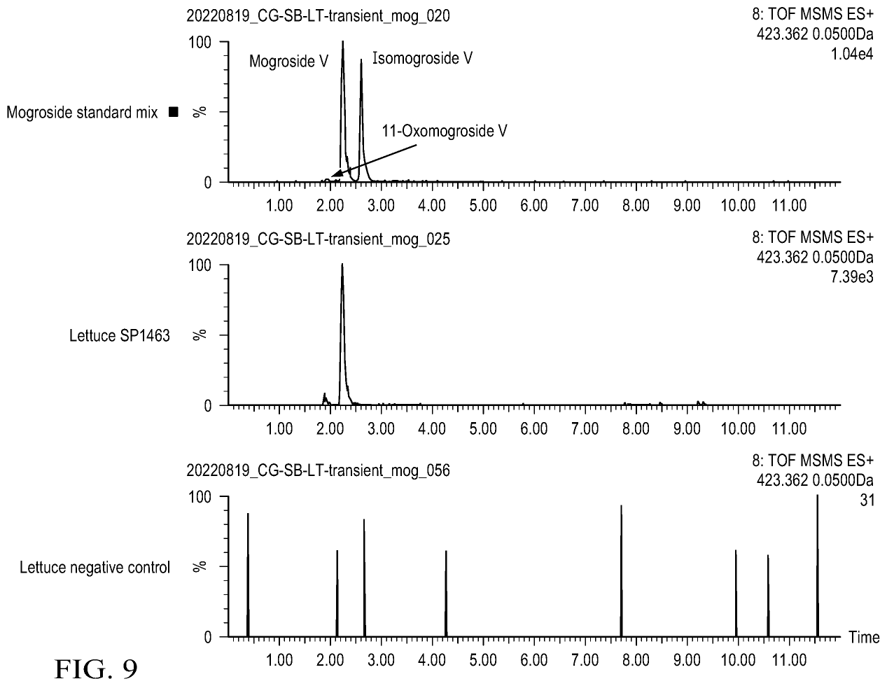

Mogroside V accumulation in transient assays from lettuce leaves infiltrated with the expression construct SP1463 is shown in FIG. 9 (middle panel). It can be seen that large amounts of mogroside V was produced, compared to the negative control (FIG. 9, bottom panel—note the difference in the scale of the middle panel and bottom panel of FIG. 9). The mogroside V eluted in the same location as mogroside V in a mogroside standard mix (FIG. 9, top panel). The mass spectrum fingerprinting of the mogroside V in transient assays from lettuce leaves infiltrated with the expression construct SP1463 is shown in FIG. 10. The mogroside V (FIG. 10, middle panel) matched the mass spectrum of the mogroside V reference standard (FIG. 10, top panel). The negative control showed no mogroside V (FIG. 10, bottom panel). A map of the SP1463 expression construct is shown in FIG. 11.

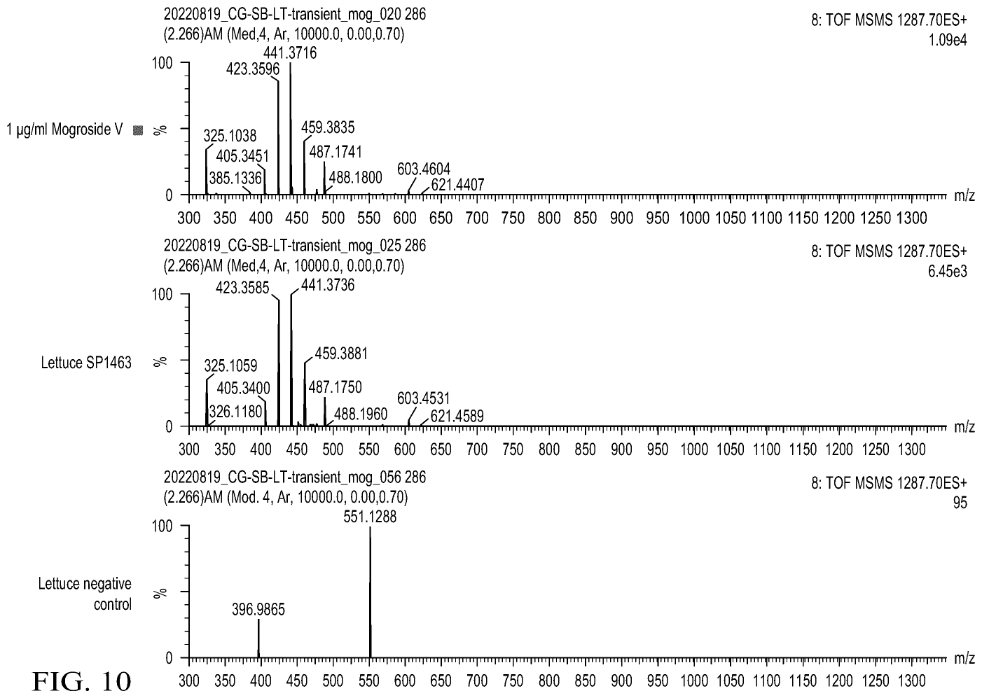

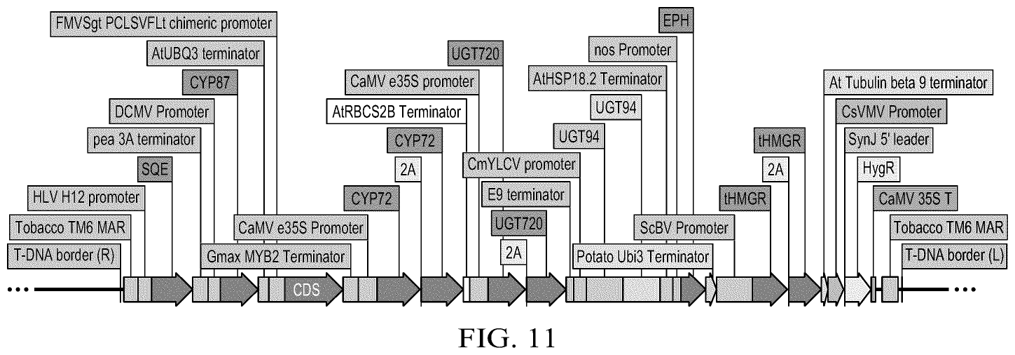

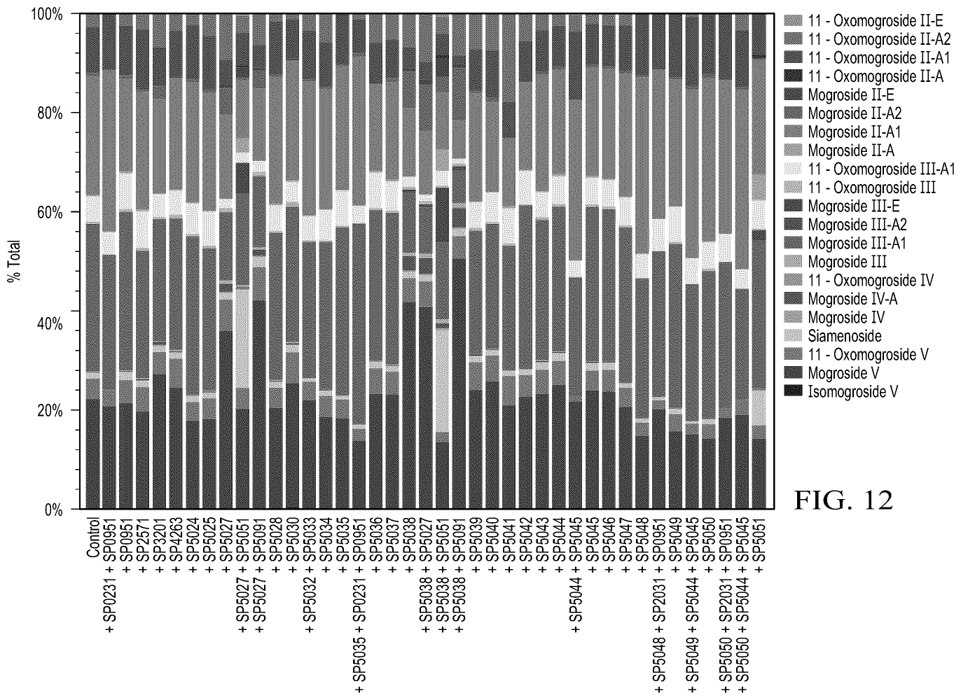

Additional studies in transient assays in lettuce leaves infiltrated with a number of different expression constructs described in Example 1, as well as certain combinations of these constructs, were performed. The production of various mogrosides and siamenoside is shown in FIG. 12. The amount of mogroside V normalized to control is shown in FIG. 13, and the amount of siamenoside normalized to control is shown in FIG. 14.

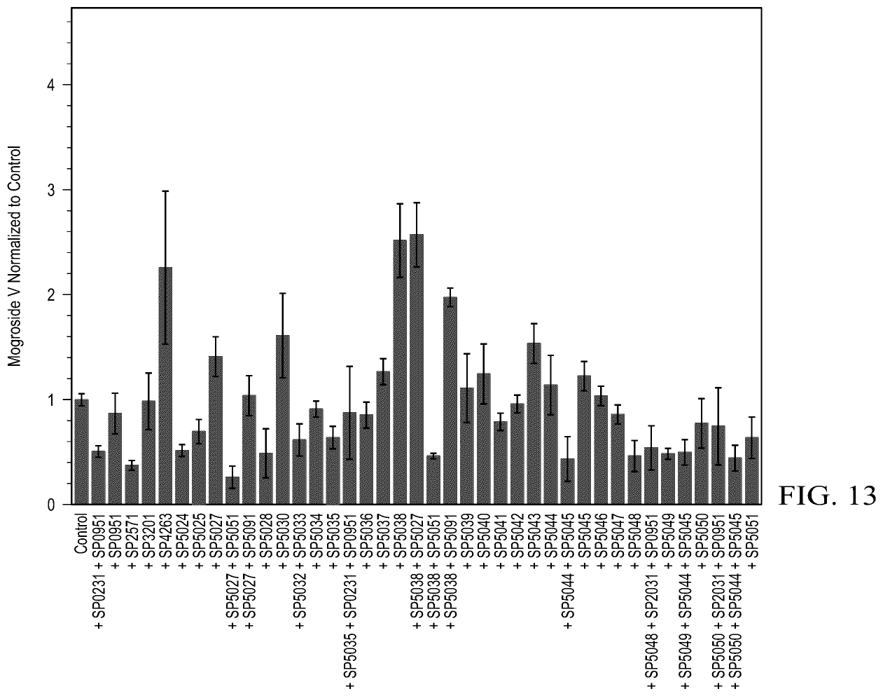

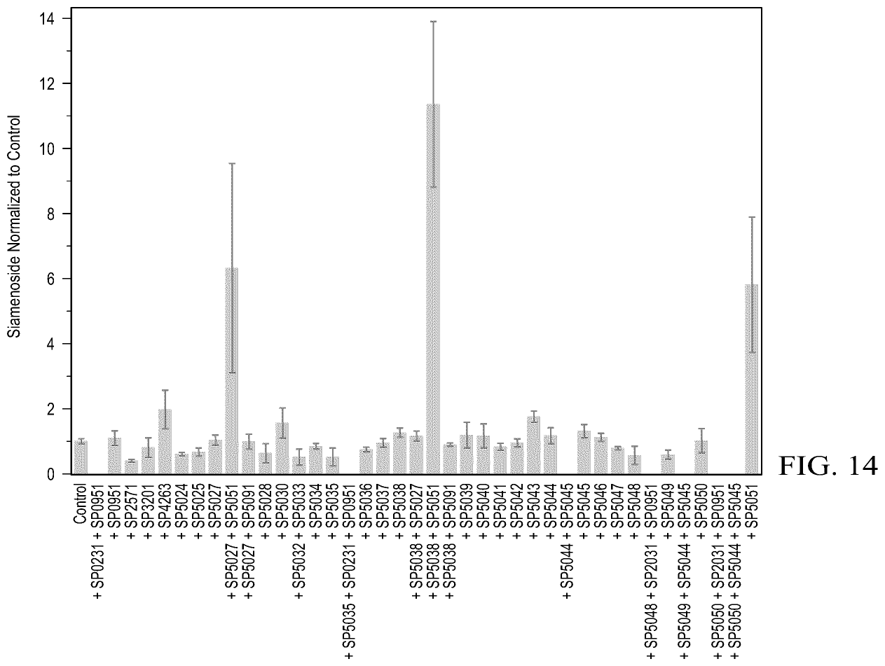

Further studies in transient assays in lettuce leaves infiltrated with a combination of SP2015, SP4332, SP5029, SP5030, SP5031, SP5032, and SP5033 constructs (control), and the combination lacking a single construct, were performed. The production of various mogrosides and siamenoside is shown in FIG. 15. The amount of mogroside V normalized to control is shown in FIG. 16.

### Example 4

### Transgenic Watermelon Plants

### Creation of Transformed Plants

Various expression cassettes selected from Example 1 were used for transforming Citrullus lanatus. Agrobacterium tumefaciens strain EHA105 was transformed with one or more expression cassette plasmids (selected from Example 1) using a free-thaw method (Weigel, CSH Protoc. 2006 Dec. 1; 2006(7): pdb.ip29. doi: 10.1101/pdb.ip29). Briefly, chemically competent Agrobacterium was prepared. After addition of the expression cassette plasmids, the mixture was alternately frozen in liquid nitrogen and thawed to liquid in a 37° C. water bath. The cells were then allowed to recover in LB medium for about 1 hour and plated out on LB plates with kanamycin.

Briefly, the transformed EHA105 Agrobacterium was grown overnight and then diluted until the OD₆₀₀ reading reached 0.12. Two commercial varieties of watermelon, Charleston Gray and Sugar Baby, were used as hosts. Five-day-old watermelon seedlings were used for preparing explants for transformation. Cotyledons were cut off from hypocotyls and collected in petri plates filled with sterile water. Two attached cotyledons were split by cutting through remaining hypocotyl segment, and cotyledonary explants were cut into 2 mm pieces ready for transformation. For transformation, Agrobacterium culture was added to these explants and placed under vacuum for 5 minutes. After infection, explants were blotted on sterile paper towels and transferred to filter disks in petri plates with MS medium. The plates were sealed and placed at 25° C. for 3 days in the dark for co-cultivation.

### Gene Expression Analysis of Target Genes in Transgenic Plants

To understand the difference in the ability to detect mogroside production in fruit, watermelons from various newly created plant lines were collected and dissected into the various fruit parts. RNA was then extracted from the various fruit part samples, and the RNA expression levels of the various newly integrated mogroside pathway genes were quantified using Q RT PCR using a standard protocol.

### Mogroside Detection

About 100 mg of plant tissue was extracted in 500 μl extraction buffer (80% methanol). After centrifugation, the supernatant was forced to pass through 0.22 μM filter in order to remove remaining particles. Waters Acquity UPLC coupled by Waters Xevo Quadrupole Time of Flight Tandem Mass Spectrometer was used for metabolite analysis. The initial LC separation of the samples was done using a Waters Quaternary Solvent Manager ACQUITY UPLC H-Class PLUS connected to a Waters Sample Manager FTN-H ACQUITY UPLC and a PDA eλ Detector ACQUITY UPLC. There were two different linear gradients, referred to as gradient A and gradient B, applied across different samples that use water 0.1% formic acid as mobile phase A (MPA) and acetonitrile 0.1% formic acid as mobile phase B (MPB) with a flow rate of 0.25 m1/minute and a column temperature of 35° C. Gradient A initializes at 10% MPB, at 1 minute it has increased to 20% MPB, 5 minutes 40% MPB, 8 minutes 70% MPB, 9 minutes 90% MPB, 12 minutes 95% MPB, 13 minutes 10% MPB, and 17 minutes 10% MPB. Gradient B initializes at 26% MPB, at 5 minutes it has increased to 35% MPB, 8.5 minutes 60% MPB, 9 minutes 90% MPB, 12 minutes 95% MPB, 13 minutes 26% MPB, and 17 minutes 26% MPB. The LC system was coupled to an Xevo G2-XS mass spectrometer in sensitivity positive mode using the MSMS scan mode with a scan range of 300-1350 m/z and collision energy of 30.

The following precursor and product ion pairs, expressed in m/z, were used to quantify the mogrosides with a 50 mDa window applied to the product ion: mogroside V (1287.7/423.3621), isomogroside V (1287.7/423.3621), mogroside IV (1125.6/423.3621), mogroside IV-A (1125.6/423.3621), siamenoside (1125.6/423.3621), mogroside III (963.6/423.3621), mogroside III-A (963.6/423.3621), mogroside III-A1 (963.6/423.3621), mogroside II-E (801.5/423.3621), mogroside II-A (801.5/423.3621), mogroside II-A1 (801.5/423.3621), 11-Oxo-mogroside V (1285.7/457.3680), and 11-Oxo-mogroside II-E (799.5/457.3680). For LockSpray mass correction, 200 pg/ml leucine-enkephalin monitoring for 556.2771 m/z was used. Data analysis was done using the Waters TargetLynx software, excel, and JMP. When creating figures, a 100 mDa or 50 mDa window was applied to the appropriate product listed above.

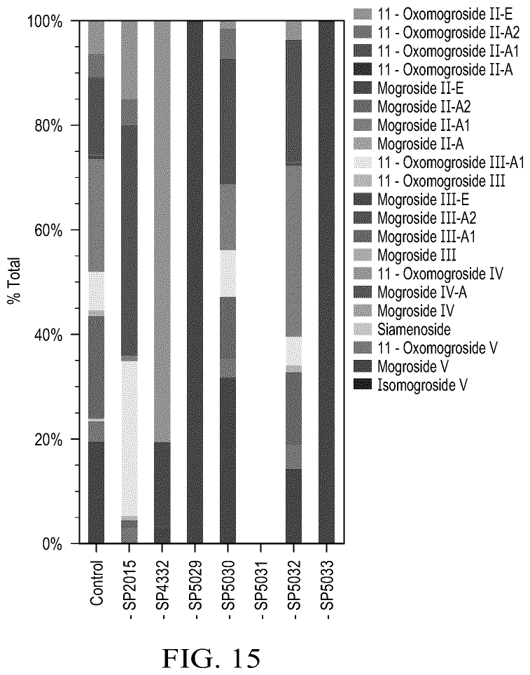

The levels of mogroside V were measured in the leaves of transgenic watermelon comprising expression cassette SP1463 The results are shown in FIG. 17 (bottom panel). The mogroside V eluted in the same location as mogroside V in a reference standard (FIG. 17, top panel). More than 3 mg/g (dry weight) of mogroside V was produced in transgenic watermelon. Interestingly, although the SP1463 construct did not produce the highest levels of mogroside V production in transient assays (see FIG. 2), extremely high levels of mogroside V was produced in transgenic watermelons comprising the SP1463 construct.

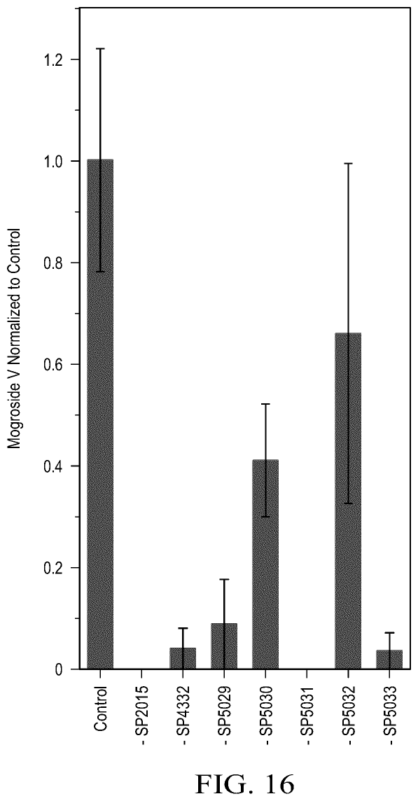

The results of mass spectrum fingerprinting are shown in FIG. 18. The mass spectrum of the mogroside V from the transgenic watermelon comprising expression cassette SP1463 (FIG. 18, bottom panel) matched the mass spectrum of the mogroside V reference standard (FIG. 18, top panel).

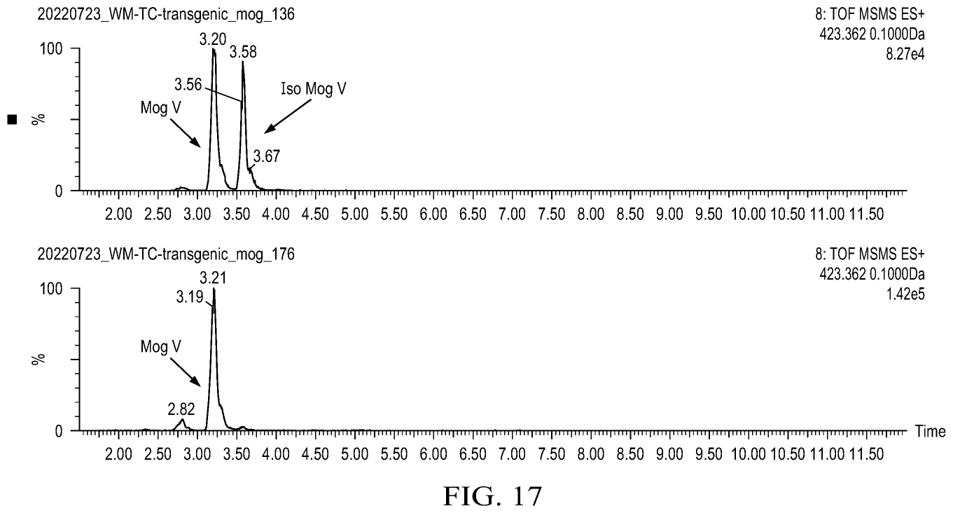

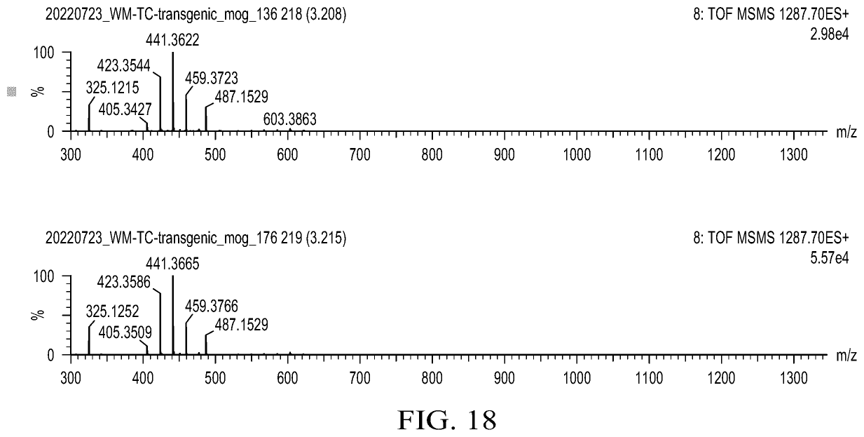

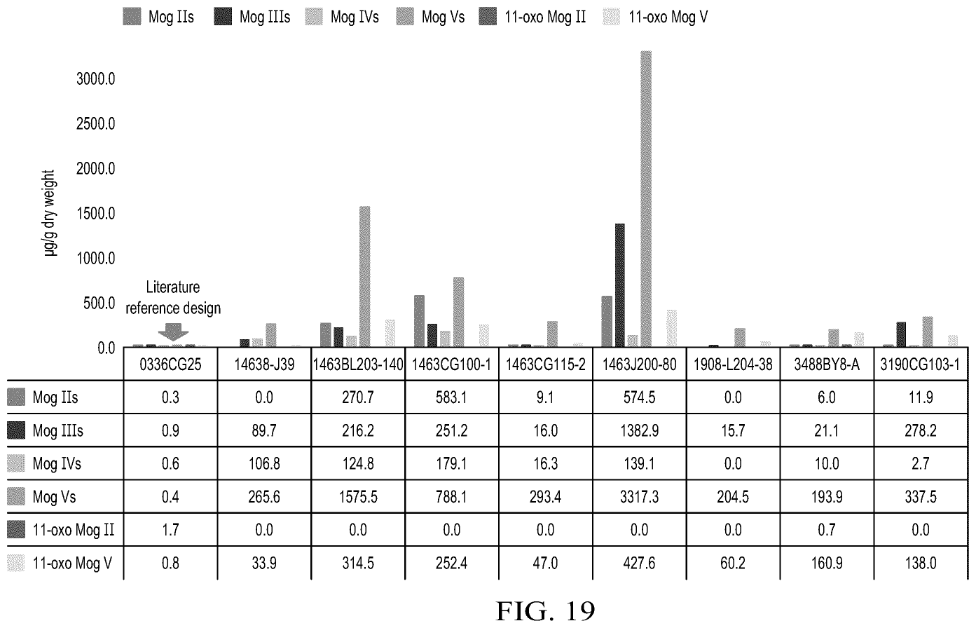

The levels of mogrosides, including mogroside V, were measured in the fruit of various transgenic watermelon lines comprising expression cassettes SP1463, SP1908, SP3190 and SP3488, and a construct (SP0336) based on the literature (Itkin et al., supra). The results are shown in FIG. 19. One of the SP1463 transgenic watermelon lines produced more than 3 mg/g (dry weight) of mogroside V. This was hundreds of thousands times more than was produced by the literature construct SP0336. A map of the SP0336 construct is shown in FIG. 20, and a map of the SP1908 construct is shown in FIG. 21.

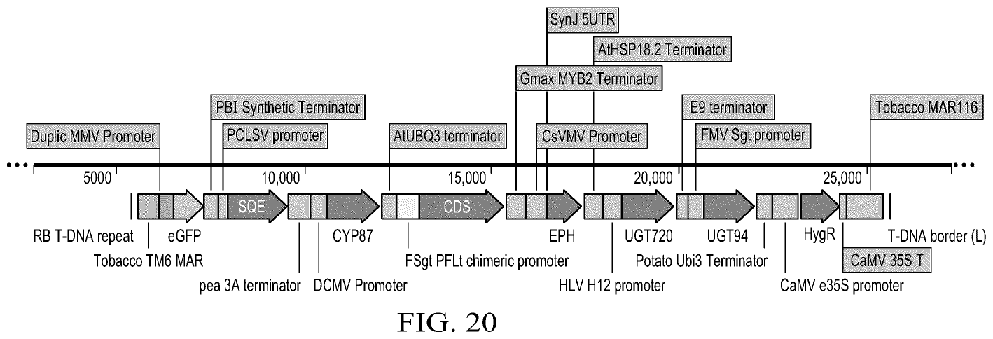

The levels of various mogrosides and siamenoside were measured in the fruit and leaves of various transgenic watermelon lines comprising expression cassettes SP0565, SP0641, SP1463, SP1908, SP3015, SP3016, SP3029, SP3190, SP3308 and SP3488. The results are shown in FIG. 22.

### Example 5

### Production of Mogrosides in a Transgenic Watermelon Cell Suspension

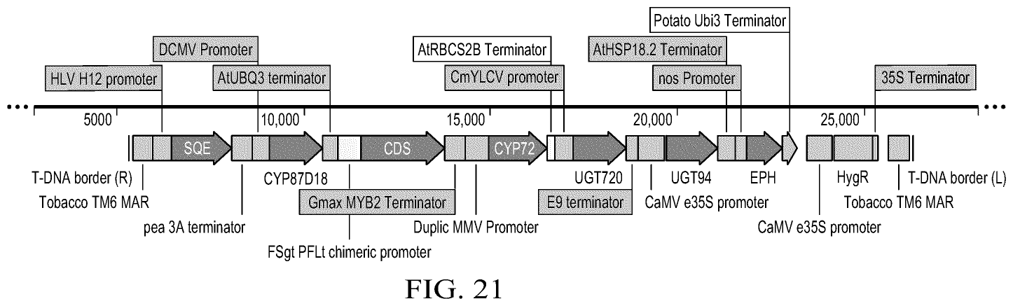

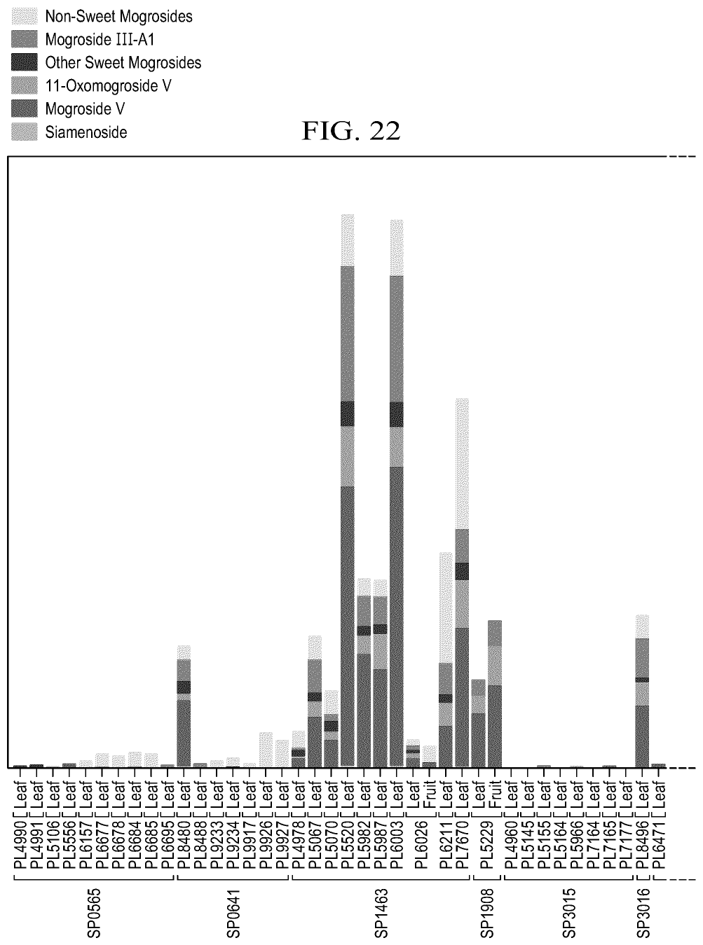

A watermelon cell suspension was established that produces mogrosides. Cells originated from anther-derived callus induced from the immature flowers of a transgenic watermelon plant (transgenic construct=SP3190). After one month, the culture increased 4-5× in volume every 7 days. To determine amount and type of mogrosides in the cell suspension, approximately 1 mL of packed settled cell volume from a 7-day old culture was transferred to a 2 mL collection tube and the contents were centrifuged at 12,000×g for 10 minutes, the supernatant was removed and the samples were flash frozen in liquid nitrogen, and the samples were then analyzed for mogrosides and siamenoside. Mogroside measurements of the cell suspension by collection date are shown in FIG. 23 (samples with the same date are technical replicates). The concentration of mogrosides and siamenoside in transgenic watermelon cell suspension is shown in FIG. 24.

### Example 6

### Transgenic Tomato Plants

Various expression cassettes selected from Example 1 were used for transforming Solanum lycopersicum. Agrobacterium tumefaciens strain EHA105 was transformed with one or more expression cassette plasmids (selected from Example 1) using a free-thaw method (Weigel, CSH Protoc. 2006 Dec. 1; 2006(7): pdb.ip29. doi: 10.1101/pdb.ip29). Briefly, chemically competent Agrobacterium was prepared. After addition of the expression cassette plasmids, the mixture was alternately frozen in liquid nitrogen and thawed to liquid in a 37° C. water bath. The cells were then allowed to recover in LB medium for about 1 hour and plated out on LB plates with kanamycin.

Briefly, the transformed EHA105 Agrobacterium was grown overnight and then diluted until the OD₆₀₀ reading reached 0.3. Two varieties of tomato, MicroTom and MoneyMaker, were used as hosts. Six-to-Seven-day-old tomato seedlings were used for preparing explants for transformation. Cotyledons and hypocotyls were cut off from seedlings and collected in petri plates filled with sterile water. Once all explants have been prepared, the water is removed from each plate by pipetting, being careful to not remove or excessively damage the explants. The cotyledons are immersed in 20-30 mL of Agrobacterium suspension, the plates are sealed and gently shaken at 50 rpm for 15 minutes. These explants, without excess bacterial, are then placed on tomato co-cultivation media (Table 1).

TABLE 1

| Ingredient or Action | Amount | |
|---|---|---|
| MS Media + Gamborg Vitamins (M404) | 1x | |
| Sucrose (3%) | 30 | g/L |
| Glucose (0.2%) | 2 | g/L |
| pH to 5.6 | | |
| 500 mL + 1.5 g Gelzan (G215) | 3 | g/L Gelzan |
| BAP | 1 | mg/L |
| NAA | 0.1 | mg/L |
| Acetosyringone | 200 | μM |

After 2 days of co-culture, transfer explants to Tomato Selection Media (TSM; Table 2), abaxial side up, 14-18 explants per plate depending on their size. Growth conditions are 16 hour photoperiod (provided by 20 watt cool-white fluorescent tubes yielding a light intensity of 301 μmol/m²/sec) at 25° C.

TABLE 2

| Ingredient or Action | Amount | |
|---|---|---|
| MS Media + Gamborg Vitamins (M404) | 1x | |
| Sucrose | 30 | g/L |
| pH to 5.6 | | |
| 500 mL + 1.5 g Gelzan (G215) | 3 | g/L Gelzan |
| Autoclave 25 minutes | | |
| IAA | 0.1 | mg/L |
| Zeatin | 2 | mg/L |
| Hygromycin | 25 | mg/L |
| Timentin | 400 | mg/L |

Subculture regularly every 2 weeks transferring to new TSM media. Transgenic events are then selected based on gene expression and mogroside accumulation.

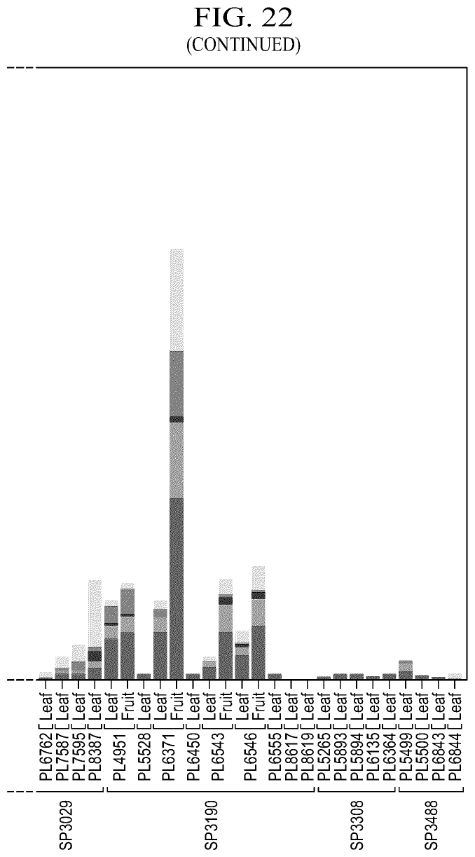

The levels of various mogrosides and siamenoside were measured in the fruit and leaves of various transgenic tomato lines comprising expression cassettes SP0641, SP1463, SP1908, SP3016, SP3029, SP3190 and SP3684. The results are shown in FIG. 25.

### Example 7

### Transgenic Potato Plants

Various expression cassettes selected from Example 1 were used for transforming potato. Agrobacterium tumefaciens strain EHA105 was transformed with one or more expression cassette plasmids (selected from Example 1) using a free-thaw method (Weigel, CSH Protoc. 2006 Dec. 1; 2006(7): pdb.ip29. doi: 10.1101/pdb.ip29). Briefly, chemically competent Agrobacterium was prepared. After addition of the expression cassette plasmids, the mixture was alternately frozen in liquid nitrogen and thawed to liquid in a 37° C. water bath. The cells were then allowed to recover in LB medium for about 1 hour and plated out on LB plates with kanamycin.

Transformation to potato was performed based on a published protocol (Chronis, Bio protocol, 2014 DOI: 10.21769/BioProtoc.1017).

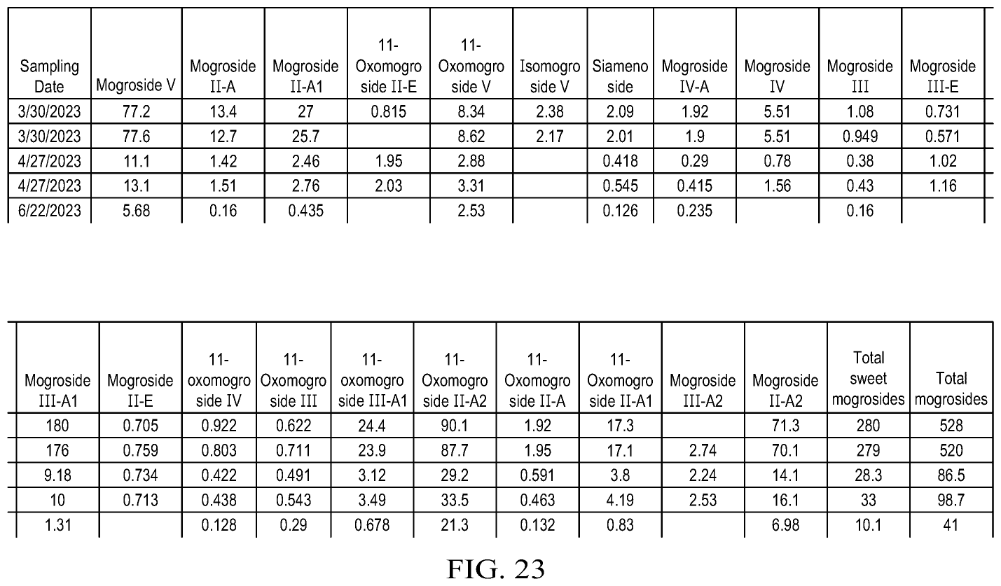

The levels of various mogrosides and siamenoside were measured in the fruit and leaves of various transgenic potato lines comprising expression cassettes SP0641, SP1463, SP3016, SP3029, SP3190 and SP3684. The results are shown in FIG. 26.

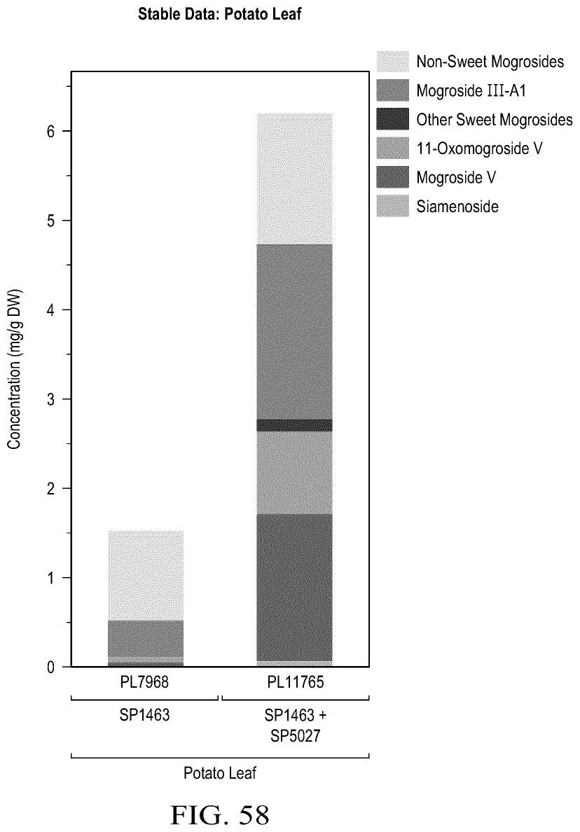

Additional studies were performed by transforming potato with vector SP1463 and then re-transforming that transgenic event with S1UGT (SP5027). The results are shown in FIG. 58. The results show that transgenic potatoes were produced with up to 6 mg/g total mogrosides and almost 5 mg/g sweet mogrosides.

### Example 8

### Transgenic Lettuce Plants

Various expression cassettes selected from Example 1 were used for transforming lettuce (Lactuca sativa). Agrobacterium tumefaciens strain EHA105 was transformed with one or more expression cassette plasmids (selected from Example 1) using a free-thaw method (Weigel, CSH Protoc. 2006 Dec. 1; 2006(7): pdb.ip29. doi: 10.1101/pdb.ip29). Briefly, chemically competent Agrobacterium was prepared. After addition of the expression cassette plasmids, the mixture was alternately frozen in liquid nitrogen and thawed to liquid in a 37° C. water bath. The cells were then allowed to recover in LB medium for about 1 hour and plated out on LB plates with kanamycin.

Briefly, the transformed EHA105 Agrobacterium was grown overnight and then diluted until the OD₆₀₀ reading reached 0.3. Five-day-old lettuce seedlings were used for preparing explants for transformation. Cotyledons were cut off from seedlings and collected in petri plates filled with sterile water. Once all explants have been prepared, the water is removed from each plate by pipetting, being careful to not remove or excessively damage the explants. The cotyledons are immersed in 20-30 mL of Agrobacterium suspension, the plates are sealed and gently shaken at 50 rpm for 10 minutes. These explants, without excess bacterial, are then placed on tomato co-cultivation media (Table 3).

TABLE 3

| Ingredient or Action | Amount | |
|---|---|---|
| MS Media + Vitamins (M519) | 1x | |
| Sucrose (3%) | 30 | g/L |
| pH to 5.7 | | |
| 500 mL + 1.5 g Gelzan | 3 | g/L Gelzan |
| Autoclave 25 minutes | | |
| BAP | 0.5 | mg/L |
| NAA | 0.1 | mg/L |
| Acetosyringone | 50 | μM |

After 3 days of co-culture, transfer explants to Lettuce Selection Media (LSM; Table 4), abaxial side up, 10 explants per plate depending on their size.

TABLE 4

| Ingredient or Action | Amount | |
|---|---|---|
| MS Media + Vitamins (M519) | 1x | |
| Sucrose | 30 | g/L |
| pH to 5.7 | | |
| 500 mL + 1.5 g Gelzan | 3 | g/L Gelzan |
| Autoclave 25 minutes | | |
| NAA | 0.1 | mg/L |
| BAP | 0.5 | mg/L |
| Hygromycin | 12.5 | mg/L |
| Timentin | 400 | mg/L |

Subculture regularly every 2 weeks transferring to new LSM media. Transgenic events are then selected based on gene expression and mogroside accumulation.

### Example 9

### Transgenic Sugar Beet Plants

Various expression cassettes selected from Example 1 were used for transforming sugar beet. Agrobacterium tumefaciens strain EHA105 was transformed with one or more expression cassette plasmids (selected from Example 1) using a free-thaw method (Weigel, CSH Protoc. 2006 Dec. 1; 2006(7): pdb.ip29. doi: 10.1101/pdb.ip29). Briefly, chemically competent Agrobacterium was prepared. After addition of the expression cassette plasmids, the mixture was alternately frozen in liquid nitrogen and thawed to liquid in a 37° C. water bath. The cells were then allowed to recover in LB medium for about 1 hour and plated out on LB plates with kanamycin.

Briefly, the transformed EHA105 Agrobacterium was grown overnight and then diluted until the OD₆₀₀ reading reached 0.6. Sugar beet calli were used as explants for transformation. The materials were immersed in 20-30 mL of Agrobacterium suspension, the plates are sealed and gently shaken at 50 rpm for 15 minutes. These explants, without excess bacteria, are then placed on sugar beet co-cultivation media (Table 5).

TABLE 5

| Ingredient or Action | Amount | |
|---|---|---|
| MS Media + Vitamins | 1x | |
| Sucrose (3%) | 30 | g/L |
| Glucose (0.2%) | 6 | g/L |
| pH to 5.7 | | |
| BAP | 1 | mg/L |
| Acetosyringone | 200 | μM |

After 3 days of co-culture, transfer explants to Sugar Beet Selection Media (SBSM; Table 6). Growth in dark at 25° C.

TABLE 6

| Ingredient or Action | Amount | |
|---|---|---|
| MS Media + MS Vitamins | 1x | |
| Sucrose | 30 | g/L |
| pH to 5.7 | | |
| Agar | 6 | g/L |
| BAP | 1 | mg/L |
| Hygromycin | 10 | mg/L |
| Timentin | 400 | mg/L |

Subculture regularly every 4 weeks transferring to new TSM media. Transgenic events are then selected based on gene expression and mogroside accumulation.

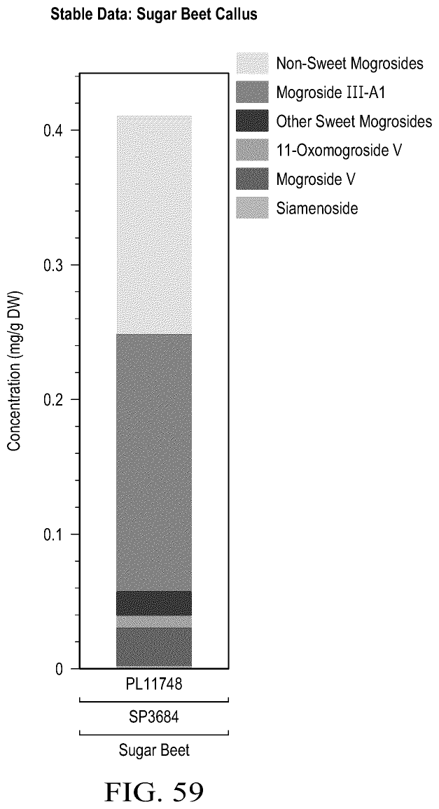

Studies were performed by transforming sugar beet with vector SP3684 and measuring mogrosides in callus. The results are shown in FIG. 59. The results show that transgenic sugar beet callus transformed with vector SP3684 produced 0.4 mg/g total mogrosides.

### Example 10

### Production of Mogrosides in Yeast

Production of mogrosides in yeast has been described previously (see, e.g., U.S. Pat. Nos. 10,465,222, 10,633,685 and 11,060,124, each of which is incorporated herein by reference in its entirety). By replacing one or more of the enzymes previously used to produce mogrosides with one or more of the enzymes disclosed herein, or by adding one of the CYP72 enzymes disclosed herein to the enzymes previously used to produce mogrosides, the present disclosure also provides methods for producing mogrosides in yeast, using control elements for expression of the nucleic acid sequences in yeast that are well-known to those of skill in the art or control elements previously described.

### Example 11

### Production of Siamenoside I

Using the presently disclosed methods for the production of mogrosides in plants and other organisms, siamenoside I can be produced by a number of different methods. First, starting with a plant or organism that produces mogroside V (such as plants and organisms disclosed herein), the plant or organism can be engineered to express enzymes such as glucan 1,3-beta-glucosidase I/II (ExgI) or DbExgI (see U.S. Pat. No. 11,180,789, incorporated herein by reference in its entirety) to convert mogroside V to siamenoside I and various mogroside II compounds, such as mogroside III E. Second, starting with a plant or organism that produces mogroside III E (such as plants and organisms disclosed above or elsewhere herein), the plant or organism can be engineered to express enzymes such as UGT-M2 (Xu, et al., J. Agric. Food Chem. 70:1601-1609, 2022) to convert mogroside III E to siamenoside I. Third, starting with a plant or organism that produces mogroside III A1 (such as plants and organisms disclosed herein), the plant or organism can be engineered to express enzymes such as UGT-74_345_2 or UGT74_406_2 (Itkin et al., Proc Nat Acad Sci USA 113:E7619-E7628, 2016) to convert mogroside III E to siamenoside I.

### Example 12

### Production of α-Siamenoside I

Starting with a plant or organism that produces mogrosides (such as plants and organisms disclosed herein), the plant or organism can be engineered to express enzymes such as those disclosed in U.S. Pat. No. 11,060,124 (incorporated herein by reference in its entirety) to convert mogrosides to compound 1 (α-siamenoside I). Alternatively, starting with a plant or organism that produces mogrosides (such as plants and organisms disclosed herein), the plant or organism can be engineered to express cyclodextrin glycosyltransferases (CGTases; Xu et al., Food Chem. 359:October 2021, doi.org/10.1016/j.foodchem.2021.129938) to convert mogrosides to α-siamenoside I.

### Example 13

### Co-Production of Mogrosides and Rebaudioside M

Biosynthesis of Rebaudioside M (RebM) in recombinant host cells has previously been described (PCT Patent Application Publication No. WO 2014/122227, incorporated herein by reference in its entirety). Starting with a plant or organism that produces mogrosides (such as plants and organisms disclosed herein), the plant or organism can be engineered to express the enzymes to produce Reb M to produce plants or organisms that produce mogrosides and Reb M, in the same cells. Alternatively, plants or organisms that are transformed to express Reb M pathway genes can be crossed with plants or organisms that express mogroside pathway genes (as disclosed herein), to produce plants or organisms that contain both pathways at the same time and produce a mixture of mogrosides and Reb M.

In addition, the following UGT enzymes can be used with rebaudioside biosynthesis genes to further improve the efficiency for biosynthesis of certain rebaudiosides in plants or other organisms: UGT76G1 T284S mutant (Liu et al., Plant Commun. 1:Jan. 13, 2020, doi.org/10.1016/j.xplc.2019.100004); UGT76G1 S195Q mutant (Yu et al., J. Funct. Foods 92: May 2022, doi.org/10.1016/j.jff.2022.105033); or Oryza sativa EUGT11 (Wang et al., Int. J. Biol. Macromol. 163: 1669-1676, 2020).

### Example 14

### Detection of Non-Canonical Mogroside Isomers

Several non-canonical mogroside isomers are observed to a varying degree when vectors like SP3684 are transformed into many species, with some like tomato yielding prominent quantities of these isomers, and others like watermelon yielding minor quantities of these isomers. Because these non-canonical mogroside isomers show up at the same mass as mogrosides with two to five glucose groups and their 11-oxo forms, they likely have a glucose group added at a different position than the mogroside standards. Various mogrosides and non-canonical mogroside isomers were detected in transient watermelon fruit co-infiltrations with the construct SP3684 enhanced in t72143 (uridine phosphorylase dependent glycosyltransferase; SEQ ID NO:138; SP5041) relative to green fluorescent protein (GFP).

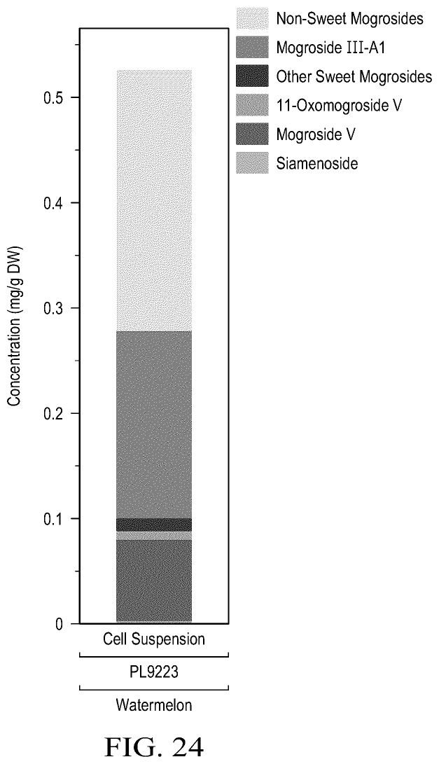

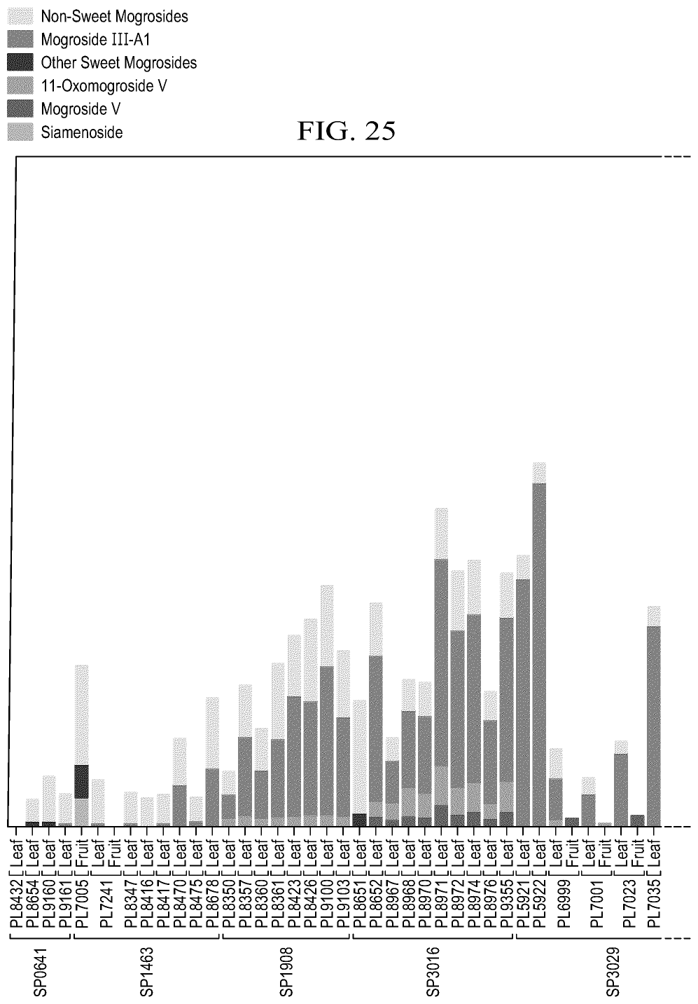

Mogroside V and isomers accumulation in transient assays is shown in FIG. 27. For mogroside V, acquire data in TofMSMS ESI positive mode, selecting for mogroside V plus a proton (1287.6580 m/z), using the collision energy 30 V, and collecting a full high-resolution mass scan (270-1350 m/z) for resulting fragments. The chromatograms shown are selected for the fragment mass 423.3621 m/z with a 0.02 Da mass window. Mogroside V fragment mass 423.3603 m/z is 4.3 ppm error compared to the theoretical 423.3621 m/z. Unknown peak is 423.3610 m/z is 2.7 ppm error compared to the theoretical 423.3621 m/z. 5 ppm is considered an acceptable cutoff for high resolution mass error. Mogroside V and isomers accumulation in transient assays is shown in FIG. 27. The production of mogroside V and isomogroside V in transient watermelon fruit infiltrated with the construct SP3684 is shown in FIG. 27, top panel. It can be seen that an unknown mogroside V isomer was produced when SP3684 was co-infiltrated with t72143 (FIG. 27, middle panel), compared to SP3684 co-infiltrated with GFP (FIG. 27, bottom panel). FIG. 28 shows the mass spectrum fingerprinting of mogroside V (top panel) and the unknown mogroside V isomer (bottom panel).

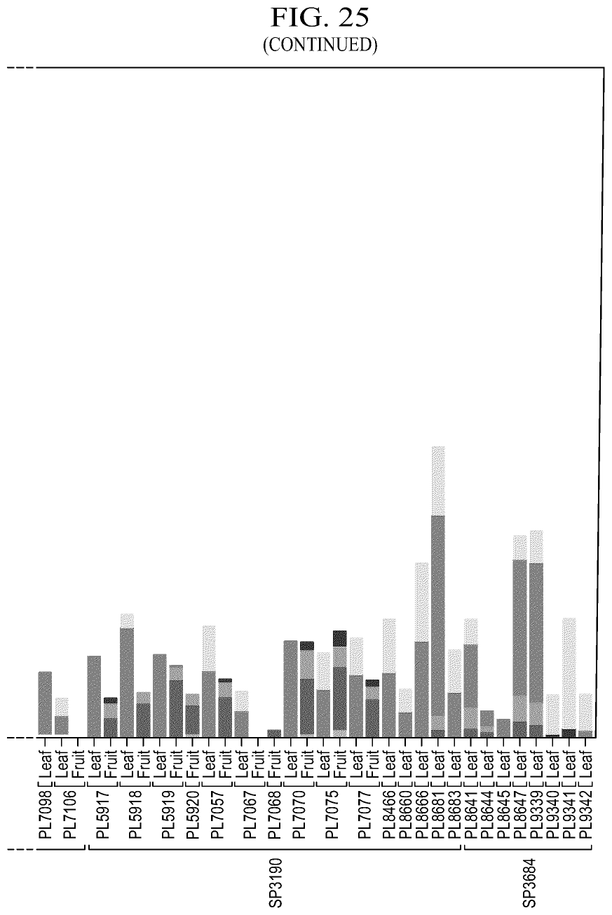

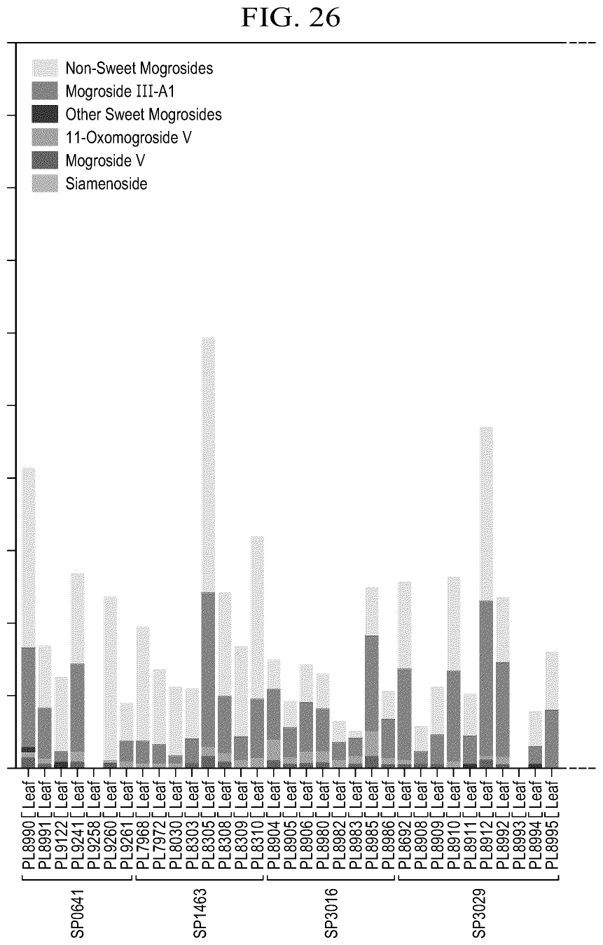

Mogroside IV and isomers accumulation in transient assays is shown in FIG. 29. For mogroside IV, acquire data in TofMSMS ESI positive mode, selecting for the mogroside IV plus a proton (1125.6051 m/z), using the collision energy 30 V, and collecting a full high-resolution mass scan (270-1350 m/z) for resulting fragments. The chromatograms shown are selected for the fragment mass 423.3621 m/z with a 0.02 Da mass window. Mogroside IV fragment mass 423.3616 m/z is 1.3 ppm error compared to the theoretical 423.3621 m/z. Unknown peak 2 is 423.3596 m/z is 6.0 ppm error compared to the theoretical 423.3621 m/z. Unknown peak 1 is 423.3615 m/z is 1.5 ppm error compared to the theoretical 423.3621 m/z. Missing peaks are likely due to the low intensity of this peak. 5 ppm is considered an acceptable cutoff for high resolution mass error. The production of mogroside IV, mogroside IV-A and siamenoside in transient watermelon fruit infiltrated with the construct SP3684 is shown in FIG. 29, top panel. It can be seen that two unknown mogroside IV isomers were produced when SP3684 was co-infiltrated with t72143 (FIG. 29, middle panel), compared to SP3684 co-infiltrated with GFP (FIG. 29, bottom panel). FIG. 30 shows the mass spectrum fingerprinting of mogroside IV (top panel) and the unknown mogroside IV isomers (middle and bottom panels).

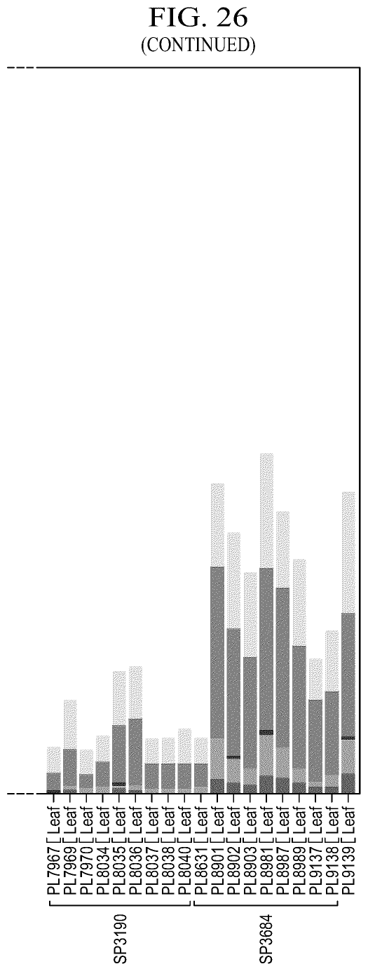

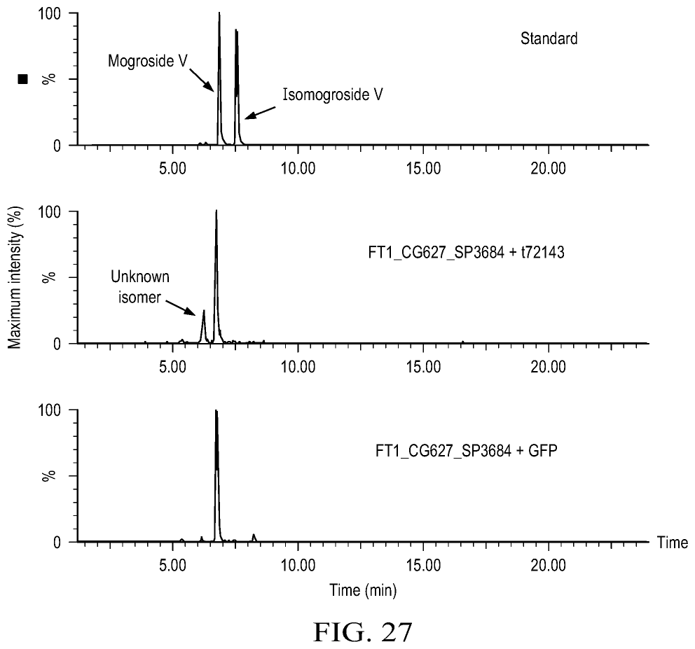

Mogroside III and isomers accumulation in transient assays is shown in FIG. 31. For mogroside III, acquire data in TofMSMS ESI positive mode, selecting for the mogroside III plus a proton (963.5523 m/z), using the collision energy 30 V, and collecting a full high-resolution mass scan (270-1350 m/z) for resulting fragments. The chromatograms shown are selected for the fragment mass 423.3621 m/z with a 0.02 Da mass window. Mogroside III-A1 fragment mass 423.3609 m/z is 2.9 ppm error compared to the theoretical 423.3621 m/z. Unknown peak is 423.3615 m/z is 1.5 ppm error compared to the theoretical 423.3621 m/z. 5 ppm is considered an acceptable cutoff for high resolution mass error. The production of mogroside III, mogroside III-E, mogroside III-A1 and mogroside III-A2 in transient watermelon fruit infiltrated with the construct SP3684 is shown in FIG. 31, top panel. It can be seen that an unknown mogroside V isomer was produced when SP3684 was co-infiltrated with t72143 (FIG. 31, middle panel), compared to SP3684 co-infiltrated with GFP (FIG. 31, bottom panel). FIG. 32 shows the mass spectrum fingerprinting of mogroside III-A1 (top panel) and the unknown mogroside III isomer (bottom panel).

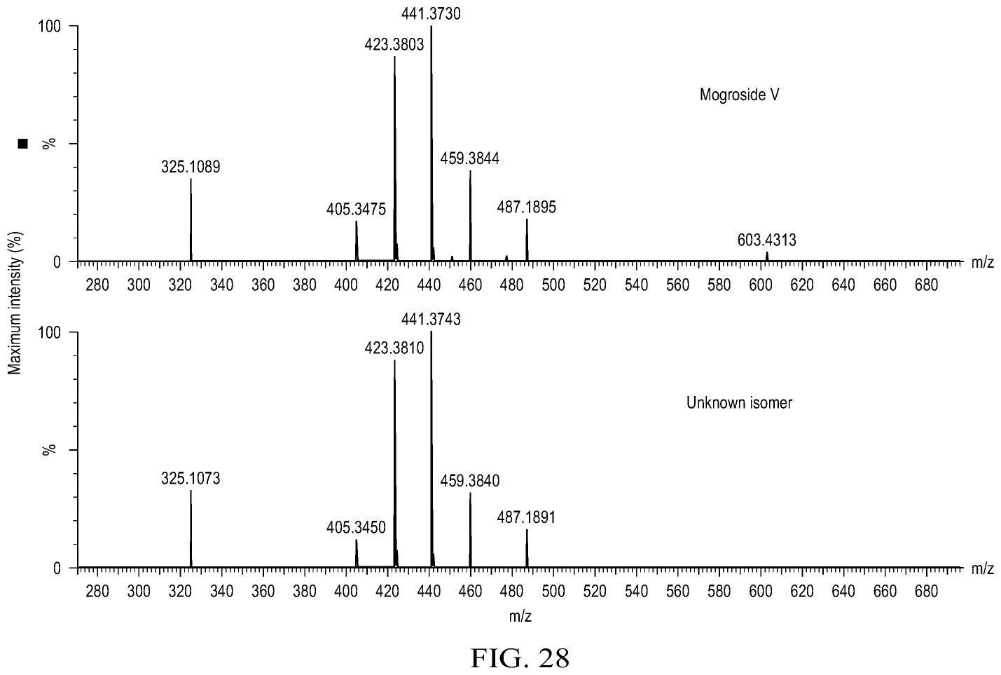

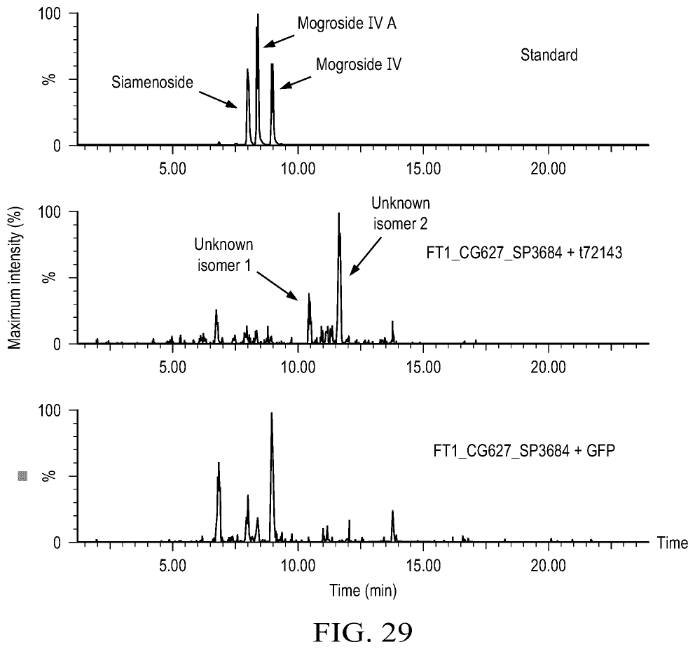

Mogroside II and isomers accumulation in transient assays is shown in FIG. 33. For mogroside II, acquire data in TofMSMS ESI positive mode, selecting for the mogroside II plus a proton (801.4995 m/z), using the collision energy 30 V, and collecting a full high-resolution mass scan (270-1350 m/z) for resulting fragments. The chromatograms shown are selected for the fragment mass 423.3621 m/z with a 0.02 Da mass window. Mogroside II-A1 fragment mass 423.3607 m/z is 3.4 ppm error compared to the theoretical 423.3621 m/z. Unknown peak is 423.3603 m/z is 4.3 ppm error compared to the theoretical 423.3621 m/z. Missing peaks are likely due to the low intensity of this peak. 5 ppm is considered an acceptable cutoff for high resolution mass error. The production of mogroside II-A, mogroside II-A1, mogroside II A2, mogroside II-E and mogroside 1-A(1-3)-glucopyranoside in transient watermelon fruit infiltrated with the construct SP3684 is shown in FIG. 33, top panel. It can be seen that an unknown mogroside II isomer was produced when SP3684 was co-infiltrated with t72143 (FIG. 33, middle panel), compared to SP3684 co-infiltrated with GFP (FIG. 33, bottom panel). FIG. 34 shows the mass spectrum fingerprinting of mogroside II-A1 (top panel) and the unknown mogroside II isomer (bottom panel).

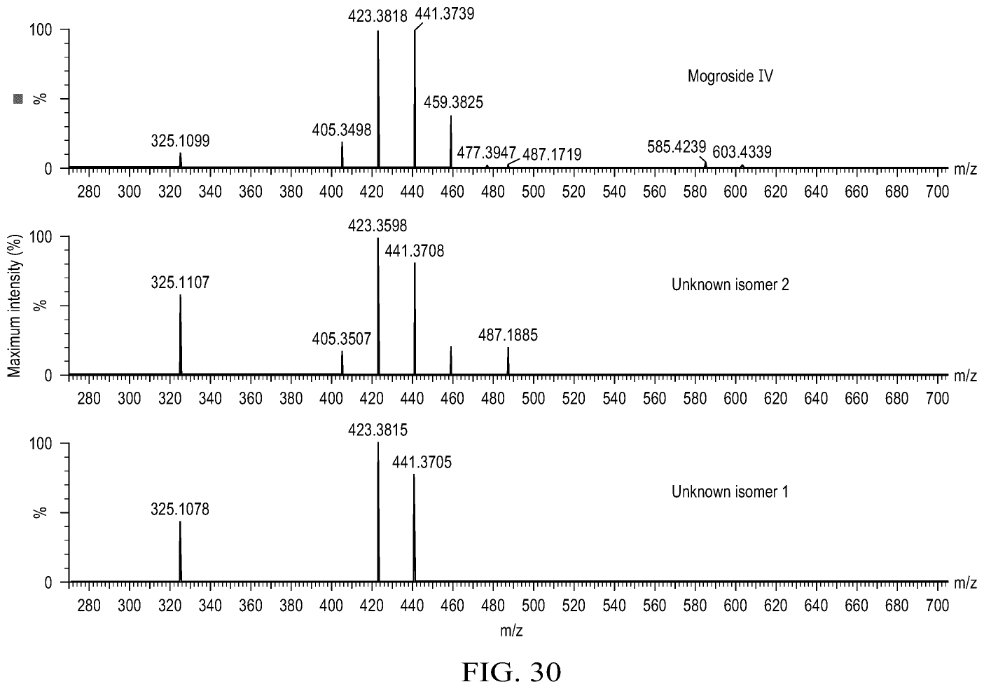

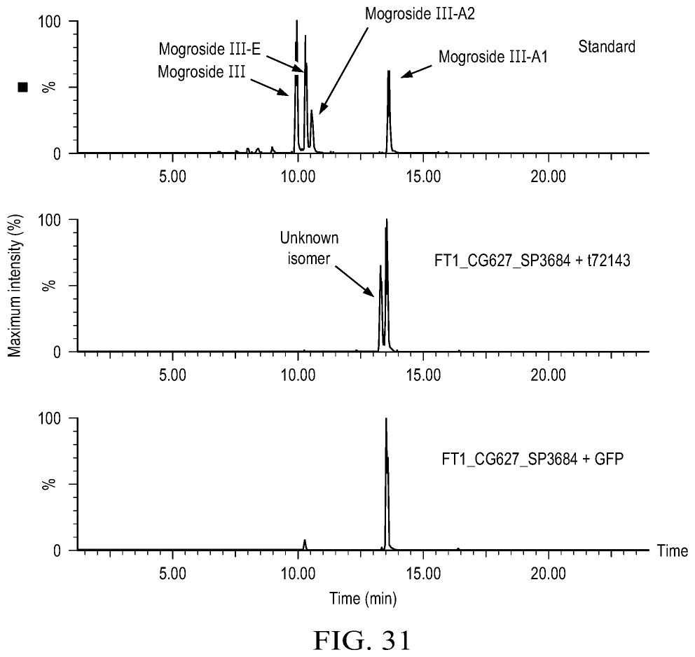

11-oxo-mogroside III and isomers accumulation in transient assays is shown in FIG. 35. For 11-oxo-mogroside III, acquire data in TofMSMS ESI positive mode, selecting for the 11-Oxo-Mogroside III plus a proton (961.5367 m/z), using the collision energy 30 V, and collecting a full high-resolution mass scan (270-1350 m/z) for resulting fragments. The chromatograms shown are selected for the fragment mass 457.3677 m/z with a 0.02 Da mass window. 11-Oxo-Mogroside III-A1 fragment mass 457.3660 m/z is 3.7 ppm error compared to the theoretical 457.3677 m/z. Unknown peak is 457.3677 m/z is 0.0 ppm error compared to the theoretical 457.3677 m/z. 5 ppm is considered an acceptable cutoff for high resolution mass error. The production of 11-oxo-mogroside III and 11-oxo-mogroside III-A1 in transient watermelon fruit infiltrated with the construct SP3684 is shown in FIG. 35, top panel. It can be seen that an unknown 11-oxo-mogroside III isomer was produced when SP3684 was co-infiltrated with t72143 (FIG. 35, middle panel), compared to SP3684 co-infiltrated with GFP (FIG. 35, bottom panel). FIG. 36 shows the mass spectrum fingerprinting of 11-oxo-mogroside III (top panel) and the unknown 11-oxo-mogroside III isomer (bottom panel).

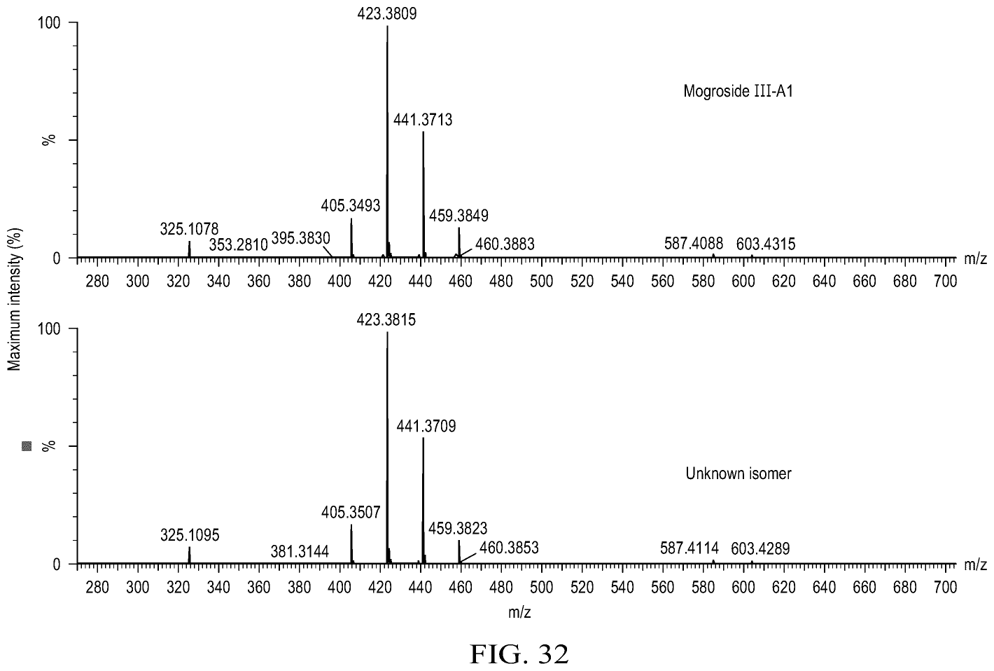

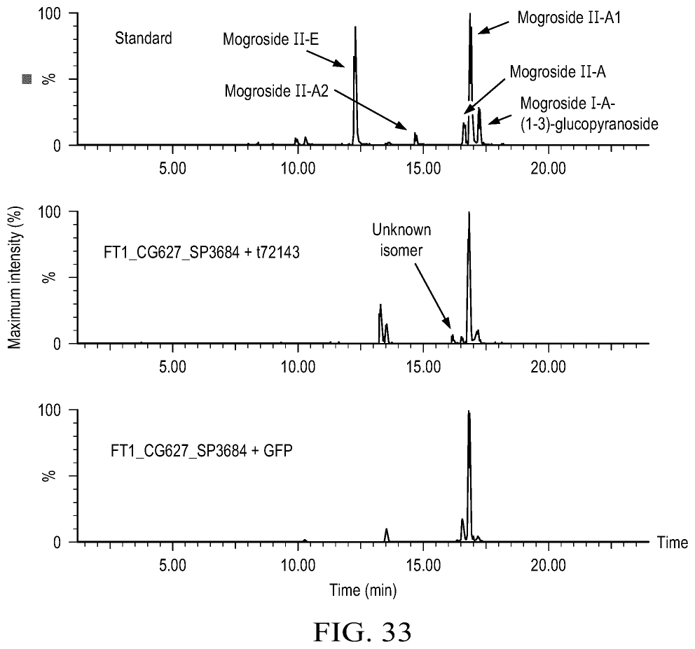

11-oxo-mogroside IV and isomers accumulation in transient assays is shown in FIG. 37. For 11-oxo-mogroside IV, acquire data in TofMSMS ESI positive mode, selecting for the 11-Oxo-Mogroside IV plus a proton (1123.5895 m/z), using the collision energy 30 V, and collecting a full high-resolution mass scan (270-1350 m/z) for resulting fragments. The chromatograms shown are selected for the fragment mass 457.3677 m/z with a 0.02 Da mass window. 11-Oxo-Mogroside IV fragment mass 457.3672 m/z is 1.1 ppm error compared to the theoretical 457.3677 m/z. Unknown peak is 457.3626 m/z is 11.1 ppm error compared to the theoretical 457.3677 m/z. Missing peaks and the low mass accuracy are likely due to the low intensity of this peak. 5 ppm is considered an acceptable cutoff for high resolution mass error. The production of 11-oxo-mogroside IV in transient watermelon fruit infiltrated with the construct SP3684 is shown in FIG. 37, top panel. It can be seen that an unknown 11-oxo-mogroside IV isomer was produced when SP3684 was co-infiltrated with t72143 (FIG. 37, middle panel), compared to SP3684 co-infiltrated with GFP (FIG. 37, bottom panel). FIG. 38 shows the mass spectrum fingerprinting of 11-oxo-mogroside IV (top panel) and the unknown 11-oxo-mogroside IV isomer (bottom panel).

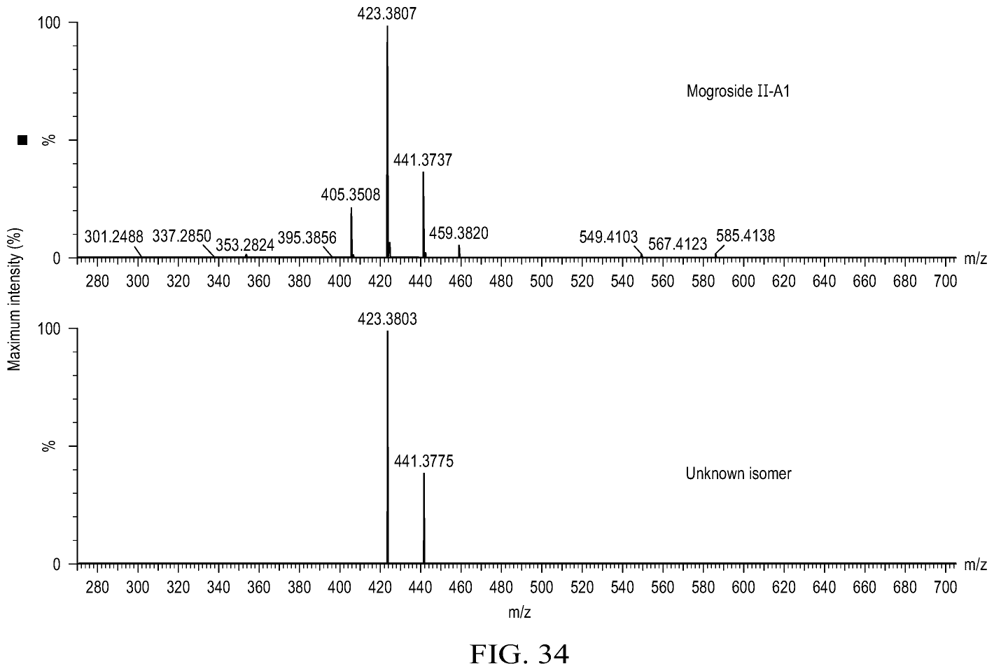

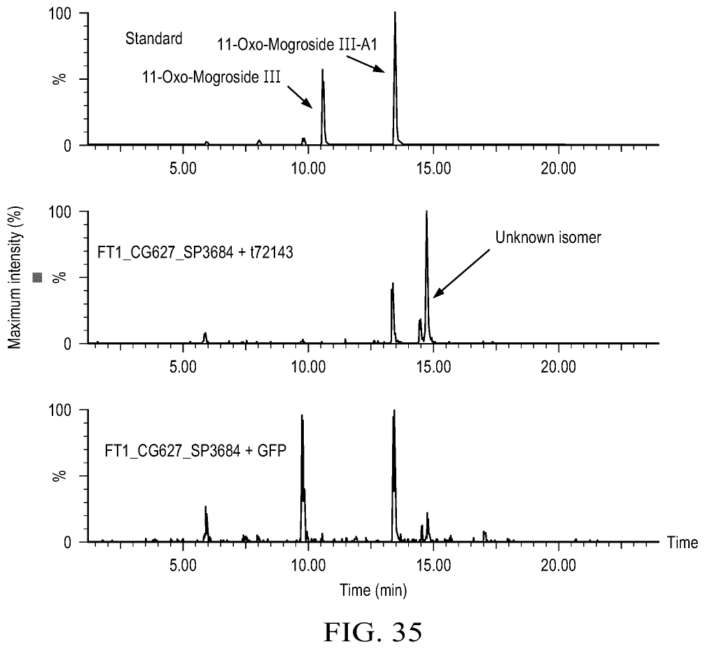

11-oxo-mogroside V and isomers accumulation in transient assays is shown in FIG. 39. For 11-oxo-mogroside V, Acquire data in TofMSMS ESI positive mode, selecting for the 11-Oxo-Mogroside V plus a proton (1285.6423 m/z), using the collision energy 30 V, and collecting a full high-resolution mass scan (270-1350 m/z) for resulting fragments. The chromatograms shown are selected for the fragment mass 457.3677 m/z with a 0.02 Da mass window. 11-Oxo-Mogroside V fragment mass 457.3665 m/z is 2.6 ppm error compared to the theoretical 457.3677 m/z. Unknown peak is 457.3659 m/z is 3.9 ppm error compared to the theoretical 457.3677 m/z. 5 ppm is considered an acceptable cutoff for high resolution mass error. The production of 11-oxo-mogroside V in transient watermelon fruit infiltrated with the construct SP3684 is shown in FIG. 39, top panel. It can be seen that an unknown 11-oxo-mogroside V isomer was produced when SP3684 was co-infiltrated with t72143 (FIG. 39, middle panel), compared to SP3684 co-infiltrated with GFP (FIG. 39, bottom panel). FIG. 40 shows the mass spectrum fingerprinting of 11-oxo-mogroside V (top panel) and the unknown 11-oxo-mogroside V isomer (bottom panel).

### Example 15

### Mogroside Blend Sensory Evaluation

This example evaluates a specific blend of mogrosides as well as some key individual mogrosides to identify their sensory attributes, and to help determine viable blends that are suitable for use as a commercial sweetener. A blend of 80% mogroside-V, 15% 11-oxo mogroside-V, and 5% mogroside IIIA-1 has a clean, sweet taste that is comparable to mogroside V. It is about 213 times as sweet as sucrose at 5 sucrose equivalent value (SEV) and reaches 10.5 SEV at 1,000 ppm. In comparison, mogroside V is 185 times as sweet as sucrose at 5 SEV and reaches 11.5 SEV at 1,000 ppm. V90 is a comparable sweetener to purified mogroside-V in taste and temporal attributes. It is slightly less potent (207× at 5 SEV) and reaches 8.9 SEV at 1,000 ppm. 11-oxo-mogroside-V exhibits slight bitterness and has a low potency (28 times as sweet as sucrose at 5 SEV) compared to mogroside-V. It does not produce high sweetness intensity and only reaches 4.2 SEV at 1,000 ppm. Mogroside IIIA-1 is strongly bitter with a strong lingering bitter aftertaste. It is only 71 times as sweet as sucrose at 5 SEV and plateaus at about 500 ppm. At 1,000 ppm, it only reaches about 5.3 SEV.

Materials and Methods

A blend composed of 80% purified mogroside-V (Chem Faces, CFS202202), 15% 11-oxo-mogroside-V (Chem Faces, CFS202201), and 5% mogroside IIIA-1 (Chem Faces, CFS202201) was diluted to six different concentrations (from 100 ppm to 650 ppm) in spring water (Ice Mountain, retail). For each solution, a set of five reference standards of sucrose dissolved in water in 0.5% (w/w) concentration increments was also prepared. Each concentration of the mogroside blend was evaluated by 6-8 trained judges. Samples were presented in 4-oz soufflé cups at room temperature, and the test sample was labeled with a random 3-digit code. Panelists tasted the blind-coded sample compared to the set of reference samples to determine the sweetness intensity score to the nearest 0.1 unit. Each concentration was evaluated in duplicate. The sweetness intensity scores were then plotted against the mogroside blend concentration to make a concentration response curve, and the best fitting Beidler equation was generated.

Mogroside Blend 80/15/5 Taste Quality: Temporal Properties

Equi-sweet solutions at about 6 SEV were made in spring water (Ice Mountain, retail) of the following sweeteners: purified mogroside-V (Chem Faces, CFS202202), rebaudioside M (95%, Tate & Lyle, lot #1802-2302-1), rebaudioside A (95%, Tate & Lyle, 210617-01), and monk fruit (50% mogroside V, Tate & Lyle, 45GF220305). Another solution was prepared at about 6 SEV of a blend composed of 80% purified mogroside-V (Chem Faces, CFS202202), 15% 11-oxo-mogroside-V (Chem Faces, CFS202201), and 5% mogroside IIIA-1 (Chem Faces, CFS202201). The concentration of each solution can be found in Table 7.

TABLE 7

| Sweetener | ppm |
|---|---|
| Mogroside V | 325 |
| Rebaudioside A | 330 |
| Rebaudioside M | 230 |
| Monk Fruit 50 | 525 |
| Mogroside Blend 80/15/5 | 315 |

A set of samples of each solution was evaluated by a panel of 7-9 trained judges. Panelists measured the time to onset of sweetness and time to reach maximum sweetness using a timer. Sweetness decay after expectorating was measured by rating sweetness intensity every 5 seconds on a 15-cm line scale graduated in evenly spaced 5-point increments and labeled low (0) to high (60). Samples were served at room temperature in 4-oz soufflé cups, labeled with random 3-digit codes. Water and saltine crackers were provided for rinsing between samples. Blind duplicates were evaluated in a second panel under the same conditions.

Mogroside Blend 80/15/5 Taste Quality: Descriptive Analysis

The same samples described above were presented to a panel of 6-9 experienced judges for descriptive analysis. Solutions were blind-coded with random 3-digit codes and served at room temperature for evaluation. Panelists individually scored attributes of each sample, such as sweetness intensity, bitterness, astringency, and the presence of side flavors such as licorice or fruitiness. Scores were based on a 15-cm line scale marked with evenly spaced 5-point increments from low (0) to high (60). Samples were evaluated in duplicate. Water and saltine crackers were provided for rinsing between samples.

V90 Characterization: Concentration Response Curve

V90 is a 90% pure mogroside V containing about 6% 11-oxo mogroside-V. Five different concentrations of V90 (Huacheng Bio, LHGE-230401), ranging from 100 ppm to 500 ppm, were diluted in spring water (Ice Mountain, retail). Each dilution was presented to a panel of 7-8 judges experienced in evaluating sweeteners and was accompanied by a range of five sucrose solution reference standards bracketing the sweetness intensity of the test sample in 0.5% (w/w) increments. Panelists assigned a sweetness intensity score to each test sample, including blind duplicates, to the nearest 0.1 unit. All samples were served at room temperature in 4-oz soufflé cups with the test samples labeled with random 3-digit codes. Water and saltine crackers were provided for rinsing between samples.

V90 Characterization: Temporal Dynamics and Descriptive Analysis

A solution of V90 was made at 410 ppm in spring water (Ice Mountain, retail) to be about 6 SEV. A second solution was made at about 6 SEV of purified mogroside-V (Chem Faces, CFS202202) at 325 ppm. A panel of 7-8 trained judges evaluated the temporal properties and taste attributes of each sample, including blind duplicates, in the same manner described previously in the Mogroside Blend 80/15/5 Taste Quality sections.

Individual Mogroside Taste Quality: Temporal Dynamics and Descriptive Analysis

Two individual mogroside components of the blend, 11-oxo mogroside-V (Chem Faces, CFS202202) and mogroside III-A1 (Chem Faces, 202301), were evaluated separately in spring water (Ice Mountain, retail) to determine the concentration needed to reach approximately 6 SEV. Solutions were then prepared at 3600 ppm of 11-oxo-mogroside-V and 600 ppm mogroside IIIA-1.

A panel of 6-7 trained judges evaluated the solutions by measuring the time to onset of sweetness and time to reach maximum sweetness using a timer. Decay of lingering aftertaste after expectorating was measured by rating sweetness intensity every 5 seconds on a 15-cm line scale graduated in evenly spaced 5-point increments and labeled low (0) to high (60). Samples were served at room temperature in 4-oz soufflé cups, labeled with random 3-digit codes, including blinded duplicates. Water and saltine crackers were provided for rinsing between samples.

The panel also evaluated the intensity of several sensory attributes such as bitterness, astringency, caramel flavor, and fruitiness. The same 15-cm line scale was used for scoring, labeled from low (0) to high (60) with evenly spaced 5-point increments. The samples were served in 4-oz soufflé cups that were labeled with random 3-digit codes. Samples were evaluated at room temperature and included blind duplicates. Water and saltine crackers were provided for rinsing between samples. A 400-ppm solution of V90 (Huacheng Bio, LHGE-230401) was tasted as a reference at the beginning of the descriptive analysis session for calibration verification among panelists.

Individual Mogroside Potency: Concentration Response Curve

Both individual mogrosides were also evaluated at five different concentrations in spring water (Ice Mountain, retail) for sweetness intensity. 11-oxo-mogroside was diluted to concentrations ranging from 200 ppm to 600 ppm and mogroside IIIA-1 was diluted to concentrations from 100 to 500 ppm. For each concentration, a set of five sucrose solution reference standards were prepared in 0.5% (w/w) increments to approximately bracket the sweetness intensity of the test sample. A panel of 6-8 trained judges evaluated each test sample in duplicate, accompanied by the corresponding reference standards, and assigned a sweetness intensity score to the sample to the nearest 0.1 unit. Samples were presented in 4-oz soufflé cups at room temperature, and the test sample was labeled with a random 3-digit code. The sweetness intensity scores were then plotted as a function of mogroside concentration to make a concentration response curve, and the best fitting Beidler equation was generated.

Results

Mogroside Blend 80/15/5 Potency: Concentration Response Curve

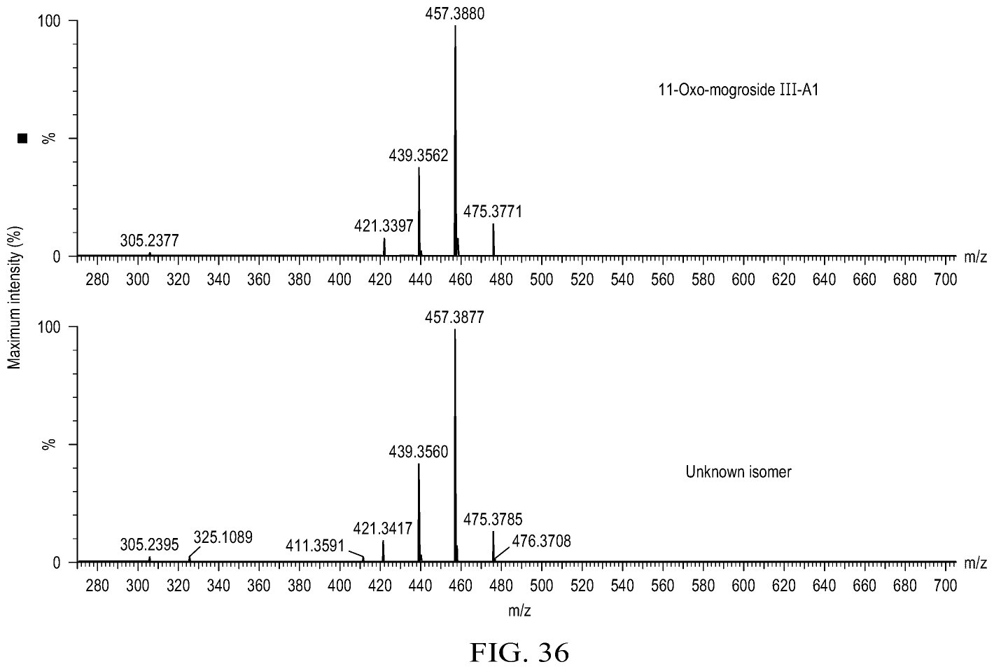

The concentration response curve data for the mogroside blend are summarized in FIG. 41. The error bars around each point represent 1 standard deviation. The data fit a Beidler equation with an r2 value of 0.961. The equation for this line is:

SEV=16.08*C/(521.2+C)
where SEV is the % sucrose equivalent sweetness as determined by sensory panel and C is the concentration of mogroside blend in ppm or mg/L.

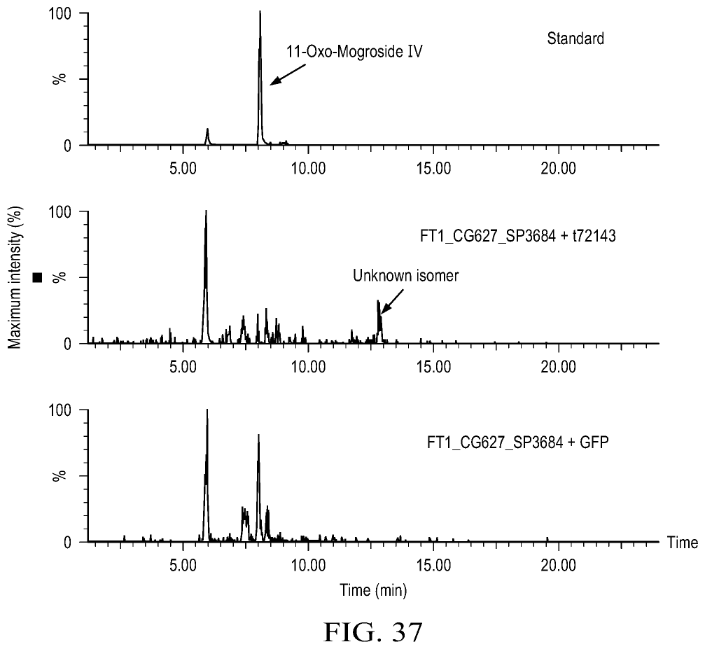

The curve is similar to that of purified mogroside-V, as seen in FIG. 42. At lower concentrations, around 400 ppm or less, the mogroside blend has a slightly higher potency than purified mogroside-V. At 5 SEV, the blend is about 213 times as sweet as sugar, whereas mogroside-V is about 185 times as sweet as sugar. However, at higher concentrations, at about 700 ppm or more, the mogroside blend begins to plateau more rapidly than mogroside-V and is slightly less sweet in comparison. The blend reaches 10.5 SEV at 1,000 ppm compared to 11.5 SEV for mogroside-V. The previously reported equation for mogroside-V is: SEV=22.03*C/(920.8+C).

Both sweeteners exhibit typical curves of high potency sweeteners with steeper slopes at low concentrations that taper off at higher concentrations as potency declines. The potency at any given concentration can be determined by:

P=SEV*10,000/C
where C is the concentration of the mogroside blend in ppm or mg/L and SEV is the % sucrose equivalent sweetness determined by sensory panel.

Mogroside Blend 80/15/5 Taste Quality: Temporal Properties

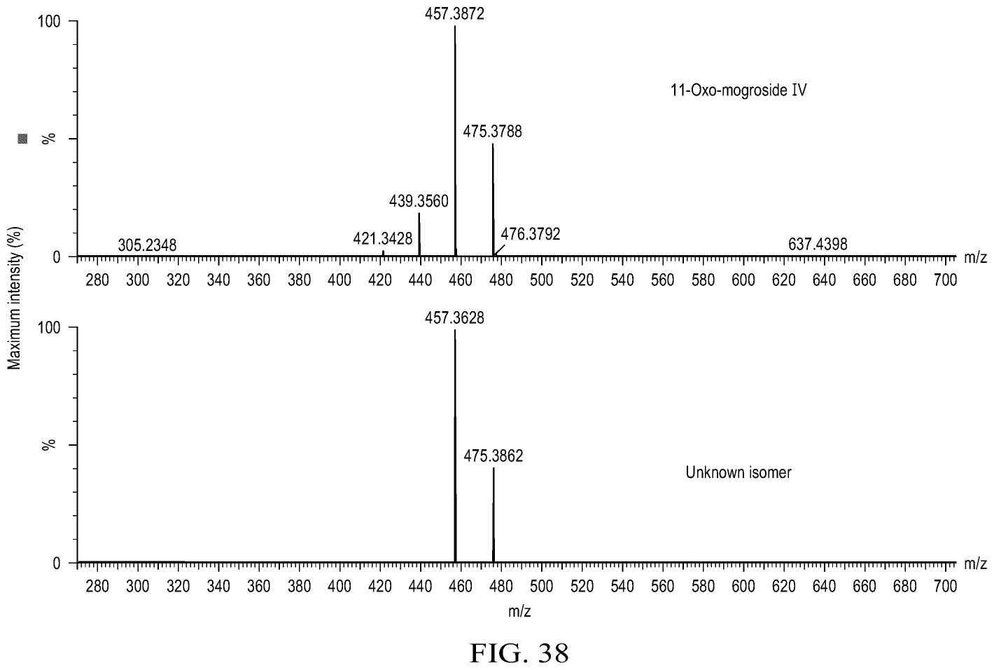

Temporal properties were evaluated for Mogroside-V, Mogroside Blend 80/15/5, Rebaudioside M, Rebaudioside A and monk fruit 50 (MF50). The onset of sweetness for all sweeteners tested was around 1 second, with maximum sweetness achieved at just under 3 seconds. This is a bit delayed compared to sucrose, which was not evaluated in this test but is considered the gold standard of sweetness, and generally has an onset of about 0.5 seconds, reaching maximum sweetness at about 1 second. The onset and time to reach maximum sweetness is shown in FIG. 43.

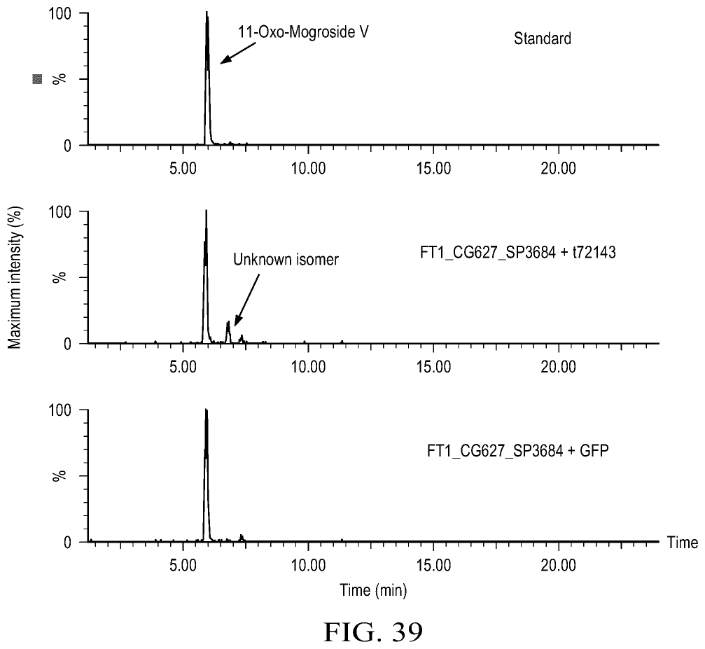

Sweetness from mogroside-V and the mogroside blend both lasted about 45 seconds after expectorating, with similar decay curves. Rebaudioside A had the least lingering sweetness, decaying in about 40 seconds, though a bitter taste continued to linger. Rebaudioside M and monk fruit 50 lingered the longest, lasting 50 and 55 seconds, respectively. Sweetness decay curves are shown in FIG. 44. All sweeteners evaluated had a longer sweet linger than is found in a 6 SEV solution of sucrose, which typically decays in about 20-25 seconds.

Mogroside Blend 80/15/5 Taste Quality: Descriptive Analysis

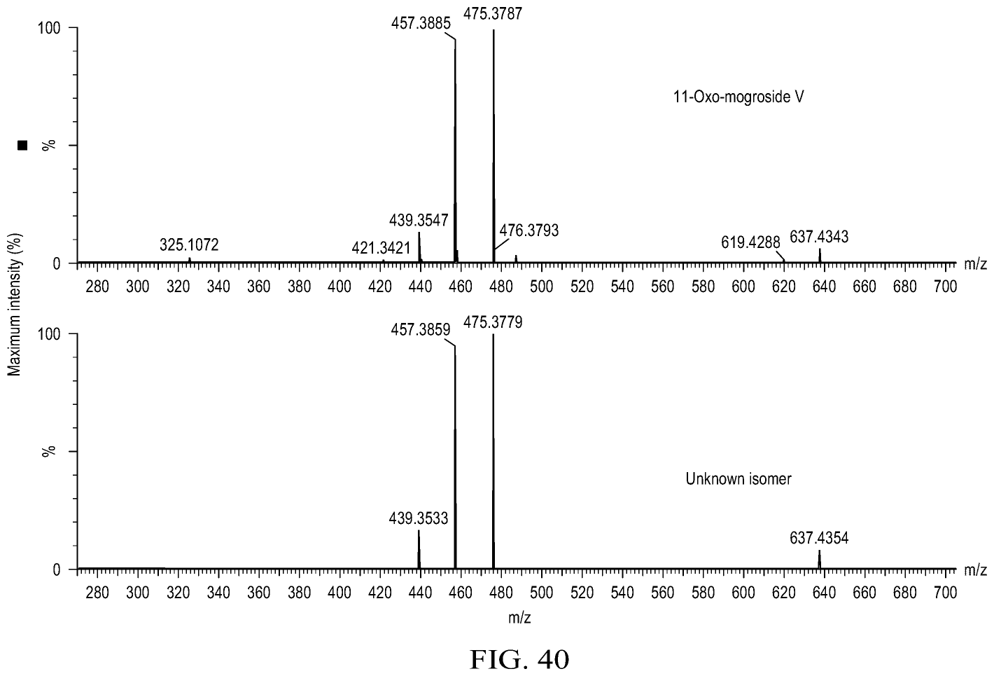

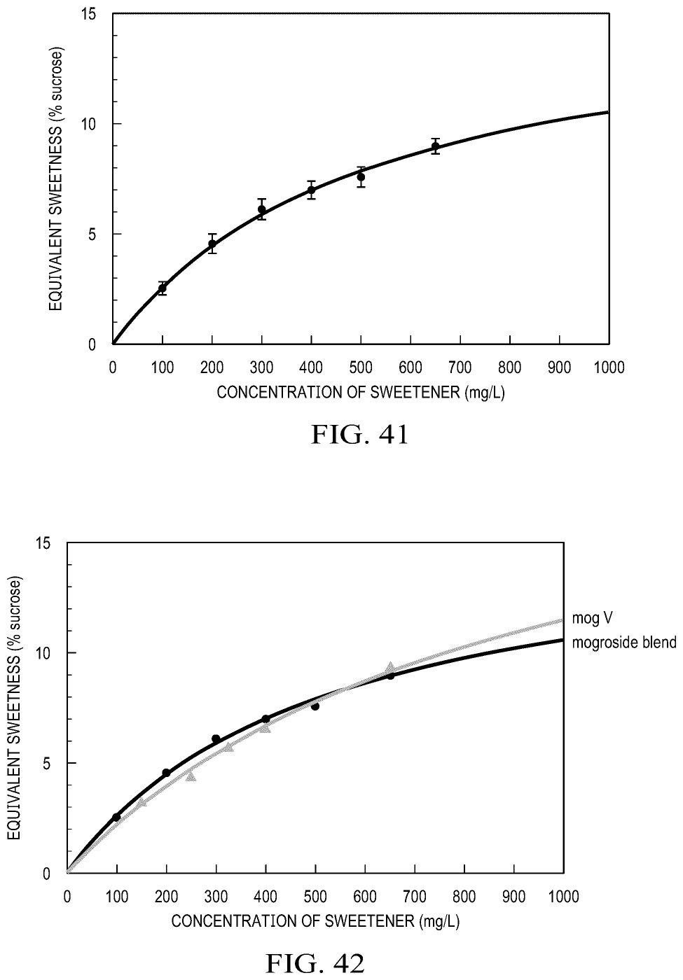

The mogroside blend was quite similar to purified mogroside-V. All sweeteners were characterized by slight caramel notes and body with low astringency. Rebaudioside A had notable bitterness compared to the other sweeteners, and monk fruit 50% had a stronger fruity side taste than the other sweeteners. Table 8 summarizes the average intensity scores for the attributes of each sweetener (0=low, 60=high). The results of the descriptive analysis are shown in FIG. 45 and FIG. 46.

TABLE 8

| | | Blend· | | | |
|---|---|---|---|---|---|
| ¤ | Mog·V¤ | 80/15/5¤ | Reb-M¤ | Reb-A¤ | MF50¤ |
| Sweetness¤ | 36.3¤ | 36.3¤ | 36.4¤ | 35.1¤ | 37.3¤ |
| Caramel¤ | 4.5¤ | 4.3¤ | 3.5¤ | 1.9¤ | 3.9¤ |
| Body¤ | 12.1¤ | 12.1¤ | 11.2¤ | 9.1¤ | 12.1¤ |
| Fruity¤ | 3.0¤ | 2.2¤ | 1.6¤ | 1.3¤ | 10.0¤ |
| Licorice¤ | 0.7¤ | 1.1¤ | 1.0¤ | 2.7¤ | 3.1¤ |
| Astringent¤ | 2.5¤ | 2.5¤ | 2.0¤ | 3.7¤ | 2.9¤ |
| Bitterness¤ | 1.4¤ | 1.6¤ | 1.7¤ | 11.2¤ | 2.7¤ |
| Sweet-Linger¤ | 14.3¤ | 12.3¤ | 17.3¤ | 9.6¤ | 18.1¤ |

V90 Characterization: Concentration Response Curve

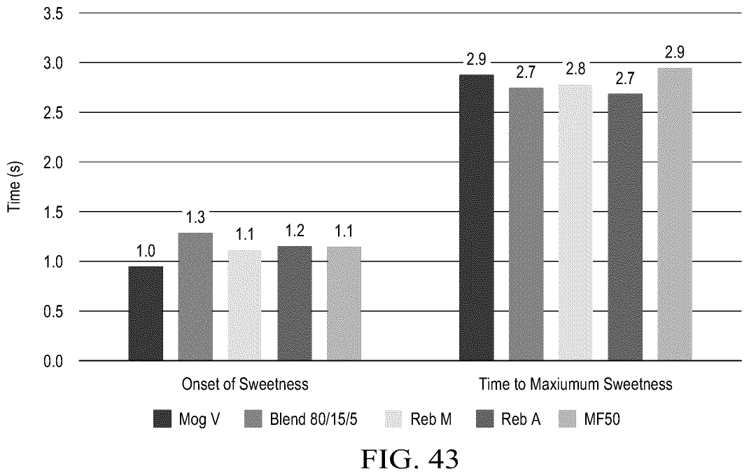

The V90 sweetness intensity scores were plotted as a function of concentration (FIG. 47) using the Beidler equation. The error bars represent 1 standard deviation. The data fit the equation well with an r2 value of 0.938. The equation for this line is:

SEV=11.94*C/(335+C)
where SEV is the % sucrose equivalent sweetness as determined by sensory panel and C is the concentration of mogroside blend in ppm or mg/L. At 5 SEV, V90 is approximately 207 times as sweet as sucrose and it reaches a sweetness of about 8.9 SEV at 1,000 ppm.

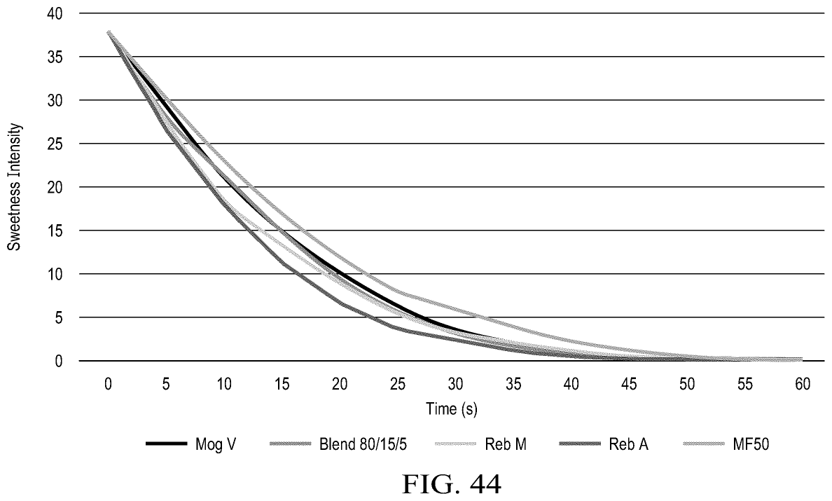

Compared to the concentration response curves of mogroside-V and the 80/15/5 mogroside blend, the curve for V90 plateaus more quickly and reaches a lower sweetness at 1,000 ppm, as seen in FIG. 48.

The sweetness intensity results are lower than expected for assuming 90% mogroside-V in V90. The supplier later reported that V90 is 90% mogroside-V dry basis, not as-is, and analytical testing showed a value closer to 84.5% mogroside-V dry weight. When accounting for these differences, the results for V90 are likely more in line with mogroside-V and the blend values.

V90 remains steeper in slope at low concentrations than Mog-V, similar to the mogroside 80/15/5 blend. Both V90 and the mogroside blend contain 11-oxo mogroside-V, which may be affecting the shape of their curves.

V90 Characterization: Temporal Dynamics and Descriptive Analysis

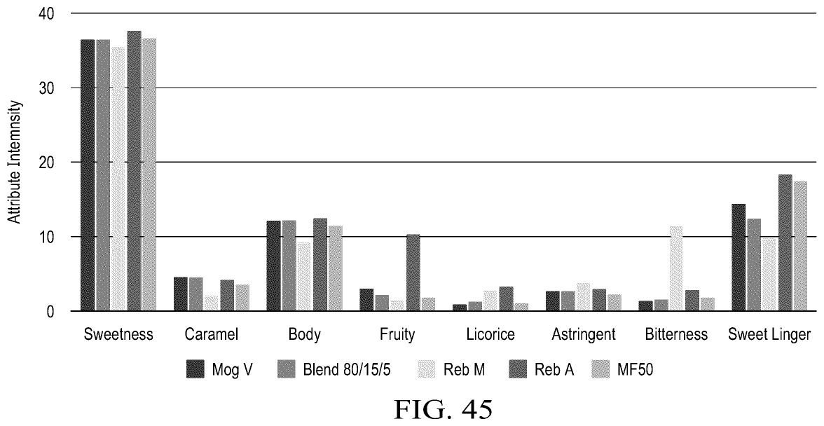

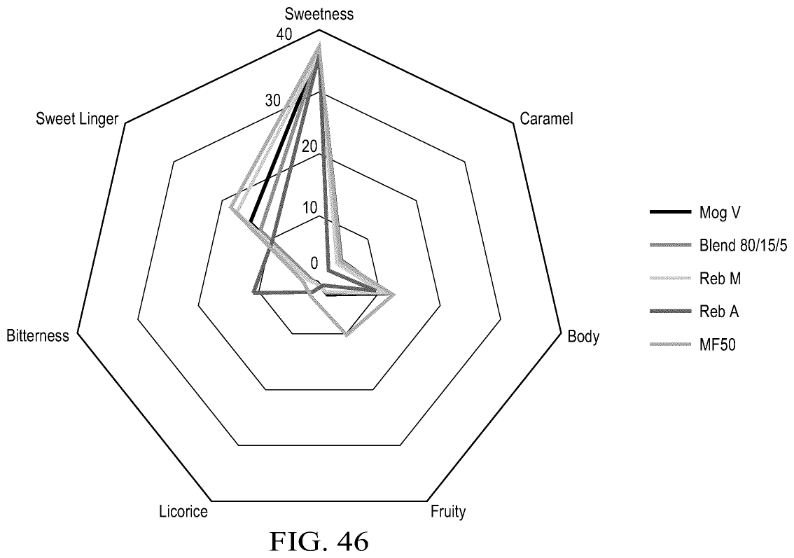

The temporal properties of mogroside-V and V90 were similar. Onset of sweetness occurred at 1.1 and 1.3 seconds, respectively, with maximum sweetness occurring at 2.5 seconds and 2.8 seconds, respectively. The onset and time to maximum sweetness for Mog V and V90 is shown in FIG. 49. The lingering sweet aftertaste of both sweeteners followed a nearly identical decay curve, with mogroside-V sweetness decaying in 40 seconds and V90 decaying in 45 seconds (FIG. 50).

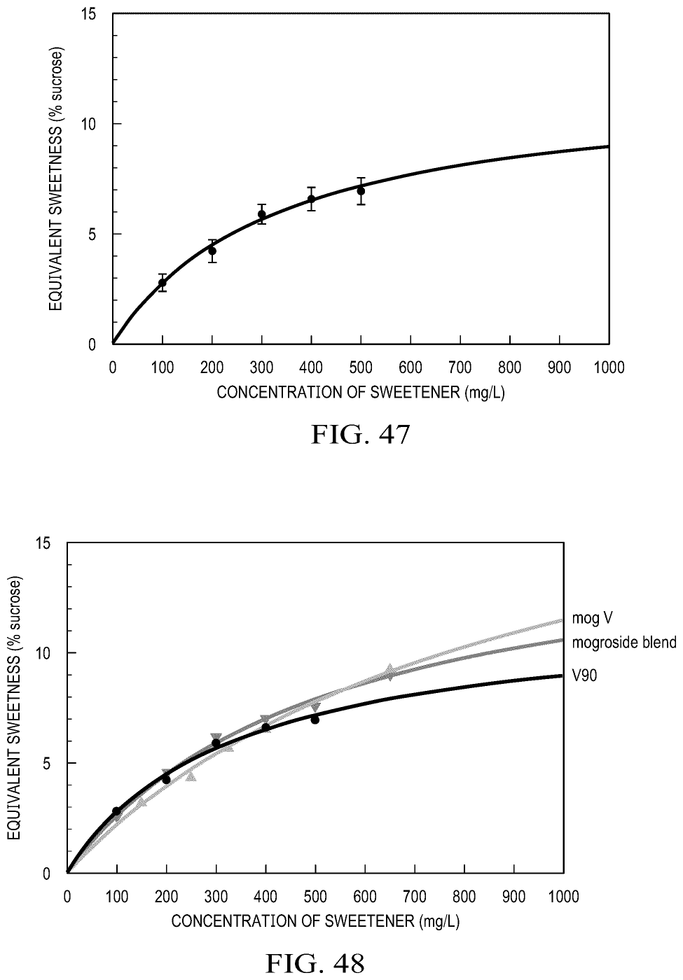

Average intensity scores for sensory attributes of the sweeteners can be found in Table 9 (0=low, 60=high). Both sweeteners had a clean, sweet profile with slight caramel note and some body/mouthfeel, which are sugar-like attributes. Both sweeteners had some lingering sweet aftertaste, which is common among high potency sweeteners. Licorice, bitterness, and astringency, which are common rebaudioside A attributes, were very low. The descriptive analysis scores are shown in FIG. 51.

TABLE 9

| Attribute | Mogroside V | V90 |
|---|---|---|
| Sweetness | 35.9 | 36.5 |
| Caramel | 4.2 | 5.9 |
| Body | 11.9 | 13.8 |
| Fruity | 2.6 | 2.3 |
| Licorice | 0.8 | 1.6 |
| Astringent | 2.9 | 2.4 |
| Bitterness | 1.1 | 2.0 |
| Sweet Linger | 16.2 | 17.1 |

Individual Mogroside Potency: Concentration Response Curve

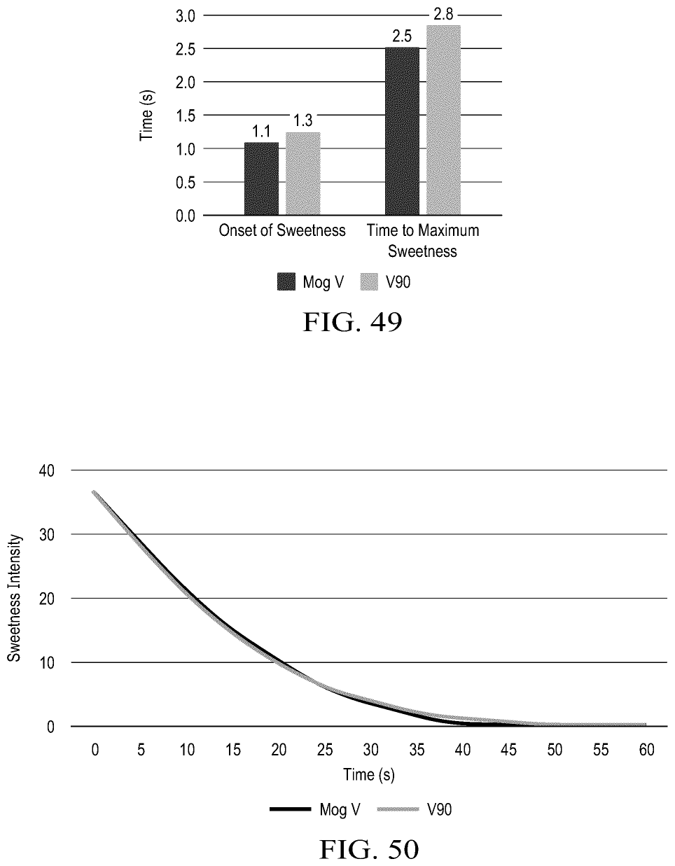

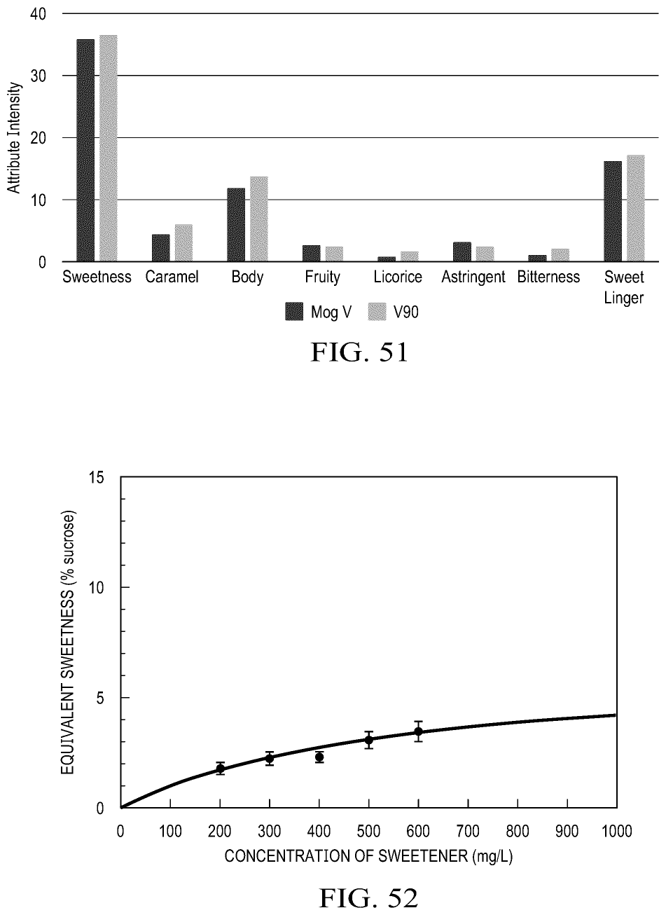

The sweetness intensity scores for both 11-oxo-mogroside-V and mogroside IIIA-1 were plotted against their concentrations to generate concentration response curves according to the Beidler equation. In FIG. 52 and FIG. 53, the error bars represent 1 standard deviation.

The curve for 11-oxo-mogroside-V omitted the data point at 400 ppm, as it appeared to be an outlier. The resultant equation fit well with the remaining data, with an r2 value of 0.946. The equation for this line is:

SEV=6.56*C/(546.3+C)

where SEV is the % sucrose equivalent sweetness as determined by sensory panel and C is the concentration of mogroside blend in ppm or mg/L. At 5 SEV, 11-oxo mogroside-V is approximately 28 times as sweet as sucrose. This potency is much lower than the potency previously seen in purified Mog-V, the mogroside blend, or V90, all of which were closer to 200 times as sweet as sucrose at 5 SEV.

The concentration response curve for mogroside IIIA-1 has a steep slope at very low concentrations (less than 100 ppm) and plateaus rapidly at a low SEV (around 4-5 SEV). This curve is typical of sweeteners that exhibit bitterness, which inhibits sweetness at higher concentrations. The data fit the equation very well with an r2 value of 0.983. The equation for this line is:

SEV=6.00*C/(139.9+C)
where SEV is the % sucrose equivalent sweetness as determined by sensory panel and C is the concentration of mogroside blend in ppm or mg/L. Mogroside IIIA-1 is approximately 71 times as sweet as sugar at 5 SEV and reaches about 5.3 SEV at 1,000 ppm.

A summary of potency and sweetness at 1,000 ppm for the mogroside sweeteners and blends in this study is shown in Table 10 below.

TABLE 10

| | Mogroside | Blend | | 11-oxo- | Mogroside |
|---|---|---|---|---|---|
| | V | 80/15/5 | V90 | mogroside V | IIIA-1 |
| Potency at 5 SEV | 185x | 213x | 207x | 28x | 71x |
| Potency at 10 SEV | 131x | 117x | 58x | n/a | n/a |
| SEV at 1,000 ppm | 11.5 | 10.5 | 8.9 | 4.2 | 5.3 (very |
| | | | | | bitter) |

Individual Mogroside Taste Quality: Temporal Dynamics and Descriptive Analysis

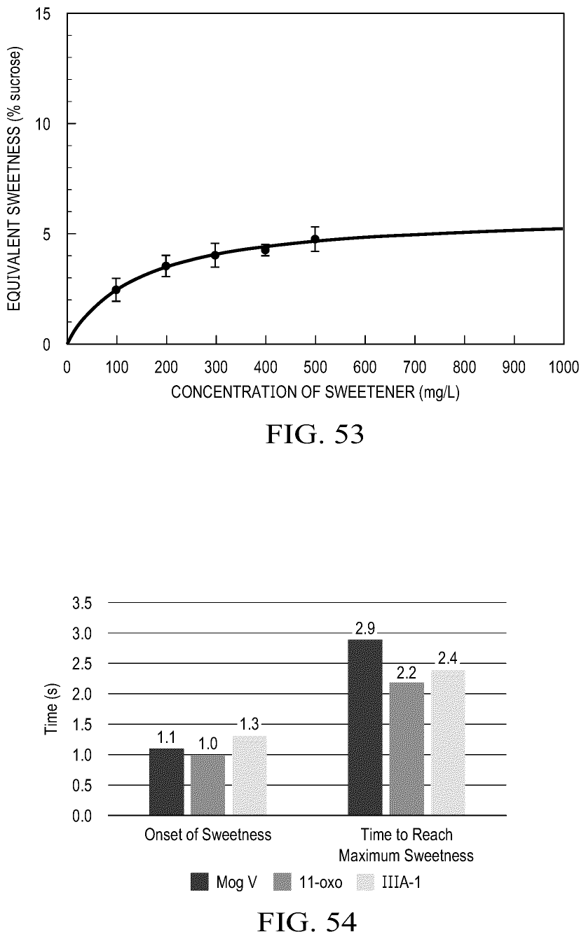

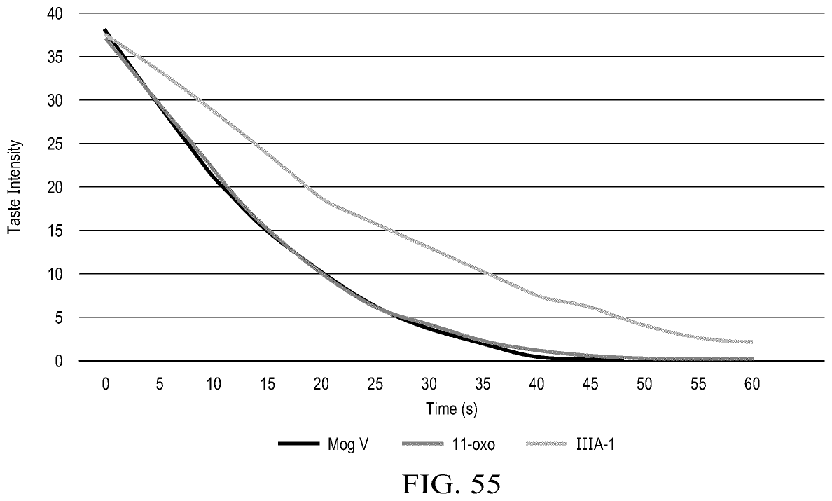

The results of the temporal profiling of 11-oxo mogroside-V and mogroside IIIA-1 can be seen in FIG. 54 and FIG. 55, compared to previous data generated by the panel for mogroside-V. Mogroside V took slightly longer to reach peak sweetness compared to the other mogrosides, but onset of sweetness was comparable across all three samples.

When evaluating the solutions of mogroside IIIA-1, the panelists noted a strong bitter aftertaste that almost immediately overwhelmed any lingering sweet sensation. In FIG. 55, intensity of bitter aftertaste is plotted for mogroside IIIA-1 in comparison to intensity of sweet aftertaste for 11-oxo mogroside-V and previously generated data for mogroside-V. Sweetness from 11-oxo mogroside-V decayed in about 45 seconds, which is on par with the length of sweet aftertaste from mogroside-V. The strong bitter taste from mogroside IIIA-1 was much more lingering and still present more than 60 seconds after expectorating. The strong bitterness of mogroside IIIA-1 was also noted in the descriptive analysis attribute scores. Average intensity scores for all attributes can be found in Table 11, shown compared to mogroside-V data generated from a previous descriptive analysis sensory panel.

TABLE 11

| | 11-oxo- | Mogroside | Mogroside |
|---|---|---|---|
| Attribute | mogroside V | IIIa-1 | V |
| Sweetness | 37.1 | 34.5 | 36.3 |
| Caramel | 4.8 | 1.9 | 4.5 |
| Body | 12.7 | 12.1 | 12.1 |
| Fruity | 2.1 | 0.7 | 3.0 |
| Licorice | 2.1 | 2.2 | 0.7 |
| Astringent | 4.3 | 5.1 | 2.5 |
| Bitterness | 7.7 | 37.3 | 1.4 |
| Sweet Linger | 17.1 | 5.7 | 14.3 |
| Bitter Linger | 0.7 | 37.3 | 0.0 |

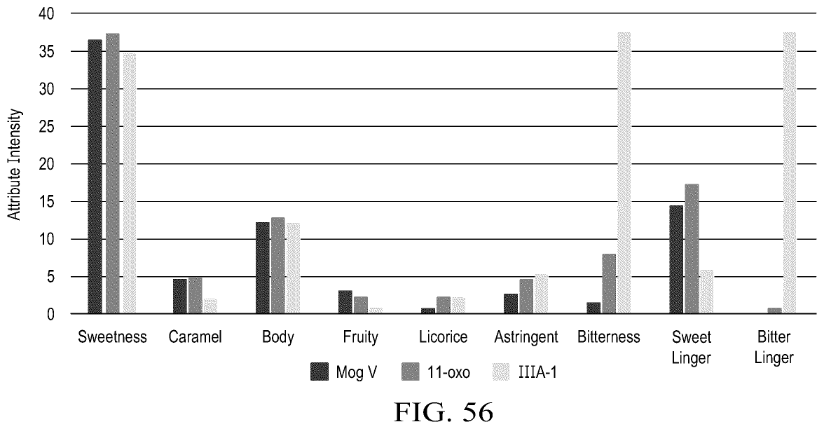

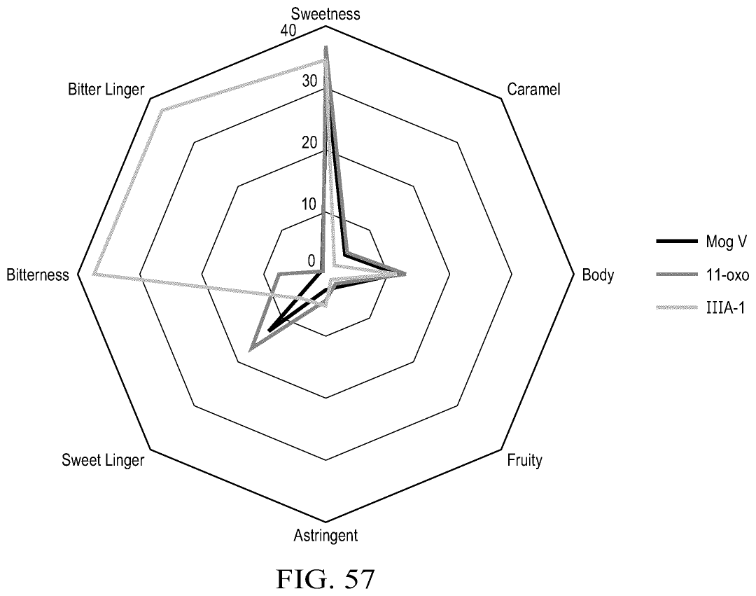

The descriptive analysis scores of sweetener attributes for Mogroside-V, 11-oxo-mogroside V and mogroside IIIA-1 are shown in FIG. 56, and the descriptive analysis of sweetener attributes for Mogroside-V, 11-oxo-mogroside V and mogroside IIIA-1 are shown in FIG. 57.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. For example, all of the disclosed components of the preferred and alternative embodiments are interchangeable providing disclosure herein of many systems having combinations of all the preferred and alternative embodiment components. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. All such similar substitutes and modifications apparent to those skilled in the art are deemed to be within the spirit, scope and concept of the disclosure as defined by the appended claims.

1. A transgenic plant, plant part or seed, comprising the recombinant DNA molecule of claim 27:
wherein the transgenic plant, plant part or seed produces at least a first mogroside compound.

2.-4. (canceled)

5. The transgenic plant, plant part or seed of claim 1, wherein the plant is a Cucurbitaceae, Solanaceae or Asteraceae plant.

6.-7. (canceled)

8. The transgenic plant, plant part or seed of claim 5, wherein the plant is a watermelon, tomato, lettuce or cucumber plant.

9.-10. (canceled)

11. The transgenic DNA molecule of claim 27, wherein the first, second, third, fourth, fifth, sixth, seventh or eighth polynucleotide sequence is operably linked to a heterologous terminator.

12. The transgenic DNA molecule of claim 11, wherein the first, second, third, fourth, fifth, sixth, seventh or eighth heterologous terminator sequence is a GmaxMYB2 (SEQ ID NO:74), 35S T (SEQ ID NO:80), ATHSP18.2 (SEQ ID NO:77), AtRBCS2b (SEQ ID NO:75), AtUBQ3 (SEQ ID NO:73), Pea E9 (SEQ ID NO:76), Pea3A (SEQ ID NO:72), potato Ubi3 (SEQ ID NO:78), AtTubB9 (SEQ ID NO:79), AtFAD2 (SEQ ID NO:265), AtNDUFA8 (SEQ ID NO:266), CsHSP17.3 (SEQ ID NO:267) or CsHSP22 (SEQ ID NO:268) terminator.

13.-15. (canceled)

16. The transgenic DNA molecule of claim 27, further comprising;
i) a ninth polynucleotide sequence that encodes an NADPH:cytochrome P450 reductase polypeptide having at least 90% sequence identity to SEQ ID NO:19;
j) a tenth polynucleotide sequence that encodes a uridine phosphorylase dependent glycosyltransferase polypeptide having at least 90% sequence identity to SEQ ID NO:21, SEQ ID NO:23, SEQ ID NO:120 or SEQ ID NO: 122;
k) an eleventh polynucleotide sequence that encodes a 3-hydroxy-3-methylglutaryl-CoA synthase polypeptide having at least 90% sequence identity to SEQ ID NO: 148;
l) a twelfth polynucleotide sequence that encodes a geranyl diphosphate synthase polypeptide having at least 90% sequence identity to SEQ ID NO: 126;
m) a thirteenth polynucleotide sequence that encodes a hydrolase biosynthetic enzyme having at least 90% sequence identity to SEQ ID NO:158; or
n) a fourteenth polynucleotide sequence that encodes a uridine phosphorylase dependent glycosyltransferase enzyme having at least 90% sequence identity to SEQ ID NO:132;
wherein the ninth, tenth, eleventh, twelfth, thirteenth, or fourteenth polynucleotide sequence is operably linked to a heterologous promoter.

17.-19. (canceled)

20. The transgenic plant, plant part or seed of claim 19 wherein the at least a first mogroside compound is mogroside II A, mogroside II A1, mogroside II A2, mogroside II E, 11-oxo-mogroside II, mogroside III, mogroside III A1, mogroside III A2, mogroside III E 11-oxo-mogroside III, mogroside IV, mogroside IV A, 11-oxo-mogroside IV, siamenoside I, mogroside V, 11-oxo-mogroside V or mogroside VI, or an isomer thereof.

21. The transgenic plant, plant part or seed of claim 1, wherein:
a) the plant produces at least 10 ng/g to 30 mg/g dry weight of the at least a first mogroside compound; or
b) the plant produces an amount of the at least a first mogroside compound that is greater than the level in a non-transgenic plant, plant part or seed of the same species.

22.-23. (canceled)

24. A processed lower calorie food or beverage product produced from the transgenic plant, plant part or seed of claim 1.

25. The processed lower calorie food or beverage product of claim 24, wherein the product is produced from a juice or extract of the transgenic plant, plant part or seed.

26. A juice or extract produced from the transgenic plant, plant part or seed of claim 1.

27. A recombinant DNA molecule comprising:
a) a first polynucleotide sequence that encodes a cytochrome P450 polypeptide having at least 91% sequence identity to SEQ ID NO:2 or at least 95% sequence identity to SEQ ID NO:86;
b) a second polynucleotide sequence that encodes a cucurbitadienol synthase polypeptide having at least 90% sequence identity to SEQ ID NO:5;
c) a third polynucleotide sequence that encodes a cytochrome P450 polypeptide having at least 90% sequence identity to SEQ ID NO:7, SEQ ID NO:25, SEQ ID NO:27, SEQ ID NO:29, SEQ ID NO:31 or SEQ ID NO:33;
d) a fourth polynucleotide sequence that encodes a uridine phosphorylase dependent glycosyltransferase polypeptide having at least 90% sequence identity to SEQ ID NO:9;
e) a fifth polynucleotide sequence that encodes a uridine phosphorylase dependent glycosyltransferase polypeptide having at least 90% sequence identity to SEQ ID NO:11;
f) a sixth polynucleotide sequence that encodes a squalene epoxidase polypeptide having at least 90% sequence identity to SEQ ID NO:13;
g) a seventh polynucleotide sequence that encodes an epoxy hydrolase polypeptide having at least 90% sequence identity to SEQ ID NO:15; or
h) an eighth polynucleotide sequence that encodes a truncated 3-hydroxy-3-methylglutaryl-CoA reductase polypeptide having at least 90% sequence identity to SEQ ID NO:17, SEQ ID NO: 275, SEQ ID NO: 277 or SEQ ID NO: 279;
wherein the first, second, third, fourth, fifth, sixth, seventh or eighth polynucleotide sequence is operably linked to a heterologous promoter.

28.-30. (canceled)

31. The recombinant DNA molecule of claim 27, wherein the heterologous promoter is a FSgt/PFLt (SEQ ID NO:62), FMVSgt (SEQ ID NO:69), CsVMV (SEQ ID NO:68), dMMV (SEQ ID NO:63), HLVH12 (SEQ ID NO:60), NOS (SEQ ID NO:66), ScBV (SEQ ID NO:67), DCMV (SEQ ID NO:61), CmYLCV (SEQ ID NO:64), FS1_1 (SEQ ID NO:70), FE_3 (SEQ ID NO:71), e35S (SEQ ID NO:65), AtUBQ10 (SEQ ID NO:259), PCLSV (SEQ ID NO:260), FS4 (SEQ ID NO:261), AtACT2 (SEQ ID NO:262), enhanced AtEf-1A (SEQ ID NO:263), FuasFScp (SEQ ID NO:264), FE4 (SEQ ID NO:269), cucumisin (SEQ ID NO:270) or SgCDS (SEQ ID NO:271) promoter.

32. The recombinant DNA molecule of claim 27, wherein first, second, third, fourth, fifth, sixth, seventh or eighth polynucleotide sequence is operably linked to a heterologous terminator.

33. The recombinant DNA molecule of claim 32, wherein the heterologous terminator sequence is a GmaxMYB2 (SEQ ID NO:74), 35S T (SEQ ID NO:80), ATHSP18.2 (SEQ ID NO:77), AtRBCS2b (SEQ ID NO:75), AtUBQ3 (SEQ ID NO:73), Pea E9 (SEQ ID NO:76), Pea3A (SEQ ID NO:72), potato Ubi3 (SEQ ID NO:78), AtTubB9 (SEQ ID NO:79), AtFAD2 (SEQ ID NO:265), AtNDUFA8 (SEQ ID NO:266), CsHSP17.3 (SEQ ID NO:267) or CsHSP22 (SEQ ID NO:268) terminator.

34. (canceled)

35. A DNA molecule comprising:
a) a polynucleotide sequence that encodes a cytochrome P450 polypeptide having at least 91% sequence identity to SEQ ID NO:2 or at least 95% sequence identity to SEQ ID NO:86; or
b) a polynucleotide sequence selected from the group consisting of:
i) a sequence with at least 90 percent sequence identity to SEQ ID NOs:70 or 71 and exhibiting promoter activity;
ii) a sequence comprising SEQ ID NOs:70 or 71; and
iii) a fragment of SEQ ID NOs:70 or 71, wherein said fragment exhibits promoter activity;
wherein the polynucleotide sequence of part b) exhibits gene regulatory functional activity, and wherein the polynucleotide sequence of part b) is operably linked to a heterologous transcribable polynucleotide molecule.

36.-37. (canceled)

38. A method of producing at least a first mogroside compound, comprising growing the transgenic plant of claim 1, wherein the transgenic plant produces the at least a first mogroside compound.

39.-40. (canceled)

41. The method of claim 38, further comprising the step of isolating the at least a first mogroside compound from the transgenic plant.

42.-43. (canceled)

44. A recombinant host cell comprising the recombinant DNA molecule of claim 27.

45. (canceled)

46. A composition comprising:
a) about 80% mogroside V, about 15% 11-oxo-mogroside V and about 5% mogroside III-A 1; or
b) about 40% siamenoside I, about 40% mogroside V and about 20% 11-oxo-mogroside V.

47. The composition of claim 46, wherein:
a) the composition is a liquid; or
b) the composition is a dry powder.

48. (canceled)


SEQUENCE LISTING (305 sequences)

SEQ ID NO: 1 - DNA, 1557 bp, synthetic construct
  source 1..1557: mol_type = other DNA
atgtcctcatcagttgcagctactatcagcatatggatggtggtggtgtgcatcgttggtgttggctggcgggtggtgaattgggtttggttgaggccaaaaaaattggagaagcggctccgcgaacagggcttggcgggcaactcgtataggttgcttttcggtgacttgaaggagagagccgctatggcggagcaagcgaactctaagcccataaatttttcccacgacatcggtcctagagtcttcccgtcaatgtacaagacgattcagaactatggaaagaattcttatatgtggctgggtccctatcctcgcgtccacatcatggacccccagcaattgaaaacggtttttacgctcgtgtacgacatccagaaacccaacctgaatccactcattaaattcttgctcgacgggattgtcacacacgagggtgagaagtgggcaaaacataggaagataataaatcccgcttttcatatggctaagcttaaagacatgatacccgctttttttcagtcgtgtaacgaaatagtctccgagtgggagcggatgatcccgaaagaaggtagctttgaacttgatgttatgccctacatgcagaacctcgccgcagacgcaatttcccgcactgctttcggttcctcctacaaggaaggacagatgatctttcagctgctgaaagagctgactgacttggtggttaaagccgcgtttggaatctatattcccggctggcggtttcttcccacgaagtcgaacaacaaaatgaaagaaataaacaggaagataaaaaccttgttgttgggcataatagataaacggcagaaagcgatgaaggagggcgaggtcgttcagaacgacctgctgggtattctcgtggaatcgaatcttaatgaaatcagaggccacgaaaataataaggatgcaggaatgtcaattgaggatgtgattgaagagtgtaaggttttttatatcgggggtcaagaaactaccgctggcctccttatctggactatgattctgctttccttccatacagagtggcaagaaagagctcggacagaagtgctcgaagtcgtgggcaacaagaagcctgactttgatggattgaatagattgaaaatagttactatgatcctctacgaagtgttgagactctatccgcctgcgtcaatgctgacccggatagtccagaaggaaacccgcataggaaagttgacattgccagctggggttactctgataatcccaacggtcctcattcatagagatcacgaattgtggggcgaagacgctaacgagttcaagccggagcggttttctgagggagtgtccaaagccgccaaggtccagccggctttttttcccttcgggtgggggccgcgcttgtgcctcggacagaattttgcaatgatcgaagctaaaatggcttttagcctcattcttcagcggtttagctttcacctctcatcatcttacgcgcatgccccaactgttgtgatgactacgcagccgcagcacggcgcccacatagtcctgcgcaaactctaa

SEQ ID NO: 2 - protein, 518 aa, Siraitia grosvenorii
MSSSVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHMAKLKDMIPAFFQSCNEIVSEWERMIPKEGSFELDVMPYMQNLAADAISRTAFGSSYKEGQMIFQLLKELTDLVVKAAFGIYIPGWRFLPTKSNNKMKEINRKIKTLLLGIIDKRQKAMKEGEVVQNDLLGILVESNLNEIRGHENNKDAGMSIEDVIEECKVFYIGGQETTAGLLIWTMILLSFHTEWQERARTEVLEVVGNKKPDFDGLNRLKIVTMILYEVLRLYPPASMLTRIVQKETRIGKLTLPAGVTLIIPTVLIHRDHELWGEDANEFKPERFSEGVSKAAKVQPAFFPFGWGPRLCLGQNFAMIEAKMAFSLILQRFSFHLSSSYAHAPTVVMTTQPQHGAHIVLRKL

SEQ ID NO: 3 - DNA, 1557 bp, synthetic construct
  source 1..1557: mol_type = other DNA
atgtcgtccagcgtggcggccacgatcagcatctggatggtggtcgtctgcatcgtcggggtggggtggcgcgtcgtcaactgggtctggctccggccgaagaagctggagaagcgcctccgggagcaggggctcgcgggcaacagctacaggctgctgttcggggacctcaaggagagggccgcgatggcggagcaggcgaacagcaagccgatcaacttcagccacgacatcgggccgcgcgtgttcccgagcatgtacaagacgatccagaactacggcaagaactcctacatgtggctcgggccgtacccgcgcgtgcacatcatggacccgcagcagctcaagaccgtgttcaccctggtctacgacatccagaagccgaacctgaacccgctgatcaagttcctcctggacggcatcgtgacccacgagggggagaagtgggcgaagcaccgcaagatcatcaacccggcgttccacatggccaagctcaaggacatgatcccggccttcttccagtcctgcaacgagatcgtctccgagtgggagcgcatgatcccgaaggagggctccttcgagctggacgtgatgccgtacatgcagaacctggccgccgacgccatctcccgcaccgccttcggctcctcctacaaggagggccagatgatcttccagctgctgaaggagctgaccgacctggtggtgaaggccgccttcggcatctacatcccgggctggcgcttcctgccgaccaagtccaacaacaagatgaaggagatcaacaggaagatcaagaccctgctgctgggcatcatcgacaagcgccagaaggccatgaaggagggggaggtcgtgcagaacgacctgctgggcatcctggtggagagcaacctgaatgagatccgcggccacgagaacaacaaggacgcgggcatgtccatcgaggacgtgatcgaggagtgcaaggtgttctacatcgggggccaggagacgacggccgggctgctgatctggaccatgatcctcctgtccttccacacggagtggcaggagagggcccgcaccgaggtgctggaggtggtggggaacaagaagccggacttcgacggcctgaaccgcctgaagatcgtgaccatgatcctgtacgaggtcctgcgcctgtacccgccggcgtccatgctgaccaggatcgtgcagaaagaaacccgcatcggcaagctgaccctgccggccggggtgaccctgatcatcccgaccgtgctgatccaccgcgaccacgagctgtggggggaggacgcgaacgagttcaagccggagaggttctccgagggggtgtccaaggcggccaaggtgcagccggcgttcttcccgttcggctggggcccgcgcctgtgcctgggccagaacttcgccatgatcgaggccaaaatggccttctccctgatcctgcagcgcttctccttccacctgtcctcctcctacgcccacgccccgacggtggtgatgaccacccagccgcagcacggcgcccacatcgtgctgaggaagctgtaa

SEQ ID NO: 4 - DNA, 2277 bp, synthetic construct
  source 1..2277: mol_type = other DNA
atgtggcgccttaaggttggggccgagtctgtcggcgagaacgatgaaaagtggctcaaatcaatcagcaatcatcttggcaggcaggtctgggaattttgtcccgatgccggaactcaacaacagttgttgcaagtccacaaagcgagaaaggcgttccatgacgatagatttcatcggaaacagtcgtcggatcttttcattactatacaatacgggaaggaagttgagaacggtgggaagacggctggcgtgaaacttaaagagggcgaagaggtgagaaaggaagccgttgagagctccttggagcgggccttgtcgttttattcaagcatccagacgtctgacggtaattgggcatcggacctcgggggccccatgttcctcctgcccgggctggtcatcgcgctctacgttaccggtgttttgaattcggtcctgtcaaaacatcacaggcaggagatgtgtcggtatgtctataatcatcagaatgaggatggcggttggggcctgcacatagaagggccgtctactatgttcggttcagcattgaactacgtggctctgcggcttctcggtgaggacgccaatgcgggtgcaatgccaaaagcgcgcgcttggattctggaccacggcggggccaccggaattacttcttggggaaagctctggctctcagttcttggcgtgtacgagtggtctggcaacaacccactccccccagaattttggctctttccgtatttccttcccttccatccgggcagaatgtggtgtcattgtcggatggtgtatttgccgatgtcctatttgtacgggaaaaggttcgttgggccgataacgccgatcgttcttagccttagaaaagagctctatgcggtcccttatcacgagatcgactggaacaaatctcggaacacatgtgcaaaagaggacttgtactacccccaccccaagatgcaggacatcttgtggggatcattgcaccatgtttacgagcccctgtttactcggtggccagcgaaaaggcttagggaaaaggcgctgcagacggctatgcaacatattcactatgaagatgaaaacactcgctatatttgcctgggcccagttaataaagtgctgaatctgctttgttgttgggtcgaagatccatactctgatgcttttaaactgcatcttcaacgcgtgcatgattacctttgggtggcagaggatggcatgaaaatgcagggatataacggttcccaactttgggataccgctttcagcattcaagcaatagtctcaacgaagttggtcgataactacggtcccactttgcggaaggcgcacgatttcgtcaaatcatcgcaaatacaacaggactgccctggcgatcccaacgtgtggtacaggcatatccacaaaggagcatggccattttctacgagggaccatgggtggctgatatcggattgtaccgccgaaggactcaaggcagcccttatgctcagcaagttgccgtccgagacagtgggtgagtcgcttgaacgcaatcggctgtgcgacgctgttaacgtcttgctgtctttgcagaacgacaacggcgggttcgcatcgtatgagcttactagatcgtatccgtggcttgaactcattaatcccgccgaaacattcggagatatcgtgattgactatccctatgtggaatgcacttctgcaacaatggaagcgcttacacttttcaaaaaacttcaccctgggcacaggactaaggaaatcgacacagcgatagtgcgcgccgcgaacttcctggagaatatgcagagaactgacggctcttggtatggctgctggggcgtttgcttcacatatgcaggctggtttggaattaaaggcctcgttgccgccgggcgcacgtacaataactgtcttgctatcaggaaagcttgtgattttctgctttccaaggagcttccgggcggtgggtggggcgagtcgtatttgagctgccaaaataaagtctatacaaacctcgagggtaaccggcctcaccttgtcaacacagcttgggttttgatggcgttgatagaggctggtcaagctgagcgggaccccacaccacttcatagggccgcaaggcttttgataaattcacagctcgaaaatggcgatttcccgcaacaggaaatcatgggagtttttaacaaaaactgcatgataacctacgccgcttataggaatatattcccgatctgggccctgggtgaatactgccatcgggtgttgacctaa

SEQ ID NO: 5 - protein, 758 aa, synthetic construct
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLT

SEQ ID NO: 6 - DNA, 1422 bp, synthetic construct
  source 1..1422: mol_type = other DNA
atgtggaccgtcgtgttgggcctcgcgaccctgttcgtcgcgtactatatccactggattaacaagtggcgcgacagcaagttcaacggcgtcctcccccccggcactatggggcttccgctcataggcgaaacaatccagctctcccggccgtccgacagcctggacgtgcacccgttcatccagaagaaggtggagcggtacgggccgatcttcaagacctgcctggccgggcgcccggtggtcgtctcggcggacgcggagttcaacaactacatcatgctccaggagggccgcgccgtcgagatgtggtatcttgacaccctgtccaaatttttcggcctggacaccgagtggctgaaggcgctgggcctgatccacaagtacatccgctccatcaccctgaaccacttcggcgccgaggcgctccgcgagcgcttcctgccgttcatcgaggcgtcctccatggaggcgctgcactcttggtctactcagccgtcggtcgaggtgaagaacgcgtccgcgctgatggtgttccgcaccagcgtgaacaagatgttcggcgaggacgcgaagaagctgtccgggaacatcccgggcaagttcacgaagctcctcgggggcttcctctcgctcccgctcaacttcccggggaccacctaccacaagtgcctcaaggacatgaaggagatccagaagaagctgcgcgaggtggtcgacgaccgcctggcgaacgtggggccggacgtggaggacttcctcggccaggcgctgaaggacaaggagagcgagaagttcatctccgaggagttcatcatccagcttcttttcagcatcagcttcgcgagcttcgagtccatcagcaccacgctcacgctgatcctcaagctgctggacgagcacccggaggtcgtgaaggagctggaggcggagcacgaggcgatccgcaaggcccgcgccgacccagacggcccgatcacctgggaggagtacaagtccatgaccttcaccctccaggtgatcaacgagacgctgcgcctggggtccgtgaccccggccctgctgaggaagaccgtcaaggacctccaggtgaagggctacatcatccccgagggctggaccatcatgctggtgacggcctcccgccaccgcgacccgaaggtgtacaaggacccgcacatcttcaacccgtggaggtggaaggacctggactccatcaccatccagaagaacttcatgccgttcggcggcggcctgcgccactgcgcgggggccgagtactccaaagtgtacctgtgcaccttcctgcacatcctgtgcaccaagtaccgctggaccaagctgggcggggggacgatcgcccgcgcccacatcctgtccttcgaggacggcctgcacgtgaagttcaccccaaaggagtga

SEQ ID NO: 7 - protein, 473 aa, Siraitia grosvenorii
MWTVVLGLATLFVAYYIHWINKWRDSKFNGVLPPGTMGLPLIGETIQLSRPSDSLDVHPFIQKKVERYGPIFKTCLAGRPVVVSADAEFNNYIMLQEGRAVEMWYLDTLSKFFGLDTEWLKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVINETLRLGSVTPALLRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGTIARAHILSFEDGLHVKFTPKE

SEQ ID NO: 8 - DNA, 1425 bp, synthetic construct
  source 1..1425: mol_type = other DNA
atggtgcagccccgggtgctgctgttccctttccccgcgctgggccacgtgaagcccttcctgtcgctggcggagctcctgtccgacgccggcatcgacgtggtcttcctctcgacggagtataaccaccgccgcatctccaacaccgaggccctggcctcccgcttccccaccctgcacttcgaaacgataccggacggcctgccccccaacgagtcgcgcgccctcgcggacggccccctgtacttcagcatgagggagggcaccaagccgcgcttccggcagctgatccagagccttaacgacggccgttggcccatcacctgtatcatcacggacatcatgctcagttcgcccatcgaggtggcggaggagttcgggatcccggtgatcgcgttctgcccgtgctccgctcgctacctctcgatccacttcttcatcccgaagctggtggaggaggggcagatcccgtacgcggacgacgaccccattggggagatccagggggtgcccctcttcgaggggcttctccgacggaaccacctcccgggatcttggtccgacaagtccgcggacatttccttctcgcacggcctcatcaaccagacgctcgcggccgggcgggcctcggccctgatcctcaacaccttcgacgagctcgaggcccctttcctcacccacctgagctccatcttcaacaagatctacaccatcggccccctccacgccctgtccaagagccggctcggggactcctcctcgtcggccagcgcgctctcggggttctggaaggaggaccgcgcgtgcatgtcctggctcgactgccagccgccgcgctcggtcgtgttcgtctccttcgggtcgaccatgaagatgaaggcggacgagcttcgggagttctggtacgggctggtgagcagcgggaagccgttcctctgcgtgttgcggtccgacgtcgtcagcggcggcgaggcggcggagctcatcgagcagatggcggaggaggagggggctgggggcaagctgggcatggtcgtcgagtgggcggcgcaggagaaggtcctctcccaccctgccgtggggggcttcctgacgcactgcggctggaactcgacggtggagagcatcgccgccggcgtccccatgatgtgctggccgatcctcggggaccagccctccaacgccacttggatcgaccgggtctggaagatcggggtcgagcgaaacaaccgggagtgggaccggttgacggtggagaagatggtccgcgcattgatggagggccagaagcgggtggagatccagcggtcgatggagaagctgtcgaagctggcgaacgagaaggtcgtccggggggggctgtctttcgacaacctcgaggtcctcgtggaggacatcaagaagctgaagccgtacaagttctag

SEQ ID NO: 9 - protein, 474 aa, Siraitia grosvenorii
MVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKF

SEQ ID NO: 10 - DNA, 1362 bp, synthetic construct
  source 1..1362: mol_type = other DNA
atggacgcgcagcggggccacacgacgacgatcctgatgttcccctggctcggctacgggcacctcagcgccttcctcgagctggcgaagagcctgtcccggcgcaacttccacatctacttctgctcgaccagcgtcaacctggacgcgatcaagcccaagctgccctccagcagctcctccgacagcatccagctggtggagctgtgcctgccctccagccccgaccagctccccccgcacctccacaccaccaacgcgctcccgccccacctcatgcccaccctccaccaggccttcagcatggcggcgcagcacttcgcggccatcctgcacaccctcgcgccccacctcctgatctacgactcgttccagccgtgggcaccccagctggcctcctcgctcaacatcccggccatcaacttcaacacgaccggggcgtccgtcctgacccggatgctgcacgcgacccactacccgagcagcaagttccccatcagcgagttcgtgctccacgactactggaaggccatgtacagcgcggccgggggcgcggtgacgaagaaggaccacaagatcggcgagacgctggcgaactgcctccacgccagctgctcggtcatcctgatcaactccttccgcgagctggaggagaagtacatggactacctgtccgtgctcctgaacaagaaggtggtgccggtggggccgctggtgtacgagccgaaccaggatggagaggacgaggggtactccagcataaagaactggctcgacaagaaggagccgagctccacggtgttcgtgtccttcggcagcgagtacttcccgagcaaggaggagatggaggagatcgcccacgggctcgaggcctctgaagttcatttcatctgggttgtgcggttcccgcagggggacaacacctccgcgatcgaggacgccctgccgaagggcttcctcgagagggtgggcgagaggggcatggtggtgaagggctgggccccgcaggcgaagatcctgaagcactggtccaccgggggcttcgtgtcccactgcggctggaacagcgtgatggagagcatgatgttcggggtgccgatcatcggcgtgccgatgcacctggaccagccgttcaacgccggcctggccgaggaggccggcgtgggggtggaggcgaagcgcgaccccgacgggaagatccagcgcgacgaggtcgccaagctgatcaaggaggtggtggtggagaagacccgggaggacgtgaggaagaaggcgcgcgagatgtccgagatcctccgcagcaagggggaggagaagatggacgagatggtggccgcgatctccctcttcctcaagatctga

SEQ ID NO: 11 - protein, 453 aa, Siraitia grosvenorii
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKI

SEQ ID NO: 12 - DNA, 1587 bp, synthetic construct
  source 1..1587: mol_type = other DNA
atggtggaccagtgcgccttgggctggatcttggcctccgcgctgggcctcgtgatcgccctgtgcttcttcgtcgccccccggcggaaccaccggggggtcgactcgaaggagagggacgagtgcgtccagagcgcggccaccacgaagggggagtgccggttcaacgaccgcgacgtcgacgtgatcgtcgttggcgccggcgtcgcgggcagcgccctcgcccacactctggggaaggacgggcggcgcgtgcacgtcattgagcgcgacctgacggagcccgaccggatcgtgggggagctcctccagcccggggggtacctcaagctcatcgagctgggtctgcaggactgcgtcgaggagatcgacgcgcagagggtgtacggctacgcgctgttcaaggacgggaagaacacccggctctcttacccgttggagaatttccactccgacgtgtcggggcggagcttccacaacggccgcttcatccagcgcatgagggagaaggccgcgtccctgcccaacgtgcggttggagcagggcacggttacctccctgctggaggagaagggcacgatcaagggcgtgcagtacaagtccaagaacggtgaggagaagacggcctacgcccccctgaccatcgtctgcgacggctgcttctcgaacctgcgccgctccctgtgcaaccctatggtggacgtgcccagctacttcgtgggcctcgtcctggagaactgcgagctacccttcgcgaaccacgggcacgttatcctcggggatccctcgcccatcctcttctaccagatcagccgtaccgagatccgttgcctcgtggacgtccccgggcagaaggtcccctccatcgcgaacggggagatggagaagtaccttaagacggtggtcgcgccccaggtgcccccgcagatctacgactccttcatcgcggcgatcgacaaggggaacatccggacgatgccgaaccgctccatgcccgctgcgccccaccccacgcccggcgccttgctcatgggcgacgcgttcaacatgcgccaccccctgaccgggggcggcatgaccgtggccctcagtgacatcgtcgtactccgcaacctgctcaagccgctgaaggacctgtcggacgcctctaccctctgcaagtacctcgagagcttctacacattgcggaagccagtggcttcgaccatcaacacgctagcgggggcgctgtacaaggtcttctgcgccagccccgaccaggcgcgaaaggagatgcggcaggcctgcttcgactacctcagcctgggcggcatcttctcaaacggccccgtctccctgcttagcggcttgaacccgcgccccctgtctctggttctccacttcttcgcggtcgcgatctacggcgtcggccgcctgctgctgccgttcccctcggtgaagggcatctggatcggcgccaggttgatctacagcgcctcgggcatcatcttccccatcatccgggccgagggcgtccggcagatgttcttccccgccacggtcccggcctactaccgctccccccccgtgttcaagcccatcgtctga

SEQ ID NO: 13 - protein, 528 aa, Siraitia grosvenorii
MVDQCALGWILASALGLVIALCFFVAPRRNHRGVDSKERDECVQSAATTKGECRFNDRDVDVIVVGAGVAGSALAHTLGKDGRRVHVIERDLTEPDRIVGELLQPGGYLKLIELGLQDCVEEIDAQRVYGYALFKDGKNTRLSYPLENFHSDVSGRSFHNGRFIQRMREKAASLPNVRLEQGTVTSLLEEKGTIKGVQYKSKNGEEKTAYAPLTIVCDGCFSNLRRSLCNPMVDVPSYFVGLVLENCELPFANHGHVILGDPSPILFYQISRTEIRCLVDVPGQKVPSIANGEMEKYLKTVVAPQVPPQIYDSFIAAIDKGNIRTMPNRSMPAAPHPTPGALLMGDAFNMRHPLTGGGMTVALSDIVVLRNLLKPLKDLSDASTLCKYLESFYTLRKPVASTINTLAGALYKVFCASPDQARKEMRQACFDYLSLGGIFSNGPVSLLSGLNPRPLSLVLHFFAVAIYGVGRLLLPFPSVKGIWIGARLIYSASGIIFPIIRAEGVRQMFFPATVPAYYRSPPVFKPIV

SEQ ID NO: 14 - DNA, 951 bp, synthetic construct
  source 1..951: mol_type = other DNA
atggaccagatcgagcacatcaccatcaacaccaacggcatcaagatgcacatcgcctccgtgggcacgggcccggtggtgctgctgctgcacggcttcccggagctgtggtactcctggcgccaccagctgctctacctgtcctccgtgggctacagggccatcgccccggacctgaggggctacggcgacaccgactccccggcctccccgaccagctacaccgccctgcacatcgtcggggacctggtgggcgccctggacgagctcggcatcgagaaggtgtttctcgtgggccacgactggggcgcgatcatcgcctggtacttctgcctgttccgcccggaccgcatcaaggccctggtgaacctgtccgtgcagttcatcccgaggaacccggccatcccgttcatcgagggcttcaggaccgccttcggcgacgacttctacatgtgcaggttccaggtgccgggcgaggccgaggaggacttcgcctccatcgacaccgcccagctgttcaagacctccctgtgcaaccgctcctccgccccgccgtgcctgccgaaggagatcgggttcagggccatcccgccgccggagaatctgccgtcctggctgacggaggaagacatcaactattatgccgccaagttcaagcagaccggcttcaccggcgccctgaactactacagggccttcgacctgacctgggagctgaccgccccgtggaccggcgcgcagatccaggtgccggtgaagttcatcgtcggcgactccgacctgacgtaccacttcccgggcgccaaggagtacatccacaacggcgggttcaagaaggacgtgccgctgctggaggaggtcgtggtggtgaaggacgcctgccacttcatcaatcaggagcgcccgcaggagatcaacgcccacatccacgacttcatcaacaagttctga

SEQ ID NO: 15 - protein, 316 aa, Siraitia grosvenorii
MDQIEHITINTNGIKMHIASVGTGPVVLLLHGFPELWYSWRHQLLYLSSVGYRAIAPDLRGYGDTDSPASPTSYTALHIVGDLVGALDELGIEKVFLVGHDWGAIIAWYFCLFRPDRIKALVNLSVQFIPRNPAIPFIEGFRTAFGDDFYMCRFQVPGEAEEDFASIDTAQLFKTSLCNRSSAPPCLPKEIGFRAIPPPENLPSWLTEEDINYYAAKFKQTGFTGALNYYRAFDLTWELTAPWTGAQIQVPVKFIVGDSDLTYHFPGAKEYIHNGGFKKDVPLLEEVVVVKDACHFINQERPQEINAHIHDFINKF

SEQ ID NO: 16 - DNA, 1275 bp, synthetic construct
  source 1..1275: mol_type = other DNA
atgaacacgaccccgctgccggaggaggacgaggaggtggtgaagatggtggtggacgggtccgtgccgtcctactcgctcgagtccaagctcggcgacccgaagcgcgcggcgtccatccggcgggaggcgctgcagcgcatgacggggcggagcatccacgggctgccgttcgagggcttcgactacgagagcatcctgggccagtgctgcgagatgccggtggggtacgtccagatcccggtgggcatcgcggggccgctgctgctcgacgggttcgagtacaccgtgccgatggccaccacggagggctgcctggtggcctccaccaacaggggctgcaaggccatctacgcctccggcggggcgacctcaatgctcctcaaagacggcatgacgagggccccggtggtcaggttcggcagcgcgaagagggcgagcgagctgaagttcttcctggaggacccgaacaacttcgacaccctggccgtggtgttcaaccgctccagccgcttcgcccgcctgcagagcatcaggtgcagcatcgcgggcaagaacctgtacgtccgcttctgctgctccaccggcgacgccatgggcatgaacatggtgtccaagggcgtgcagaacgtgctggagttcctgcagcacgacttctccgacatggaggtgatcggcatctccggcaacttctgcgccgacaagaagccggccgccgtgaactggatcgagggcaggggcaagtccgtggtgtgcgaggccattatcaaggacgaggtggtgcgcaaggtgctcaagaccagcgtggcgagcctggtggagctgaacatgctgaagaacctgaccggctccgccatggccggggccctcggcggcttcaacgcgcacagcagcaacatcgtgtccgccatcttcctggccaccggccaggacccggcccagaacgtggagtcctcccactgcatcacgatgatggagccggtcaacaacggcaaggacctccacatctccgtgaccatgccgtccatcgaggtcgggaccgtggggggcggcacccagctggcgtcccagagcgcgtgcctgaacctgctgggggtcaagggcgcgtccaaggagagcccgggggccaacagcaggctgctggcgaccatcgtggccggctccgtgctggccggcgagctctccctgatgtccgccatcgcggcgggccagctggtgaggtcccacatgaagtacaaccgctcctccagggatgtgtccaaggtggagagctaa

SEQ ID NO: 17 - protein, 424 aa, synthetic construct
MNTTPLPEEDEEVVKMVVDGSVPSYSLESKLGDPKRAASIRREALQRMTGRSIHGLPFEGFDYESILGQCCEMPVGYVQIPVGIAGPLLLDGFEYTVPMATTEGCLVASTNRGCKAIYASGGATSMLLKDGMTRAPVVRFGSAKRASELKFFLEDPNNFDTLAVVFNRSSRFARLQSIRCSIAGKNLYVRFCCSTGDAMGMNMVSKGVQNVLEFLQHDFSDMEVIGISGNFCADKKPAAVNWIEGRGKSVVCEAIIKDEVVRKVLKTSVASLVELNMLKNLTGSAMAGALGGFNAHSSNIVSAIFLATGQDPAQNVESSHCITMMEPVNNGKDLHISVTMPSIEVGTVGGGTQLASQSACLNLLGVKGASKESPGANSRLLATIVAGSVLAGELSLMSAIAAGQLVRSHMKYNRSSRDVSKVES

SEQ ID NO: 18 - DNA, 2127 bp, synthetic construct
  source 1..2127: mol_type = other DNA
atgcagtccgagtccaggtccatgaaggtgtccccgctggagctgatgtccgccatcatcaggaaggccatggacccgtccagggagtcctccgagtccgtgagggaggtggccaccctgatcctggagaacagggagttcgtgatgatcctgaccaccctgctggccgtgctgatcggctgcgtggtggtgctggtgtggaagaggtcctccggccagaaggccaagccgttcgagccgccgaagcagctgatcgtgaaggagccggagccggaggtggacgacggcaagaagaaggtgaccgtgttcttcggcacccagaccggcaccgccgagggcttcgccaaggccctggccgaggaggccaaggccaggtacgagaaggccaccttcagggtggtggacctggacgactacgccgccgacgacgacgagtacgaggagaagctgaagaaggaaaccctggccatcttcttcctggccacctacggcgacggcgagccgaccgacaacgccgccaggttctacaagtggttctccgagggcaaggagaagggcgactggatctccaacctgcagtacgccgtgttcggcctgggcaacaggcagtacgagcacttcaacaagatcgccaaggtggtggacgagcagctggccgagcagggcggcaagaggctggtgccggtgggcctgggcgacgacgaccagtgcatcgaggacgacttctccgcctggagggaggccctgtggccggagctggacaagctgctgagggacgacgacgactccaccaccgtggccaccccgtacaccgccgccgtgctggagtacagggtggtgttctacgacgccgccgacgtgtccgtggaggacaagaggtgggccttcgccaacggccacgccgtgtacgacgcccagcacccgtgcagggccaacgtggccatgaggaaggagctgcacaccccggcctccgacaggtcctgcatccacctggagttcgacatctccggcaccggcctgacctacgaaaccggcgaccacgtgggcgtgttctgcgagaacctggacgaaaccgtggaggacgccatcaggctgatcggcctgtccccggaaacctacttctccatccacaccgacaaggacgacggcaccccgctgggcggctcctccctgccgccgccgttcgccccgtgcaccctgaggaccgccctgacccagtacgccgacctgctgtcctccccgaagaagtccgccctggtggccctggccgcccacgcctccgacccggccgaggccgacaggctgaggcacctgtcctccccggccggcaaggacgagtacgcccagtggatcatcgcctcccagaggtccctgctggaggtgatggccgagttcccgtccgccaagccgccgctgggcgtgttcttcgccgccgtggccccgaggctgcagccgaggtactactccatctcctcctccccgaggatggccccgtccaggatccacgtgacctgcgccctggtgtacgacaagaccccgaccggcaggatccacaagggcgtgtgctccacctggatgaagaacgccgtgccgctggaggagtcccaggcctgctcctgggccccgatctacgtgaggcagtccaacttcaagctgccgaccgactccaagctgccgatcatcatgatcggcccgggcaccggcctggccccgttcaggggcttcctgcaggagaggctggccctgaaggaggccggcgtggagctgggccactccatcctgttcttcggctgcaggaacaggaagatggactacatctacgaggacgagctgtccaacttcgccgaaaccggcgccctgtccgagctgatcgtggccttctccagggagggcccgaccaaggagtacgtgcagcacaagatggtggacaaggcctccgacatctggaacatcctgtcccagggcggctacatctacgtgtgcggcgacgccaagggcatggccagggacgtgcacaggaccctgcacaacatcgtgcaggagcagggctccctggactcctccaaggccgagtccatggtgaagaacctgcagatgtccggcaggtacctgagggacgtgtggtga

SEQ ID NO: 19 - protein, 708 aa, Siraitia grosvenorii
MQSESRSMKVSPLELMSAIIRKAMDPSRESSESVREVATLILENREFVMILTTLLAVLIGCVVVLVWKRSSGQKAKPFEPPKQLIVKEPEPEVDDGKKKVTVFFGTQTGTAEGFAKALAEEAKARYEKATFRVVDLDDYAADDDEYEEKLKKETLAIFFLATYGDGEPTDNAARFYKWFSEGKEKGDWISNLQYAVFGLGNRQYEHFNKIAKVVDEQLAEQGGKRLVPVGLGDDDQCIEDDFSAWREALWPELDKLLRDDDDSTTVATPYTAAVLEYRVVFYDAADVSVEDKRWAFANGHAVYDAQHPCRANVAMRKELHTPASDRSCIHLEFDISGTGLTYETGDHVGVFCENLDETVEDAIRLIGLSPETYFSIHTDKDDGTPLGGSSLPPPFAPCTLRTALTQYADLLSSPKKSALVALAAHASDPAEADRLRHLSSPAGKDEYAQWIIASQRSLLEVMAEFPSAKPPLGVFFAAVAPRLQPRYYSISSSPRMAPSRIHVTCALVYDKTPTGRIHKGVCSTWMKNAVPLEESQACSWAPIYVRQSNFKLPTDSKLPIIMIGPGTGLAPFRGFLQERLALKEAGVELGHSILFFGCRNRKMDYIYEDELSNFAETGALSELIVAFSREGPTKEYVQHKMVDKASDIWNILSQGGYIYVCGDAKGMARDVHRTLHNIVQEQGSLDSSKAESMVKNLQMSGRYLRDVW

SEQ ID NO: 20 - DNA, 1383 bp, synthetic construct
  source 1..1383: mol_type = other DNA
atggacgaaaccaccgtgaacggcggcaggagggcctccgacgtggtggtgttcgccttcccgaggcacggccacatgtccccgatgctgcagttctccaagaggctggtgtccaagggcctgagggtgaccttcctgatcaccacctccgccaccgagtccctgaggctgaacctgccgccgtcctcctccctggacctgcaagtgatctccgacgtgccggagtccaacgacatcgccaccctggagggctacctgaggtccttcaaggccaccgtgtccaagaccctggccgacttcatcgacggcatcggcaacccgccgaagttcatcgtgtacgactccgtgatgccgtgggtgcaggaggtggccaggggcaggggcctggacgccgccccgttcttcacccagtcctccgccgtgaaccacatcctgaaccacgtgtacggcggctccctgtccatcccggccccggagaacaccgccgtgtccctgccgtccatgccggtgctgcaggccgaggacctgccggccttcccggacgacccggaggtggtgatgaacttcatgacctcccagttctccaacttccaggacgccaagtggatcttcttcaacaccttcgaccagctggagtgcaagaagcagtcccaggtggtgaactggatggccgacaggtggccgatcaagaccgtgggcccgaccatcccgtccgcctacctggacgacggcaggctggaggacgacagggccttcggcctgaacctgctgaagccggaggacggcaagaacaccaggcagtggcagtggctggactccaaggacaccgcctccgtgctgtacatctccttcggctccctggccatcctgcaggaggagcaagtgaaggagctggcctacttcctgaaggacaccaacctgtccttcctgtgggtgctgagggactccgagctgcagaagctgccgcacaacttcgtgcaggaaacctcccacaggggcctggtggtgaactggtgctcccagctgcaagtgctgtcccacagggccgtgtcctgcttcgtgacccactgcggctggaactccaccctggaggccctgtccctgggcgtgccgatggtggccatcccgcagtgggtggaccagaccaccaacgccaagttcgtggccgacgtgtggagggtgggcgtgagggtgaagaagaaggacgagaggatcgtgaccaaggaggagctggaggcctccatcaggcaagtggtgcagggcgagggcaggaacgagttcaagcacaacgccatcaagtggaagaagctggccaaggaggccgtggacgagggcggctcctccgacaagaacatcgaggagttcgtgaagaccatcgcctga

SEQ ID NO: 21 - protein, 460 aa, Siraitia grosvenorii
MDETTVNGGRRASDVVVFAFPRHGHMSPMLQFSKRLVSKGLRVTFLITTSATESLRLNLPPSSSLDLQVISDVPESNDIATLEGYLRSFKATVSKTLADFIDGIGNPPKFIVYDSVMPWVQEVARGRGLDAAPFFTQSSAVNHILNHVYGGSLSIPAPENTAVSLPSMPVLQAEDLPAFPDDPEVVMNFMTSQFSNFQDAKWIFFNTFDQLECKKQSQVVNWMADRWPIKTVGPTIPSAYLDDGRLEDDRAFGLNLLKPEDGKNTRQWQWLDSKDTASVLYISFGSLAILQEEQVKELAYFLKDTNLSFLWVLRDSELQKLPHNFVQETSHRGLVVNWCSQLQVLSHRAVSCFVTHCGWNSTLEALSLGVPMVAIPQWVDQTTNAKFVADVWRVGVRVKKKDERIVTKEELEASIRQVVQGEGRNEFKHNAIKWKKLAKEAVDEGGSSDKNIEEFVKTIA

SEQ ID NO: 22 - DNA, 1374 bp, synthetic construct
  source 1..1374: mol_type = other DNA
atgggcggcagggagggcaaggtgcacgtgctggtggtgctgttcccggacggccagggccacatcaacccgatcctgcagttctccaagaggctggccttcaagggcctggccgtgaccctgctgaacgtgttcaggtcccaggagtcccacaccctgaacctgcacccgtccatcaacgtggagcacaggccgaggctgccgtaccagggcaccgagccggagtccctggagtcccacatgcacaggctgcagacctccatctgcttccacatgaccaagctgatctcccaccaccagtcctccggctccccgatcgcctgcgtggtgtacgactccgtgaccccgtgggtgctggacgtggccaagggcttcggcgtgccgggcgccccgttcttcaccgagtcctccgccgtgaacgccatcttctaccacgtgcacaggggctggctgaagatcccgatcgacgactcctccgtgtccctgccgggcctgccgctgctgcagatctccgacctgccgtccatgatcccgaacccgcagcagtacccgatcttcctgaggctgatgatcgaccagttctccaacaagccggactggatgttcatcaacaccttcaactccctggagccggaggtgctgcagtggatccagacccacaccccgctgaagaccgtgggcccgaccgtgccgtccatcatcaccgacaagaggctgaaggacgaccagcactacggcatcgacctgttcaagtccaccggcgacgagatcaagaccatcgagtggctggactccaaggactccggctccgtgatctacgtgtccttcggctccgtgtccgacctgggcgaggagcagatggaggagctggcctggggcctgaagtccaccaacaagtccttcctgtgggtggtgagggaaaccggcacctccaagctgccgtccaacctgctggaggagatgaaggaaaccggcatggtggtgaactggtgctcccagctgcaagtgctggcccacaagtccgtgggctgcttcatcacccactgcggctggaactccgtgctggaggccctgtccctgggcatcccgatggtggccatgccgcagtggaccgaccagatgaccaacgccaagttcgtggaggacgtgtggaaggtgggcgtgagggtgtcctccaaggagaacgccatcgccggcagggaggagatcgagctgtgcatcaggcaagtgatggagggcgagaggtccgccgagatgagggagaacgcctccaggtggatgaagctggccaaggaggccgtggacgagggcggctcctccgacaagaacatcgacgagttcgtggcccagctgaccaacaggaacccgaactga

SEQ ID NO: 23 - protein, 457 aa, Siraitia grosvenorii
MGGREGKVHVLVVLFPDGQGHINPILQFSKRLAFKGLAVTLLNVFRSQESHTLNLHPSINVEHRPRLPYQGTEPESLESHMHRLQTSICFHMTKLISHHQSSGSPIACVVYDSVTPWVLDVAKGFGVPGAPFFTESSAVNAIFYHVHRGWLKIPIDDSSVSLPGLPLLQISDLPSMIPNPQQYPIFLRLMIDQFSNKPDWMFINTFNSLEPEVLQWIQTHTPLKTVGPTVPSIITDKRLKDDQHYGIDLFKSTGDEIKTIEWLDSKDSGSVIYVSFGSVSDLGEEQMEELAWGLKSTNKSFLWVVRETGTSKLPSNLLEEMKETGMVVNWCSQLQVLAHKSVGCFITHCGWNSVLEALSLGIPMVAMPQWTDQMTNAKFVEDVWKVGVRVSSKENAIAGREEIELCIRQVMEGERSAEMRENASRWMKLAKEAVDEGGSSDKNIDEFVAQLTNRNPN

SEQ ID NO: 24 - DNA, 1425 bp, synthetic construct
  source 1..1425: mol_type = other DNA
atgtggaccatcttcctgggcttcgtgaccctgaccatcgtgtactacatctactggaccaacaagtggaaggactccaagttcaaccgcgtgctgccgccgggcaccatgggcctcccgctgatcggcgaaaccctgcagttcctgaggccgtcctactccctggacctgcacccgttcttccagaccaaggtgaagaggtacggcccgatcttcaagacctgcctggccggcaggccggtggtggtgtccaccgacgccgagttcaaccactacatcatgctgcaggagggcaaggccgtggagatgtggtacctggacgccttctccaagttcttcggcctggacaccgagtggaccaaggccctgggcctgatccacaagtacatcaggtccatcaccctgaaccactacggcgtggagtccctgagggagaggttcctgccgaggatcgaggagtccgtgagggaaaccctgcactactgggccacccagccgtccgtggaggtgaaggactccgccgccgccatggtgctgaagacctccgtgatcaagctgttctccgaggactccaacaagctgctgtccggcggcctgctgaagaagttcaccaagctgctgggcggcttcctgaccctgccgctgaacctgccgggcaccacctaccacaggtgcatcaaggacatgaaggagatccagaagaagctgaaggagatcctggaggagaggctggccaggggcaacggctccgacgaggacttcctgggccaggtgatcaaggacaaggagtcccagaagttcatctccgaggagttcatcatccagctgctgttctccatgtccttcgccaccttcgagtccatctccgcctccctgaccctgatcctgatcctgctggccgaccacccggaggtggccaaggagctggaggtggagcacgaggccatcaggaaggcgcgcgtggacccggacggcctgatcacctgggaggagtacaagtccatgaccttcacccacaacgtgatctacgaaaccctgcgcctgggctccgtgatcccgtccctgccccggaagaccaccaaggagatccagatcaagggctacaccatcccggagggctggaccatcatgctggtgatcgcctcccggcaccgaaacccgaaggtgtacaaggacccggacaccttcaacccgtggcggtggaaggagctggactccatcaccatccagaagaacttcatgccgttcggcggcggcctgcgccactgcgccggcgccgagtacaccaaggtgtacctgtgcaccttcctgcacatcctgttcaccaagtaccgctggaccaagctgaagggcggcaagatcgcccgcgccctgatcctcaagttcgtggacggcctgcacgtcaacttcaccccgaaggagtga

SEQ ID NO: 25 - protein, 474 aa, Citrullus lanatus
MWTIFLGFVTLTIVYYIYWTNKWKDSKFNRVLPPGTMGLPLIGETLQFLRPSYSLDLHPFFQTKVKRYGPIFKTCLAGRPVVVSTDAEFNHYIMLQEGKAVEMWYLDAFSKFFGLDTEWTKALGLIHKYIRSITLNHYGVESLRERFLPRIEESVRETLHYWATQPSVEVKDSAAAMVLKTSVIKLFSEDSNKLLSGGLLKKFTKLLGGFLTLPLNLPGTTYHRCIKDMKEIQKKLKEILEERLARGNGSDEDFLGQVIKDKESQKFISEEFIIQLLFSMSFATFESISASLTLILILLADHPEVAKELEVEHEAIRKARVDPDGLITWEEYKSMTFTHNVIYETLRLGSVIPSLPRKTTKEIQIKGYTIPEGWTIMLVIASRHRNPKVYKDPDTFNPWRWKELDSITIQKNFMPFGGGLRHCAGAEYTKVYLCTFLHILFTKYRWTKLKGGKIARALILKFVDGLHVNFTPKE

SEQ ID NO: 26 - DNA, 1422 bp, synthetic construct
  source 1..1422: mol_type = other DNA
atgtggaccgtggtgctgggcctggccaccctgttcgtggcctactacatccactggatcaacaagtggagggactccaagttcaacggcgtgctgccgccgggcaccatgggcctgccgctgatcggcgaaaccctgcagttcctgaggccgtccgactccctggacgtgcacccgttcttccagaagaaggtggagaggtacggcccgatcttcaagacctgcctggccggcaggccggtggtggtgtccgccgacgccgagttcaacaactacatcatgctgcaggagggcagggccgtggagatgtggtacctggacaccctgtccaagttcttcggcctggacaccgagtggaccaaggccctgggcctgatccacaagtacatcaggtccatcaccctgaaccacttcggcgccgaggccctgagggagaggttcctgccgttcatcgaggcctcctccatggaggccctgcactcctggtccacccagccgtccgtggaggtgaagaacgcctccgccctgatggtgttcaggacctccgtgaacaagatgttcggcgaggacgccaagaagctgtccggcaacatcccgggcaagttcaccaagctgctgggcggcttcctgtccctgccgctgaacttcccgggcaccacctaccacaagtgcctgaaggacatgaaggagatccagaagaagctgagggaggtggtggacgacaggctggccaacgtgggcccggacgtggaggacttcctgggccaggccctgaaggacaaggagtccgagaagttcatctccgaggagttcatcatccagctgctgttctccatctccttcgcctccttcgagtccatctccaccaccctgaccctgatcctgaagctgctggacgagcacccggaggtggtgaaggagctggaggccgagcacgaggccatcaggaaggccagggccgacccggacggcccgatcacctgggaggagtacaagtccatgaccttcaccctgcaagtgatctacgaaaccctgaggctgggctccgtgatcccggccctgccgaggaagaccgtgaaggacctgcaagtgaagggctacatcatcccggagggctggaccatcatgctggtgaccgcctccaggcacagggacccgaaggtgtacaaggacccgcacatcttcaacccgtggaggtggaaggacctggactccatcaccatccagaagaacttcatgccgttcggcggcggcctgaggcactgcgccggcgccgagtactccaaggtgtacctgtgcaccttcctgcacatcctgtgcaccaagtacaggtggaccaagctgggcggcggcaccatcgccagggcccacatcctgtccttcgaggacggcctgcacgtgaagttcaccccgaaggagtga

SEQ ID NO: 27 - protein, 473 aa, synthetic construct
MWTVVLGLATLFVAYYIHWINKWRDSKFNGVLPPGTMGLPLIGETLQFLRPSDSLDVHPFFQKKVERYGPIFKTCLAGRPVVVSADAEFNNYIMLQEGRAVEMWYLDTLSKFFGLDTEWTKALGLIHKYIRSITLNHFGAEALRERFLPFIEASSMEALHSWSTQPSVEVKNASALMVFRTSVNKMFGEDAKKLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVIYETLRLGSVIPALPRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGTIARAHILSFEDGLHVKFTPKE

SEQ ID NO: 28 - DNA, 1425 bp, synthetic construct
  source 1..1425: mol_type = other DNA
atgtggaccgtggtgctgggcctggccaccctgttcgtggcctactacatccactggatcaacaagtggagggactccaagttcaacggcgtgctgccgccgggcaccatgggcctgccgctgatcggcgaaaccctgcagttcctgaggccgtccgactccctggacgtgcacccgttcttccagaagaaggtggagaggtacggcccgatcttcaagacctgcctggccggcaggccggtggtggtgtccgccgacgccgagttcaacaactacatcatgctgcaggagggcagggccgtggagatgtggtacctggacaccctgtccaagttcttcggcctggacaccgagtggaccaaggccctgggcctgatccacaagtacatcaggtccatcaccctgaaccacttcggcgccgaggccctgagggagaggttcctgccgttcatcgaggcctcctccatggaggccctgcactcctggtccacccagccgtccgtggaggtgaagaacgcctccgccctgatggtgttcaggacctccgtgaacaagatgttcggcgaggacgccaagaagctgctgtccggcaacatcccgggcaagttcaccaagctgctgggcggcttcctgtccctgccgctgaacttcccgggcaccacctaccacaagtgcctgaaggacatgaaggagatccagaagaagctgagggaggtggtggacgacaggctggccaacgtgggcccggacgtggaggacttcctgggccaggccctgaaggacaaggagtccgagaagttcatctccgaggagttcatcatccagctgctgttctccatctccttcgcctccttcgagtccatctccaccaccctgaccctgatcctgaagctgctggacgagcacccggaggtggtgaaggagctggaggccgagcacgaggccatcaggaaggccagggccgacccggacggcccgatcacctgggaggagtacaagtccatgaccttcaccctgcaagtgatctacgaaaccctgaggctgggctccgtgatcccggccctgccgaggaagaccgtgaaggacctgcaagtgaagggctacatcatcccggagggctggaccatcatgctggtgaccgcctccaggcacagggacccgaaggtgtacaaggacccgcacatcttcaacccgtggaggtggaaggacctggactccatcaccatccagaagaacttcatgccgttcggcggcggcctgaggcactgcgccggcgccgagtactccaaggtgtacctgtgcaccttcctgcacatcctgtgcaccaagtacaggtggaccaagctgggcggcggcaccatcgccagggcccacatcctgtccttcgaggacggcctgcacgtgaagttcaccccgaaggagtga

SEQ ID NO: 29 - protein, 474 aa, synthetic construct
MWTVVLGLATLFVAYYIHWINKWRDSKFNGVLPPGTMGLPLIGETLQFLRPSDSLDVHPFFQKKVERYGPIFKTCLAGRPVVVSADAEFNNYIMLQEGRAVEMWYLDTLSKFFGLDTEWTKALGLIHKYIRSITLNHFGAEALRERFLPFIEASSMEALHSWSTQPSVEVKNASALMVFRTSVNKMFGEDAKKLLSGNIPGKFTKLLGGFLSLPLNFPGTTYHKCLKDMKEIQKKLREVVDDRLANVGPDVEDFLGQALKDKESEKFISEEFIIQLLFSISFASFESISTTLTLILKLLDEHPEVVKELEAEHEAIRKARADPDGPITWEEYKSMTFTLQVIYETLRLGSVIPALPRKTVKDLQVKGYIIPEGWTIMLVTASRHRDPKVYKDPHIFNPWRWKDLDSITIQKNFMPFGGGLRHCAGAEYSKVYLCTFLHILCTKYRWTKLGGGTIARAHILSFEDGLHVKFTPKE

SEQ ID NO: 30 - DNA, 1425 bp, synthetic construct
  source 1..1425: mol_type = other DNA
atgtggaccatcctgctgggcctggccaccctggccatcgcctactacatccactgggtgaacaagtggaaggactccaagttcaacggcgtgctgccgccgggcaccatgggcctgccgctgatcggcgaaaccctgcagttcctgaggccgtccgactccctggacgtgcacccgttcttccagaggaaggtgaagaggtacggcccgatcttcaagacctgcctggccggcaggccggtggtggtgtccaccgacgccgagttcaaccactacatcatgctgcaggagggcagggccgtggagatgtggtacctggacaccctgtccaagttcttcggcctggacaccgagtggaccaaggccctgggcctgatccacaagtacatcaggtccatcaccctgaaccacttcggcgccgagtccctgagggagaggttcctgccgaggatcgaggagtccgccagggaaaccctgcactactggtccacccagacctccgtggaggtgaaggagtccgccgccgccatggtgttcaggacctccatcgtgaagatgttctccgaggactcctccaagctgctgaccgagggcctgaccaagaagttcaccggcctgctgggcggcttcctgaccctgccgctgaacctgccgggcaccacctaccacaagtgcatcaaggacatgaagcagatccagaagaagctgaaggacatcctggaggagaggctggccaagggcgtgaagatcgacgaggacttcctgggccaggccatcaaggacaaggagtcccagcagttcatctccgaggagttcatcatccagctgctgttctccatctccttcgcctccttcgagtccatctccaccaccctgaccctgatcctgaacttcctggccgaccacccggacgtggtgaaggagctggaggccgagcacgaggccatcaggaaggccagggccgacccggacggcccgatcacctgggaggagtacaagtccatgaacttcaccctgaacgtgatctacgaaaccctgaggctgggctccgtgatcccggccctgccgaggaagaccaccaaggagatccagatcaagggctacaccatcccggagggctggaccgtgatgctggtgaccgcctccaggcacagggacccggaggtgtacaaggacccggacaccttcaacccgtggaggtggaaggagctggactccatcaccatccagaagaacttcatgccgttcggcggcggcctgaggcactgcgccggcgccgagtactccaaggtgtacctgtgcaccttcctgcacatcctgttcaccaagtacaggtggaggaagctgaagggcggcaagatcgccagggcccacatcctgaggttcgaggacggcctgtacgtgaacttcaccccgaaggagtga

SEQ ID NO: 31 - protein, 474 aa, synthetic construct
MWTILLGLATLAIAYYIHWVNKWKDSKFNGVLPPGTMGLPLIGETLQFLRPSDSLDVHPFFQRKVKRYGPIFKTCLAGRPVVVSTDAEFNHYIMLQEGRAVEMWYLDTLSKFFGLDTEWTKALGLIHKYIRSITLNHFGAESLRERFLPRIEESARETLHYWSTQTSVEVKESAAAMVFRTSIVKMFSEDSSKLLTEGLTKKFTGLLGGFLTLPLNLPGTTYHKCIKDMKQIQKKLKDILEERLAKGVKIDEDFLGQAIKDKESQQFISEEFIIQLLFSISFASFESISTTLTLILNFLADHPDVVKELEAEHEAIRKARADPDGPITWEEYKSMNFTLNVIYETLRLGSVIPALPRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARAHILRFEDGLYVNFTPKE

SEQ ID NO: 32 - DNA, 1422 bp, synthetic construct
  source 1..1422: mol_type = other DNA
atgtggaccatcctgctgggcctggccaccctggccatcgcctactacatccactgggtgaacaagtggaaggactccaagttcaacggcgtgctgccgccgggcaccatgggcctgccgctgatcggcgaaaccctgcagttcctgaggccgtccgactccctggacgtgcacccgttcttccagaggaaggtgaagaggtacggcccgatcttcaagacctgcctggccggcaggccggtggtggtgtccaccgacgccgagttcaaccactacatcatgctgcaggagggcagggccgtggagatgtggtacctggacaccctgtccaagttcttcggcctggacaccgagtggaccaaggccctgggcctgatccacaagtacatcaggtccatcaccctgaaccacttcggcgccgagtccctgagggagaggttcctgccgaggatcgaggagtccgccagggaaaccctgcactactggtccacccagacctccgtggaggtgaaggagtccgccgccgccatggtgttcaggacctccatcgtgaagatgttctccgaggactcctccaagctgaccgagggcctgaccaagaagttcaccggcctgctgggcggcttcctgaccctgccgctgaacctgccgggcaccacctaccacaagtgcatcaaggacatgaagcagatccagaagaagctgaaggacatcctggaggagaggctggccaagggcgtgaagatcgacgaggacttcctgggccaggccatcaaggacaaggagtcccagcagttcatctccgaggagttcatcatccagctgctgttctccatctccttcgcctccttcgagtccatctccaccaccctgaccctgatcctgaacttcctggccgaccacccggacgtggtgaaggagctggaggccgagcacgaggccatcaggaaggccagggccgacccggacggcccgatcacctgggaggagtacaagtccatgaacttcaccctgaacgtgatctacgaaaccctgaggctgggctccgtgatcccggccctgccgaggaagaccaccaaggagatccagatcaagggctacaccatcccggagggctggaccgtgatgctggtgaccgcctccaggcacagggacccggaggtgtacaaggacccggacaccttcaacccgtggaggtggaaggagctggactccatcaccatccagaagaacttcatgccgttcggcggcggcctgaggcactgcgccggcgccgagtactccaaggtgtacctgtgcaccttcctgcacatcctgttcaccaagtacaggtggaggaagctgaagggcggcaagatcgccagggcccacatcctgaggttcgaggacggcctgtacgtgaacttcaccccgaaggagtga

SEQ ID NO: 33 - protein, 473 aa, synthetic construct
MWTILLGLATLAIAYYIHWVNKWKDSKFNGVLPPGTMGLPLIGETLQFLRPSDSLDVHPFFQRKVKRYGPIFKTCLAGRPVVVSTDAEFNHYIMLQEGRAVEMWYLDTLSKFFGLDTEWTKALGLIHKYIRSITLNHFGAESLRERFLPRIEESARETLHYWSTQTSVEVKESAAAMVFRTSIVKMFSEDSSKLTEGLTKKFTGLLGGFLTLPLNLPGTTYHKCIKDMKQIQKKLKDILEERLAKGVKIDEDFLGQAIKDKESQQFISEEFIIQLLFSISFASFESISTTLTLILNFLADHPDVVKELEAEHEAIRKARADPDGPITWEEYKSMNFTLNVIYETLRLGSVIPALPRKTTKEIQIKGYTIPEGWTVMLVTASRHRDPEVYKDPDTFNPWRWKELDSITIQKNFMPFGGGLRHCAGAEYSKVYLCTFLHILFTKYRWRKLKGGKIARAHILRFEDGLYVNFTPKE

SEQ ID NO: 34 - DNA, 3177 bp, synthetic construct
  source 1..3177: mol_type = other DNA
atgtcgtccagcgtggcggccacgatcagcatctggatggtggtcgtctgcatcgtcggggtggggtggcgcgtcgtcaactgggtctggctccggccgaagaagctggagaagcgcctccgggagcaggggctcgcgggcaacagctacaggctgctgttcggggacctcaaggagagggccgcgatggcggagcaggcgaacagcaagccgatcaacttcagccacgacatcgggccgcgcgtgttcccgagcatgtacaagacgatccagaactacggcaagaactcctacatgtggctcgggccgtacccgcgcgtgcacatcatggacccgcagcagctcaagaccgtgttcaccctggtctacgacatccagaagccgaacctgaacccgctgatcaagttcctcctggacggcatcgtgacccacgagggggagaagtgggcgaagcaccgcaagatcatcaacccggcgttccacatggccaagctcaaggacatgatcccggccttcttccagtcctgcaacgagatcgtctccgagtgggagcgcatgatcccgaaggagggctccttcgagctggacgtgatgccgtacatgcagaacctggccgccgacgccatctcccgcaccgccttcggctcctcctacaaggagggccagatgatcttccagctgctgaaggagctgaccgacctggtggtgaaggccgccttcggcatctacatcccgggctggcgcttcctgccgaccaagtccaacaacaagatgaaggagatcaacaggaagatcaagaccctgctgctgggcatcatcgacaagcgccagaaggccatgaaggagggggaggtcgtgcagaacgacctgctgggcatcctggtggagagcaacctgaatgagatccgcggccacgagaacaacaaggacgcgggcatgtccatcgaggacgtgatcgaggagtgcaaggtgttctacatcgggggccaggagacgacggccgggctgctgatctggaccatgatcctcctgtccttccacacggagtggcaggagagggcccgcaccgaggtgctggaggtggtggggaacaagaagccggacttcgacggcctgaaccgcctgaagatcgtgaccatgatcctgtacgaggtcctgcgcctgtacccgccggcgtccatgctgaccaggatcgtgcagaaagaaacccgcatcggcaagctgaccctgccggccggggtgaccctgatcatcccgaccgtgctgatccaccgcgaccacgagctgtggggggaggacgcgaacgagttcaagccggagaggttctccgagggggtgtccaaggcggccaaggtgcagccggcgttcttcccgttcggctggggcccgcgcctgtgcctgggccagaacttcgccatgatcgaggccaaaatggccttctccctgatcctgcagcgcttctccttccacctgtcctcctcctacgcccacgccccgacggtggtgatgaccacccagccgcagcacggcgcccacatcgtgctgaggaagctgggaagcggagctactaacttcagcctgctgaagcaggctggagacgtggaggagaaccctggacctatgtccagctcagtcgcggctaccataagcatatggatggtcgttgtctgtatagtgggtgtcgggtggcgggtggtcaactgggtttggcttcgccccaaaaaacttgagaagcggctccgggaacaaggtctggctggaaattcgtatcggctccttttcggggaccttaaagaaagagccgcgatggctgaacaggcaaattcaaagccaattaacttttcgcatgatatagggccacgggtttttccgtcgatgtataaaactattcagaactatggtaagaactcttacatgtggttgggaccttatccgagggttcatatcatggatcctcagcagctgaagactgtgtttacgcttgtgtacgacatacagaagcccaatcttaatcccttgattaaatttttgttggatggaattgttacgcatgaaggggaaaagtgggctaaacaccgcaaaatcattaaccccgcattccatatggcaaagttgaaagatatgattccagcttttttccaatcctgtaatgaaattgtcagcgaatgggagaggatgatcccgaaggaggggagcttcgaattggatgtgatgccctatatgcagaatctggccgctgacgctattagccggactgccttcggaagcagctataaggaaggtcaaatgatttttcagttgctcaaagagttgaccgatcttgtggtgaaagcagccttcggcatttatatccccggatggagattcctccccacgaaaagcaacaataaaatgaaggagataaaccggaagattaaaactcttttgcttggtattatagataagaggcagaaggcaatgaaagaaggcgaggtcgtccagaatgaccttcttgggattttggtcgagtcgaaccttaacgagataagagggcatgagaataataaagacgcggggatgtccatcgaagatgtgatagaagagtgtaaggtgttctacataggtggccaggagacaactgcaggcctgttgatatggaccatgatattgctcagcttccatacggaatggcaggaacgcgctagaaccgaagttctcgaagtcgttgggaacaaaaaacctgatttcgacggattgaatcggctgaagatagttacaatgattctctatgaggtgctccggttgtatcctcccgcctctatgttgacccgcattgtccaaaaggaaacgagaatagggaagcttactcttccggctggagtcacactgataatcccaacggtgttgatacaccgcgatcacgaattgtggggcgaggacgcgaatgaattcaagcccgaacgcttcagcgagggagttagcaaggccgctaaggtgcagcctgcgttcttcccgttcggatgggggcccagactttgtctcgggcaaaacttcgcaatgattgaggccaaaatggcgttttctctgattctgcaaagattctctttccacctctcatcatcatatgcacacgcgcctaccgtcgttatgacaactcagcctcagcatggcgctcatatagttctcagaaagctctaa

SEQ ID NO: 35 - protein, 539 aa, synthetic construct
MSSSVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHMAKLKDMIPAFFQSCNEIVSEWERMIPKEGSFELDVMPYMQNLAADAISRTAFGSSYKEGQMIFQLLKELTDLVVKAAFGIYIPGWRFLPTKSNNKMKEINRKIKTLLLGIIDKRQKAMKEGEVVQNDLLGILVESNLNEIRGHENNKDAGMSIEDVIEECKVFYIGGQETTAGLLIWTMILLSFHTEWQERARTEVLEVVGNKKPDFDGLNRLKIVTMILYEVLRLYPPASMLTRIVQKETRIGKLTLPAGVTLIIPTVLIHRDHELWGEDANEFKPERFSEGVSKAAKVQPAFFPFGWGPRLCLGQNFAMIEAKMAFSLILQRFSFHLSSSYAHAPTVVMTTQPQHGAHIVLRKLGSGATNFSLLKQAGDVEENPG

SEQ ID NO: 36 - protein, 519 aa, synthetic construct
PMSSSVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHMAKLKDMIPAFFQSCNEIVSEWERMIPKEGSFELDVMPYMQNLAADAISRTAFGSSYKEGQMIFQLLKELTDLVVKAAFGIYIPGWRFLPTKSNNKMKEINRKIKTLLLGIIDKRQKAMKEGEVVQNDLLGILVESNLNEIRGHENNKDAGMSIEDVIEECKVFYIGGQETTAGLLIWTMILLSFHTEWQERARTEVLEVVGNKKPDFDGLNRLKIVTMILYEVLRLYPPASMLTRIVQKETRIGKLTLPAGVTLIIPTVLIHRDHELWGEDANEFKPERFSEGVSKAAKVQPAFFPFGWGPRLCLGQNFAMIEAKMAFSLILQRFSFHLSSSYAHAPTVVMTTQPQHGAHIVLRKL

SEQ ID NO: 37 - protein, 1058 aa, synthetic construct
MSSSVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHMAKLKDMIPAFFQSCNEIVSEWERMIPKEGSFELDVMPYMQNLAADAISRTAFGSSYKEGQMIFQLLKELTDLVVKAAFGIYIPGWRFLPTKSNNKMKEINRKIKTLLLGIIDKRQKAMKEGEVVQNDLLGILVESNLNEIRGHENNKDAGMSIEDVIEECKVFYIGGQETTAGLLIWTMILLSFHTEWQERARTEVLEVVGNKKPDFDGLNRLKIVTMILYEVLRLYPPASMLTRIVQKETRIGKLTLPAGVTLIIPTVLIHRDHELWGEDANEFKPERFSEGVSKAAKVQPAFFPFGWGPRLCLGQNFAMIEAKMAFSLILQRFSFHLSSSYAHAPTVVMTTQPQHGAHIVLRKLGSGATNFSLLKQAGDVEENPGPMSSSVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHMAKLKDMIPAFFQSCNEIVSEWERMIPKEGSFELDVMPYMQNLAADAISRTAFGSSYKEGQMIFQLLKELTDLVVKAAFGIYIPGWRFLPTKSNNKMKEINRKIKTLLLGIIDKRQKAMKEGEVVQNDLLGILVESNLNEIRGHENNKDAGMSIEDVIEECKVFYIGGQETTAGLLIWTMILLSFHTEWQERARTEVLEVVGNKKPDFDGLNRLKIVTMILYEVLRLYPPASMLTRIVQKETRIGKLTLPAGVTLIIPTVLIHRDHELWGEDANEFKPERFSEGVSKAAKVQPAFFPFGWGPRLCLGQNFAMIEAKMAFSLILQRFSFHLSSSYAHAPTVVMTTQPQHGAHIVLRKL

SEQ ID NO: 38 - DNA, 2913 bp, synthetic construct
  source 1..2913: mol_type = other DNA
atggtgcagccccgggtgctgctgttccctttccccgcgctgggccacgtgaagcccttcctgtcgctggcggagctcctgtccgacgccggcatcgacgtggtcttcctctcgacggagtataaccaccgccgcatctccaacaccgaggccctggcctcccgcttccccaccctgcacttcgaaacgataccggacggcctgccccccaacgagtcgcgcgccctcgcggacggccccctgtacttcagcatgagggagggcaccaagccgcgcttccggcagctgatccagagccttaacgacggccgttggcccatcacctgtatcatcacggacatcatgctcagttcgcccatcgaggtggcggaggagttcgggatcccggtgatcgcgttctgcccgtgctccgctcgctacctctcgatccacttcttcatcccgaagctggtggaggaggggcagatcccgtacgcggacgacgaccccattggggagatccagggggtgcccctcttcgaggggcttctccgacggaaccacctcccgggatcttggtccgacaagtccgcggacatttccttctcgcacggcctcatcaaccagacgctcgcggccgggcgggcctcggccctgatcctcaacaccttcgacgagctcgaggcccctttcctcacccacctgagctccatcttcaacaagatctacaccatcggccccctccacgccctgtccaagagccggctcggggactcctcctcgtcggccagcgcgctctcggggttctggaaggaggaccgcgcgtgcatgtcctggctcgactgccagccgccgcgctcggtcgtgttcgtctccttcgggtcgaccatgaagatgaaggcggacgagcttcgggagttctggtacgggctggtgagcagcgggaagccgttcctctgcgtgttgcggtccgacgtcgtcagcggcggcgaggcggcggagctcatcgagcagatggcggaggaggagggggctgggggcaagctgggcatggtcgtcgagtgggcggcgcaggagaaggtcctctcccaccctgccgtggggggcttcctgacgcactgcggctggaactcgacggtggagagcatcgccgccggcgtccccatgatgtgctggccgatcctcggggaccagccctccaacgccacttggatcgaccgggtctggaagatcggggtcgagcgaaacaaccgggagtgggaccggttgacggtggagaagatggtccgcgcattgatggagggccagaagcgggtggagatccagcggtcgatggagaagctgtcgaagctggcgaacgagaaggtcgtccggggggggctgtctttcgacaacctcgaggtcctcgtggaggacatcaagaagctgaagccgtacaagttcggaagcggagctactaacttcagcctgctgaagcaggctggagacgtggaggagaaccctggacctatggtccaaccccgggtcctgctctttccgtttccggctcttggccatgttaaaccattcctttcccttgccgagctcctgtcggacgcaggaatagatgtcgtctttttgtcaacggagtataatcatcggaggatctcaaatacagaggcacttgcaagccggtttcccactcttcacttcgaaaccattccagatgggctgccgccaaatgagagcagggctttggccgacggccctctttacttctcgatgagggaggggacaaaaccgaggtttcgccagcttatacagagcctgaatgatggtcggtggcccattacttgtataattacggatataatgctttccagccccatcgaagttgcggaggagtttgggataccggtgatagcattttgtccttgctcggctcgctacttgtctatacacttctttatcccgaagctcgtcgaagaaggccagataccatacgcggatgacgaccccattggagaaatacaaggagtgcctctttttgaggggctcctgcgcagaaatcatctgcctggttcttggagcgataaatccgctgacatctcattcagccatgggctgataaatcagacgcttgcggccggtcgggcatcggcactgattcttaacacctttgatgaacttgaggccccatttcttactcacctctcgtcgatttttaacaagatttacacaatcgggccacttcatgctctttccaaatcacggcttggagattcgagcagcagcgcatccgctctgtcgggattctggaaagaggacagagcgtgcatgtcttggcttgattgccaaccaccccggtcggttgtttttgtttcctttgggtcgacaatgaagatgaaggcggatgagttgcgggagttttggtacgggcttgtctcttccggaaagcccttcctctgtgtgctgagatcagatgttgtctcgggcggtgaggcagccgagctcatagagcaaatggcggaagaggagggggccgggggtaaactcggtatggtggtcgagtgggcggctcaggagaaagtgttgtcacatcctgctgtcgggggattcctgacacattgcggatggaattctactgtcgagtcaattgcggcgggcgtgcctatgatgtgctggcccatactgggcgaccagccctctaatgcgacttggatagaccgggtctggaaaattggcgttgaacggaacaaccgcgaatgggaccgcctgacagttgagaagatggttagagcactcatggaagggcaaaagagagtcgaaattcaacgctctatggaaaaattgagcaaacttgcaaatgaaaaggtcgtgagaggtgggctgtcgtttgataatctggaggttctggttgaggacataaaaaagcttaagccctacaaattttga

SEQ ID NO: 39 - protein, 495 aa, synthetic construct
MVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKFGSGATNFSLLKQAGDVEENPG

SEQ ID NO: 40 - protein, 475 aa, synthetic construct
PMVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKF

SEQ ID NO: 41 - protein, 970 aa, synthetic construct
MVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKFGSGATNFSLLKQAGDVEENPGPMVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKF

SEQ ID NO: 42 - DNA, 2787 bp, synthetic construct
  source 1..2787: mol_type = other DNA
atggacgcgcagcggggccacacgacgacgatcctgatgttcccctggctcggctacgggcacctcagcgccttcctcgagctggcgaagagcctgtcccggcgcaacttccacatctacttctgctcgaccagcgtcaacctggacgcgatcaagcccaagctgccctccagcagctcctccgacagcatccagctggtggagctgtgcctgccctccagccccgaccagctccccccgcacctccacaccaccaacgcgctcccgccccacctcatgcccaccctccaccaggccttcagcatggcggcgcagcacttcgcggccatcctgcacaccctcgcgccccacctcctgatctacgactcgttccagccgtgggcaccccagctggcctcctcgctcaacatcccggccatcaacttcaacacgaccggggcgtccgtcctgacccggatgctgcacgcgacccactacccgagcagcaagttccccatcagcgagttcgtgctccacgactactggaaggccatgtacagcgcggccgggggcgcggtgacgaagaaggaccacaagatcggcgagacgctggcgaactgcctccacgccagctgctcggtcatcctgatcaactccttccgcgagctggaggagaagtacatggactacctgtccgtgctcctgaacaagaaggtggtgccggtggggccgctggtgtacgagccgaaccaggatggagaggacgaggggtactccagcataaagaactggctcgacaagaaggagccgagctccacggtgttcgtgtccttcggcagcgagtacttcccgagcaaggaggagatggaggagatcgcccacgggctcgaggcctctgaagttcatttcatctgggttgtgcggttcccgcagggggacaacacctccgcgatcgaggacgccctgccgaagggcttcctcgagagggtgggcgagaggggcatggtggtgaagggctgggccccgcaggcgaagatcctgaagcactggtccaccgggggcttcgtgtcccactgcggctggaacagcgtgatggagagcatgatgttcggggtgccgatcatcggcgtgccgatgcacctggaccagccgttcaacgccggcctggccgaggaggccggcgtgggggtggaggcgaagcgcgaccccgacgggaagatccagcgcgacgaggtcgccaagctgatcaaggaggtggtggtggagaagacccgggaggacgtgaggaagaaggcgcgcgagatgtccgagatcctccgcagcaagggggaggagaagatggacgagatggtggccgcgatctccctcttcctcaagatcggaagcggagctactaacttcagcctgctgaagcaggctggagacgtggaggagaaccctggacctatggatgcccagcgcggtcatacgactacgatcctcatgtttccatggctgggttacggacacttgagcgcgtttctggaactcgccaagtcgctgtcaaggagaaactttcatatatatttttgctcgacatctgtcaacctggacgccatcaaaccaaaactgccttcttcgtccagctcggacagcattcagctggttgaactttgtctgccatcatctccggaccaactccctccccatcttcacacaaccaacgctttgccgccgcacctgatgcccacactccaccaagcgttctcaatggcggcgcagcatttcgcagctatactgcacacccttgctccgcacctcctgatttacgattcatttcaaccgtgggccccccaactggcgtcgagcttgaatatccccgcaatcaattttaacaccaccggagcatccgtgcttaccagaatgttgcacgctacacattatccgtcctccaagtttcctatatccgaatttgtcctgcatgactattggaaagctatgtactcagccgctggaggagctgtgacaaaaaaggaccataagataggagagacactcgcgaactgcttgcacgcttcttgttcagtcattctcatcaatagcttcagagagctcgaggagaagtatatggactacctctcagttcttcttaataaaaaggtggtcccggtgggaccgctcgtctatgaaccaaaccaggacggtgaagacgaaggatattccagcatcaagaactggctggataaaaaggaaccttcatcgactgttttcgtttccttcggatcggagtatttcccttctaaggaagaaatggaagaaatcgcccacgggcttgaagcgtcggaagtccattttatttgggtggtcagatttccgcaaggagataacacaagcgcaatagaggacgcattgccaaagggattccttgaacgcgtcggggaaagggggatggtcgtgaaaggatgggcgccgcaggcgaagatccttaagcactggtcaacaggcggatttgtcagccattgtgggtggaattctgttatggaatcaatgatgttcggggtgcccataataggagtcccgatgcatttggatcagccctttaacgctgggcttgcagaggaggccggtgtgggtgtcgaggctaagcgcgaccccgatggtaaaatccagagggatgaagtggcaaaacttataaaagaagtggtcgtggagaaaactcgcgaggacgtgcggaagaaggcgcgggaaatgtcagagatattgaggagcaagggggaagagaaaatggacgagatggttgcagccatctccctgttcttgaagatctga

SEQ ID NO: 43 - protein, 474 aa, synthetic construct
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKIGSGATNFSLLKQAGDVEENPG

SEQ ID NO: 44 - protein, 454 aa, synthetic construct
PMDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKI

SEQ ID NO: 45 - protein, 928 aa, synthetic construct
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKIGSGATNFSLLKQAGDVEENPGPMDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKI

SEQ ID NO: 46 - DNA, 2613 bp, synthetic construct
  source 1..2613: mol_type = other DNA
atgaacacgaccccgctgccggaggaggacgaggaggtggtgaagatggtggtggacgggtccgtgccgtcctactcgctcgagtccaagctcggcgacccgaagcgcgcggcgtccatccggcgggaggcgctgcagcgcatgacggggcggagcatccacgggctgccgttcgagggcttcgactacgagagcatcctgggccagtgctgcgagatgccggtggggtacgtccagatcccggtgggcatcgcggggccgctgctgctcgacgggttcgagtacaccgtgccgatggccaccacggagggctgcctggtggcctccaccaacaggggctgcaaggccatctacgcctccggcggggcgacctcaatgctcctcaaagacggcatgacgagggccccggtggtcaggttcggcagcgcgaagagggcgagcgagctgaagttcttcctggaggacccgaacaacttcgacaccctggccgtggtgttcaaccgctccagccgcttcgcccgcctgcagagcatcaggtgcagcatcgcgggcaagaacctgtacgtccgcttctgctgctccaccggcgacgccatgggcatgaacatggtgtccaagggcgtgcagaacgtgctggagttcctgcagcacgacttctccgacatggaggtgatcggcatctccggcaacttctgcgccgacaagaagccggccgccgtgaactggatcgagggcaggggcaagtccgtggtgtgcgaggccattatcaaggacgaggtggtgcgcaaggtgctcaagaccagcgtggcgagcctggtggagctgaacatgctgaagaacctgaccggctccgccatggccggggccctcggcggcttcaacgcgcacagcagcaacatcgtgtccgccatcttcctggccaccggccaggacccggcccagaacgtggagtcctcccactgcatcacgatgatggagccggtcaacaacggcaaggacctccacatctccgtgaccatgccgtccatcgaggtcgggaccgtggggggcggcacccagctggcgtcccagagcgcgtgcctgaacctgctgggggtcaagggcgcgtccaaggagagcccgggggccaacagcaggctgctggcgaccatcgtggccggctccgtgctggccggcgagctctccctgatgtccgccatcgcggcgggccagctggtgaggtcccacatgaagtacaaccgctcctccagggatgtgtccaaggtggagagcggaagcggagctactaacttcagcctgctgaagcaggctggagacgtggaggagaaccctggacctatgaacacgacccctttgccggaggaggatgaagaagttgtgaaaatggtggttgatggcagcgtgccatcgtactcgctcgagtcaaagctcggcgacccaaaacgggcagcctcaatccggcgggaggcattgcagcggatgacaggtcggagcatccacgggctgccttttgaaggctttgactatgaatctatacttgggcaatgctgcgaaatgcccgtcggttatgtccagattccggttggcatcgctggacctctgttgctcgatgggtttgaatatacggtgcccatggcaactacggagggttgccttgtggcttctactaatcgcggttgcaaagctatctacgcctcaggaggggcgacctcaatgctcctcaaagacggtatgacgagggcacctgttgtcagattcggtagcgcgaagagagcgtcagagttgaaattctttcttgaagacccgaataatttcgacaccttggctgtcgtgttcaatcggtcttcgagatttgcgagattgcaatcgatcaggtgcagcatcgcgggcaaaaatctctatgttagattctgttgctcaacaggagatgcaatgggtatgaacatggtttccaaaggggtccaaaacgtgcttgaatttctgcaacacgattttagcgatatggaagttataggcatttcaggcaatttctgcgccgataagaagcccgctgccgttaattggatcgagggtcgcggaaaatctgttgtctgtgaggcaattatcaaggatgaggttgtgcgcaaagttctcaagactagcgtcgcaagcttggtcgaattgaacatgctgaagaacctgactggctccgcgatggcaggggccttgggtggtttcaacgctcacagcagcaacatagtttctgccatatttttggcgaccggacaggacccggcacaaaatgttgagtcttctcactgcatcacgatgatggaaccagtcaacaatggaaaagacctccatatttctgtcacgatgccttcaatcgaagtcgggacagttggtggaggaacacaattggcttcacagagcgcgtgccttaatttgctgggtgtcaagggtgcctccaaggaatcgcctggggcgaattctaggcttttggcgaccatcgttgcaggaagcgttttggcgggggagctctcgctgatgtccgccatcgcggcgggtcagttggtgagatcgcacatgaaatataacagaagctccagagatgtgtcaaaagttgaaagctaa

SEQ ID NO: 47 - protein, 445 aa, synthetic construct
MNTTPLPEEDEEVVKMVVDGSVPSYSLESKLGDPKRAASIRREALQRMTGRSIHGLPFEGFDYESILGQCCEMPVGYVQIPVGIAGPLLLDGFEYTVPMATTEGCLVASTNRGCKAIYASGGATSMLLKDGMTRAPVVRFGSAKRASELKFFLEDPNNFDTLAVVFNRSSRFARLQSIRCSIAGKNLYVRFCCSTGDAMGMNMVSKGVQNVLEFLQHDFSDMEVIGISGNFCADKKPAAVNWIEGRGKSVVCEAIIKDEVVRKVLKTSVASLVELNMLKNLTGSAMAGALGGFNAHSSNIVSAIFLATGQDPAQNVESSHCITMMEPVNNGKDLHISVTMPSIEVGTVGGGTQLASQSACLNLLGVKGASKESPGANSRLLATIVAGSVLAGELSLMSAIAAGQLVRSHMKYNRSSRDVSKVESGSGATNFSLLKQAGDVEENPG

SEQ ID NO: 48 - protein, 425 aa, synthetic construct
PMNTTPLPEEDEEVVKMVVDGSVPSYSLESKLGDPKRAASIRREALQRMTGRSIHGLPFEGFDYESILGQCCEMPVGYVQIPVGIAGPLLLDGFEYTVPMATTEGCLVASTNRGCKAIYASGGATSMLLKDGMTRAPVVRFGSAKRASELKFFLEDPNNFDTLAVVFNRSSRFARLQSIRCSIAGKNLYVRFCCSTGDAMGMNMVSKGVQNVLEFLQHDFSDMEVIGISGNFCADKKPAAVNWIEGRGKSVVCEAIIKDEVVRKVLKTSVASLVELNMLKNLTGSAMAGALGGFNAHSSNIVSAIFLATGQDPAQNVESSHCITMMEPVNNGKDLHISVTMPSIEVGTVGGGTQLASQSACLNLLGVKGASKESPGANSRLLATIVAGSVLAGELSLMSAIAAGQLVRSHMKYNRSSRDVSKVES

SEQ ID NO: 49 - protein, 870 aa, synthetic construct
MNTTPLPEEDEEVVKMVVDGSVPSYSLESKLGDPKRAASIRREALQRMTGRSIHGLPFEGFDYESILGQCCEMPVGYVQIPVGIAGPLLLDGFEYTVPMATTEGCLVASTNRGCKAIYASGGATSMLLKDGMTRAPVVRFGSAKRASELKFFLEDPNNFDTLAVVFNRSSRFARLQSIRCSIAGKNLYVRFCCSTGDAMGMNMVSKGVQNVLEFLQHDFSDMEVIGISGNFCADKKPAAVNWIEGRGKSVVCEAIIKDEVVRKVLKTSVASLVELNMLKNLTGSAMAGALGGFNAHSSNIVSAIFLATGQDPAQNVESSHCITMMEPVNNGKDLHISVTMPSIEVGTVGGGTQLASQSACLNLLGVKGASKESPGANSRLLATIVAGSVLAGELSLMSAIAAGQLVRSHMKYNRSSRDVSKVESGSGATNFSLLKQAGDVEENPGPMNTTPLPEEDEEVVKMVVDGSVPSYSLESKLGDPKRAASIRREALQRMTGRSIHGLPFEGFDYESILGQCCEMPVGYVQIPVGIAGPLLLDGFEYTVPMATTEGCLVASTNRGCKAIYASGGATSMLLKDGMTRAPVVRFGSAKRASELKFFLEDPNNFDTLAVVFNRSSRFARLQSIRCSIAGKNLYVRFCCSTGDAMGMNMVSKGVQNVLEFLQHDFSDMEVIGISGNFCADKKPAAVNWIEGRGKSVVCEAIIKDEVVRKVLKTSVASLVELNMLKNLTGSAMAGALGGFNAHSSNIVSAIFLATGQDPAQNVESSHCITMMEPVNNGKDLHISVTMPSIEVGTVGGGTQLASQSACLNLLGVKGASKESPGANSRLLATIVAGSVLAGELSLMSAIAAGQLVRSHMKYNRSSRDVSKVES

SEQ ID NO: 50 - DNA, 3045 bp, synthetic construct
  source 1..3045: mol_type = other DNA
atggtgcagccccgggtgctgctgttccctttccccgcgctgggccacgtgaagcccttcctgtcgctggcggagctcctgtccgacgccggcatcgacgtggtcttcctctcgacggagtataaccaccgccgcatctccaacaccgaggccctggcctcccgcttccccaccctgcacttcgaaacgataccggacggcctgccccccaacgagtcgcgcgccctcgcggacggccccctgtacttcagcatgagggagggcaccaagccgcgcttccggcagctgatccagagccttaacgacggccgttggcccatcacctgtatcatcacggacatcatgctcagttcgcccatcgaggtggcggaggagttcgggatcccggtgatcgcgttctgcccgtgctccgctcgctacctctcgatccacttcttcatcccgaagctggtggaggaggggcagatcccgtacgcggacgacgaccccattggggagatccagggggtgcccctcttcgaggggcttctccgacggaaccacctcccgggatcttggtccgacaagtccgcggacatttccttctcgcacggcctcatcaaccagacgctcgcggccgggcgggcctcggccctgatcctcaacaccttcgacgagctcgaggcccctttcctcacccacctgagctccatcttcaacaagatctacaccatcggccccctccacgccctgtccaagagccggctcggggactcctcctcgtcggccagcgcgctctcggggttctggaaggaggaccgcgcgtgcatgtcctggctcgactgccagccgccgcgctcggtcgtgttcgtctccttcgggtcgaccatgaagatgaaggcggacgagcttcgggagttctggtacgggctggtgagcagcgggaagccgttcctctgcgtgttgcggtccgacgtcgtcagcggcggcgaggcggcggagctcatcgagcagatggcggaggaggagggggctgggggcaagctgggcatggtcgtcgagtgggcggcgcaggagaaggtcctctcccaccctgccgtggggggcttcctgacgcactgcggctggaactcgacggtggagagcatcgccgccggcgtccccatgatgtgctggccgatcctcggggaccagccctccaacgccacttggatcgaccgggtctggaagatcggggtcgagcgaaacaaccgggagtgggaccggttgacggtggagaagatggtccgcgcattgatggagggccagaagcgggtggagatccagcggtcgatggagaagctgtcgaagctggcgaacgagaaggtcgtccggggggggctgtctttcgacaacctcgaggtcctcgtggaggacatcaagaagctgaagccgtacaagttcggaagcggagctactaacttcagcctgctgaagcaggctggagacgtggaggagaaccctggacctatgtccagctcagtcgcggctaccataagcatatggatggtcgttgtctgtatagtgggtgtcgggtggcgggtggtcaactgggtttggcttcgccccaaaaaacttgagaagcggctccgggaacaaggtctggctggaaattcgtatcggctccttttcggggaccttaaagaaagagccgcgatggctgaacaggcaaattcaaagccaattaacttttcgcatgatatagggccacgggtttttccgtcgatgtataaaactattcagaactatggtaagaactcttacatgtggttgggaccttatccgagggttcatatcatggatcctcagcagctgaagactgtgtttacgcttgtgtacgacatacagaagcccaatcttaatcccttgattaaatttttgttggatggaattgttacgcatgaaggggaaaagtgggctaaacaccgcaaaatcattaaccccgcattccatatggcaaagttgaaagatatgattccagcttttttccaatcctgtaatgaaattgtcagcgaatgggagaggatgatcccgaaggaggggagcttcgaattggatgtgatgccctatatgcagaatctggccgctgacgctattagccggactgccttcggaagcagctataaggaaggtcaaatgatttttcagttgctcaaagagttgaccgatcttgtggtgaaagcagccttcggcatttatatccccggatggagattcctccccacgaaaagcaacaataaaatgaaggagataaaccggaagattaaaactcttttgcttggtattatagataagaggcagaaggcaatgaaagaaggcgaggtcgtccagaatgaccttcttgggattttggtcgagtcgaaccttaacgagataagagggcatgagaataataaagacgcggggatgtccatcgaagatgtgatagaagagtgtaaggtgttctacataggtggccaggagacaactgcaggcctgttgatatggaccatgatattgctcagcttccatacggaatggcaggaacgcgctagaaccgaagttctcgaagtcgttgggaacaaaaaacctgatttcgacggattgaatcggctgaagatagttacaatgattctctatgaggtgctccggttgtatcctcccgcctctatgttgacccgcattgtccaaaaggaaacgagaatagggaagcttactcttccggctggagtcacactgataatcccaacggtgttgatacaccgcgatcacgaattgtggggcgaggacgcgaatgaattcaagcccgaacgcttcagcgagggagttagcaaggccgctaaggtgcagcctgcgttcttcccgttcggatgggggcccagactttgtctcgggcaaaacttcgcaatgattgaggccaaaatggcgttttctctgattctgcaaagattctctttccacctctcatcatcatatgcacacgcgcctaccgtcgttatgacaactcagcctcagcatggcgctcatatagttctcagaaagctctaa

SEQ ID NO: 51 - protein, 495 aa, synthetic construct; identical to SEQ ID NO: 39

SEQ ID NO: 52 - protein, 519 aa, synthetic construct; identical to SEQ ID NO: 36

SEQ ID NO: 53 - protein, 1014 aa, synthetic construct
MVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGGLSFDNLEVLVEDIKKLKPYKFGSGATNFSLLKQAGDVEENPGPMSSSVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLIKFLLDGIVTHEGEKWAKHRKIINPAFHMAKLKDMIPAFFQSCNEIVSEWERMIPKEGSFELDVMPYMQNLAADAISRTAFGSSYKEGQMIFQLLKELTDLVVKAAFGIYIPGWRFLPTKSNNKMKEINRKIKTLLLGIIDKRQKAMKEGEVVQNDLLGILVESNLNEIRGHENNKDAGMSIEDVIEECKVFYIGGQETTAGLLIWTMILLSFHTEWQERARTEVLEVVGNKKPDFDGLNRLKIVTMILYEVLRLYPPASMLTRIVQKETRIGKLTLPAGVTLIIPTVLIHRDHELWGEDANEFKPERFSEGVSKAAKVQPAFFPFGWGPRLCLGQNFAMIEAKMAFSLILQRFSFHLSSSYAHAPTVVMTTQPQHGAHIVLRKL

SEQ ID NO: 54 - DNA, 2700 bp, synthetic construct
  source 1..2700: mol_type = other DNA
atggacgcgcagcggggccacacgacgacgatcctgatgttcccctggctcggctacgggcacctcagcgccttcctcgagctggcgaagagcctgtcccggcgcaacttccacatctacttctgctcgaccagcgtcaacctggacgcgatcaagcccaagctgccctccagcagctcctccgacagcatccagctggtggagctgtgcctgccctccagccccgaccagctccccccgcacctccacaccaccaacgcgctcccgccccacctcatgcccaccctccaccaggccttcagcatggcggcgcagcacttcgcggccatcctgcacaccctcgcgccccacctcctgatctacgactcgttccagccgtgggcaccccagctggcctcctcgctcaacatcccggccatcaacttcaacacgaccggggcgtccgtcctgacccggatgctgcacgcgacccactacccgagcagcaagttccccatcagcgagttcgtgctccacgactactggaaggccatgtacagcgcggccgggggcgcggtgacgaagaaggaccacaagatcggcgagacgctggcgaactgcctccacgccagctgctcggtcatcctgatcaactccttccgcgagctggaggagaagtacatggactacctgtccgtgctcctgaacaagaaggtggtgccggtggggccgctggtgtacgagccgaaccaggatggagaggacgaggggtactccagcataaagaactggctcgacaagaaggagccgagctccacggtgttcgtgtccttcggcagcgagtacttcccgagcaaggaggagatggaggagatcgcccacgggctcgaggcctctgaagttcatttcatctgggttgtgcggttcccgcagggggacaacacctccgcgatcgaggacgccctgccgaagggcttcctcgagagggtgggcgagaggggcatggtggtgaagggctgggccccgcaggcgaagatcctgaagcactggtccaccgggggcttcgtgtcccactgcggctggaacagcgtgatggagagcatgatgttcggggtgccgatcatcggcgtgccgatgcacctggaccagccgttcaacgccggcctggccgaggaggccggcgtgggggtggaggcgaagcgcgaccccgacgggaagatccagcgcgacgaggtcgccaagctgatcaaggaggtggtggtggagaagacccgggaggacgtgaggaagaaggcgcgcgagatgtccgagatcctccgcagcaagggggaggagaagatggacgagatggtggccgcgatctccctcttcctcaagatcggaagcggagctactaacttcagcctgctgaagcaggctggagacgtggaggagaaccctggacctatgaacacgacccctttgccggaggaggatgaagaagttgtgaaaatggtggttgatggcagcgtgccatcgtactcgctcgagtcaaagctcggcgacccaaaacgggcagcctcaatccggcgggaggcattgcagcggatgacaggtcggagcatccacgggctgccttttgaaggctttgactatgaatctatacttgggcaatgctgcgaaatgcccgtcggttatgtccagattccggttggcatcgctggacctctgttgctcgatgggtttgaatatacggtgcccatggcaactacggagggttgccttgtggcttctactaatcgcggttgcaaagctatctacgcctcaggaggggcgacctcaatgctcctcaaagacggtatgacgagggcacctgttgtcagattcggtagcgcgaagagagcgtcagagttgaaattctttcttgaagacccgaataatttcgacaccttggctgtcgtgttcaatcggtcttcgagatttgcgagattgcaatcgatcaggtgcagcatcgcgggcaaaaatctctatgttagattctgttgctcaacaggagatgcaatgggtatgaacatggtttccaaaggggtccaaaacgtgcttgaatttctgcaacacgattttagcgatatggaagttataggcatttcaggcaatttctgcgccgataagaagcccgctgccgttaattggatcgagggtcgcggaaaatctgttgtctgtgaggcaattatcaaggatgaggttgtgcgcaaagttctcaagactagcgtcgcaagcttggtcgaattgaacatgctgaagaacctgactggctccgcgatggcaggggccttgggtggtttcaacgctcacagcagcaacatagtttctgccatatttttggcgaccggacaggacccggcacaaaatgttgagtcttctcactgcatcacgatgatggaaccagtcaacaatggaaaagacctccatatttctgtcacgatgccttcaatcgaagtcgggacagttggtggaggaacacaattggcttcacagagcgcgtgccttaatttgctgggtgtcaagggtgcctccaaggaatcgcctggggcgaattctaggcttttggcgaccatcgttgcaggaagcgttttggcgggggagctctcgctgatgtccgccatcgcggcgggtcagttggtgagatcgcacatgaaatataacagaagctccagagatgtgtcaaaagttgaaagctaa

SEQ ID NO: 55 - protein, 474 aa, synthetic construct; identical to SEQ ID NO: 43

SEQ ID NO: 56 - protein, 425 aa, synthetic construct; identical to SEQ ID NO: 48

SEQ ID NO: 57 - protein, 899 aa, synthetic construct
MDAQRGHTTTILMFPWLGYGHLSAFLELAKSLSRRNFHIYFCSTSVNLDAIKPKLPSSSSSDSIQLVELCLPSSPDQLPPHLHTTNALPPHLMPTLHQAFSMAAQHFAAILHTLAPHLLIYDSFQPWAPQLASSLNIPAINFNTTGASVLTRMLHATHYPSSKFPISEFVLHDYWKAMYSAAGGAVTKKDHKIGETLANCLHASCSVILINSFRELEEKYMDYLSVLLNKKVVPVGPLVYEPNQDGEDEGYSSIKNWLDKKEPSSTVFVSFGSEYFPSKEEMEEIAHGLEASEVHFIWVVRFPQGDNTSAIEDALPKGFLERVGERGMVVKGWAPQAKILKHWSTGGFVSHCGWNSVMESMMFGVPIIGVPMHLDQPFNAGLAEEAGVGVEAKRDPDGKIQRDEVAKLIKEVVVEKTREDVRKKAREMSEILRSKGEEKMDEMVAAISLFLKIGSGATNFSLLKQAGDVEENPGPMNTTPLPEEDEEVVKMVVDGSVPSYSLESKLGDPKRAASIRREALQRMTGRSIHGLPFEGFDYESILGQCCEMPVGYVQIPVGIAGPLLLDGFEYTVPMATTEGCLVASTNRGCKAIYASGGATSMLLKDGMTRAPVVRFGSAKRASELKFFLEDPNNFDTLAVVFNRSSRFARLQSIRCSIAGKNLYVRFCCSTGDAMGMNMVSKGVQNVLEFLQHDFSDMEVIGISGNFCADKKPAAVNWIEGRGKSVVCEAIIKDEVVRKVLKTSVASLVELNMLKNLTGSAMAGALGGFNAHSSNIVSAIFLATGQDPAQNVESSHCITMMEPVNNGKDLHISVTMPSIEVGTVGGGTQLASQSACLNLLGVKGASKESPGANSRLLATIVAGSVLAGELSLMSAIAAGQLVRSHMKYNRSSRDVSKVES

SEQ ID NO: 58 - DNA, 1029 bp, synthetic construct
  source 1..1029: mol_type = other DNA
atgtccaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgttcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatctgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 59 - protein, 342 aa, Escherichia coli
MSKKPELTATSVEKFLIEKFDSVSDLMQLSEGEESRAFSFDVGGRGYVLRVNSCADGFYKDRYVYRHFASAALPIPEVLDIGEFSESLTYCISRRSQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKK

SEQ ID NO: 60 - DNA, 500 bp, Horseradish latent virus
  source 1..500: mol_type = genomic DNA
tggtagactatgaaacactagtctactcaaagaacttgaagaagacgactcaggaagacaggagcgtcatcaacaagtttcagcaaaagctgattagtggaaaaatccttggattccactctccagcaatctgccagcacataaaggtgacagcagaaaaagaagattgtgactaccactgcaatcagtgcgaatcttcaaaaggaaaggctatcgtttgcgataagcctgccgacagtggtccagccgacaatggagggccccagacacgtgaagacgcgtctgccgacagtgggtctaggacaacagacaccacgcactcagcaagtggatgaaataattcatctgctgacgtaagggatgacgatcaatcccactatcccaagacccttcacttctatataagtgaagttgcttcatttggagaaggcatctcgaaatctcaacacaactcgagctctcccttctctcttctttatctctctaaatgtgtgagtaga

SEQ ID NO: 61 - DNA, 439 bp, Dahlia mosaic virus
  source 1..439: mol_type = genomic DNA
tcgacgggaagacaaaaatcaagatcaattacgcacaggaaaaagtattgatcatgagtcactacggagcaccaatctccagacaagatgtcgacatcttatcttcctttgaagaaagcattgcacatgccaaagttgatgtagggacatctacaaaattattgctttgcaaacagctgaagaaattattggccgacaaccataaatgctcagcatgtgaaagtggggcctccaccagcaataatacaagctttacagcttgcagtgactcatcctccaataatggagaaaaagacgtcagcagtgacgaacaagggtcgaaagacttgcctatataagagcattctcccctcagttgaagatcatcgaaagttggagcaataaactctctcttcaacaaatctatcttttgtctagaatcatgtgtgagtagtaaccc

SEQ ID NO: 62 - DNA, 585 bp, synthetic construct
  source 1..585: mol_type = other DNA
gtaatcttgtcaacatcgagcagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccagagtttttacttcggacagtcaaaaatgagtttaacttctcagccgaggtaaaacaagaaatatgcttacgtctacagagggatttctctgaagatcatgtttgccagctatgcgaacaatcatcgggagatcctgagccaatcaaagaggagtgatgtagacctaaagcaataatggagccatgacgtaagggcttacgccattacgaaataattaaaggctgatgtgacctgtcggtctatcagaacctttactttttatatttggcgtgtatttttaaatttccacggcaatgacgatgtgacctgtgcatccgctttgcctataaataagttttagtttgtattgatcgacacgatcgagaagacacggc

SEQ ID NO: 63 - DNA, 621 bp, synthetic construct
  source 1..621: mol_type = other DNA
tcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaggatcttcgtccacagacatcaacatcttatcgtcctttgaagataagataataatgttgaagataagagtgggagccaccactaaaacattgctttgtcaaaagctaaaaaagatgatgcccgacagccacttgtgtgaagcatgtgaagccggtccctccactaagaaaattagtgaagcatcttccagtggtccctccactcacagctcaatcagtgagcaacaggacgaaggaaatgacgtaagccatgacgtctaggatcccacaagaatttccttatataaggaacacaaatcagaaggaagagatcaatcgaaatcaaaatcggaatcgaaatcaaaatcggaatcgaaatctctcatct

SEQ ID NO: 64 - DNA, 465 bp, Cestrum yellow leaf curling virus
  source 1..465: mol_type = genomic DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 65 - DNA, 742 bp, Cauliflower mosaic virus
  source 1..742: mol_type = genomic DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctacaaatctatctctctcgagctttcgcagatctgtcgatcgaccaac

SEQ ID NO: 66 - DNA, 299 bp, Agrobacterium tumefaciens
  source 1..299: mol_type = genomic DNA
gatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgca

SEQ ID NO: 67 - DNA, 1369 bp, Sugarcane bacilliform badnavirus
  source 1..1369: mol_type = genomic DNA
atccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcaccacatgatcatttgagcaagtttgcttgaataaaagaactatcattccgcatacctgatcctatagtcctagcttgagaacaagagcgaagtctatagttgagatcctaagagaaactcgaggtttttcgggtttcctgggcgcgttccctggtatcagagcgaggtatgatttctgtatccgctatgttctaaatttcttagataaggggccaagggctctgctgatgagtttaaggacaactacttgtgcaagttacatagcatgatacgtcgaaaggctgaaaatatccaaaatactgtctattgtttggaaaactaggttgttctaggggagaacgttaatgaggggtaagcttagttcattctgaaaatcaaggtctgtgattgtagttgagctcagtaattaagtgctgaaggaagtaagatctaggtaggacaaaagtacccgtcaaggcaggaggccgctaaggggaaaagagccagacgatcaaagctttttcagcacggttgttgagtttagctatcaagaaatagccttgagactaagattcatcactaagaactgcctactcaagcctccctgaatccgcctattagtacaaacgagacaacagtataaggagaaactatatgcctgtaagacttaagtgcaaaagtaacctctggaactgggtagaagtctag

SEQ ID NO: 68 - DNA, 514 bp, Cassava vein mosaic virus
  source 1..514: mol_type = genomic DNA
cagaaggtaattatccaagatgtagcatcaagaatccaatgtttacgggaaaaactatggaagtattatgtgaactcagcaagaagcagatcaatatgcggcacatattcaacctatgttcaaaaatgaagaatgtacagatacaagatcctatactgccagaatacgaagaagaatacatagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgtaagtttg

SEQ ID NO: 69 - DNA, 400 bp, Figwort mosaic virus
  source 1..400: mol_type = genomic DNA
ttcaaacaagcagaaaagaattatcacagcaatgataaagaattactagcagtcaagcaagtaatcaccaagttttcagcttacctaacaccagtcaggtttacagtaagaactgataacaaaaattttacttatttccttagaattaatcttaaaggtgatagtaaacaaggacgattagtccgttggcaaaattggttcagcaagtatcaatttgatgtcgaacatcttgaaggtgtaaaaaacgttttagcagattgcctcacgagagattttaatgcttaaaaacgtaagcgctgacgtatgatttcaaaaaacgcagctataaaagaagccctccagcttcaaagttttcatcaacacaaattctaaaaacaaaattttttagagagggggagtg

SEQ ID NO: 70 - DNA, 1932 bp, Citrullus lanatus
  source 1..1932: mol_type = genomic DNA
tcaacataaacgcattagtgattaatacatttatttatgttttttgaagttagaaatttaaattcttaaatgtacctttgtaaaaaaaaatatatatatatatatatattgtgcccatgtttatatcctagagcgcattgtaatcacaaatcagataaatatatatcagattaaaataataataataataataataataatataatcccttataattatgcatttctaactttaaaaaataatccgtccataaacaacccattaatattaaccaagttgttgcaaattttgaaaacatatatatttgaaagccgtgtgaatgtaaaatactaaataaaatgatacatattttagtttttgtttgaacatatttcatagcctaacaaattcaaaagaaagaaaaaataattcttcccaaatttgaacaaagttttttctttattctttagatggagaaaatagtgtgagtaggttaaaaattttgggagagtgatattattattggggtcgataagatatttttgtggaattgtaataatttttcacatgttgaaatttttatggtctgtagttttcttttttctgaagtttttctatattaatgcttgtgtttctaattttattgctttctttcaatttctttgttgtttttcttctcattcatatagtagtaatgggaggtttttcccaaaagtatacacattattttttaattttattttctaatttttcacataacatttatatattatttttgttttcaatttttttagatggtgtaatatgtgtttatactattttaatttttagtacacttgaaatcaattttaaaaaattaattctaattttatttttctcacatatacatatgcatatacaaatatttttaatttttagtaaatgattttatgaaaattagttgtataaaaaacttctattttgaaatttagataaatatttcaagcttcattatttgcctatatattttggaaaaattatcttaaatgataaaactggtgaaatatttacaattaatagcaaaatacatagtctattgcaataaacaataaaattttgctatatttataaatatttgggtttcgtttgctatatttgaaaatagccttatattttgccatggataattgtgtaaagggttattatgattttaaaactaatatatgaatattagattaaagttgagttcctaaatttctaaatttatgtctaatagaatctctaaatttttaattttgtgtataattaagtcttttaaaaatgtctaatagatctataaacttttatctaatagtttcaaacatacttaatttttttttatttaaaaaaaaaaaaagggatgaactgttatattgggcataaaatttaattttaggtttaaatgaccctcacattttaaatcttctaactaataaatatattcattttttaaaatgtaacccgttagatacgtaaagttaaattttatatttaatataacatttaattttcaataattagataaaaattgaaactatatgaatctattaaatacaagattaagaacttagagacacattgaaaattaattttaaagtttatggacaatatatatcaacagaaatttaaaacttcataataagctaaacttgtaagctttatatggatttgtgtccctgtcatgaagttatggattcgtcactcaatgtctggtctaattaaaaagatgaatatgaaaaacgttttaacgattagcaaacaccaaaaccctaattaattatctctctctctcctccttagatttggctttttccttagcgtgaaaagcccaacttgttgtagtgaaactcagtcctataaatacaagaaagaaaaaaccctcttcaaaacccaaaccataaagaaaagaaaaaaaaaaaaaaaaaaaaaaagaga

SEQ ID NO: 71 - DNA, 2000 bp, Citrullus lanatus
  source 1..2000: mol_type = genomic DNA
tctctgtgtaaaaaaataaaaaataaaaaactatttttttaaatatttgtattaaaatacattaaaattatataagctttttactattattgtcatacattatttaatcaaatagaagcagggcaggctagaagagtagaagaattttggcgggaataagttcttgattcctcaaaataattcgtgatccgtgatccgtgaacatttaaaaatcaaattgaaataaaattcaaaacataaagactaaaagtgtaatattttaaaatacaatggttaaattaaaacatgtatatatatatttaatttttggaggaaaatagaaaaagaaaggaaaggtgtaacacacaacaaataataagaatttgaagtcaagaaatgagaacacacttccccgtttcgtgtgttttggttgaacaacttcaacctcaatctcccaagtcccaacatgtgaaatctcagttttaccccccaccgcctttgaattaaacgccgcgttttcttctttccttcttcaacccaaacgccaagctcctcctttcttttttcctttcaacagacgacagcccacaaacagacctatggttttaaaacttttacacatatgtatacttcattagatgtcatattaagcaaatcaacgattaaaattaatgcatagaggatttaatttccaaagtaattcttgactaataacaaaggctaaaaattaatacaacactcttaaacaaacatgaagtaatttagctttaaccatatcaattaaatttcaattttaacatcttttaagaacaaattttcttaccgcgtggagtctaacgctgtagaccttattggtttgcttagtttttaagtagtttgaacttactgaattagatatttttgttgtatccaccaaatgctaaatacttttgtttctctctcagaaaggagaataaagttgcttactctgttgcaactttcacatcatctcttaaggattcttttaggtggaaggataaattttataattgtattatctctatcattaatggagagttcgtgtcgtatttggggttggatgctctatttgtatctttgtgtcttttttttttttcaatctcaatgttgtctagaacatgtctctaatttttgtttttttaaaccataagatttctaaacattttgttctgtgtctaatagatttataacttctttttaaaaaaatgtttaacaagtccttgtaagttgttgttaatttaattttagacatttatttgacactataactatgatatatgatctttaaacgcaaaatgtcaatttgagcacaattcaattgattaaaatattattcgtaacattaataaaaagattaaactactaaaaaatgttaattttagcgactgtatagagcgttgaattaagttctaaaattatgtgagaatcatagtattaattttccgtcaacatctctatattttttttgtttccatagatttgatatcgacattagaggtgaatcgaaatctccattatatcgtagaaaaatccacgaaacataaaagataatcacgaaatgttactaatataaatataatttaaataatagacatgtcaacttgttcaaaaaatcataaaacatttatttactaatttaaatttattttttattttttaattttatcacttaaaataaaaaaatttaaaaaaaaattcttcaataaatatgttaacttcaattttcaacggtattgaaattgagaaaggaaaaaaaaaaaaaacacaatttgcagcgaacacaaataatttaattagtttttagtttttttgtattccacgtggccacaaatctcacgcgacttttcaccacgcaattggacgcctagtcccggtggggtccacacgccttccggccaccctataaataagctccgaatacaacgctctcatttcaccgactcacaatttccagaattcacacacattccttttcttctcttaagtaatttctttcactttca

SEQ ID NO: 72 - DNA, 561 bp, Pisum sativum
  source 1..561: mol_type = genomic DNA
gtttcattgcccacacaccagaatcctactaagtttgagtattatggcattggaaaagctgttttcttctatcatttgttctgcttgtaatttactgtgttctttcagtttttgttttcggacatcaaaatgcaaatggatggataagagttaataaatgatatggtccttttgttcattctcaaattattattatctgttgtttttactttaatgggttgaatttaagtaagaaaggaactaacagtgtgatattaaggtgcaatgttagacatataaaacagtctttcacctctctttggttatgtcttgaattggtttgtttcttcacttatctgtgtaatcaagtttactatgagtctatgatcaagtaattatgcaatcaagttaagtacagtataggctttttgtgtcaacatttattcactttcctttctgcaattttcaaatcatgtttgtagtttttattggacttgatgaatctctcttaccaccaaaatagaacattttttcttgatataccagaatctcaatatttgtctgataattattctagctgca

SEQ ID NO: 73 - DNA, 405 bp, Arabidopsis thaliana
  source 1..405: mol_type = genomic DNA
tttttgtgatctgatgataagtggttggttcgtgtctcatgcacttgggaggtgatctatttcacctggtgtagtttgtgtttccgtcagttggaaaaacttatccctatcgatttcgttttcattttctgcttttcttttatgtaccttcgtttgggcttgtaacgggcctttgtatttcaactctcaataataatccaagtgcatgttaaacaatttgtcatctgtttcggctttgatatactactggtgaagatgggccgtactactgcatcacaacgaaaaataataataagatgaaaaacttgaagtggaaaaaaaaaaaaacttgaatgttcactactactcattgaccataatgtttaacatacatagctcaatagtatttttgtgaatatggccaac

SEQ ID NO: 74 - DNA, 540 bp, Glycine max
  source 1..540: mol_type = genomic DNA
acgggaattaaacctataaacataaatataaataatatatataaacctaagtgtctaagttccataaattaagctgtagtctctggcttaaaacatgttaggtttgtttatacaagtagttggatgtttggagtacttcggtcttttgcgtaccatcaatatttaagaactaagttagttatgttccgtaacttatgggctcttaattaaactatatctgcacaaaattatatatatatcaaatgtgatggtatgtggactataaaaagatatggttgagaaccacaaactttgaaacttcgaataatatattgccagtgacagtcttgttgatttgttatagcaagtcctattttcttaatcattgctttgttttaacgtacctagatttcataacttttgtctttgtctcaagctgaacctaatgatgatagtaatattaacttattgtataggggtatttcataggataaaaaatgatgtgcaattacgtgtagaccaaatattacttgatgacagatggataaatattttaaaact

SEQ ID NO: 75 - DNA, 210 bp, Arabidopsis thaliana
  source 1..210: mol_type = genomic DNA
tcccctttctggaatattcagcgttgattattctggaacccatttctatgtggtcaatgcaaatttaagaaattatttgccgacttaacagttgaggaactattgtttgaaagtgaaaatgttattcctatcagtttctctataattatagttatcatttcatttcatttttgcccttaaatctttgaaatcttatttttcgtttagctc

SEQ ID NO: 76 - DNA, 295 bp, Pisum sativum
  source 1..295: mol_type = genomic DNA
attatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacatcccaaac

SEQ ID NO: 77 - DNA, 486 bp, Arabidopsis thaliana
  source 1..486: mol_type = genomic DNA
gaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagactacgtatcgggaatcgcctaattaaagcattaatgcgaacctg

SEQ ID NO: 78 - DNA, 414 bp, Solanum tuberosum
  source 1..414: mol_type = genomic DNA
tttaaactgattttaatgtttagcaaatgtcttatcagttttctctttttgtcgaacggtaatttagagttttttttgctatatggattttcgtttttgatgtatgtgacaaccctcgggattgttgatttatttcaaaactaagagtttttgtcttattgttctcgtctattttggatatcaatcttagttttatatcttttctagttctctacgtgttaaatgttcaacacactagcaatttggcctgccagcgtatggattatggaactatcaagtgtgtgggatcgataaatatgcttctcaggaatttgagattttacagtctttatgctcattcccttgagtataatatagtaaaaaaatagtaaatttaagcaataatgttaggtgctatgtgtctgtcgagactat

SEQ ID NO: 79 - DNA, 220 bp, Arabidopsis thaliana
  source 1..220: mol_type = genomic DNA
agaaaagatgattgattattacttttgtaaggctgtaagttcgtcgcactcatttccttttacctctttttctgaattgggattctctctttttgtgctttgtgtgttttgttattgaatttctttcccaaacctaagatttgttccagagtgtaatcacatttgcctatgcttctttgttttgtctatgaaaatatttcaatgtcaaaagcctatacaa

SEQ ID NO: 80 - DNA, 175 bp, Cauliflower mosaic virus
  source 1..175: mol_type = genomic DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc

SEQ ID NO: 81 - DNA, 27 bp, synthetic construct
  source 1..27: mol_type = other DNA
acacgctggaattctagtatactaaac

SEQ ID NO: 82 - DNA, 543 bp, Nicotiana tabacum
  source 1..543: mol_type = genomic DNA
ttatggctttatcaaatataggttttgaagattcagagagccaagaaaagctagaacccaagaactaggaagttagagtaattcacaataccataacgtgatataaaactttttattgtaactcaaatcggtaatattttttgctttagtcttaatcgataaattatttttttatattgattagttataggaggctcacaaagttgggaataattaaaatatcatattttgtatttgaacaatttatgaaatagtaattggtaaaaaatcactttaaatttttatcctatatccagaaggattatggtgtctggcatagttgtttggaagatttgaatcagggtaaaagtatgttgtaatttttattttgttataggcattttttgtgcttgattgttttgttgtcattatattttattatttggaagtgtatatatatgtttgattaaaatatagataatcaattttataagaaatttgcaacaattacacaaggataaagtctacaatatgcgagtaaaatttgattgaacttaggatgtc

SEQ ID NO: 83 - DNA, 66 bp, synthetic construct
  source 1..66: mol_type = other DNA
ggcagcggcgccacaaacttctctctgctaaagcaagcaggtgatgttgaagaaaaccccgggcct

SEQ ID NO: 84 - protein, 22 aa, synthetic construct
GSGATNFSLLKQAGDVEENPGP

SEQ ID NO: 85 - DNA, 1554 bp, synthetic construct
  source 1..1554: mol_type = other DNA
atgtcgtcgtgtgttgcagctacgatttcaatatggatggttgtggtgtgcatagtgggagtgggatggagagttgtgaactgggtttggttgaggccgaagaagcttgagaagcggctgagagagcaaggcctcgccggaaactcttaccggcttctgttcggagacttgaaggagagggcggcgatggcggagcaggccaactccaagcccatcaacttctcccatgatatcggaccacgtgtcttcccctccatgtacaaaaccatccagaattacggtaagaattcgtacatgtggcttggcccatatccaagagtgcacatcatggaccctcagcaacttaaaactgtttttactctagtctatgatatccaaaagccaaatttgaacccccttgtcaagtttcttttggatggaatagtaactcatgaaggagaaaaatgggctaaacacagaaagataatcaaccctgcatttcatttggaaaagttgaaggatatgataccagcattctttcatagttgtaatgagatagttaacgaatgggaaagattaatctcgaaagagggttcgtgtgagttggatgttatgccatatctgcaaaatttggcagctgatgccatttctcgaactgcatttgggagtagctatgaagaagggaaaatgatcttccaacttctaaaagaactaactgatttggtggttaaagttgcatttggagtttatattcccggatggaggtttctaccaactaagtcaaacaataaaatgaaagaaataaatagaaaaataaaaagtttgcttttgggtattataaacaaaaggcaaaaggctatggaagaaggtgaagctggacaaagtgatttattaggcattctcatggaatccaattcaaacgaaattcaaggagaaggaaacaataaagaagatggaatgagcatagaagatgttattgaagaatgcaaggttttctatattggtggccaagaaaccacagccagattactgatttggaccatgattttgttgagttcacacacggaatggcaagagcgagcaagaactgaggtattaaaagtatttggtaataagaagccagattttgatggtttgaatcgtctaaaaattgtaactatgattttgaacgaggttctcaggttatacccaccagcaagtatgcttactcgtattattcaaaaggaaacaagacttggaaaattgactctaccagctggtgtgatattgatcatgccaattattcttatccatcgtgatcatgacctatggggtgaagatgcaaacgaatttaaaccagaaagattttctgagggagtctctaaagcagcaaaagttcaacccgctttcttcccatttggatggggtcctcgaatatgcatggggcagaactttgcgatgattgaagcaaaaacggcattatcattaattctacaacgcttctcatttgagctttcttcgtcgtatgttcatgctcctaccgtcgttttcactactcaacctcaacatggagctcatatcgtcctgcgcaaactg

SEQ ID NO: 86 - protein, 518 aa, Siraitia grosvenorii
MSSCVAATISIWMVVVCIVGVGWRVVNWVWLRPKKLEKRLREQGLAGNSYRLLFGDLKERAAMAEQANSKPINFSHDIGPRVFPSMYKTIQNYGKNSYMWLGPYPRVHIMDPQQLKTVFTLVYDIQKPNLNPLVKFLLDGIVTHEGEKWAKHRKIINPAFHLEKLKDMIPAFFHSCNEIVNEWERLISKEGSCELDVMPYLQNLAADAISRTAFGSSYEEGKMIFQLLKELTDLVVKVAFGVYIPGWRFLPTKSNNKMKEINRKIKSLLLGIINKRQKAMEEGEAGQSDLLGILMESNSNEIQGEGNNKEDGMSIEDVIEECKVFYIGGQETTARLLIWTMILLSSHTEWQERARTEVLKVFGNKKPDFDGLNRLKIVTMILNEVLRLYPPASMLTRIIQKETRLGKLTLPAGVILIMPIILIHRDHDLWGEDANEFKPERFSEGVSKAAKVQPAFFPFGWGPRICMGQNFAMIEAKTALSLILQRFSFELSSSYVHAPTVVFTTQPQHGAHIVLRKL

SEQ ID NO: 87 - DNA, 1584 bp, synthetic construct
  source 1..1584: mol_type = other DNA
atggtggatcagtgcgcgttgggatggatcttggcctccgcgctgggcctcgtaattgcgctttgtttcttcgtggctccgaggaggaatcacagaggagtggattcgaaggagagggacgagtgcgtccaaagcgctgcaaccacgaagggagaatgcagattcaacgatcgcgacgttgacgttatcgtcgttggcgccggtgttgccggttccgctcttgctcacactcttggcaaggatggtcgtcgagttcatgtaattgaaagagacttgacagagcctgacagaatcgttggtgaattattacaacctgggggttacctcaaattgattgaattaggacttcaagactgcgtcgaggagattgatgctcaaagggtgtatggctacgcccttttcaaggatggaaagaacactcgactctcttacccattggaaaattttcactctgatgtatctggaaggagctttcacaacgggcgcttcatacagagaatgagggagaaggctgcttcccttcccaatgtcagattggagcaagggacagttacttcgctgcttgaagaaaagggaacgatcaaaggtgtgcagtataagtctaaaaatggtgaagagaaaacagcatatgcacctctgaccattgtttgtgatggctgcttctcaaacttgcgccgctctctctgcaaccctatggttgatgttccctcttattttgtgggattagttcttgagaattgtgagcttccttttgcaaatcacgggcacgttatcctcggagatccttctcccattttattctaccagattagcaggaccgagatccgttgtttggttgatgttcctggtcagaaggttccttctatagcaaatggtgaaatggagaaatatttgaagactgtagtagctcctcaggttcccccgcaaatctacgattcctttatcgctgctatcgacaagggtaatataaggacaatgccaaacagaagcatgcctgctgctccccacccaacgcccggtgccttactgatgggtgatgctttcaacatgcgccaccctcttaccggtggaggaatgaccgtagcattgtctgatatagttgtattgcggaacctcctcaagcctctgaaggacttgagtgatgcatctaccctctgcaagtatcttgaatccttttacactttgcgaaagccagtggcttcgaccatcaacacattggcaggggcattatacaaggtcttttgtgcatcaccagatcaagctaggaaggaaatgcgacaagcctgcttcgattacttgagccttggaggaatattctcaaatggacctgtctccttgctttcagggttgaatcctcgccccttaagtttggttctccatttctttgccgtcgcgatatacggagttggtcgcttattacttccatttccttcagtgaaaggcatctggattggagctagattgatctatagcgcatcaggtatcatattcccaattatacgggcggaaggagttagacagatgttcttccctgcaactgttcctgcttattatagaagtccaccagtgtttaaacccatagta

SEQ ID NO: 88 - DNA, 1419 bp, synthetic construct
  source 1..1419: mol_type = other DNA
atgtggactgtcgtgctcggtttggcgacgctgtttgtcgcctactacatccattggattaacaaatggagagattccaagttcaacggagttctgccgccgggcaccatgggtttgccgctcatcggagagacgattcaactgagccgacccagtgactccctcgacgttcaccctttcatccagaaaaaagttgaaagatacgggccgatcttcaaaacctgtctggccggaaggccggtggtggtgtcggcggacgcagagttcaacaactacataatgcttcaggaaggaagagcagtggaaatgtggtatttggatacgctctccaaatttttcggcctcgacaccgagtggctcaaagctctgggcctcatccacaagtacatcagaagcattactctcaatcacttcggcgccgaggccctgcgggagagatttcttccttttattgaagcatcctccatggaagcccttcactcctggtctactcaacctagcgtcgaagtcaaaaatgcctccgctctcatggtttttaggacctcggtgaataagatgttcggtgaggatgcgaagaagctatcgggaaatatccctgggaagttcacgaagctgctaggaggatttctcagtttaccactgaattttcccggcaccacctaccacaaatgcttgaaggatatgaaggaaatccagaagaagctaagagaggttgtagacgatagattggctaatgtgggccctgatgtggaagatttcttggggcaagcccttaaagataaggaatcagagaagttcatttcagaggagttcatcatccaactgttgttttctatcagttttgccagctttgagtccatctccaccactcttactttgattctcaagctccttgatgaacacccagaagtagtgaaagagttggaagctgaacacgaggcgattcgaaaagctagagcagatccagatggaccaattacttgggaagaatacaaatccatgacttttacattacaagtcatcaatgaaaccctaaggttggggagtgtcacaccagccttgttgaggaaaacagttaaagaccttcaagtaaaaggatacataatcccggaaggatggacaataatgcttgtcaccgcttcacgtcacagagatccaaaagtctataaggaccctcatatcttcaatccatggcgttggaaggacttggactcaattaccatccaaaagaacttcatgccttttgggggaggcttaaggcattgtgctggtgctgagtactctaaagtctacttgtgcaccttcttgcacatcctctgtaccaaataccgatggaccaaacttgggggaggaacgattgcaagagcgcatatattgagttttgaagatgggttacacgtgaagttcacacccaaggaa

SEQ ID NO: 89 - DNA, 2277 bp, synthetic construct
  source 1..2277: mol_type = other DNA
atgtggaggttaaaggtcggagcagaaagcgttggggagaatgatgagaaatggttgaagagcataagcaatcacttgggacgccaggtgtgggagttctgtccggatgccggcacccaacaacagctcttgcaagtccacaaagctcgtaaagcgttccacgatgaccgtttccaccgaaagcaatcttccgatctctttatcactattcagtatggaaaggaagtagaaaatggtggaaagacagcgggagtgaaattgaaagaaggggaagaggtgaggaaagaggcagtagagagtagcttagagagggcattaagtttctactcaagcatccagacaagcgatgggaactgggcttcggatcttggggggcccatgtttttacttccgggtctggtgattgccctctacgttacaggcgtcttgaactctgttttatccaagcaccaccggcaagagatgtgcagatatgtttacaatcaccagaatgaagatggggggtggggactccacatcgagggcccaagcaccatgtttggttccgcactgaattatgttgcactcaggctgcttggagaagacgccaacgccggggcaatgccaaaagcacgtgcttggatcttggaccacggtggcgccaccggaatcacttcctggggcaaattgtggctttctgtacttggagtctacgaatggagtggcaataatcctcttccacccgaattttggttatttccttacttcctaccatttcatccaggaagaatgtggtgccattgtcgaatggtttatctaccaatgtcatacttatatggaaagagatttgttgggccaatcacacccatagttctgtctctcagaaaagaactctacgcagttccatatcatgaaatagactggaataaatctcgcaatacctgtgcaaaggaggatctgtactatccacatcccaagatgcaagatattctgtggggatctctccaccacgtgtatgagcccttgtttactcgttggcctgccaaacgcctgagagaaaaggctttgcagactgcaatgcaacatattcactatgaagatgagaatacccgatatatatgccttggccctgtcaacaaggtactcaatctgctttgttgttgggttgaagatccctactccgacgccttcaaacttcatcttcaacgagtccatgactatctctgggttgctgaagatggcatgaaaatgcagggttataatgggagccagttgtgggacactgctttctccatccaagcaatcgtatccaccaaacttgtagacaactatggcccaaccttaagaaaggcacacgacttcgttaaaagttctcagattcagcaggactgtcctggggatcctaatgtttggtatcgtcacattcataaaggtgcatggccattttcaactcgcgatcatggatggctcatctctgactgtacagcagagggattaaaggctgctttgatgttatccaaacttccatccgaaacagttggggaatcattagaacggaatcgcctttgcgatgctgtaaacgttctcctttctttgcaaaacgataatggtggctttgcatcatatgagttgacaagatcatacccttggttggagttgatcaaccccgcagaaacgtttggagatattgtcattgattatccgtatgtggagtgcacctcagccacaatggaagcactgacgttgtttaagaaattacatcccggccataggaccaaagaaattgatactgctattgtcagggcggccaacttccttgaaaatatgcaaaggacggatggctcttggtatggatgttggggggtttgcttcacgtatgcggggtggtttggcataaagggattggtggcagctggaaggacatataataattgccttgccattcgcaaggcttgcgattttttactatctaaagagctgcccggcggtggatggggagagagttacctttcatgtcagaataaggtatacacaaatcttgaaggaaacagaccgcacctggttaacacggcctgggttttaatggccctcatagaagctggccaggctgagagagatccaacaccattgcatcgtgcagcaaggttgttaatcaattcccagttggagaatggtgatttcccccaacaggagatcatgggagtctttaataaaaattgcatgatcacatatgctgcataccgaaacatttttcccatttgggctcttggagagtattgccatcgggttttgactgaa

SEQ ID NO: 90 - DNA, 948 bp, synthetic construct
  source 1..948: mol_type = other DNA
atggaccagattgagcatatcaccatcaacaccaatggcatcaaaatgcacattgcctctgtagggacgggcccagtagttcttcttctgcatggcttcccggaactctggtattcatggcgccaccagcttctgtatctttcttccgtaggatatcgagctattgcgccggacctccgcggctatggcgacacggactcgccggcgtctcctacctcctacaccgctctccacatcgtcggcgatttggttggggctctggacgagcttgggatcgagaaggtgtttctggtcggacatgactggggggcgatcatcgcctggtacttttgcttgttcaggcccgatagaatcaaggcgctggtgaatctgagcgtccagttcatacccagaaacccagcgattcctttcattgagggtttcagaactgcgttcggtgatgacttctatatgtgcaggtttcaggttccaggagaggcagaagaagattttgcctccatcgacacagctcagctgttcaagacatcattatgtaatagaagttctgcacctccatgcttgcctaaagaaattggatttcgtgcgatcccacctccagagaaccttccttcttggctgacagaagaagatatcaactattatgctgccaaatttaagcagacaggcttcaccggagcgttgaactactatcgagcttttgacctaacttgggaactcacggcgccttggacaggagcacagattcaggttccggtgaagttcatcgtcggggattcggatctaacttaccattttccgggagccaaggaatatatccataatggcggattcaaaaaggacgtgccgttgctggaggaagtagttgtagtaaaagatgcttgtcacttcatcaaccaagaaaggccacaagaaatcaatgctcacatccatgacttcatcaataaattc

SEQ ID NO: 91 - DNA, 1422 bp, synthetic construct
  source 1..1422: mol_type = other DNA
atggtgcaacctcgggtactgctgtttcctttcccggcactgggccacgtgaagcccttcttatcactggcggagctgctttccgacgccggcatagacgtcgtcttcctcagcaccgagtataaccaccgtcggatctccaacactgaagccctagcctcccgcttcccgacgcttcatttcgaaactataccggatggcctgccgcctaatgagtcgcgcgctcttgccgacggcccactgtatttctccatgcgtgagggaactaaaccgagattccggcaactgattcaatctcttaacgacggtcgttggcccatcacctgtattatcactgacatcatgttatcttctccgattgaagtagcggaagaatttgggattccagtaattgccttctgcccatgcagtgctcgctacttatcgattcacttttttataccgaagctcgttgaggaaggtcaaattccatacgcagatgacgatccgattggagagatccagggggtgcccttgttcgaaggtcttttgcgacggaatcatttgcctggttcttggtctgataaatccgcagatatatctttctcgcatggcttgattaatcagacccttgcagctggtcgagcctcggctcttatactcaacaccttcgacgaactcgaagctccatttctgacccatctctcttccattttcaacaaaatctacaccattggacccctccatgctctgtccaaatcaaggctcggcgactcctcctcctccgcttctgccctctccggattctggaaagaggatagagcctgcatgtcctggctcgactgtcagccgccgaggtctgtggttttcgtcagtttcgggagtacgatgaagatgaaagccgatgaattgagagagttctggtatgggttggtgagcagcgggaaaccgttcctctgcgtgttgagatccgacgttgtttccggcggagaagcggcggaattgatcgaacagatggcggaggaggagggagctggagggaagctgggaatggtagtggagtgggcagcgcaagagaaggtcctgagccaccctgccgtcggtgggtttttgacgcactgcgggtggaactcaacggtggaaagcattgccgcgggagttccgatgatgtgctggccgattctcggcgaccaacccagcaacgccacttggatcgacagagtgtggaaaattggggttgaaaggaacaatcgtgaatgggacaggttgacggtggagaagatggtgagagcattgatggaaggccaaaagagagtggagattcagagatcaatggagaagctgtcaaagttggcaaatgagaaggttgtcaggggtgggttgtcttttgataacttggaagttctcgttgaagacatcaaaaaattgaaaccatataaattt

SEQ ID NO: 92 - DNA, 1356 bp, synthetic construct
  source 1..1356: mol_type = other DNA
atggatgctgcccaacaaggtgacaccacaaccattttgatgcttccatggctcggctatggccatctttcagcttttctcgagctggccaaaagcctctcaaggaggaacttccatatctacttctgttcaacctctgttaatcttgacgccattaaaccaaagcttccttcttctttctctgattccattcaatttgtggagctccatctcccttcttctcctgagttccctcctcatcttcacacaaccaacggccttccccctaccctcatgcccgctctccaccaagccttctccatggctgcccagcactttgagtccattttacaaacacttgccccgcaccttctcatttatgactctcttcaaccttgggctcctcgggtagcttcatccctcaaaattccggccatcaacttcaataccacgggagttttcgtcatttctcaagggcttcaccctattcactacccacattctaaattcccattctcagagttcgttcttcacaatcattggaaagccatgtactccactgccgatggagcttctaccgaaagaacccgcaaacgtggagaagcgtttctgtattgcttgcatgcttcttgtagtgtaattctaatcaatagtttcagagagctcgaggggaaatatatggattatctctctgttctcttgaacaagaaagttgttccggttggtcctttggtttacgaaccgaatcaagacggggaagatgaaggttattcaagcatcaaaaattggcttgacaaaaaggaaccgtcctccaccgtcttcgtgtcatttggaagcgaatacttcccgtcaaaggaagaaatggaagagatagcccatgggttagaggcgagcgaggttaatttcatctgggtcgttaggtttcctcaaggagacaacaccagcggcattgaagatgccttgccgaagggttttctggagagggcgggagagagagggatggtggtgaagggttgggctcctcaggcgaagatactgaagcattggagcacagggggattcgtgagccactgtggatggaactcggtgatggagagcatgatgtttggcgttcccataataggggttccgatgcatgtggaccagccctttaacgccggactcgtggaagaagctggcgtcggcgtggaggccaagcgagatccagacggcaaaattcaaagagacgaagttgcaaagttgatcaaagaagtggtggttgagaaaaccagagaagatgtgcggaagaaagcaagagaaatgagtgagattttgaggagcaagggagaggagaagtttgatgagatggtcgctgaaatttctctcttgcttaaaata

SEQ ID NO: 93 - DNA, 1029 bp, synthetic construct
  source 1..1029: mol_type = other DNA
atggcggatctcaagtcaaccttcctcaaggtctactcggttctgaaggatgagcttcttcaggaccctgcgtttgagtttacggatgcttctcgccaatggatcgaacggatgctggactacaatgtgcctggaggcaagcttaaccgagggctctccgtcatcgatagctacaaattattgaaagaagggaaggaattgactgaagatgaaatctttcttgctagtgttcttggttggtgtattgaatggctccaggcgtattttcttgttctcgatgatattatggataactctgtcacacgacgtggtcaaccttgctggtttagagtacccaaggttgggatgattgcagtaaacgatggagttctacttcgaaatcatattcctagaatgctaaaaaatcacttcaaagggaagccgtattacgttgatctgcttgatttgtttaacgaggttgagtttcaaacagcatcaggacaaatgattgatttgataaccactatcgatggagaaaaagatctttccaaatatgcattgtcacttcatcggcgcattgtacagtacaagactgcctattattcattttaccttccagttgcttgtgcactagttatgtcaggtgaagatcttgagaaacatacagtggttaaagacattcttgttcagatgggtgtctattttcaagttcaggatgattatctagattgttttggcgatcctgaaaccataggaaaggtgggaacggacattgaagatttcaaatgctcttggttggttgtgaaagctctggaactttgcaacgaggatcagaagaagttattacacgaggcttatgggaaaccagacccagaaaatgttgccaaagtaaaggccatctacaaggatctcgatcttcagggtgtatttgcggagtatgagagccagagctacgctaaactgaatgcctccattgaagctcatcccaacaaatcaattcaagcagttcttaagtctttcttgggtaaaatatacaggcgccagaagtga

SEQ ID NO: 94 - protein, 342 aa, Citrullus lanatus
MADLKSTFLKVYSVLKDELLQDPAFEFTDASRQWIERMLDYNVPGGKLNRGLSVIDSYKLLKEGKELTEDEIFLASVLGWCIEWLQAYFLVLDDIMDNSVTRRGQPCWFRVPKVGMIAVNDGVLLRNHIPRMLKNHFKGKPYYVDLLDLFNEVEFQTASGQMIDLITTIDGEKDLSKYALSLHRRIVQYKTAYYSFYLPVACALVMSGEDLEKHTVVKDILVQMGVYFQVQDDYLDCFGDPETIGKVGTDIEDFKCSWLVVKALELCNEDQKKLLHEAYGKPDPENVAKVKAIYKDLDLQGVFAEYESQSYAKLNASIEAHPNKSIQAVLKSFLGKIYRRQK

SEQ ID NO: 95 - DNA, 1425 bp, synthetic construct; identical to SEQ ID NO: 30
  source 1..1425: mol_type = other DNA

SEQ ID NO: 96 - protein, 474 aa, Siraitia grosvenorii; identical to SEQ ID NO: 31

SEQ ID NO: 97 - DNA, 1167 bp, synthetic construct
  source 1..1167: mol_type = other DNA
atggggagtttgggggcaattttgaaacacccagatgatttttacccgcttttgaaactgaaaatggccgctagacatgcggagaagcagatcccacctgagcctcattggggattttgctataccatgctgcataaagtctcaagaagttttgcactcgttattcagcagcttaagcccgagcttcgcaatgctgtatgcatattttatcttgttctgcgagccctcgatactgttgaggatgatacaggcatacaaacagacatcaaagtgcccattctgaaagcttttcactgtcacatatataaccgtgattggcatttttcatgtggcacaaaggactataaagttcttatggatgagtttcatcatgtttcaactgcgtttctagaacttgggaaagggtaccaggaagcaattgaggatatcacaaaacggatgggtgcaggcatggctaaattcatttgccaagaggttgagacagttggtgattatgatgaatattgccactatgtagctggacttgtcggactaggtttatctaagctgttccacgctgcgaagtcagaggatttggcacctgattctctttcaaattctatgggattgtttcttcagaaaacaaacattatccgagattacttggaggacattaatgagataccaaagtctagaatgttttggcctcgtgagatttggggcaaatatgctgataaattagaggattttaaatatgaggagaattcagtcaaggccgtgcaatgcctcaatgatttggtcactaatgctttgaaccatgtggaggattgtctgaaatacatgtctaacttaagagatctttccatttttcgattttgtgcaatccctcagattatggcgattggaactctagcattgtgctacaataatgtcgaagtcttcagaggggtggtcaaaatgcgtcggggtcttactgctaaggtcattgatcgaacaaaaacgatggctgacgtctatggagctttctttgatttctctgttatgctgaaggctaaggtcaacagcagtgatcctaatgcttctaaaaccctgagcaggatcgaggcaatacagcaaacatgcaagcagtcggggctcttgaacaagaggaaattgtatgtagtaagaagtgagccaatgtacaattga

SEQ ID NO: 98 - protein, 388 aa, Citrullus lanatus
MGSLGAILKHPDDFYPLLKLKMAARHAEKQIPPEPHWGFCYTMLHKVSRSFALVIQQLKPELRNAVCIFYLVLRALDTVEDDTGIQTDIKVPILKAFHCHIYNRDWHFSCGTKDYKVLMDEFHHVSTAFLELGKGYQEAIEDITKRMGAGMAKFICQEVETVGDYDEYCHYVAGLVGLGLSKLFHAAKSEDLAPDSLSNSMGLFLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTKTMADVYGAFFDFSVMLKAKVNSSDPNASKTLSRIEAIQQTCKQSGLLNKRKLYVVRSEPMYN

SEQ ID NO: 99 - DNA, 1425 bp, synthetic construct; identical to SEQ ID NO: 28
  source 1..1425: mol_type = other DNA

SEQ ID NO: 100 - protein, 474 aa, Siraitia grosvenorii; identical to SEQ ID NO: 29

SEQ ID NO: 101 - DNA, 1422 bp, synthetic construct; identical to SEQ ID NO: 26
  source 1..1422: mol_type = other DNA

SEQ ID NO: 102 - protein, 473 aa, Siraitia grosvenorii; identical to SEQ ID NO: 27

SEQ ID NO: 103 - DNA, 2127 bp, synthetic construct; identical to SEQ ID NO: 18
  source 1..2127: mol_type = other DNA

SEQ ID NO: 104 - protein, 708 aa, Siraitia grosvenorii; identical to SEQ ID NO: 19

SEQ ID NO: 105 - DNA, 1374 bp, synthetic construct; identical to SEQ ID NO: 22
  source 1..1374: mol_type = other DNA

SEQ ID NO: 106 - protein, 457 aa, Siraitia grosvenorii; identical to SEQ ID NO: 23

SEQ ID NO: 107 - DNA, 1383 bp, synthetic construct; identical to SEQ ID NO: 20
  source 1..1383: mol_type = other DNA

SEQ ID NO: 108 - protein, 460 aa, Siraitia grosvenorii; identical to SEQ ID NO: 21

SEQ ID NO: 109 - DNA, 1425 bp, synthetic construct; identical to SEQ ID NO: 24
  source 1..1425: mol_type = other DNA

SEQ ID NO: 110 - protein, 474 aa, Citrullus lanatus; identical to SEQ ID NO: 25

SEQ ID NO: 111 - DNA, 1422 bp, synthetic construct; identical to SEQ ID NO: 32
  source 1..1422: mol_type = other DNA

SEQ ID NO: 112 - protein, 473 aa, Siraitia grosvenorii; identical to SEQ ID NO: 33

SEQ ID NO: 113 - DNA, 1401 bp, synthetic construct
  source 1..1401: mol_type = other DNA
atggctgatgatcagctccatatttttttctttccattgatggcgcacggccacataattcctactttggatatggcgaagttgttcgtttcacatggtgcaaaggcaactattatcaccactcctctgaacttacccatcttcgttcaatccagtgaaaaaatcaaacatttggggctggaaatttatgtcaaagctatcagattcccagcagtggaagctggtttgcctgaaggatgtgaaagaatggatcaattgacttctgatgatttggtacccaaattcttcaaggccaccaccatgcttcaacaacaactagagctattactccatgaatgtcaacccaacgctctcgttgctgacatgttcttcccctgggctacagaatctgctgccaaatttgatatcccaagattggtatttcatgggatgggcttctttgcgctttgtgctttggagaaccttaggcattacaagcctcagaagaatgtgtgttcggatacagaacctttcgttgtcccgaatcttccccataaggtaaagctcacaaggatgaaattagctccgtatgatcgagaagaaacagaaatgactaagtttattgaacaagtgaagggatccgagttcacaagttttggggttattgtcaatagcttctatgagcttgaaccagattatgctgattactacagaaaggttttgggtaaaagatcatggcacgttggaccctttttgctctgcaacagaaaaaacgaagaaaaaatccacaaaagcgatgataaacatcatgagtgcttgcaatggctggacacgaagaaacaaaattcagtaatctatgtttgtttcgggagcatgtctaactttactgattcccaaatcgaagaaattgcattaggcctcgaagcttcagagcaagaattcatatgggtaattaggaaaggcaagattccagaaggattcgaagagcgaacaaaagaaaaaggattcatcataagaggatgggcaccccaaacgctgattcttgatcacgaggctataggagcctttttaactcactgtggatggaattcgacacttgaaggcgtatcagcaggggttccaatggtgacttggcctatatttgcagagcaattcttgaatgagcaattgataactaaagtactacaaataggagtggcagttggggcagaggaatggagcatagtggtggataatgtgaaaagtgaagcaattacaaaggcagtgaagtgtgtaatggctggggaagaatcaatggaaatgaggaggagagcagaagaagttaagaaaatggcgaacaaggctgttgaagaaggtggatcatcttacagtgatttgaatgcactgtttcaagaactaagaaccattaatttgcacaactaa

SEQ ID NO: 114 - protein, 466 aa, Solanum lycopersicum
MADDQLHIFFFPLMAHGHIIPTLDMAKLFVSHGAKATIITTPLNLPIFVQSSEKIKHLGLEIYVKAIRFPAVEAGLPEGCERMDQLTSDDLVPKFFKATTMLQQQLELLLHECQPNALVADMFFPWATESAAKFDIPRLVFHGMGFFALCALENLRHYKPQKNVCSDTEPFVVPNLPHKVKLTRMKLAPYDREETEMTKFIEQVKGSEFTSFGVIVNSFYELEPDYADYYRKVLGKRSWHVGPFLLCNRKNEEKIHKSDDKHHECLQWLDTKKQNSVIYVCFGSMSNFTDSQIEEIALGLEASEQEFIWVIRKGKIPEGFEERTKEKGFIIRGWAPQTLILDHEAIGAFLTHCGWNSTLEGVSAGVPMVTWPIFAEQFLNEQLITKVLQIGVAVGAEEWSIVVDNVKSEAITKAVKCVMAGEESMEMRRRAEEVKKMANKAVEEGGSSYSDLNALFQELRTINLHN

SEQ ID NO: 115 - DNA, 1440 bp, synthetic construct
  source 1..1440: mol_type = other DNA
atggagaatgaaaaagataaacttcatgttgtgatgtttccatggctagccatgggacatatcatacctttttttcatctatcaaagtgtttagcacgaaggggccatagagtcaccttcatttcaactccaagaaacattgaaagagtcgtcaataaagttccctctgatcttgctcatctgttgcatattgtgtggtttcaattgcctaaagttgaaaacttgccggatgatgcagagtcatcgatggatattccttaccaaaaagctcagttcttgaagatagctttcgattcgctcgaaatcccactgaccaattatcttgagagttcatcaaatcccaaaccagattggattgtctatgactatgcttctcactggctacctcaaattgcaggtaaattaggcgtttctcgcgccttttttagtctgtttactgctgcaaccatgtcattttatggacctccttgtgctttgttaaatgatgaaaggtcaatagctgaggactatacagttgttccaaaatggattcctttcgaaacgaaagttgtgtataggctgcatgagattaagaagaatttcgaagcaccagctgatgaatcaggtacatctgatggagccagatttggtgcttcaattgatgcaagtgatgtcatattgtttcgaaattgtgttgagtttgaatccgaatggttcagcttggtttctgagctttatcagaaacctattatatccattggtgtgctacctccatctgttgtagttgatcaagaacaagatgacagcaatgatgcgtcgtggtcaggaatcaagagttggctagataagcacaatcaagattctgttgtttatgttgcacttggaactgaagcaacactgaatcaacaagagctgaatgagctagctttggggttagaaaaatgcggattaccattcgtttgggtattaagagatcagccaaaacataatcaacaagattcgtgtattcagctgcctgatggatacgaagacagagtcaaaaacagaggtgtaatatacaaaggatgggtaccacagacgaagatactgagtcattcgtcagtaggtggattcttgactcattgtggatggaattcggtcatagaagcgctatgttttgggcgggttttggttatgttcccggtgttgaatgatcaaggattgaatacaaggttgttgcaagagaaaggagttggtgtagagataccaagaaatgaaaaagatggatttttcactagtgattcagtggctgaagctgtgaagtttggtgtagtgagtgaagagggtgagttgttgagagctaatgcaagacaaatgagttgtttgtttggagataggaagagaaatgaacaacttattgatgattgtgttggttatttcatggaaaataggataagcaaatctaatagctccatcggttga

SEQ ID NO: 116 - protein, 479 aa, Solanum lycopersicum
MENEKDKLHVVMFPWLAMGHIIPFFHLSKCLARRGHRVTFISTPRNIERVVNKVPSDLAHLLHIVWFQLPKVENLPDDAESSMDIPYQKAQFLKIAFDSLEIPLTNYLESSSNPKPDWIVYDYASHWLPQIAGKLGVSRAFFSLFTAATMSFYGPPCALLNDERSIAEDYTVVPKWIPFETKVVYRLHEIKKNFEAPADESGTSDGARFGASIDASDVILFRNCVEFESEWFSLVSELYQKPIISIGVLPPSVVVDQEQDDSNDASWSGIKSWLDKHNQDSVVYVALGTEATLNQQELNELALGLEKCGLPFVWVLRDQPKHNQQDSCIQLPDGYEDRVKNRGVIYKGWVPQTKILSHSSVGGFLTHCGWNSVIEALCFGRVLVMFPVLNDQGLNTRLLQEKGVGVEIPRNEKDGFFTSDSVAEAVKFGVVSEEGELLRANARQMSCLFGDRKRNEQLIDDCVGYFMENRISKSNSSIG

SEQ ID NO: 117 - DNA, 1413 bp, synthetic construct
  source 1..1413: mol_type = other DNA
atggtgcaaccccatgtcctattggtgacatttcctgcacaaggtcatatcaatccatctcttcaatttgccaagaggctaatcgaaatgggcattgaggtgacattcaccactagtgttttcgcccaccgtcgtatggcgaaaatcgcggcttccacggcacccaagggcttaaacttggcggcattctctgatggatttgatgatggtttcaagtctaacgtggatgattctaaacgttacatgtcagagataagaagtcgtgggtcccaaacattgagggatgtcattttgaagagttcagacgaaggacgtcctgtcacgtccctcgtctacactcttctgctaccttgggcagctgaggtagcgcgtgaactccatatcccatccgcgttactatggattcaaccagcaactgtgctagacatatactactattacttcaatggctatgaggatgaaatgaagtgtagtagctcaaatgatccaaattggagtatccaattgccaaggcttccattactaaaaagccaagatcttccatcttttttagtttcatctagctcaaaagatgataagtatagttttgctctaccaacattcaaagagcaactagacacattagatggtgaagaaaatccaaaggtacttgtgaatacatttgatgcattagagctagagccactcaaagctattgaaaaatacaatttaattggaattggaccattgattccttcatcattcttgggtggaaaagattcattggaatcttcatttggtggtgatctttttcaaaagtcaaatgatgactacatggaatggttaaacacaaagcctaaatcatcaattgtttatatctcatttgggagtctattgaatttatcaagaaaccaaaaggaggagattgcaaaagggttgatagagatccaaaggccattcttatgggtaataagagatcaagaagaagaaaaagaagaagagaaattaagttgcatgatggaattagagaagcaagggaaaatagtaccatggtgttcacaacttgaagtcctgacacatccatctttaggatgttttgtctcgcactgtggatggaattcgactctagaaagcctatcgtcaggtgtacctgtggtggcatttcctcattggacagatcaagggactaatgcaaagttgattgaagatgtttggaagacaggtgtcaggatgagagtgaatgaagatggtgtggttgagagtgatgaaataaaaaggtgcatagaaattgtaatggatggtggagaaaaaggagaagaaatgagaaaaaatgcacaaaaatggaaagaattggcaagagcagctgtgaaagaaggtggatcttcagaagtgaatctaaaagcttttgttctacaagttagcaaaagttgttga

SEQ ID NO: 118 - protein, 470 aa, Solanum lycopersicum
MVQPHVLLVTFPAQGHINPSLQFAKRLIEMGIEVTFTTSVFAHRRMAKIAASTAPKGLNLAAFSDGFDDGFKSNVDDSKRYMSEIRSRGSQTLRDVILKSSDEGRPVTSLVYTLLLPWAAEVARELHIPSALLWIQPATVLDIYYYYFNGYEDEMKCSSSNDPNWSIQLPRLPLLKSQDLPSFLVSSSSKDDKYSFALPTFKEQLDTLDGEENPKVLVNTFDALELEPLKAIEKYNLIGIGPLIPSSFLGGKDSLESSFGGDLFQKSNDDYMEWLNTKPKSSIVYISFGSLLNLSRNQKEEIAKGLIEIQRPFLWVIRDQEEEKEEEKLSCMMELEKQGKIVPWCSQLEVLTHPSLGCFVSHCGWNSTLESLSSGVPVVAFPHWTDQGTNAKLIEDVWKTGVRMRVNEDGVVESDEIKRCIEIVMDGGEKGEEMRKNAQKWKELARAAVKEGGSSEVNLKAFVLQVSKSC

SEQ ID NO: 119 - DNA, 1428 bp, synthetic construct
  source 1..1428: mol_type = other DNA
atgaaaatagaaagaaaacagagtgtagtactggtgccacacccataccaggggcatttaacaccaatgctacagcttggtagtatccttcattcacaaggcttttctgttatagttgcacatactcaatacaatactcctaattattccaatcatccacaattcgtcttccattctatggatgacggattacagggaatcgacatgtcattcccgagtttagaaaacatatatgatatgaacgaaaactgcaaggcgcctctcagaaactaccttgttagtatgatggaagaggaaggtgatcagcttgcttgtatcgtttatgacaacgtcatgttctttgtcgatgatgtagcgactcagttgaagcttcccagcattgtcctgcgcactttcagcgctgcgtatttgcactctatgatcaccattttacagcaacctgaaatatatttaccttttgaagattctcagctgctggatccgttaccggagcttcatcccttgagatttaaggatgtaccgtttcctatcatcaataatacagttccagaaccgatactagacttctgtagagcaatgagtgatataggatcatctgtcgcgactatatggaacacgatgcaagacttggagagttcaatgttgttacgccttcaagaacattacaaggtgcccttttttccaataggcccggtacacaaaatggcatctttggtgtcatcgactagcatactagaagaagacaatagctgcatcgagtggctcgatagacaagcccctaactctgttctctatgtcagcttgggtagcctagtgaggattgatcacaaagagttgattgagactgcttggggattagctaatagcgatcaaccgttcttgtgggttattcgacctggctctgtctctggctttcaatgtgctgaggcactgcctgatggttttgagaaaatggtaggagaaagaggacgaatagtgaaatgggcaccacaaaaacaggtgcttgcacatcccgcggtagcagggtttttcactcattgtggttggaattctacgcttgaaagtatatgtgaagaagtccctatggtgtgcaggccatttctagcagaccaactggtgaacgcaaggtatctgagccaaatatacaaggttgggttcgaattggaggttatcgagagaacggtcatagagaaaacaataagaaaactcatgttaagtgaagaaggcaaagatgtgaagaaaagagtagcagacatgaaacaaaagatagttgctggaatgcagattgattgcacttcacataagaatctgaatgatttggtagacttcatttctgccttgccatcacgactcgctccgccaacgcctgtctttggggcaatcatgagctcaaaccacatagcaagcaagtgtatcatagagtcttga

SEQ ID NO: 120 - protein, 475 aa, Solanum lycopersicum
MKIERKQSVVLVPHPYQGHLTPMLQLGSILHSQGFSVIVAHTQYNTPNYSNHPQFVFHSMDDGLQGIDMSFPSLENIYDMNENCKAPLRNYLVSMMEEEGDQLACIVYDNVMFFVDDVATQLKLPSIVLRTFSAAYLHSMITILQQPEIYLPFEDSQLLDPLPELHPLRFKDVPFPIINNTVPEPILDFCRAMSDIGSSVATIWNTMQDLESSMLLRLQEHYKVPFFPIGPVHKMASLVSSTSILEEDNSCIEWLDRQAPNSVLYVSLGSLVRIDHKELIETAWGLANSDQPFLWVIRPGSVSGFQCAEALPDGFEKMVGERGRIVKWAPQKQVLAHPAVAGFFTHCGWNSTLESICEEVPMVCRPFLADQLVNARYLSQIYKVGFELEVIERTVIEKTIRKLMLSEEGKDVKKRVADMKQKIVAGMQIDCTSHKNLNDLVDFISALPSRLAPPTPVFGAIMSSNHIASKCIIES

SEQ ID NO: 121 - DNA, 1476 bp, synthetic construct
  source 1..1476: mol_type = other DNA
atgggtgttcttactatagagccacattttgtgttgtttcctttcatggcacaaggccatacaatccctatgattgatattgcgcgattactatcacaacgcggagttattatcacaatcgttacaacacatttgaacgcgaatagattcaagaaagtcattgatcgcgctatagaatcaggactgaaaattcaggtggttcatctctactttccaagcttagaggctggactgcctgaaggatgtgaaaatttcgacatgcttccatcgatggatttggggctgaaattcttcgatgctacggaaagacttcagccccaagtggaagaaatgttgcgagaaatgaaaccttcaccaagttgtataatatctgatatgtgtttcccatggacaactaatgttgcacaaaagttcaacattcctagaattgttttccatggaatgggttgcttttctttgttatgtttacataatttgaaagattgggaagggttagaaaagatcgagtctgatacagagtattttcgagtgcctggattgttcgacaagattgaactaaccaaaaatcagcttggaaatgctgccaggccgagaaatgaagagtggcgagtaatgagtgaaaaaatgaagaaagcggaggaggaagcttatggaatggtggtgaatacctttgaggatttggaaaaagaatacatcgagggtttaatgaatgcgaaaaacaagaaaatttggaccattggccctgtttcactctgtaacaaagaaaaacaggacaaagctgaaagagggaacgaggctgcaattgatgaacacaagtgtctaaactggcttgattcgtgggaacaaaactctgtactctttgtatgtctcgggagcctatcgcgcctttccacgtctcagatggttgagctagggcttgggttagaatcatcgagacgaccctttatttgggttgttagacacatgtcagatgagttcaagaattggctagttgaagaagattttgaggaaagagttaaagggcaaggactattaatccgcggttgggcgccacaagtactactcttgtcacatccttcaataggtgcgttcttgactcactgtggatggaattcgagcctggaaggtataaccgctggcgtggccatgatcacttggccaatgtttgctgagcagttttgtaatgagaggttaatagtggatgtactcaagacaggagtgaggtcaggcatagagaggcaagtgatgtttggagaagaagaaaaattgggaacacaagtaagcaaagatgacattaagaaggtgattgaacaagtgatggatgaagaaatggaaggtgaaatgagaagaaaaagagcaaaagaattaggagaaaaggcaaagagggctatggaggaagagggttcatctcatttcaacttgacacaattgattcaagatgtgacagagcaagcaaaaattttaaaacctatgtag

SEQ ID NO: 122 - protein, 491 aa, Solanum lycopersicum
MGVLTIEPHFVLFPFMAQGHTIPMIDIARLLSQRGVIITIVTTHLNANRFKKVIDRAIESGLKIQVVHLYFPSLEAGLPEGCENFDMLPSMDLGLKFFDATERLQPQVEEMLREMKPSPSCIISDMCFPWTTNVAQKFNIPRIVFHGMGCFSLLCLHNLKDWEGLEKIESDTEYFRVPGLFDKIELTKNQLGNAARPRNEEWRVMSEKMKKAEEEAYGMVVNTFEDLEKEYIEGLMNAKNKKIWTIGPVSLCNKEKQDKAERGNEAAIDEHKCLNWLDSWEQNSVLFVCLGSLSRLSTSQMVELGLGLESSRRPFIWVVRHMSDEFKNWLVEEDFEERVKGQGLLIRGWAPQVLLLSHPSIGAFLTHCGWNSSLEGITAGVAMITWPMFAEQFCNERLIVDVLKTGVRSGIERQVMFGEEEKLGTQVSKDDIKKVIEQVMDEEMEGEMRRKRAKELGEKAKRAMEEEGSSHFNLTQLIQDVTEQAKILKPM

SEQ ID NO: 123 - DNA, 1359 bp, synthetic construct
  source 1..1359: mol_type = other DNA
atggatgctgcccaacaaggtgacaccacaaccattttgatgcttccatggctcggctatggccatctttcagcttttctcgagctggccaaaagcctctcaaggaggaacttccatatctacttctgttcaacctctgttaatcttgacgccattaaaccaaagcttccttcttctttctctgattccattcaatttgtggagctccatctcccttcttctcctgagttccctcctcatcttcacacaaccaacggccttccccctaccctcatgcccgctctccaccaagccttctccatggctgcccagcactttgagtccattttacaaacacttgccccgcaccttctcatttatgactctcttcaaccttgggctcctcgggtagcttcatccctcaaaattccggccatcaacttcaataccacgggagttttcgtcatttctcaagggcttcaccctattcactacccacattctaaattcccattctcagagttcgttcttcacaatcattggaaagccatgtactccactgccgatggagcttctaccgaaagaacccgcaaacgtggagaagcgtttctgtattgcttgcatgcttcttgtagtgtaattctaatcaatagtttcagagagctcgaggggaaatatatggattatctctctgttctcttgaacaagaaagttgttccggttggtcctttggtttacgaaccgaatcaagacggggaagatgaaggttattcaagcatcaaaaattggcttgacaaaaaggaaccgtcctccaccgtcttcgtgtcatttggaagcgaatacttcccgtcaaaggaagaaatggaagagatagcccatgggttagaggcgagcgaggttaatttcatctgggtcgttaggtttcctcaaggagacaacaccagcggcattgaagatgccttgccgaagggttttctggagagggcgggagagagagggatggtggtgaagggttgggctcctcaggcgaagatactgaagcattggagcacagggggattcgtgagccactgtggatggaactcggtgatggagagcatgatgtttggcgttcccataataggggttccgatgcatgtggaccagccctttaacgccggactcgtggaagaagctggcgtcggcgtggaggccaagcgagatccagacggcaaaattcaaagagacgaagttgcaaagttgatcaaagaagtggtggttgagaaaaccagagaagatgtgcggaagaaagcaagagaaatgagtgagattttgaggagcaagggagaggagaagtttgatgagatggtcgctgaaatttctctcttgcttaaaatataa

SEQ ID NO: 124 - protein, 452 aa, Siraitia grosvenorii
MDAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 125 - DNA, 906 bp, synthetic construct
  source 1..906: mol_type = other DNA
atggcgatttctctcaatcttcttcaacttcaaccaaatccagcgcggcccaagttcttcaaatggaaccccgtccgaccggcggttcatttcagagctccggcgaggttgaccgtcgtcatgtcacagatcactccgtcgtactgggcggccataaatgaagatattgaggatcatctccggcgggtcattccggcaaaggacccgccggcggtgtacgagccgttgggccattttgtcttctcggccccacgaaattccgctcctgcactgtgtattgccgcctgcgagctcgtcggcggcggccgagatcaggccattgtggcggctactgcactgcaactcatccacgctgcaacctatactcgggaataccctccactgacggcacgtgaaatctcacgcgccgccgtcgttgaccgcgtttatggccccaacatccagctgttgacaggggacggaatagcaccgatggtgttcgagattctagcgggatcttgcgattggagcgagagcgggtcggagcggattcgacgcgtgatggtggaaatggcacgtgcgatgggtacggaagggtggattggggggcaatttcggggattggaggagacggcaacggcggaggaagtgagcgagaagacggaaggtgggctacacgcgtgtgggggcgcgtgtggggtgatattgggaggtggaaatgaagaggaaatagagaagatgaggaaatatgggaaattggtggggatggtgaaaggaatgtcaaaaaatgtgtttggagggaaagaaaatgagagaaagaaagagaaaattgaggagttgaagtatttggctttcaaggaattgaaagatttcaaaggagaaaatgttcatgagatctatggattccttgattttgagggactttag

SEQ ID NO: 126 - protein, 301 aa, Citrullus lanatus
MAISLNLLQLQPNPARPKFFKWNPVRPAVHFRAPARLTVVMSQITPSYWAAINEDIEDHLRRVIPAKDPPAVYEPLGHFVFSAPRNSAPALCIAACELVGGGRDQAIVAATALQLIHAATYTREYPPLTAREISRAAVVDRVYGPNIQLLTGDGIAPMVFEILAGSCDWSESGSERIRRVMVEMARAMGTEGWIGGQFRGLEETATAEEVSEKTEGGLHACGGACGVILGGGNEEEIEKMRKYGKLVGMVKGMSKNVFGGKENERKKEKIEELKYLAFKELKDFKGENVHEIYGFLDFEGL

SEQ ID NO: 127 - DNA, 1461 bp, synthetic construct
  source 1..1461: mol_type = other DNA
atgggcgacaacggcgacggcggcgagaagaaggagctgaaggagaacgtgaagaagggcaaggagctgggcaggcaggccatcggcgagggctacatcaacccgagcctgcagctggccaggaggctgatcagcctgggcgtgaacgtgaccttcgccaccaccgtgctggccggcaggaggatgaagaacaagacccaccagaccgccaccaccccgggcctgagcttcgccaccttcagcgacggcttcgacgacgaaaccctgaagccgaacggcgacctgacccactacttcagcgagctgaggaggtgcggcagcgagagcctgacccacctgatcaccagcgccgccaacgagggcaggccgatcaccttcgtgatctacagcctgctgctgagctgggccgccgacatcgccagcacctacgacatcccgagcgccctgttcttcgcccagccggccaccgtgctggccctgtacttctactacttccacggctacggcgacaccatctgcagcaagctgcaggacccgagcagctacatcgagctgccgggcctgccgctgctgaccagccaggacatgccgagcttcttcagcccgagcggcccgcacgccttcatcctgccgccgatgagggagcaggccgagttcctgggcaggcagagccagccgaaggtgctggtgaacaccttcgacgccctggaggccgacgccctgagggccatcgacaagctgaagatgctggccatcggcccgctgatcccgagcgccctgctgggcggcaacgacagcagcgacgccagcttctgcggcgacctgttccaggtgagcagcgaggactacatcgagtggctgaacagcaagccggacagcagcgtggtgtacatcagcgtgggcagcatctgcgtgctgagcgacgagcaggaggacgagctggtgcacgccctgctgaacagcggccacaccttcctgtgggtgaagaggagcaaggagaacaacgagggcgtgaagcaggaaaccgacgaggagaagctgaagaagctggaggagcagggcaagatggtgagctggtgcaggcaagtggaggtgctgaagcacccggccctgggctgcttcctgacccactgcggctggaacagcaccatcgagagcctggtgagcggcctgccggtggtggccttcccgcagcagatcgaccaggccaccaacgccaagctgatcgaggacgtgtggaagaccggcgtgagggtgaaggccaacaccgagggcatcgtggagagggaggagatcaggaggtgcctggacctggtgatgggcagcagggacggccagaaggaggagatcgagaggaacgccaagaagtggaaggagctggccaggcaggccatcggcgagggcggcagcagcgacagcaacctgaagaccttcctgtgggagatcgacctggagatctaa

SEQ ID NO: 128 - protein, 486 aa, Siraitia grosvenorii
MGDNGDGGEKKELKENVKKGKELGRQAIGEGYINPSLQLARRLISLGVNVTFATTVLAGRRMKNKTHQTATTPGLSFATFSDGFDDETLKPNGDLTHYFSELRRCGSESLTHLITSAANEGRPITFVIYSLLLSWAADIASTYDIPSALFFAQPATVLALYFYYFHGYGDTICSKLQDPSSYIELPGLPLLTSQDMPSFFSPSGPHAFILPPMREQAEFLGRQSQPKVLVNTFDALEADALRAIDKLKMLAIGPLIPSALLGGNDSSDASFCGDLFQVSSEDYIEWLNSKPDSSVVYISVGSICVLSDEQEDELVHALLNSGHTFLWVKRSKENNEGVKQETDEEKLKKLEEQGKMVSWCRQVEVLKHPALGCFLTHCGWNSTIESLVSGLPVVAFPQQIDQATNAKLIEDVWKTGVRVKANTEGIVEREEIRRCLDLVMGSRDGQKEEIERNAKKWKELARQAIGEGGSSDSNLKTFLWEIDLEI

SEQ ID NO: 129 - DNA, 1362 bp, synthetic construct
  source 1..1362: mol_type = other DNA
atggagaagggcgacacccacatcctggtgttcccgttcccgagccagggccacatcaacccgctgctgcagctgagcaagaggctgatcgccaagggcatcaaggtgagcctggtgaccaccctgcacgtgagcaaccacctccagctgcagggcgcctacagcaacagcgtgaagatcgaggtgatcagcgacggcagcgaggacaggctggaaaccgacaccatgaggcagaccctggacaggttcaggcagaagatgaccaagaacctggaggacttcctgcagaaggccatggtgagcagcaacccgccgaagttcatcctgtacgacagcaccatgccgtgggtgctggaggtggccaaggagttcggcctggacagggccccgttctacacccagagctgcgccctgaacagcatcaactaccacgtgctgcacggccagctgaagctgccgccggaaaccccgaccatcagcctgccgagcatgccgctgctgaggccgagcgacctgccggcctacgacttcgacccggccagcaccgacaccatcatcgacctgctgaccagccagtacagcaacatccaggacgccaacctgctgttctgcaacaccttcgacaagctggagggcgagatcatccagtggatggaaaccctgggcaggccggtgaagaccgtgggcccgaccgtgccgagcgcctacctggacaagagggtggagaacgacaagcactacggcctgagcctgttcaagccgaacgaggacgtgtgcctgaagtggctggacagcaagccgagcggcagcgtgctgtacgtgagctacggcagcctggtggagatgggcgaggagcagctgaaggagctggccctgggcatcaaggaaaccggcaagttcttcctgtgggtggtgagggacaccgaggccgagaagctgccgccgaacttcgtggagagcgtggccgagaagggcctggtggtgagctggtgcagccagctggaggtgctggcccacccgagcgtgggctgcttcttcacccactgcggctggaacagcaccctggaggccctgtgcctgggcgtgccggtggtggccttcccgcagtgggccgaccaggtgaccaacgccaagttcctggaggacgtgtggaaggtgggcaagagggtgaagaggaacgagcagaggctggccagcaaggaggaggtgaggagctgcatctgggaggtgatggagggcgagagggccagcgagttcaagagcaacagcatggagtggaagaagtgggccaaggaggccgtggacgagggcggcagcagcgacaagaacatcgaggagttcgtggccatgctgaagcagacc

SEQ ID NO: 130 - protein, 454 aa, Siraitia grosvenorii
MEKGDTHILVFPFPSQGHINPLLQLSKRLIAKGIKVSLVTTLHVSNHLQLQGAYSNSVKIEVISDGSEDRLETDTMRQTLDRFRQKMTKNLEDFLQKAMVSSNPPKFILYDSTMPWVLEVAKEFGLDRAPFYTQSCALNSINYHVLHGQLKLPPETPTISLPSMPLLRPSDLPAYDFDPASTDTIIDLLTSQYSNIQDANLLFCNTFDKLEGEIIQWMETLGRPVKTVGPTVPSAYLDKRVENDKHYGLSLFKPNEDVCLKWLDSKPSGSVLYVSYGSLVEMGEEQLKELALGIKETGKFFLWVVRDTEAEKLPPNFVESVAEKGLVVSWCSQLEVLAHPSVGCFFTHCGWNSTLEALCLGVPVVAFPQWADQVTNAKFLEDVWKVGKRVKRNEQRLASKEEVRSCIWEVMEGERASEFKSNSMEWKKWAKEAVDEGGSSDKNIEEFVAMLKQT

SEQ ID NO: 131 - DNA, 1365 bp, synthetic construct
  source 1..1365: mol_type = other DNA
atggagaagggcgacacccacatcctggtgttcccgttcccggcccagggccacatcaacccgctgctgcagctgagcaagcacctgatcgccaagggcatcaaggtgagcctggtgaccaccctgcacgtgagcaacaggatgcagctgcagggcgcctacagcaacagcgtgaagatcgaggtgatcagcgacggcagcgaggacaggctggaaaccgacaccctgaggcagtacctggacaggttcaggcagaagatgaccaagaacctggaggacttcctgcagaaggccatggtgagcagcaacccgccgaagttcatcatctacgacagcaccatgccgtgggtgctggaggtggccaaggagttcggcctggacagggccccgttctacacccagagctgcgccctgaacagcatcaactaccacgtgctgcacggccagctgaagctgccgccggaaaccccgaccatcagcctgccgagcatgccgctgctgaggccgagcgacctgccggcctacgacttcgacccggccagcaccgacaccatcatcgacctgctgaccagccagtacagcaacatccaggacgccaacctgctgttctgcaacaccttcgacaagctggagggcgagatcatccagtggatggaaaccctgggcaggccggtgaagaccgtgggcccgaccgtgccgagcgcctacctggacaagagggtggagaacgacaagcactacggcctgagcctgttcaagccgaacgaggacgtgtgcctgaagtggctggacagcaagccgagcggcagcgtgctgtacgtgagctacggcagcctggtggagatgggcgaggagcagctgaaggagctggccctgggcatcaaggaaaccggcaagttcttcctgtgggtggtgagggacaccgaggccgagaagctgccgccgaacttcgtggagagcgtggccgagaagggcctggtggtgagctggtgcagccagctggaggtgctggcccacccgagcgtgggctgcttcttcacccactgcggctggaacagcaccctggaggccctgtgcctgggcgtgccggtggtggccttcccgcagtgggccgaccaggtgaccaacgccaagttcctggaggacgtgtggaaggtgggcaagagggtgaagaggaacgagcagaggctggccagcaaggaggaggtgaggagctgcatctgggaggtgatggagggcgagagggccagcgagttcaagagcaacagcatggagtggaagaagtgggccaaggaggccgtggacgagggcggcagcagcgacaagaacatcgaggagttcgtggccatgctgaagcagacctaa

SEQ ID NO: 132 - protein, 454 aa, Siraitia grosvenorii
MEKGDTHILVFPFPAQGHINPLLQLSKHLIAKGIKVSLVTTLHVSNRMQLQGAYSNSVKIEVISDGSEDRLETDTLRQYLDRFRQKMTKNLEDFLQKAMVSSNPPKFIIYDSTMPWVLEVAKEFGLDRAPFYTQSCALNSINYHVLHGQLKLPPETPTISLPSMPLLRPSDLPAYDFDPASTDTIIDLLTSQYSNIQDANLLFCNTFDKLEGEIIQWMETLGRPVKTVGPTVPSAYLDKRVENDKHYGLSLFKPNEDVCLKWLDSKPSGSVLYVSYGSLVEMGEEQLKELALGIKETGKFFLWVVRDTEAEKLPPNFVESVAEKGLVVSWCSQLEVLAHPSVGCFFTHCGWNSTLEALCLGVPVVAFPQWADQVTNAKFLEDVWKVGKRVKRNEQRLASKEEVRSCIWEVMEGERASEFKSNSMEWKKWAKEAVDEGGSSDKNIEEFVAMLKQT

SEQ ID NO: 133 - DNA, 1452 bp, synthetic construct
  source 1..1452: mol_type = other DNA
atggaccagatggcaaaaattgatgagaagaaaccccacgttgtgtttataccattcccggcgcaaagtcatatcaagtgtatgctcaaactggcccgcattttgcatcaaaagggattatatattacatttatcaataccgatacgaaccacgaacgacttgtcgcttccgggggcactcaatggttagaaaatgctcctggtttctggtttaaaacagtacctgacggtttcggttctgcaaaagatgatggagttaagccaactgacgccttgagagaactaatggattacctaaaaactaactttttcgatctctttttggacttagtattaaagttagaggtccccgctacctgcataatttgtgatggctgcatgacattcgcgaatacgatcagagctgctgaaaaattgaatataccagttatacttttttggacaatggcagcctgtggttttatggcattttatcaggccaaagtgctgaaggaaaaagagattgttcctctgaaagacgaaacttacttgaccaacggatatttagatatggaaattgattggatcccaggtatgaaacgtattaggcttagagatctacccgagttcattctggccactaagcaaaattatttcgcatttgaatttttatttgaaacagctcaattggcggacaaagtatcgcatatgataatccataccttcgaagaattggaagctagcttagtgtcagaaatcaagtctatatttccgaacgtttacactattggacctttgcagctattgctgaataagataaccctcaaagagactaataatgactcatatagtctatggaaggaagagccagaatgtgttgagtggcttaactctaaggagccaaactccgtcgtgtacgttaacttcggttccttggctgtgatgtctttacaagatctcgtcgaatttgggtggggcttggttaattcaaatcactatttcttatggatcattagagccaatctgattgatggtaagccagctgttatgcctcaagaactcaaagaagccatgaacgagaaagggtttgtaggttcgtggtgttcgcaggaagaggtccttaatcatcctgtggtaggcggtttcttgacgcattgcggttggggaagtatcatagagagcctttcagcaggcgtgcctatgttaggatggccgagcattggggaccagagggcaaactgccggcaaatgtgtaaagaatgggaagtgggcatggagataggtaaaaatgttaagagagatgaagttgaaaaactggttcgtatgttgatggaaggtttggaaggagagcgaatgagaaaaaaggctttagagtggaaaaagtccgctacattagcaacgtgttgtaacgggtcttcatcactggacgtcgaaaaattggccaatgaaattaagaaattatcgcgtaattaa

SEQ ID NO: 134 - protein, 483 aa, Stevia rebaudiana
MDQMAKIDEKKPHVVFIPFPAQSHIKCMLKLARILHQKGLYITFINTDTNHERLVASGGTQWLENAPGFWFKTVPDGFGSAKDDGVKPTDALRELMDYLKTNFFDLFLDLVLKLEVPATCIICDGCMTFANTIRAAEKLNIPVILFWTMAACGFMAFYQAKVLKEKEIVPLKDETYLTNGYLDMEIDWIPGMKRIRLRDLPEFILATKQNYFAFEFLFETAQLADKVSHMIIHTFEELEASLVSEIKSIFPNVYTIGPLQLLLNKITLKETNNDSYSLWKEEPECVEWLNSKEPNSVVYVNFGSLAVMSLQDLVEFGWGLVNSNHYFLWIIRANLIDGKPAVMPQELKEAMNEKGFVGSWCSQEEVLNHPVVGGFLTHCGWGSIIESLSAGVPMLGWPSIGDQRANCRQMCKEWEVGMEIGKNVKRDEVEKLVRMLMEGLEGERMRKKALEWKKSATLATCCNGSSSLDVEKLANEIKKLSRN

SEQ ID NO: 135 - DNA, 1488 bp, synthetic construct
  source 1..1488: mol_type = other DNA
atggtatcagaaactactaaatcttctccattgcattttgtattgttcccatttatggcccaaggtcatatgatcccaatggttgacatcgctagattattggctcaaagaggtgtcatcatcacaatagtcacaactccacataacgctgctagatttaagaatgtgttgaacagagcaattgaatcaggtttgccaatcaatttggtacaagtgaaattcccatatcttgaagcaggtttgcaagagggtcaagaaaacatcgattctttagatacaatggaaagaatgatcccattttttaaagctgtaaacttcttggaagaaccagtacagaaattgattgaggaaatgaatccaagaccatcatgcttgatttccgatttctgtcttccatatacttccaagattgccaaaaagtttaatatcccaaaaatcttattccatggtatgggttgtttttgccttctgtgtatgcatgtcttgagaaaaaatagagaaatactagataatttgaaatctgataaggaattattcactgttccagattttccagatagagttgaatttacaagaacacaagtaccagttgaaacttacgtgccagctggtgattggaaagatatatttgacggtatggtggaagctaacgaaacttcctatggagttattgtaaattcttttcaagaattggaacctgcttatgccaaagattataaggaagtcagaagtggtaaggcctggactattggtccagtatctttatgcaataaagttggtgcagataaggcagaaagaggtaacaaatctgacatagaccaagatgaatgtttaaaatggttggattccaagaaacatggtagtgtgttgtatgtctgccttggttcaatctgcaacttgccattgtcacaattaaaggaattgggtctaggtttggaagaatcacaaagaccattcatttgggtaattagaggttgggaaaaatataaagaattggtggaatggttttcagagtctggatttgaagacagaatacaagacagaggattattaatcaaaggttggtcccctcaaatgttaatattgtctcatcctagtgtcggtggttttttgacccattgtggttggaactctacattagaaggtataactgctggtttgccacttctaacttggccattgtttgctgatcagttctgcaacgaaaaattggttgttgaagtcctaaaagccggtgttagatctggtgtggaacaaccaatgaagtggggtgaagaagaaaagattggtgtgttggtggataaggaaggtgttaagaaggccgttgaggaattgatgggtgaatccgatgacgccaaggagcgccgccgcagagcaaaagaattaggtgatagtgcccataaagctgttgaagaaggtggatcttctcattctaacatatcttttttgttacaagacattatggagttagctgaaccaaacaactaa

SEQ ID NO: 136 - protein, 495 aa, Stevia rebaudiana
MVSETTKSSPLHFVLFPFMAQGHMIPMVDIARLLAQRGVIITIVTTPHNAARFKNVLNRAIESGLPINLVQVKFPYLEAGLQEGQENIDSLDTMERMIPFFKAVNFLEEPVQKLIEEMNPRPSCLISDFCLPYTSKIAKKFNIPKILFHGMGCFCLLCMHVLRKNREILDNLKSDKELFTVPDFPDRVEFTRTQVPVETYVPAGDWKDIFDGMVEANETSYGVIVNSFQELEPAYAKDYKEVRSGKAWTIGPVSLCNKVGADKAERGNKSDIDQDECLKWLDSKKHGSVLYVCLGSICNLPLSQLKELGLGLEESQRPFIWVIRGWEKYKELVEWFSESGFEDRIQDRGLLIKGWSPQMLILSHPSVGGFLTHCGWNSTLEGITAGLPLLTWPLFADQFCNEKLVVEVLKAGVRSGVEQPMKWGEEEKIGVLVDKEGVKKAVEELMGESDDAKERRRRAKELGDSAHKAVEEGGSSHSNISFLLQDIMELAEPNN

SEQ ID NO: 137 - DNA, 1488 bp, synthetic construct
  source 1..1488: mol_type = other DNA
atggccttcgagaaaaacaacgaaccatttccactacattttgtcttgttccctttcatggctcagggtcatatgataccaatggttgatatcgctagattgctggctcagagaggtgttcttataacaatcgttacaaccccacataacgctgctagattcaaaaatgtcttgaatagagcaattgaatctggtttgccaattaacttggtccaagtcaagttcccataccaagaagccggtctgcaagagggacaagagaacatggatttgttgactactatggaacaaattacttcattttttaaggctgtgaacctgttaaaggaaccagtccaaaatttaatagaagaaatgtctccaagaccatcatgccttatatctgatatgtgtttgtcttacacttctgagatcgctaagaagttcaaaatcccaaagatattgttccatggtatgggttgtttctgcttattatgcgtgaatgttttgagaaaaaacagagaaatattagacaacttgaagtctgataaggaatatttcatcgtaccatatttcccagatagagtggaatttactagaccacaagttccagtcgagacttacgtcccagctggttggaaagaaatcttggaagacatggttgaagcagataagacatcatacggtgtaattgtaaactctttccaagaattggaaccagcttacgctaaagacttcaaagaagctagatcaggaaaggcatggactatcggtccagtgtccttatgtaataaggttggtgttgacaaggctgaaagaggtaataaatcagacattgatcaagatgaatgtttggagtggttggattcaaaagagccaggttcagtattatatgtgtgtttgggttctatctgtaaccttcctttatctcaattgttggagctaggtcttggtttagaggaatctcaaagaccatttatttgggtcattagaggttgggaaaaatacaaagagctggtcgaatggttttctgagagtggttttgaagacagaatccaagatagaggtttattaattaagggttggtcccctcaaatgttaattctttctcatccatctgtaggtggtttcttaactcattgcggttggaatagtactttagagggtatcactgctggtttacctatgttaacctggccattatttgctgatcaattttgtaatgagaaattagttgtacaaatactaaaagtaggtgtatctgctgaagtaaaagaggtgatgaaatggggtgaagaagaaaaaattggtgtgctagttgataaagagggcgtcaagaaggccgtcgaggagttgatgggtgaatccgatgatgccaaggaacgccgccgcagagctaaagaattgggtgagtctgctcataaggctgtagaagaaggtggttcttctcattcaaatattacattcctgttgcaagatataatgcaattggcacaatctaataactaa

SEQ ID NO: 138 - protein, 495 aa, Stevia rebaudiana
MAFEKNNEPFPLHFVLFPFMAQGHMIPMVDIARLLAQRGVLITIVTTPHNAARFKNVLNRAIESGLPINLVQVKFPYQEAGLQEGQENMDLLTTMEQITSFFKAVNLLKEPVQNLIEEMSPRPSCLISDMCLSYTSEIAKKFKIPKILFHGMGCFCLLCVNVLRKNREILDNLKSDKEYFIVPYFPDRVEFTRPQVPVETYVPAGWKEILEDMVEADKTSYGVIVNSFQELEPAYAKDFKEARSGKAWTIGPVSLCNKVGVDKAERGNKSDIDQDECLEWLDSKEPGSVLYVCLGSICNLPLSQLLELGLGLEESQRPFIWVIRGWEKYKELVEWFSESGFEDRIQDRGLLIKGWSPQMLILSHPSVGGFLTHCGWNSTLEGITAGLPMLTWPLFADQFCNEKLVVQILKVGVSAEVKEVMKWGEEEKIGVLVDKEGVKKAVEELMGESDDAKERRRRAKELGESAHKAVEEGGSSHSNITFLLQDIMQLAQSNN

SEQ ID NO: 139 - DNA, 1344 bp, synthetic construct
  source 1..1344: mol_type = other DNA
atggaggagcacgggggccgccggcggcccctggtcgtgctcacgtcgtgcccctttcatggacatatgattccaacattacaattagctacaacattacatgcaaaaggatttactattgctattgcacactctaagctcaatccgcctaacccgtccaatcatcctagcgattttatttttcttccactctcagatgacattcccgctatcgataattccggttctttcacagattttatacgaaaacttaataacaactgtaagccatcattcaaggaacacctaacccggctcatatcggagggcaacaaatccatagtcgtggtttacgacaacgtccttcattttgccggtatagtagctgttgacctaaatttagctgccgtaatgttcaggagtagttcagcggcttattttccagcatttttagcccgtcaacaattgagtcaaaggggacgattcctagaagaggactttaaaatggatgagatggtccctaatcattacccaatgcgttacaaggatctccctttctctaagtctccaattgaagattggcggcaattatactcaaatttttcccaacaggcacatccatccgctgtcatctggaatactataaaattccttgaacacgaatctctgacccaagttcacaactactatcaggttcctgtctttgctgttggcccactgcataagatgactcctacgtcctatatcgggtcacatgaggaagataatggttgcattacctggctagacaaacaacctcctaaatctgttgtatacattagctttggaagtttagcaacaatggaagccaaaatattgactgagatggcgttcggtctggccaaatcaaatcagaggttcctttgggcagtgcgtcctggactgatttctggttctgggtgggttgaatttttaccagaaggttttgtagaagaaactcgtggtcgaggtttgatcgtaaaatgggcacctcaaaaagaagtgctagctcattttgcagtaggtggtttctggtcacactgtggttggaacagttgcttggaatctatcagttctggcgtagttatgatgtgtcaaccctttatagcagatcaaggagtcaacgctcgatatgtgagttacgtatggaagataggcctcgaattggagcgtgttgaaagaggtgagatagaaaggatgataaagagagtaatggtagacgacgaaggcgaagaaatgagagtgagagtaaacgacatgaaaaagatggttaaagaagctcttgagattggtggttcgtcacaggaatcctttgaagggttagtagaatttcttttatcttgctaa

SEQ ID NO: 140 - protein, 447 aa, Stevia rebaudiana
MEEHGGRRRPLVVLTSCPFHGHMIPTLQLATTLHAKGFTIAIAHSKLNPPNPSNHPSDFIFLPLSDDIPAIDNSGSFTDFIRKLNNNCKPSFKEHLTRLISEGNKSIVVVYDNVLHFAGIVAVDLNLAAVMFRSSSAAYFPAFLARQQLSQRGRFLEEDFKMDEMVPNHYPMRYKDLPFSKSPIEDWRQLYSNFSQQAHPSAVIWNTIKFLEHESLTQVHNYYQVPVFAVGPLHKMTPTSYIGSHEEDNGCITWLDKQPPKSVVYISFGSLATMEAKILTEMAFGLAKSNQRFLWAVRPGLISGSGWVEFLPEGFVEETRGRGLIVKWAPQKEVLAHFAVGGFWSHCGWNSCLESISSGVVMMCQPFIADQGVNARYVSYVWKIGLELERVERGEIERMIKRVMVDDEGEEMRVRVNDMKKMVKEALEIGGSSQESFEGLVEFLLSC

SEQ ID NO: 141 - DNA, 1425 bp, synthetic construct
  source 1..1425: mol_type = other DNA
atggtacagccgagagttttactgttcccttttcccgcgttggggcatgtgaaaccatttctatccctcgctgagcttttatcggacgccggaatcgatgtcgtttttctaagtacagaatataatcaccggcgtatttcaaacacggaagcacttgctagccgcttcccaactttacatttcgaaaccatacctgatggtttgccacctaatgaatctagagcattggccgacggccccctgtacttttctatgagggagggtactaagccaagattccgacaactcattcaatcactaaacgatggtaggtggcccataacatgcattatcacggatattatgttgtctagtcctatagaggttgctgaagaatttggcattccagtaatcgcattctgtccgtgtagcgctagatatctgtcaattcatttttttattcctaaattggtcgaggaaggacaaataccatacgcggatgacgatcctatcggtgaaatccagggagtgccgttatttgaagggttgcttcgtaggaatcacttaccaggttcctggtccgacaagtctgctgatatttcgttctctcacggtttaatcaaccaaactttagccgcaggaagagctagcgccttaatacttaataccttcgacgagctcgaagctccttttttgacacatttaagttcgatatttaacaaaatttatacgattggcccactgcatgcattgtcaaaaagtcgcctaggtgattccagttcttcagcttcggcgctttccggtttttggaaggaagacagagcctgcatgagctggttagattgtcaacccccccgatcggttgttttcgtgtcgttcggcagcactatgaaaatgaaggcagatgaattaagagagttttggtacggtctagtctcttcggggaaaccatttttatgcgttttacgttcagatgtagtctctggtggggaagcagctgaattgattgaacagatggcagaagaagagggtgctggcggaaagctgggcatggttgtggagtgggctgcgcaagaaaaagtattgtcacatcctgccgttggcggatttcttacacattgtggttggaatagcaccgtcgaaagcatcgcagctggtgttccgatgatgtgttggccaatactcggagatcaaccgtctaatgcaacttggatagacagagtgtggaagatcggcgtagagaggaacaatagagaatgggatcgactaacggtagaaaagatggttcgggccttgatggaaggtcagaaacgtgtcgagattcaacggtccatggaaaaattgtccaaactggctaatgaaaaagtcgttagagggggtttaagtttcgataacttagaggtgctcgttgaggacattaagaaactgaagccttataaattttaa

SEQ ID NO: 142 - protein, 474 aa, Stevia rebaudiana; identical to SEQ ID NO: 9

SEQ ID NO: 143 - DNA, 1029 bp, synthetic construct
  source 1..1029: mol_type = other DNA
atgagtgagttcaggtccaaattcttggaggtttactctgtgttgaaatccgagctcctcaacgaccctgctttcgagttcactgacgattcgcgccaatgggtcgagcggatgttggattacaatgtaccaggaggaaagttgaacagaggactctctgtcattgatagctataagttactaaaacaaggggaaaagctaagtgatgatgaagtgtttctggcatctgcacttggttggtgcattgaatggcttcaagcatattttcttgttcttgatgatatcatggataattctgttacacgccgtggacaaccttgctggttcagggtaccaaaggttggtatggttgcggtaaatgatggcattatgttacggaaccatatcccgagaattctaaaaaatcatttcagagaaaagccttattacgtggatcttttggacttgtttaatgaggtggaatttcaaacagcctcaggacagatgatagatttaattacgacactagaaggagagaaaaatctatcaaagtactcattacctcttcatcatcgcattgttcggtacaaaactgcatattactcgttctatcttccggttgcatgtgcattacttatggcaggcgaaaatttggataatcataatgatgtcaagaatattcttattgatatggggacctatttccaagtgcaggatgattatctggattgtttcggtcatcccgatgtgatagggaagattgggacagatattgaagatttcaagtgctcttggttggttgtgaaagcacttgaacttagtgatgagaaacagaagactttactacatgagaattatggaaaagcagataaagattgtgttgccaaagtgaaagagttgtataaagccctcaatctagagggcgtgtttgctgaatacgagagcaaaagttacgagaagcttataaaatccatagacattcatccaagtaatgcagtacaggcagtgttgaaatctttccttgcaaagatatacaagaggcagaaatag

SEQ ID NO: 144 - protein, 342 aa, Siraitia grosvenorii
MSEFRSKFLEVYSVLKSELLNDPAFEFTDDSRQWVERMLDYNVPGGKLNRGLSVIDSYKLLKQGEKLSDDEVFLASALGWCIEWLQAYFLVLDDIMDNSVTRRGQPCWFRVPKVGMVAVNDGIMLRNHIPRILKNHFREKPYYVDLLDLFNEVEFQTASGQMIDLITTLEGEKNLSKYSLPLHHRIVRYKTAYYSFYLPVACALLMAGENLDNHNDVKNILIDMGTYFQVQDDYLDCFGHPDVIGKIGTDIEDFKCSWLVVKALELSDEKQKTLLHENYGKADKDCVAKVKELYKALNLEGVFAEYESKSYEKLIKSIDIHPSNAVQAVLKSFLAKIYKRQK

SEQ ID NO: 145 - DNA, 1254 bp, synthetic construct
  source 1..1254: mol_type = other DNA
atgggcagcttgggggcgattctgagacacccagatgacttttacccgcttctgaaactgaagatggccgcgaggcatgcggagaagcagatcccgccggagcctcattggggattctgctacaccatgctgcacaaggtatcccggagttttgctctcgttattcagcagcttgcacccgagcttcggaatgctatatgcatattttatcttgttctgcgagccctcgacaccgttgaggatgatacaagtatacaaacagatatcaaagtgcccattctgaaagcttttcactgccacatatataaccgtgattggcatttttcatgtggcacaaaggattataaagttcttatggaccagtttcatcatgtttcaactgcgtttctagaacttgggaaagggtatcaggaggcaattgaggatatcacgaaacggatgggtgcaggaatggctaaattcatttgcaaagaggtagagacagttgatgattacgatgaatattgccactacgtagctggacttgttggactaggtttgtctaagctgttccatgcttcggatttagaggatttggcacctgattcgctttcaaattctatgggattgtttcttcagaaaacaaacattattcgagattacttggaggacatcaacgagataccaaagtctcggatgttttggccccgtgagatttggggcaaatatgccaataagctagaggattttaaatatgaggagaattcggtcaaggccgtgcaatgcctcaacgatttggtcactaatgctttgaatcatgtggaggattgtctgaaatacatgtcaaacttaagagatctttccatatttcgattttgtgcaattcctcagattatggcgattggaactctagcattatgctacaacaacgttgaagtcttcaggggggtggtcaaaatgcggcggggtcttactgctaaggtcattgatcgtacacaaacgatggctgacgtctatggagctttcttcgatttttctgttatgctgaaggctaaggtcaacagcagtgatcctaatgctactaaaaccctgagcaggatcgaagcaatacagaaaacatgcgagcagtcgggactcttgaacaaaaggaaattatatgcagttaaaagtgagccaatgttcaacccgactctgattgtcatacttttcagcctattgtgcatcattctcgcatatctctctgccaaacggttaccggctaaccaacctgtatga

SEQ ID NO: 146 - protein, 417 aa, Siraitia grosvenorii
MGSLGAILRHPDDFYPLLKLKMAARHAEKQIPPEPHWGFCYTMLHKVSRSFALVIQQLAPELRNAICIFYLVLRALDTVEDDTSIQTDIKVPILKAFHCHIYNRDWHFSCGTKDYKVLMDQFHHVSTAFLELGKGYQEAIEDITKRMGAGMAKFICKEVETVDDYDEYCHYVAGLVGLGLSKLFHASDLEDLAPDSLSNSMGLFLQKTNIIRDYLEDINEIPKSRMFWPREIWGKYANKLEDFKYEENSVKAVQCLNDLVTNALNHVEDCLKYMSNLRDLSIFRFCAIPQIMAIGTLALCYNNVEVFRGVVKMRRGLTAKVIDRTQTMADVYGAFFDFSVMLKAKVNSSDPNATKTLSRIEAIQKTCEQSGLLNKRKLYAVKSEPMFNPTLIVILFSLLCIILAYLSAKRLPANQPV

SEQ ID NO: 147 - DNA, 1386 bp, synthetic construct
  source 1..1386: mol_type = other DNA
atggcgaagaacgtagggatattggctatggacatctatttccctcccacctgtgttcaacaggaagctttggaagctcatgatggagcaagcaaagggaaatacaccattggacttggtcaagattgtttagctttttgcactgagcttgaagatgttatctcaatgagtttcaatgcggtgacatctcttcttgaaaagtacaagattgaccccaagcaaattgggcgtcttgaagtaggcagtgagactgtcattgacaagagcaagtccatcaaaaccttcttgatgcaactctttgagaaatgtggaaacactgatgtagaaggtgttgactcgaccaatgcttgttatggtggaactgcggctttgctaaactgtgtcaattgggttgagagtaactcctgggatggacgctatggccttgtcatttgtactgacagtgcggtttatgcagaagggcccgcaaggcccactggaggagcagctgccattgctatgttgataggaccagatgctcctattgtttttgagagcaaattgagaggaagccacatggctaatgtctatgacttttacaagccaaatcttgctagcgagtacccggttgttgatggtaagctttcacagacatgctaccttatggcacttgactcttgctataaacatttatgcaacaagtttgagaaacttgagggcaaagagttctccatcaatgatgctgattactttgtcttccattctccatacaacaaacttgtgcagaaaagctttgctcgtctcttgtacaacgacttcttgagaaacgcaagctccattgatgaggctgccaaagaaaagttcactccttattcctccttgtccctcgatgagagttaccaaagccgtgatcttgaaaaggtgtcacaacaactagcaaaaacgtattatgatgctaaagtgcaaccaacgactttagtaccaaaacaagtcggtaacatgtacactgcttctctctacgcagcatttgcttccctcgtccacaacaaacacagcgatttggcgggaaagcgggtggttatgttctcatatggagctggttcaaccgcaacaatgttctcattgcgtctctgcgagaaccagtctcctttcagcttatcaaacatcgcttctgtaatggatgttggcggtaaattgaaggctagacatgagtatgcacctgagaagtttgtggagacaatgaagctaatggaacataggtacggagcaaaggagtttgtgacgagcaaggaaggcatcttagaccttttggctccggggacttattatctgaaagaggttgattcattgtaccggagattctatggcaagaaaggagacgatggatccataaccaatggacactga

SEQ ID NO: 148 - protein, 461 aa, Brassica juncea
MAKNVGILAMDIYFPPTCVQQEALEAHDGASKGKYTIGLGQDCLAFCTELEDVISMSFNAVTSLLEKYKIDPKQIGRLEVGSETVIDKSKSIKTFLMQLFEKCGNTDVEGVDSTNACYGGTAALLNCVNWVESNSWDGRYGLVICTDSAVYAEGPARPTGGAAAIAMLIGPDAPIVFESKLRGSHMANVYDFYKPNLASEYPVVDGKLSQTCYLMALDSCYKHLCNKFEKLEGKEFSINDADYFVFHSPYNKLVQKSFARLLYNDFLRNASSIDEAAKEKFTPYSSLSLDESYQSRDLEKVSQQLAKTYYDAKVQPTTLVPKQVGNMYTASLYAAFASLVHNKHSDLAGKRVVMFSYGAGSTATMFSLRLCENQSPFSLSNIASVMDVGGKLKARHEYAPEKFVETMKLMEHRYGAKEFVTSKEGILDLLAPGTYYLKEVDSLYRRFYGKKGDDGSITNGH

SEQ ID NO: 149 - DNA, 900 bp, synthetic construct
  source 1..900: mol_type = other DNA
atgacggccagcgctgagcaccagacgttcaccaccgccatcgtgcccgcagccggcctgggcacacgcttcctccccacgaccaaatcggtccccaaagagctgctgccggtcgtggacaccccggcgatcgagctggtcgccgacgaagccaggcaagctggtgccgagcgtctggtcatcgtcacctccccggcgaaacagtccatcgccgcctacttccggcccgcaccggaattggaacggtcactggaggagaaaggcaagaccggccaactggccaagatccgccgggccccggaactgctcgaggtcgaagtcgccatccaggaacaggcactcggcctcggccacgcggtggcgtgcgccgagccgaacctcgggccggaggacgacgtggtggccgtactgctgccggacgacctcgtgctgccccacgggatcctggagcggatggccaaggtacgcgccgagcacggcggcagcgtcctgtgtgctttcgacatcccgaaggaggaaatctccgcctacggagtcttcgacgtcagcgacaccgacgacgccgacgtcaagcgggtccacggcatggtggagaagccgcctgccgagcaagccccctcgacgttcgccgcggccggccgttacctgctcgaccgggcgatcttcgacgcgctgcgccggatcgagccgggcgccgggggcgagctccagctcaccgacgcggtggcgctgttgatccaggaagggcatccggtgcacgtggtggtgcaccgcggtgaccgtcacgacttgggcaaccccggcggcttcctgcgtgccgcggtcgacttcgccctgcaggatccggactatgggccggaactgcgcgcctggttgacggacagaatcgcacgaccatga

SEQ ID NO: 150 - protein, 299 aa, Thermocrispum agreste
MTASAEHQTFTTAIVPAAGLGTRFLPTTKSVPKELLPVVDTPAIELVADEARQAGAERLVIVTSPAKQSIAAYFRPAPELERSLEEKGKTGQLAKIRRAPELLEVEVAIQEQALGLGHAVACAEPNLGPEDDVVAVLLPDDLVLPHGILERMAKVRAEHGGSVLCAFDIPKEEISAYGVFDVSDTDDADVKRVHGMVEKPPAEQAPSTFAAAGRYLLDRAIFDALRRIEPGAGGELQLTDAVALLIQEGHPVHVVVHRGDRHDLGNPGGFLRAAVDFALQDPDYGPELRAWLTDRIARP

SEQ ID NO: 151 - DNA, 942 bp, synthetic construct
  source 1..942: mol_type = other DNA
atgtccacagaaacccattggatttctccatctctaatggcgatttctctcaatcttcttcaacttcaaccaaatccagcgcggcccaagttcttcaaatggaaccccgtccgaccggcggttcatttcagagctccggcgaggttgaccgtcgtcatgtcacagatcactccgtcgtactgggcggccataaatgaagatattgaggatcatctccggcgggtcattccggcaaaggacccgccggcggtgtacgagccgttgggccattttgtcttctcggccccacgaaattccgctcctgcactgtgtattgccgcctgcgagctcgtcggcggcggccgtgaccaggccattgtggcggctactgcactgcaactcatccacgctgcaacctatactcgggaataccctccactgacggcacgtgaaatctcacgcgccgccgtcgttgaccgcgtttatggccccaacatccagctgttgacaggggacggaatagcaccgatggtgttcgagattctagcgggatcttgcgattggagcgagagcgggtcggagcggattcgacgcgtgatggtggaaatggcacgtgcgatgggtacggaagggtggattggggggcaatttcggggattggaggagacggcaacggcggaggaagtgagcgagaagacggaaggtgggctacacgcgtgtgggggcgcgtgtggggtgatattgggaggtggaaatgaagaggaaatagagaagatgaggaaatatgggaaattggtggggatggtgaaaggaatgtcaaaaaatgtgtttggagggaaagaaaatgagagaaagaaagagaaaattgaggagttgaagtatttggctttcaaggaattgaaagatttcaaaggagaaaatgttcatgagatctatggattccttgattttgagggactttag

SEQ ID NO: 152 - protein, 313 aa, Citrullus lanatus
MSTETHWISPSLMAISLNLLQLQPNPARPKFFKWNPVRPAVHFRAPARLTVVMSQITPSYWAAINEDIEDHLRRVIPAKDPPAVYEPLGHFVFSAPRNSAPALCIAACELVGGGRDQAIVAATALQLIHAATYTREYPPLTAREISRAAVVDRVYGPNIQLLTGDGIAPMVFEILAGSCDWSESGSERIRRVMVEMARAMGTEGWIGGQFRGLEETATAEEVSEKTEGGLHACGGACGVILGGGNEEEIEKMRKYGKLVGMVKGMSKNVFGGKENERKKEKIEELKYLAFKELKDFKGENVHEIYGFLDFEGL

SEQ ID NO: 153 - DNA, 942 bp, synthetic construct
  source 1..942: mol_type = other DNA
atggccattaatctctcccatatcaactccaaaacatgtttccctctcaaaacaagatctgatctcagccgttcttcttccgcgcgttgcatgccaactgccgccgctgccgccttccccactatcgccaccgccgcccaaagtcagccgtactgggccgccatcgaggccgacatagagagatacctgaagaaatccatcacaataaggccgccggagacagttttcgggcccatgcaccacctcaccttcgccgccccagccaccgccgcctccaccctatgcttggcggcgtgcgagctcgtcggcggcgaccgaagccaagccatggcagccgcggcggcgatccatctcgtgcacgcggcagcctacgtccacgagcacctccctctaaccgacgggtcgaggcccgtatccaagcccgcaatccagcacaagtacggcccgaacgtcgagctcctcaccggagacgggattgtcccgttcgggtttgagttgctggccgggtcagtggacccggcccgaacagacgacccggataggattctgagagttataatagagatcagtcgggccggcgggccggagggaatgataagcgggctgcatagggaagaagaaattgttgatggaaatacgagtttagacttcattgaatatgtgtgcaagaaaaaatacggcgagatgcatgcttgcggcgcggcttgtggagccatattgggcggcgcagccgaggaggagattcagaagctgaggaatttcgggctttatcaaggaactctcagaggaatgatggaaatgaaaaattctcatcaattaattgatgagaatataattggaaaattgaaagaattggctctcgaggagttgggaggcttccacgggaagaacgctgagctgatgtcgagccttgtagccgagccgagcctttacgcggcttag

SEQ ID NO: 154 - protein, 313 aa, Mentha X piperita
  source 1..313: note = Peppermint (Mentha x piperita)
MAINLSHINSKTCFPLKTRSDLSRSSSARCMPTAAAAAFPTIATAAQSQPYWAAIEADIERYLKKSITIRPPETVFGPMHHLTFAAPATAASTLCLAACELVGGDRSQAMAAAAAIHLVHAAAYVHEHLPLTDGSRPVSKPAIQHKYGPNVELLTGDGIVPFGFELLAGSVDPARTDDPDRILRVIIEISRAGGPEGMISGLHREEEIVDGNTSLDFIEYVCKKKYGEMHACGAACGAILGGAAEEEIQKLRNFGLYQGTLRGMMEMKNSHQLIDENIIGKLKELALEELGGFHGKNAELMSSLVAEPSLYAA

SEQ ID NO: 155 - DNA, 897 bp, synthetic construct
  source 1..897: mol_type = other DNA
atggctgccatcctaaaccatttcacaagcaatccaatacgccctctcctccccctaaacaagctaaaacccctctcatcttctacaaagaccattcaagtgaccactacagacagtaaccccaccaactggtccaccataaacgcagacgtggacgcccgcctcaagcaggccctcccaatccgcgaccccatcgacgtccatcaacctatgcacgatctcgtcttctccgcgcccaagaccttggttccagctctctgcaccgcagcatgcgagctcgtgggaggggagcataaagacggtgtggacgtcgcctcggccctaaacctcatgctggctgcttcctacacccatcaatacctccccaaccttttacagaggccaaatcccccctccttccctcacaacattgagcttctaacaggggatggcatctaccctcttgggttcgagctgctagccagccctgatggtgtggggggtgatgctgagcgcgctctgagggtaattttggaggtcgcacgtgccatgggtgggcaggggttggtagaggcacagtacatgaaggtgaagagggccaagggggaattggacgttgactgcgcgttgatgcgcatgtgcgagagaggagaaggcagtgtttactcatgtgcagcggcatgtggggctattgtaggtggggcctgcgaagaggatgtggaaaagttaaggaggtatgggttttacgtgggtatgatccatgggctgttggatggtgttggcagagaaggcgatgtggaagaagaagttaaaaggcttaggttcttggctctcaaggaattggaagggtttgatgcagagaaggttcgtgccatttccagcctagttgatgctgataccactcgctgtgtacagtag

SEQ ID NO: 156 - protein, 298 aa, Litsea cubeba
MAAILNHFTSNPIRPLLPLNKLKPLSSSTKTIQVTTTDSNPTNWSTINADVDARLKQALPIRDPIDVHQPMHDLVFSAPKTLVPALCTAACELVGGEHKDGVDVASALNLMLAASYTHQYLPNLLQRPNPPSFPHNIELLTGDGIYPLGFELLASPDGVGGDAERALRVILEVARAMGGQGLVEAQYMKVKRAKGELDVDCALMRMCERGEGSVYSCAAACGAIVGGACEEDVEKLRRYGFYVGMIHGLLDGVGREGDVEEEVKRLRFLALKELEGFDAEKVRAISSLVDADTTRCVQ

SEQ ID NO: 157 - DNA, 1272 bp, synthetic construct
  source 1..1272: mol_type = other DNA
atgaagtttattttattgtccacaattttgagtggtcttctctcgtttgctgctgctgctccaatcattttaaagaaacgtgattgggattacacaaaagataaaattcatggtgtgaatattggaggttggcttgttctagagccatacattacaccttcgctatttgagacgtttggtgacgatgcaccagtggatgaatatcactacactcaacagctaggcaaggaagaggcttctaagagattacaagagcactgggctaactggattacagagaaggacttcgaatatattgccaatctgggtcttaacatggttaggattccaattggatactgggcttttgaattgcaggacaatgatccatacgtccagggacagcaagcgtatttggacaaggctcttggctgggcagaaaaaaatggcttgaaagtttggatcgatttgcatggtgttccaggctctcaaaatggatttgataattctggtttgcgtgatcaaatcaactggcaaactggttccggaaatgtcgatttcacattaaacgtgttgaaagaaatgattgagaaatattccgattccgattacagcgacacaattattggaattgagttgttgaacgagcctttaggatcctccttgaacatggatgaacttaaggtcttctacagtagtggctatgaacttgttagagatcaggatgcttccattccagtcattattcaagatgctttccagtccgattactactgggatgactttttaaatactgagcaagacacaaacatttacggcgttgttgttgatcatcatcactaccaggtcttttctactggtgaacttcagagagacatggatacacgcattcaaaccgcatgtaactggggtaagcaggaaggtggggagtaccactggaatgtctgcggagagttctcagcagcccttacggattgtgccaagtggctcaatggtttaggaaggggtgccagatatgactcttcatatggaggtggtgcttatgttggaagttgtgatgatttgtacacgtacgatgaagactactggaacaatagtgatgtcatcaagcaatacaggcagtatgtggaggcccaaatggatgcatttgagaatggaaagatgggaggatgggttttctggtgttggaagacagaatccgctgttgagtgggacttccagaggttggctggccttaacgtcattcctcagcctttggatgacaggttatatcctaaccagtgtagcttctaa

SEQ ID NO: 158 - protein, 423 aa, Dekkera bruxellensis
MKFILLSTILSGLLSFAAAAPIILKKRDWDYTKDKIHGVNIGGWLVLEPYITPSLFETFGDDAPVDEYHYTQQLGKEEASKRLQEHWANWITEKDFEYIANLGLNMVRIPIGYWAFELQDNDPYVQGQQAYLDKALGWAEKNGLKVWIDLHGVPGSQNGFDNSGLRDQINWQTGSGNVDFTLNVLKEMIEKYSDSDYSDTIIGIELLNEPLGSSLNMDELKVFYSSGYELVRDQDASIPVIIQDAFQSDYYWDDFLNTEQDTNIYGVVVDHHHYQVFSTGELQRDMDTRIQTACNWGKQEGGEYHWNVCGEFSAALTDCAKWLNGLGRGARYDSSYGGGAYVGSCDDLYTYDEDYWNNSDVIKQYRQYVEAQMDAFENGKMGGWVFWCWKTESAVEWDFQRLAGLNVIPQPLDDRLYPNQCSF

SEQ ID NO: 159 - DNA, 1524 bp, synthetic construct
  source 1..1524: mol_type = other DNA
atggctgtggttgcttcagctccagggaaagttttaatggccggaggttaccttgtcttggagaaaccgaatgcagggctcgtacttagtactaatgctcgtttttatgcaattgtaaaacccgtttacgaggaaatcaagcctgacagctgggcatggtcatggacagatgttaagttgacatctccccagctcctgagagagagcatctacaaattgtcattgaaaaaccaatctcttcagagtatctctccaagtcaatcagggaacccttttgtagaaagagcagtggaatattgtgtagcagctgcatgtgcaaaatttgttgacaaggacaataaggatgcattacacaaactgctcttgcaaggtcttgatatcactattctaggttgcaatgagttctactcttatcggagtcagattgaggctcgtggacttccactaacatcagaatcattggcttccttgcctcctttttcatcaattacattcaatgatgaggaatcatatgggaaaacttgcaagcctgaagttgcaaaaactggattgggctcatctgcagcaatgacagctgctgtagttgctgctttacttaactacctaggggttgtaaatctttccttattgacaggtgaccaagaagagagaggcagtgcggatcttgatttggtgcatataatagctcaaagtgcccactgcattgcacagggaaaagttggcagtggctttgatgtcagttctgcagtttatggcagtcaacgctatgttcggttttcaccagaagtgctgtcttctgctcaggctgccaagaatggagtacccatagaagaggtcattggcgatatcttaaatcagaagtgggaccatgagaggactaagttctctttgcctccattaatgactcttgtacttggggagccaggagtgggagggtcatccacgccttcaatggtaggtgctgtcaaaaagtggcaaaaatctgaccctcaagggtccctagacacgtggacaaagttgtcagaagggaactcagaacttgaaactcaactcaacacgttgagtaaacttgcagctgaacactgggatgcttataaatatatcataaacagttgttccaagctcaccttggagaagtggattcaccaagccacagaaccaagccaccaagccatcatcaaagcattattaggagcaagaaatgctatgcttcagatcaggcatcatatgcgcttaatgggtgaagctgctggtgttccgattgagccagattcacaaaccaagcttttagatactaccatgaatatggaaggagtcctgttagccggtgttccaggggcaggcgggtttgacgctgtttttgctgtgaccttgggagactcaaccagcaatgtgattaaatcatggagttcactggatattttggctctccttgttagagaagatccccgtggtgttgctttggaaagttgtgatccccgaaccaaggcaattacatcagctgtttcatcagttcatatttag

SEQ ID NO: 160 - protein, 507 aa, Citrullus lanatus
MAVVASAPGKVLMAGGYLVLEKPNAGLVLSTNARFYAIVKPVYEEIKPDSWAWSWTDVKLTSPQLLRESIYKLSLKNQSLQSISPSQSGNPFVERAVEYCVAAACAKFVDKDNKDALHKLLLQGLDITILGCNEFYSYRSQIEARGLPLTSESLASLPPFSSITFNDEESYGKTCKPEVAKTGLGSSAAMTAAVVAALLNYLGVVNLSLLTGDQEERGSADLDLVHIIAQSAHCIAQGKVGSGFDVSSAVYGSQRYVRFSPEVLSSAQAAKNGVPIEEVIGDILNQKWDHERTKFSLPPLMTLVLGEPGVGGSSTPSMVGAVKKWQKSDPQGSLDTWTKLSEGNSELETQLNTLSKLAAEHWDAYKYIINSCSKLTLEKWIHQATEPSHQAIIKALLGARNAMLQIRHHMRLMGEAAGVPIEPDSQTKLLDTTMNMEGVLLAGVPGAGGFDAVFAVTLGDSTSNVIKSWSSLDILALLVREDPRGVALESCDPRTKAITSAVSSVHI

SEQ ID NO: 161 - DNA, 1497 bp, synthetic construct
  source 1..1497: mol_type = other DNA
atggccctagttgcctcagctcctgggaaagtattattgacaggaggatacctaattttggagaggccaaatgctggtttggtactcagtacaaatgctcggttttatgcaattgtgaaaccacttcacgagcaaactgagccagaaagtggggatggggaatgggtagatgtgaagttgacctcaccacaactttctcgagaaagtctgtataaattatcgcccgagaacctcatgattcagtgcatctccttgagtgagtcaaggaaccctttcatagaacatgctgtgcagtattccctagctgctgcacatggaatatttgtcaagaacaagaaagattcactgcacaggttgctactacaaggtcttgatatcacaatcttaggatgcaatgatttctactcttatcggaatcagattgaagcacatggacttcctttgacaccagaatcactggtttcttttccaccatttgcatcaattaccttcaatgctgagccatcaaatagtgaaaattgcaagcctgaagttgcaaagacaggattgggatcatctgctgcaatgacaactgctgttgttgctgccctacttaattatcttggagttgtaaatctttcctcatcaagaggtcaagagaaagaaaatgatatgaaggatcttgatatggtgcatgcaatagcccaaactgctcattgcattgcacagggaaaagttggaagtggatttgatgtcagctctgccgtctatggcagccaacggtatgttcgtttctcacctcacgtgctggccactgctcaggcagctgttaaaggaccgctagaagaggtcattgtcgatatcttaaaaggaaagtgggaccatgagaggactgagttttccttgcctccattaatgaatcttttattaggggaaccaggagctggaggatcatccacaccatctatggtaggtgctgttaaaaagtggcaaaagtctgaccctcagaaatctcaagaaacatggaataagttgtcgaaagcaaatattgatttcgagaagcaattaaatttactgagaaaacttgcagcagaacaatgggatgtgtataaatgcatcataagtaaatgcagcacacttggatcaaagaagtggatagaacaagccactgaaccaagccaacaagcaattgttaaagcattgttaggagcaagagatgccatgctcgaaatacgaaattgtatgaagcaaatgggcgaagctgcaggcgttccaattgaaccaaagccacaaactgaacttttagatgcaaccatggaattggaaggagttttgttggctggagttccaggggcaggcgggtttgatgctgtatttgctattacattgggtgattctagcagccatgtgattaagtcatggacttcactcaatgttcttgccctcttagttagagaagatcccgatggagttgctcttgaaagtgatgatcctagaggtaagagctgtactgtttaa

SEQ ID NO: 162 - protein, 498 aa, Citrullus lanatus
MALVASAPGKVLLTGGYLILERPNAGLVLSTNARFYAIVKPLHEQTEPESGDGEWVDVKLTSPQLSRESLYKLSPENLMIQCISLSESRNPFIEHAVQYSLAAAHGIFVKNKKDSLHRLLLQGLDITILGCNDFYSYRNQIEAHGLPLTPESLVSFPPFASITFNAEPSNSENCKPEVAKTGLGSSAAMTTAVVAALLNYLGVVNLSSSRGQEKENDMKDLDMVHAIAQTAHCIAQGKVGSGFDVSSAVYGSQRYVRFSPHVLATAQAAVKGPLEEVIVDILKGKWDHERTEFSLPPLMNLLLGEPGAGGSSTPSMVGAVKKWQKSDPQKSQETWNKLSKANIDFEKQLNLLRKLAAEQWDVYKCIISKCSTLGSKKWIEQATEPSQQAIVKALLGARDAMLEIRNCMKQMGEAAGVPIEPKPQTELLDATMELEGVLLAGVPGAGGFDAVFAITLGDSSSHVIKSWTSLNVLALLVREDPDGVALESDDPRGKSCTV

SEQ ID NO: 163 - DNA, 975 bp, synthetic construct
  source 1..975: mol_type = other DNA
atggatctaaccaagccactccgctgcatcgtaaaattggggggtgcggccattacttgcaaaaatgaattggaaaccatacacgaagaaaatttggcgacggtttcgtcacatctgaggaaaactatggttagtggctcttcttctgaaaatacctttggaatggattggagcaggcagcctggaaaatccgacattgcatgtaatgttgatgatttaagagaacatgaagttggccttgcgagtccctttatagttgttcatggagctggttcttttgggcattttcaggcaagtaaatcaggggttcataaaggaggacttgatagatccttagtcaaggctggatttgttgccacacgtatatccgttacttcactcaatctccaaattgttagagcactcgcgagagagggtattccatccgttggaatgtctccattttcatgtggatggtccacttgtgaaagaaatatggatgcagttgatttacgtggtgtttctaaagctattaactccggatttgttcctgttctgcatggagatgctgtttttgacaattccctggggtgcaccatattaagtggagatgtaataatacgacatcttgcagagcaactaaagcctgattatgttgtattccttacagatgttttaggtgtatatgatcgtcctccaacagaacctaatgcagtccttttaagagagattgctgtgggtgaagatggtcgttggtctgttatacaacctatactgcacaacggtaacacagaagttgagataagtgtagccgctcatgatacaactgggggaatggtgactaagatatcagaagcagcaatgattgcaaagcttggcattgaagtctacattgtaaaggcagcaacagtccactccttgagagctctaagtggggaaatccgaggcaaaattccagatgactggcttggaacggtcattagattttcaggataa

SEQ ID NO: 164 - protein, 324 aa, Citrullus lanatus
MDLTKPLRCIVKLGGAAITCKNELETIHEENLATVSSHLRKTMVSGSSSENTFGMDWSRQPGKSDIACNVDDLREHEVGLASPFIVVHGAGSFGHFQASKSGVHKGGLDRSLVKAGFVATRISVTSLNLQIVRALAREGIPSVGMSPFSCGWSTCERNMDAVDLRGVSKAINSGFVPVLHGDAVFDNSLGCTILSGDVIIRHLAEQLKPDYVVFLTDVLGVYDRPPTEPNAVLLREIAVGEDGRWSVIQPILHNGNTEVEISVAAHDTTGGMVTKISEAAMIAKLGIEVYIVKAATVHSLRALSGEIRGKIPDDWLGTVIRFSG

SEQ ID NO: 165 - DNA, 1275 bp, synthetic construct
  source 1..1275: mol_type = other DNA
atggcgatttctagagaattggcgggtgaaaaatgggtgttgttgaccactgcacaaacgccgacgaacattgcggtgattaagtattgggggaagagggacgaggccttaattttacctgtcaatgatagcatcagtgttacgcttgatccgagtcatctctgcaccatcaccaccgtcgctgttagtcccgactttgagaaggatcggatgtggctcaatcgcaaggagatatccctttctggagccaggtaccaaacttgtttgagggaaatacgcagtcgagctaatgatgttgaggataaagaaaaggggattaagatagcaaaaaaagactgggagaagttgcatgtatatattgattcatacaataatttcccaactgctgctggcttggcttcctctgctgccggttttgcttgtcttgtctttgctttggcaaatttaatgaacgtcaaggaagatcatagccagctatctgctattgcaaggcaaggttcgggaagtgcatgccgtagtctatacggtggatttgtgaagtggagcatgggaaaagaaaaggatggaagtgatagtcttgcaattcaacttgcggatgagaagcactgggacgatcttgtaattattattgctgtggtaagttcgcgacagaaggaaacaagtagtacatcaggaatgcgggaaaccgtagaaacaagtttgcttttacaacatagagctaaggaaattgtaccaaagcgtgtgttagctatggaagaagctattaaaaatcgggattttgcatcctttgcacaactgacatgcaacgatagcaaccaatttcatgcagtctgtctcgatacgtctcccccgatattttacatgaatgatacgtcccacaggataattagtcttgttgagaaatggaaccgttctgaaggagaacctcaggtggcatatactttcgatgcagggccgaattcggttctgattgcacgaaacagaaaaattgctgtttctttgcttcagaggttgcttttccaattccctccaaacccagaaacggaattaaaaagttatgttcttggcgacaagtcaattcttcaagatgctgggattaatagtgttgaggatattgaatccctgccacaacctccagaatttagcagttcattccagaaataccagggagatgtcagttacttcatctgtaccagacccggaaaagggccagtcattctccccgagagtgaagctctactcgacccgaaaactgggctgccaaagaacctctag

SEQ ID NO: 166 - protein, 424 aa, Citrullus lanatus
MAISRELAGEKWVLLTTAQTPTNIAVIKYWGKRDEALILPVNDSISVTLDPSHLCTITTVAVSPDFEKDRMWLNRKEISLSGARYQTCLREIRSRANDVEDKEKGIKIAKKDWEKLHVYIDSYNNFPTAAGLASSAAGFACLVFALANLMNVKEDHSQLSAIARQGSGSACRSLYGGFVKWSMGKEKDGSDSLAIQLADEKHWDDLVIIIAVVSSRQKETSSTSGMRETVETSLLLQHRAKEIVPKRVLAMEEAIKNRDFASFAQLTCNDSNQFHAVCLDTSPPIFYMNDTSHRIISLVEKWNRSEGEPQVAYTFDAGPNSVLIARNRKIAVSLLQRLLFQFPPNPETELKSYVLGDKSILQDAGINSVEDIESLPQPPEFSSSFQKYQGDVSYFICTRPGKGPVILPESEALLDPKTGLPKNL

SEQ ID NO: 167 - DNA, 1029 bp, synthetic construct
  source 1..1029: mol_type = other DNA
atggcggatctcaagtcaaccttcctcaaggtctactcggttctgaaagaagagcttctccaggaccctgccttcgagtttactgacgcttctcgccaatggatcgaacggatgctggactacaatgtgcctggaggaaagcttaaccgagggctctccgtaattgatagctacagattgttgaaagaagggaaggaattgacggaagatgaaatctttcttgccagtgttcttggatggtgtattgaatggctccaggcatattttcttgttcttgatgatattatggataactccgtcacacgacgcggtcaaccctgttggtttagagtgcccaaggttgggatgattgcagtaaatgatggagttctacttcgaaatcatattcctagaattctcaagaatcacttcaaggggaagccatattacgtggatctgcttgatttgtttaacgaggtcgagtttcaaacagcatcaggacaaatgatagatttgataaccactattgaaggagaaaaagatctttccaaatacgcgttgtcaattcatcggcgaattgtacagtacaagactgcctattattcattttaccttccagtcgcatgtgcactagttatatcaggtgaagatcttgacaaacatactgtggttaaggacattcttgtccagatgggtgtctatttccaagttcaggatgactatctagattgttttggcgatcctgaaaccatcggaaaggtgggaacagacattgaagatttcaaatgttcttggttggttgtgaaggctttggaactttgcaacgaggaacagaataaattattacatgaggcttatgggaaaccagacccagaaaatgttgccaaagtaaaggccctctacaaggagcttgatcttcagggtgtatttgcgaaatatgagagccagagctacgctaaactgaatgcctccattgaagctcatcccagcaaatcaattcaagcagttctcaagtctttcttgggaaaaatatacaagcgccagaagtga

SEQ ID NO: 168 - protein, 342 aa, Siraitia grosvenorii
MADLKSTFLKVYSVLKEELLQDPAFEFTDASRQWIERMLDYNVPGGKLNRGLSVIDSYRLLKEGKELTEDEIFLASVLGWCIEWLQAYFLVLDDIMDNSVTRRGQPCWFRVPKVGMIAVNDGVLLRNHIPRILKNHFKGKPYYVDLLDLFNEVEFQTASGQMIDLITTIEGEKDLSKYALSIHRRIVQYKTAYYSFYLPVACALVISGEDLDKHTVVKDILVQMGVYFQVQDDYLDCFGDPETIGKVGTDIEDFKCSWLVVKALELCNEEQNKLLHEAYGKPDPENVAKVKALYKELDLQGVFAKYESQSYAKLNASIEAHPSKSIQAVLKSFLGKIYKRQK

SEQ ID NO: 169 - DNA, 1254 bp, synthetic construct
  source 1..1254: mol_type = other DNA
atgggcagcttgggggcgattctgagacacccagatgacttttacccgcttctgaaactgaagatggccgcgaggcatgcggagaagcagatcccgccggagccccattggggattctgctacaccatgctgcacaaggtatcccggagttttgctctcgttattcagcagcttgcacccgagcttcggaatgctatatgcatattttatcttgttctgcgagccctcgacaccgttgaggatgatacaagtatacaaacagatatcaaagtgcccattctgaaagcttttcactgccacatatataaccgtgattggcatttttcatgtggcacaaaggattataaagttcttatggaccagtttcatcatgtttcaactgcgtttctagaacttgggaaagggtatcaggaggcaattgaggatatcacgaaacggatgggggcaggaatggctaaattcatttgcaaagaggtagagacagttgatgattacgatgaatattgccactacgtagctggacttgttggactaggtttgtctaagctgttccatgcttcggatttagaggatttggcacctgattctctttcaaattctatgggattgtttcttcagaaaacaaacattattcgagattacttggaggacatcaacgagataccaaagtctcggatgttttggccccgtgagatttggggcaaatatgccgataaactagaggattttaaatatgaggagaattcggtcaaggccgtgcaatgcctcaacgatttggtcactaatgctttgaatcatgtggaggattgtctgaaatacatgtcaaacttaagagatctttccatatttcgattttgtgcaattcctcagattatggcgattggaactctagcattatgctacaacaacgttgaagttttcaggggggtggtcaaaatgcggcggggtcttactgctaaggtcattgatcgtacacaaacgatggctgacgtctatggagctttcttcgatttttctgttatgctgaaggctaaggtcaacagcagtgatcctaatgctactaaaaccctgagcaggatcgaagcaatacagaaaacatgcgagcagtcgggactcttgaacaaaaggaaattatatgcagttaaaagtgagccaatgttcaacccgactctgattgtcatacttttcagcctattgtgcatcattctcgcatatctctctgccaaacggttaccggctaaccaacctgtatga

SEQ ID NO: 170 - protein, 417 aa, Siraitia grosvenorii
MGSLGAILRHPDDFYPLLKLKMAARHAEKQIPPEPHWGFCYTMLHKVSRSFALVIQQLAPELRNAICIFYLVLRALDTVEDDTSIQTDIKVPILKAFHCHIYNRDWHFSCGTKDYKVLMDQFHHVSTAFLELGKGYQEAIEDITKRMGAGMAKFICKEVETVDDYDEYCHYVAGLVGLGLSKLFHASDLEDLAPDSLSNSMGLFLQKTNIIRDYLEDINEIPKSRMFWPREIWGKYADKLEDFKYEENSVKAVQCLNDLVTNALNHVEDCLKYMSNLRDLSIFRFCAIPQIMAIGTLALCYNNVEVFRGVVKMRRGLTAKVIDRTQTMADVYGAFFDFSVMLKAKVNSSDPNATKTLSRIEAIQKTCEQSGLLNKRKLYAVKSEPMFNPTLIVILFSLLCIILAYLSAKRLPANQPV

SEQ ID NO: 171 - DNA, 1503 bp, synthetic construct
  source 1..1503: mol_type = other DNA
atggccgtagttgcttcagctcctgggaaagtcctattgacgggaggatacctgattttggagaggccaaatgctggtttggtactcagtacaaatgctcggttttatgcaattgtgaaaccacttcacgagcataccgagccagaaagtgggggtgggaaatgggtagatgtgaaattgacctcaccacagctctgccgtgaaagtctatataagttgtcaccagagaactttacgattcagtgtatctcctcgattgagttgaggaaccctttcgtggaacatgcagttcaatattctgtagccgccgcacatggaatatttgacaagaacaagaaagattcactgcacagattgcttctgcaaggcctcgatatcacaattttaggttgcaatgatttctactcttatcggaatcagattgaagcacgtggacttcctttgacaccagaatcactgatttcttttccaccatttgcatcaattaccttcaatgctgaagaatcaaatagtgaaaattgcaagcctgaagttgcgaagactggattgggatcttctgctgcaatgacaactgctgttgttgctgccctacttaattatcttggagttgtaaatctttcctcaccaagaggtcaagacaaagaaaatgatattaaggatcttgatatggtgcatgtaatagcccaaactgctcattgcattgcacaggggaaagttggaagtggatttgatgtctgttctgcagtctatggcagccaacgttatgttcgtttttcacctgaagtgcttgctactgctcaggtagcagttaaaggaccactagaagaggtcattgtcaatatcctaaaaggaaagtgggaccatgagaggactgagttttccttgcctccattaatgaatctcttattaggggaaccaggagcaggaggatcatcgacaccatctatggtaggtgctgttaaaaagtggcaaaagtctgaccctcagaaatctcaagaaacatggaataagttgtcggaagcaaatttgggcctcgaaaaacaactaaacttactaagaaaacttgcagcagaacactgggatgtatataaatgcatcataagcggatgcagcatacttggatcaaagaagtggatggaacaagccactgaacaaagccaacaagccatcgtcaaagcattgttaggagcaagagacgccatgctcgagatacggaatcatatgcggcaaatgggcgaagctgccggtgttccaattgagccaaagtcacaaactcagcttctggatgcaaccatggaattggaaggagtcctgttggctggagttccaggggccggtgggtttgatgctgtattcgctattaccttgggtgattctagcaggcatgtgattgagtcatggacttcactcaatgttcttgccctcttagttagagaagatcccaatggagttgctctagaaagtggtgatcctagaagtaagaatattgcaccttctgtttaa

SEQ ID NO: 172 - protein, 500 aa, Siraitia grosvenorii
MAVVASAPGKVLLTGGYLILERPNAGLVLSTNARFYAIVKPLHEHTEPESGGGKWVDVKLTSPQLCRESLYKLSPENFTIQCISSIELRNPFVEHAVQYSVAAAHGIFDKNKKDSLHRLLLQGLDITILGCNDFYSYRNQIEARGLPLTPESLISFPPFASITFNAEESNSENCKPEVAKTGLGSSAAMTTAVVAALLNYLGVVNLSSPRGQDKENDIKDLDMVHVIAQTAHCIAQGKVGSGFDVCSAVYGSQRYVRFSPEVLATAQVAVKGPLEEVIVNILKGKWDHERTEFSLPPLMNLLLGEPGAGGSSTPSMVGAVKKWQKSDPQKSQETWNKLSEANLGLEKQLNLLRKLAAEHWDVYKCIISGCSILGSKKWMEQATEQSQQAIVKALLGARDAMLEIRNHMRQMGEAAGVPIEPKSQTQLLDATMELEGVLLAGVPGAGGFDAVFAITLGDSSRHVIESWTSLNVLALLVREDPNGVALESGDPRSKNIAPSV

SEQ ID NO: 173 - DNA, 1275 bp, synthetic construct
  source 1..1275: mol_type = other DNA
atggccccggagaagatgccggaggaggacgaggagatcgtcgccggggtggtcgcgggcaagatcccctcctacgtcctggaaacccggctgggcgactgccggcgggcggcggggatccgccgcgaggcgctccggcggatcaccgggcgggagatcgacggcctgcccctggacgggttcgactacgacagcatcctggggcagtgctgcgagatgccggtgggctacgtccagctgccggtgggcgtggccggcccgctcgtgctggacggccgccgcatctacgtgccgatggccacgaccgaggggtgcctgatcgcgagcacgaaccgcggctgcaaggcgatcgcggagagcgggggcgcgtccagcgtcgtctaccgggacggcatgacccgggcgccggtcgccaggttcccctccgcccggcgcgccgccgagctgaagggcttcctggagaaccccgccaactacgacaccctgtccgtggtcttcaaccggtcctcccgcttcgcgaggctgcagggcgtgaagtgcgcgatggcggggcggaacctgtacatgcgcttcacgtgctccaccggcgacgcgatgggcatgaacatggtgagcaagggggtgcagaacgtgctcgactacctccaggaggacttcccggacatggacgtcgtgtccatctccggcaacttctgctccgacaagaagtccgcggccgtgaactggatcgaggggcgcgggaagagcgtcgtctgcgaggcggtgatccgggaggaggtggtgcacaaggtgctgaagaccaacgtgcagtccctggtggagctgaacgtgatcaagaacctggcgggcagcgccgtggcgggggcgctgggcgggttcaacgcccacgcctccaacatcgtgacggcgatcttcatcgcgacgggccaggacccggcgcagaacgtggagtcctcccagtgcatcaccatgctcgaggcggtcaacgacgggagggacctgcacatcagcgtgacgatgccgagcatcgaggtggggaccgtcggcggcgggacccagctcgcctcccagtccgcgtgcctcgacctgctcggggtgaagggggccaaccgcgaatccccgggatctaacgcacgccttctggccaccgtggtcgccggcgcggtgctggcgggggagctctccctgatcagcgcccaggccgcgggccacctggtccagtcccacatgaagtacaacaggtcctccaaggacatgtccaagatcgcctgctga

SEQ ID NO: 174 - protein, 424 aa, Avena strigosa
MAPEKMPEEDEEIVAGVVAGKIPSYVLETRLGDCRRAAGIRREALRRITGREIDGLPLDGFDYDSILGQCCEMPVGYVQLPVGVAGPLVLDGRRIYVPMATTEGCLIASTNRGCKAIAESGGASSVVYRDGMTRAPVARFPSARRAAELKGFLENPANYDTLSVVFNRSSRFARLQGVKCAMAGRNLYMRFTCSTGDAMGMNMVSKGVQNVLDYLQEDFPDMDVVSISGNFCSDKKSAAVNWIEGRGKSVVCEAVIREEVVHKVLKTNVQSLVELNVIKNLAGSAVAGALGGFNAHASNIVTAIFIATGQDPAQNVESSQCITMLEAVNDGRDLHISVTMPSIEVGTVGGGTQLASQSACLDLLGVKGANRESPGSNARLLATVVAGAVLAGELSLISAQAAGHLVQSHMKYNRSSKDMSKIAC

SEQ ID NO: 175 - DNA, 1572 bp, synthetic construct
  source 1..1572: mol_type = other DNA
atggaggacaggaacgccatggacatgagcaggatcaagtacaggccgcagccgctgaggccggccagcatggtgcagccgagggtgctgctgttcccgttcccggccctgggccacgtgaagccgttcctgagcctggccgagctgctgagcgacgccggcatcgacgtggtgttcctgagcaccgagtacaaccacaggaggatcagcaacaccgaggccctggccagcaggttcccgaccctgcacttcgaaaccatcccggacggcctgccgccgaacgagagcagggccctggccgacggcccgctgtacttcagcatgagggagggcaccaagccgaggttcaggcagctgatccagagcctgaacgacggtaggtggccgatcacctgtatcatcaccgacatcatgctgagcagcccgatcgaggtggccgaggagttcggcatcccggtgatcgccttctgcccgtgcagcgccaggtacctgagcatccacttcttcatcccgaagctggtggaggagggccagatcccgtacgccgacgacgacccgatcggcgagatccagggcgtgccgctgttcgagggcctgctgaggaggaaccatctgccgggcagctggagcgacaagagcgccgacatcagcttcagccacggcctgatcaaccagaccctggccgccggcagggccagcgccctgatcctgaacaccttcgacgagctggaggccccgttcctgacccacctgagcagcatcttcaacaagatctacaccatcggcccgctgcacgccctgagcaagagcaggctgggcgacagcagcagcagcgccagcgccctgagcggcttctggaaggaggacagggcctgcatgagctggctggactgccagccgccgaggagcgtggtgttcgtgagcttcggcagcaccatgaagatgaaggccgacgagctgagggagttctggtacggcctggtgagcagcggcaagccgttcctgtgcgtgctgaggagcgacgtggtgagcggcggcgaggccgccgagctgatcgagcagatggccgaggaggagggcgccggcggcaagctgggcatggtggtggagtgggccgcccaggagaaggtgctgagccacccggccgtgggcggcttcctgacccactgcggctggaacagcaccgtggagagcatcgccgccggcgtgccgatgatgtgctggccgatcctgggcgaccagccgagcaacgccacctggatcgacagggtgtggaagatcggcgtggagaggaacaacagggagtgggacaggctgaccgtggagaagatggtgagggccctgatggagggccagaagagggtggagatccagaggagcatggagaagctgagcaagctggccaacgagaaggtggtgaggggcatcaacctgcacccgaccatcagcctgaagaaggacaccccgaccaccagcgagcacccgaggcacgagttcgagaacatgaggggcatgaactacgagatgctggtgggcaacgccatcaagagcccgaccctgaccaagaagtaa

SEQ ID NO: 176 - protein, 523 aa, Siraitia grosvenorii
MEDRNAMDMSRIKYRPQPLRPASMVQPRVLLFPFPALGHVKPFLSLAELLSDAGIDVVFLSTEYNHRRISNTEALASRFPTLHFETIPDGLPPNESRALADGPLYFSMREGTKPRFRQLIQSLNDGRWPITCIITDIMLSSPIEVAEEFGIPVIAFCPCSARYLSIHFFIPKLVEEGQIPYADDDPIGEIQGVPLFEGLLRRNHLPGSWSDKSADISFSHGLINQTLAAGRASALILNTFDELEAPFLTHLSSIFNKIYTIGPLHALSKSRLGDSSSSASALSGFWKEDRACMSWLDCQPPRSVVFVSFGSTMKMKADELREFWYGLVSSGKPFLCVLRSDVVSGGEAAELIEQMAEEEGAGGKLGMVVEWAAQEKVLSHPAVGGFLTHCGWNSTVESIAAGVPMMCWPILGDQPSNATWIDRVWKIGVERNNREWDRLTVEKMVRALMEGQKRVEIQRSMEKLSKLANEKVVRGINLHPTISLKKDTPTTSEHPRHEFENMRGMNYEMLVGNAIKSPTLTKK

SEQ ID NO: 177 - DNA, 1458 bp, synthetic construct
  source 1..1458: mol_type = other DNA
atggccgagcaggcccacgacctgctgcacgtgctgctgttcccgttcccggccgagggccacatcaagccgttcctgtgcctggccgagctgctgtgcaacgccggcttccacgtgaccttcctgaacaccgactacaaccacaggaggctgcacaacctgcacctactggccgccaggttcccgagcctgcacttcgagagcatcagcgacggcctgccgccggaccagccgagggacatcctggacccgaagttcttcatcagcatctgccaggtgaccaagccgctgttcagggagctgctgctgagctacaagaggatcagcagcgtgcagaccggcaggccgccgatcacatgcgtgatcaccgacgtgatcttcaggttcccgatcgacgtggccgaggagctggacatcccggtgttcagcttctgcaccttcagcgccaggttcatgttcctgtacttctggatcccgaagctgatcgaggacggccagctgccgtacccgaacggcaacatcaaccagaagctgtacggcgtggccccggaggccgagggcctgctgaggtgcaaggacctgccgggccactgggccttcgccgacgagctgaaggacgaccagctgaacttcgtggaccagaccaccgccagcagcaggagcagcggcctgatcctgaacaccttcgacgacctggaggccccgttcctgggcaggctgagcaccatcttcaagaagatctacgccgtgggcccgatccacagcctgctgaacagccaccactgcggcctgtggaaggaggaccacagctgcctggcctggctggacagcagggccgccaagagcgtggtgttcgtgagcttcggcagcctggtgaagatcaccagcaggcagctgatggagttctggcacggcctgctgaacagcggcaagagcttcctgttcgtgctgaggagcgacgtggtggagggcgacgacgagaagcaagtggtgaaggagatctacgaaaccaaggccgagggcaagtggctggtggtgggctgggccccgcaggagaaggtgctggcccacgaggccgtgggcggcttcctgacccacagcggctggaacagcatcctggagagcatcgccgccggcgtgccgatgatcagctgcccgaagatcggcgaccagagcagcaactgcacctggatcagcaaggtgtggaagatcggcctggagatggaggacaggtacgacagggtgagcgtggaaaccatggtgaggagcatcatggagcaggagggcgagaagatgcagaagaccatcgccgagctggccaagcaggccaagtacaaggtgagcaaggacggcaccagctaccagaacctggagtgcctgatccaggacatcaagaagctgaaccagatcgagggcttcatcaacaacccgaacttcagcgacctgctgagggtgtaa

SEQ ID NO: 178 - protein, 485 aa, Siraitia grosvenorii
MAEQAHDLLHVLLFPFPAEGHIKPFLCLAELLCNAGFHVTFLNTDYNHRRLHNLHLLAARFPSLHFESISDGLPPDQPRDILDPKFFISICQVTKPLFRELLLSYKRISSVQTGRPPITCVITDVIFRFPIDVAEELDIPVFSFCTFSARFMFLYFWIPKLIEDGQLPYPNGNINQKLYGVAPEAEGLLRCKDLPGHWAFADELKDDQLNFVDQTTASSRSSGLILNTFDDLEAPFLGRLSTIFKKIYAVGPIHSLLNSHHCGLWKEDHSCLAWLDSRAAKSVVFVSFGSLVKITSRQLMEFWHGLLNSGKSFLFVLRSDVVEGDDEKQVVKEIYETKAEGKWLVVGWAPQEKVLAHEAVGGFLTHSGWNSILESIAAGVPMISCPKIGDQSSNCTWISKVWKIGLEMEDRYDRVSVETMVRSIMEQEGEKMQKTIAELAKQAKYKVSKDGTSYQNLECLIQDIKKLNQIEGFINNPNFSDLLRV

SEQ ID NO: 179 - DNA, 1380 bp, synthetic construct
  source 1..1380: mol_type = other DNA
atggatgcccagcaaggtcacaccaccaccattttgatgcttccatgggtcggctacggccatctcttgcctttcctcgagctggccaaaagcctctccaggaggaaattattccacatctacttctgttcaacgtctgttagcctcgacgccattaaaccaaagcttcctccttctatctcttctgatgattccatccaacttgtggaacttcgtctcccttcttctcctgagttacctcctcatcttcacacaaccaacggccttccctctcacctcatgcccgctctccaccaagccttcgtcatggctgcccaacactttcaggtcattttacaaacacttgccccgcatctcctcatttatgacattctccaaccttgggctcctcaagtggcttcatccctcaacattccagccatcaacttcagtactaccggagcttcaatgctttctcgaacgcttcaccccactcactacccaagttctaaattcccaatctcagagtttgttcttcacaatcactggagagccatgtacaccaccgccgatggggctcttacagaagaaggccacaaaattgaagaaaaacttacgaattgcttgcatacttcttgcggggtagttttggtcaatagtttcagagagcttgagacgaaatatatcgattatctctctgttctcttgaacaagaaagttgttccggtcggtcctttggtttacgaaccgaatcaagaaggggaagatgaaggttattcaagcatcaaaaattggcttgacgaaaaggaaccgtcctcaaccgtcttcgtttcatttggaaccgaatacttcccgtcaaaggaagaaacggaagagatagcgtacgggttagagctgagcgaggttaatttcatctgggtccttagatttcctcaaggagacagaaccagcaccattgaagacgccttgccgaaggggtttctggagagagcgggagagagggcgatggtggtgaagggttgggctcctcaggcgaagatactgaaacattggagcacaggggggcttgtgagtcactgtggatggaactcgatgatggagggcatgatgtttggcgtacccataatagcggttccgatgcatctggaccagccctttaacgccggactcgtggaagaagctggcgtcggcgtggaagccaagcgagattcggacggcaaaattcaaagagaagaagttgcaaagtcgatcaaagaagtggtgattgagaaaaccagggaagacgtgaggaagaaagcaagagaaatgggtgagattttgaggagtaaaggagatgagaaaattgatgtgttggtggctgaaatttctcttttgcgcaaaaaggctccatgttcaatttaa

SEQ ID NO: 180 - protein, 459 aa, Siraitia grosvenorii
MDAQQGHTTTILMLPWVGYGHLLPFLELAKSLSRRKLFHIYFCSTSVSLDAIKPKLPPSISSDDSIQLVELRLPSSPELPPHLHTTNGLPSHLMPALHQAFVMAAQHFQVILQTLAPHLLIYDILQPWAPQVASSLNIPAINFSTTGASMLSRTLHPTHYPSSKFPISEFVLHNHWRAMYTTADGALTEEGHKIEEKLTNCLHTSCGVVLVNSFRELETKYIDYLSVLLNKKVVPVGPLVYEPNQEGEDEGYSSIKNWLDEKEPSSTVFVSFGTEYFPSKEETEEIAYGLELSEVNFIWVLRFPQGDRTSTIEDALPKGFLERAGERAMVVKGWAPQAKILKHWSTGGLVSHCGWNSMMEGMMFGVPIIAVPMHLDQPFNAGLVEEAGVGVEAKRDSDGKIQREEVAKSIKEVVIEKTREDVRKKAREMGEILRSKGDEKIDVLVAEISLLRKKAPCSI

SEQ ID NO: 181 - DNA, 1422 bp, synthetic construct
  source 1..1422: mol_type = other DNA
atggatgctcaacaaggacatactactactattcttatgcttccttgggttggatatggacatcttcttccttttcttgaacttgctaagtctctttctagaagaaagctttttcatatttatttttgttctacttctgtttctcttgatgctattaagcctaagcttcctccttctatttcttctgatgattctattcaacttgttgaacttagacttccttcttctcctgaacttcctcctcatcttcatactactaatggacttccttctcatcttatgcctgctcttcatcaagcttttgttatggctgctcaacattttcaagttattcttcaaactcttgctcctcatcttcttatttatgatattcttcaaccttgggctcctcaagttgcttcttctcttaatattcctgctattaatttttctactactggagcttctatgctttctagaactcttcatcctactcattatccttcttctaagtttcctatttctgaatttgttcttcataatcattggagagctatgtatactactgctgatggagctcttactgaagaaggacataagattgaagaaactcttgctaattgtcttcatacttcttgtggagttgttcttgttaattcttttagagaacttgaaactaagtatattgattatctttctgttcttcttaataagaaggttgttcctgttggacctcttgtttatgaacctaatcaagaaggagaagatgaaggatattcttctattaagaattggcttgataagaaggaaccttcttctactgtttttgtttcttttggaactgaatattttccttctaaggaagaaatggaagaaattgcttatggacttgaactttctgaagttaattttatttgggttcttagatttcctcaaggagattctacttctactattgaagatgctcttcctaagggatttcttgaaagagctggagaaagagctatggttgttaagggatgggctcctcaagctaagattcttaagcattggtctactggaggacttgtttctcattgtggatggaattctatgatggaaggaatgatgtttggagttcctattattgctgttcctatgcatcttgatcaaccttttaatgctggacttgttgaagaagctggagttggagttgaagctaagagagattctgatggaaagattcaaagagaagaagttgctaagtctattaaggaagttgttattgaaaagactagagaagatgttagaaagaaggctagagaaatggatactaagcatggacctacttatttttctagatctaaggtttcttcttttggaagactttataagattaatagacctactactcttactgttggaagattttggtctaagcaaattaagatgaagagagaataa

SEQ ID NO: 182 - protein, 473 aa, Siraitia grosvenorii
MDAQQGHTTTILMLPWVGYGHLLPFLELAKSLSRRKLFHIYFCSTSVSLDAIKPKLPPSISSDDSIQLVELRLPSSPELPPHLHTTNGLPSHLMPALHQAFVMAAQHFQVILQTLAPHLLIYDILQPWAPQVASSLNIPAINFSTTGASMLSRTLHPTHYPSSKFPISEFVLHNHWRAMYTTADGALTEEGHKIEETLANCLHTSCGVVLVNSFRELETKYIDYLSVLLNKKVVPVGPLVYEPNQEGEDEGYSSIKNWLDKKEPSSTVFVSFGTEYFPSKEEMEEIAYGLELSEVNFIWVLRFPQGDSTSTIEDALPKGFLERAGERAMVVKGWAPQAKILKHWSTGGLVSHCGWNSMMEGMMFGVPIIAVPMHLDQPFNAGLVEEAGVGVEAKRDSDGKIQREEVAKSIKEVVIEKTREDVRKKAREMDTKHGPTYFSRSKVSSFGRLYKINRPTTLTVGRFWSKQIKMKRE

SEQ ID NO: 183 - DNA, 1422 bp, synthetic construct
  source 1..1422: mol_type = other DNA
atgactatttttttttctgttgaaattcttgttcttggaattgctgaatttgctgctattgctatggatgctgcccaacaaggtgacaccacaaccattttgatgcttccatggctcggctatggccatctttcagcttttctcgagctggccaaaagcctctcaaggaggaacttccatatctacttctgttcaacctctgttaatcttgacgccattaaaccaaagcttccttcttctttctctgattccattcaatttgtggagctccatctcccttcttctcctgagttccctcctcatcttcacacaaccaacggccttccccctaccctcatgcccgctctccaccaagccttctccatggctgcccagcactttgagtccattttacaaacacttgccccgcaccttctcatttatgactctcttcaaccttgggctcctcgggtagcttcatccctcaaaattccggccatcaacttcaataccacgggagttttcgtcatttctcaagggcttcaccctattcactacccacattctaaattcccattctcagagttcgttcttcacaatcattggaaagccatgtactccactgccgatggagcttctaccgaaagaacccgcaaacgtggagaagcgtttctgtattgcttgcatgcttcttgtagtgtaattctaatcaatagtttcagagagctcgaggggaaatatatggattatctctctgttctcttgaacaagaaagttgttccggttggtcctttggtttacgaaccgaatcaagacggggaagatgaaggttattcaagcatcaaaaattggcttgacaaaaaggaaccgtcctccaccgtcttcgtgtcatttggaagcgaatacttcccgtcaaaggaagaaatggaagagatagcccatgggttagaggcgagcgaggttaatttcatctgggtcgttaggtttcctcaaggagacaacaccagcggcattgaagatgccttgccgaagggttttctggagagggcgggagagagagggatggtggtgaagggttgggctcctcaggcgaagatactgaagcattggagcacagggggattcgtgagccactgtggatggaactcggtgatggagagcatgatgtttggcgttcccataataggggttccgatgcatgtggaccagccctttaacgccggactcgtggaagaagctggcgtcggcgtggaggccaagcgagatccagacggcaaaattcaaagagacgaagttgcaaagttgatcaaagaagtggtggttgagaaaaccagagaagatgtgcggaagaaagcaagagaaatgagtgagattttgaggagcaagggagaggagaagtttgatgagatggtcgctgaaatttctctcttgcttaaaatataa

SEQ ID NO: 184 - protein, 473 aa, Siraitia grosvenorii
MTIFFSVEILVLGIAEFAAIAMDAAQQGDTTTILMLPWLGYGHLSAFLELAKSLSRRNFHIYFCSTSVNLDAIKPKLPSSFSDSIQFVELHLPSSPEFPPHLHTTNGLPPTLMPALHQAFSMAAQHFESILQTLAPHLLIYDSLQPWAPRVASSLKIPAINFNTTGVFVISQGLHPIHYPHSKFPFSEFVLHNHWKAMYSTADGASTERTRKRGEAFLYCLHASCSVILINSFRELEGKYMDYLSVLLNKKVVPVGPLVYEPNQDGEDEGYSSIKNWLDKKEPSSTVFVSFGSEYFPSKEEMEEIAHGLEASEVNFIWVVRFPQGDNTSGIEDALPKGFLERAGERGMVVKGWAPQAKILKHWSTGGFVSHCGWNSVMESMMFGVPIIGVPMHVDQPFNAGLVEEAGVGVEAKRDPDGKIQRDEVAKLIKEVVVEKTREDVRKKAREMSEILRSKGEEKFDEMVAEISLLLKI

SEQ ID NO: 185 - DNA, 1599 bp, synthetic construct
  source 1..1599: mol_type = other DNA
atgttaatgcatgcattgactcctacagctccatttttctccataaagccaaataccgaacctccatcagccactactcgtcagccaccaatggattctccaccacaaaaacctcacttcttgttgtttccattcatggctcaaggtcacatgatccctatgattgacttagctaagttgttagcccaaagaggggctattatcaccgtcgttaccactccacataacgctgccagatatcactctgttttggctagagctattgattccggtttgcatattcacgtcttgcaattacaattcccatgtaacgagggtggtttgccagaaggttgcgaaaactttgacttgttgccatctttaggttcagcctctactttctttagagcaaccttcttattgtacgaaccttctgaaaaggttttcgaagaattgatcccaagacctacatgtatcatatccgacatgtgtttaccatggacagtccgtttggctcagaaataccacgttccaagattggttttttattccttgtcatgttttttcttgttgtgcatgagatctttgaagaataaccaagctttaatttcttctaagtccgattctgagttggtcactttttccgatttgccagatccagtcgaatttttaaaatcacaattgcctaagtctaatgacgaagaaatggctaagttcggttacgagatcggtgaagctgatagacaatcccatggtgttattgttaacgttttcgaagagatggaaccaaaatacttagcagaatatagaaaggaaagagaatctcctgaaaaggtctggtgtgttggtccagtctccttgtgtaatgacaacaagttagacaaagcccaacgtggtaacaaggcttcaattgatgaaagagaatgtatcgagtggttggacggtcaacaaccatcttctgttgtttacgtctctttaggttctttgtgtaacttagttaccgcccaattgattgagttgggtttgggtttagaagcttctaacaagccattcatttgggttatcagaaaaggtaatattaccgaagaattacagaaatggttggtcgaatatgacttcgaggaaaagactaagggtcgtggtttggttatattgggttgggctccacaagtcttgattttatcccatccagcaatcggttgctttttaacacactgtgggtggaactcctccatcgaaggtatctcagccggtatgccaatgattacctggccattgttcgctgatcaagttttcaatgaaaagttgatcgttgaaatcttgagaataggtgtttctgtcggtatggaaaccgctatgcattggggtgaagaagaagaaaagggtgtcgtcgttaagagagaaaaagtcagagaggcaatagaaagagcaatggatggtgacgaaagagaagaacgtcgtgagagatgcaaggagttggccgaaatggctaagagagctgttgaagaaggtggttcatctcacagaaacttgactttgttgactgaagatattttggtcaatggtggtggtcaggaaagaatggatgacgccgatgacttcccaactatcgtcaactaa

SEQ ID NO: 186 - protein, 532 aa, Siraitia grosvenorii
MLMHALTPTAPFFSIKPNTEPPSATTRQPPMDSPPQKPHFLLFPFMAQGHMIPMIDLAKLLAQRGAIITVVTTPHNAARYHSVLARAIDSGLHIHVLQLQFPCNEGGLPEGCENFDLLPSLGSASTFFRATFLLYEPSEKVFEELIPRPTCIISDMCLPWTVRLAQKYHVPRLVFYSLSCFFLLCMRSLKNNQALISSKSDSELVTFSDLPDPVEFLKSQLPKSNDEEMAKFGYEIGEADRQSHGVIVNVFEEMEPKYLAEYRKERESPEKVWCVGPVSLCNDNKLDKAQRGNKASIDERECIEWLDGQQPSSVVYVSLGSLCNLVTAQLIELGLGLEASNKPFIWVIRKGNITEELQKWLVEYDFEEKTKGRGLVILGWAPQVLILSHPAIGCFLTHCGWNSSIEGISAGMPMITWPLFADQVFNEKLIVEILRIGVSVGMETAMHWGEEEEKGVVVKREKVREAIERAMDGDEREERRERCKELAEMAKRAVEEGGSSHRNLTLLTEDILVNGGGQERMDDADDFPTIVN

SEQ ID NO: 187 - DNA, 1377 bp, synthetic construct
  source 1..1377: mol_type = other DNA
atggaaaataaaactgaaacaactgttagaagaagaagaagaatcatcttgttcccagtcccattccaaggtcatattaacccaatcttgcaactggctaatgtgctgtactccaaaggtttttctatcactatttttcatactaattttaataaacctaaaacttctaattacccacatttcacatttagatttattttggacaatgacccacaagacgagagaatctccaacctgccaacccatggtccattggccggcatgcgcatcccaatcatcaacgaacatggtgccgatgaattgcgcagagaacttgaattgttgatgttagcatctgaagaagacgaggaagtttcctgtctgattactgacgctttgtggtattttgctcaatctgttgctgattctttgaatttgagaagattggttttgatgacatcttctttgtttaattttcatgctcatgttagtctaccacagtttgatgagcttggttacttagatccagatgataagactagactagaagaacaagcttctggtttcccaatgctaaaggtaaaggatatcaaatccgcttattccaattggcaaatcctaaaggaaatattgggtaaaatgatcaagcagacaagagcttcttctggtgttatctggaactcttttaaagagctagaggagtctgaattggaaacagtcattagagaaatcccagctccatcttttctaattccattgccaaaacatttgaccgcatcatcttcttctctgctagaccatgatagaactgttttccaatggttggatcaacaaccaccatcttccgtcttgtatgtttcttttggctccacttcagaggtggacgagaaagattttctggaaattgctagaggtttagtagatagtaaacagagttttttatgggtagtaagaccaggttttgttaaaggttctacatgggttgagccactacctgatggttttttgggtgaaagaggtagaatagttaaatgggtaccacaacaagaagtcttggcacatggtgccattggtgctttctggacacattctggatggaactctacacttgaatctgtttgtgaaggtgttccaatgattttctccgattttggtttggatcaaccattgaatgctagatacatgtcagacgtcctgaaggttggtgtttacctggaaaacggttgggaacgcggtgaaatcgccaacgccattcgcagagtcatggtggacgaggaaggtgaatacattagacaaaatgccagagtacttaagcaaaaagcagatgtctctttgatgaaaggtggttcttcttatgagtctttggaatcattagtgtcttatatctctagtctctaa

SEQ ID NO: 188 - protein, 458 aa, Siraitia grosvenorii
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 189 - DNA, 1401 bp, synthetic construct
  source 1..1401: mol_type = other DNA
atgctgctccagtccaacccgatggagaacgagacggagacgacggtgagccgccggcgcagaatattattatttcctgtaccatttcaaggtcatataaatccaatgttacaacttgcaaatgtcctttatagcaaaggattctccatcactattttccataccaatttcaacagtccaaagacctcaaattacccccactttacttttcgatttatcctagacaatgacccccaagataaaagaatttccaatctccccacacacggcccagcagcttcaatcaggattccatttttcaatgagtacagagccgatgaattaagacgtgagctagaattactcatgttggcttccgaagaagatggagaggtaagttgcctgattgcagatcagatatggtattttactcaatctgtcgcggatagcctcaatcttagaagattagtcttggttacatcctcattgttcaacttccacgcacatgtaagcctaccgcaattcgatgagttaggatatttggaccctgatgataaaactcggctagaagagcaagcttcaggatttcctatgctaaaagtaaaagatataaaatgtagcttttctatgtggaagaagtacaaggaatatttcgagaacattactaagcagacgaaagcgtcatccggcgtgatttggaatagctttaaagaattggaagagtctgagcttgaaaccgtaattcgggaaataccagccccttcttttcttattcctctaccaaaacacttaacggcatcttcgtcgtctttattagatcacgatcgtacagtgttcccgtggctggatcaacaacctagcaggtcggtcttatacgtatcatttggaagtggtactgaggtgttagatgaaaaagactttttagaaatagctagaggtttggttgactcaaagcaatcttttttgtgggtcgtcagacccggcttcgttaaaggttcaacgtgggtcgaacctctaccagatggtttcttgggtgaaagaggtagaattgttaagtgggtaccacaacaagaagtcctagctcatggagctataggtgcattttggactcactcaggctggaactcaacattggaatctgtgtgcgagggagtaccaatgatcttctctgatttcggtttagatcagcctctgaacgcaaggtacatgtcggatgtactaaaggtaggggtctatttggaaaatggatgggagagaggagagattgctaatgctatcagacgcgtaatggttgatgaggaaggtgaatatatcagacaaaatgccagagtgttaaagcaaaaagccgatgttagtttaatgaagggaggtagttcttacgagtcgttggagagtcttgtctcatacatatcttccttgtaa

SEQ ID NO: 190 - protein, 466 aa, Siraitia grosvenorii
MLLQSNPMENETETTVSRRRRILLFPVPFQGHINPMLQLANVLYSKGFSITIFHTNFNSPKTSNYPHFTFRFILDNDPQDKRISNLPTHGPAASIRIPFFNEYRADELRRELELLMLASEEDGEVSCLIADQIWYFTQSVADSLNLRRLVLVTSSLFNFHAHVSLPQFDELGYLDPDDKTRLEEQASGFPMLKVKDIKCSFSMWKKYKEYFENITKQTKASSGVIWNSFKELEESELETVIREIPAPSFLIPLPKHLTASSSSLLDHDRTVFPWLDQQPSRSVLYVSFGSGTEVLDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 191 - DNA, 1524 bp, synthetic construct
  source 1..1524: mol_type = other DNA
atggccgtcgttgcttcggctccagggaaagttttaatgacgggaggttaccttgtcttggagaggccgaatgcagggctcgtacttagtactaatgctcggttttatgcaattgtaaaaccactttacgaggaaatcaagcctgacagctgggcatggtcatggacagatgttaaattgacatctcctcaactcctgagagagagcatctacaaattgtcattgaaaaaccaatctcttcagagtgtctctccaagtcaatcaagaaacccttttgtagaaaaagcagtagaatattgtgtagcagctgcatgtgcaaaatttgttgacaaggacaatgaggatgcattgcacaaactgctcttgcatggtcttgatatcactattttaggttgcaatgagttctactcctatcgaagtcagattgaggctcgtggactcccattaacatcagaatctttggcttccctgcctcctttttcatcaattaccttcaatgatgaggaatcatatgggaaaaattgcaagcctgaagttgcaaaaactggattgggctcatctgctgcaatgacagctgctgtagttgctgctttacttaactaccttggggttgtaaatgtttccttattgacaggtgaccaagaacagaaaggcagtgcggatcttgatttggtgcatatgatagctcaaagtgcccactgcattgcacaaggaaaagttggcagtggctttgatgtcagttctgcagtttatggcagtcagcgctacattcggttttcaccagaagtgctatcttctgctcaggctgccacggaaggaatacccatacaagaggttattgtcggtatcttaaaccagaagtgggaccatgagaggactaagttctctttgcctccattaatgactcttttacttggagagccaggagtgggagggtcgtccacaccttcaatggtaggtgctgtcagaaaatggcaaaaatctgaccctcaaaattccctggacacgtggaggaagttgtcagaagcaaactcggaacttgaaactcagctcaatatgttgcgtaaacttgcagcagaacactgggatgcttataaatgtatcataaacagttgttgcaagttgacctccgagaagtggattcatcaagccactgaaccaagccagcaagccatcatcaaagcattattaggagcaagatatgctatgcttgagatcaggcatcatatgcacctgatgggtgaagctgctggtgttccaattgagccggattcacaaaccaagcttttagatgctaccatgaatatggaaggagtcctgttagccggagttccgggcgcaggcgggtttgatgctgtttttgctgttaccttgggagattcaaccagcaacgtgattaaatcatggagttcactcgatattttggctctccttgttagagaagatcctcgtggtattgctttggaaagtggcgatcctcgaaccaaggcaattacatcagctgtttcatcagttcatatttag

SEQ ID NO: 192 - protein, 507 aa, Siraitia grosvenorii
MAVVASAPGKVLMTGGYLVLERPNAGLVLSTNARFYAIVKPLYEEIKPDSWAWSWTDVKLTSPQLLRESIYKLSLKNQSLQSVSPSQSRNPFVEKAVEYCVAAACAKFVDKDNEDALHKLLLHGLDITILGCNEFYSYRSQIEARGLPLTSESLASLPPFSSITFNDEESYGKNCKPEVAKTGLGSSAAMTAAVVAALLNYLGVVNVSLLTGDQEQKGSADLDLVHMIAQSAHCIAQGKVGSGFDVSSAVYGSQRYIRFSPEVLSSAQAATEGIPIQEVIVGILNQKWDHERTKFSLPPLMTLLLGEPGVGGSSTPSMVGAVRKWQKSDPQNSLDTWRKLSEANSELETQLNMLRKLAAEHWDAYKCIINSCCKLTSEKWIHQATEPSQQAIIKALLGARYAMLEIRHHMHLMGEAAGVPIEPDSQTKLLDATMNMEGVLLAGVPGAGGFDAVFAVTLGDSTSNVIKSWSSLDILALLVREDPRGIALESGDPRTKAITSAVSSVHI

SEQ ID NO: 193 - DNA, 1026 bp, synthetic construct
  source 1..1026: mol_type = other DNA
atggaagagggaaatcgaacgcacccattctccaacaacagtatcaaagctgcggaactggctagaccagtccgctgcattgtgaaattgggaggtgcagctattacttgcaaaaatgaattggagacgatcaatgaagaaaatttgtcgacagtttcgtctcatatgaggaagatgatggttagtggctcgtcttctgcaaggaccattggaatggattggagcaagcaacctggaaaatccgacattgcatgtactatagatgattccggagaacaagaagttggccttgcgagcccctttatagttgttcatggagctggttcttttgggcacttccaggcaagtaaatcaggggttcataaaggaggactcgatagatccttggtcaaggctggatttgttgccacacgtatatctgttacttcgctcaatctccaaattgttagagcactagcgagagagggtattccttcggttggaatgtctccattttcatgtggatggtccacttgtgaaagaaatatagccgcagttgatttacgtggagtttctaaagctattaacttcggatttattcctgttctgcatggagatgccgttttggatgattccctggggtgcaccatattaagcggagatgtaataatacgacatcttgcagagcaactaaagcctgattatgttgttttcctcacagatgtcttaggtgtatatgatcgtcctcctacagaacctaatgcagtccttttgagagagattgttgtgggtgaagatggtcgttggtctgttatacgacctatactagagaacataaatacagaagttgagattagcgtagctgcccatgatacaactgggggaatggttactaagatatcagaggcggcaatgattgcaaagcttggtattgaagtctacatcgtaaaggcagcaactgtccactccttgagagccctaagtggggaaatccgaggcaaaattcctgatgactggcttgggacggtcatccggttttcaagataa

SEQ ID NO: 194 - protein, 341 aa, Siraitia grosvenorii
MEEGNRTHPFSNNSIKAAELARPVRCIVKLGGAAITCKNELETINEENLSTVSSHMRKMMVSGSSSARTIGMDWSKQPGKSDIACTIDDSGEQEVGLASPFIVVHGAGSFGHFQASKSGVHKGGLDRSLVKAGFVATRISVTSLNLQIVRALAREGIPSVGMSPFSCGWSTCERNIAAVDLRGVSKAINFGFIPVLHGDAVLDDSLGCTILSGDVIIRHLAEQLKPDYVVFLTDVLGVYDRPPTEPNAVLLREIVVGEDGRWSVIRPILENINTEVEISVAAHDTTGGMVTKISEAAMIAKLGIEVYIVKAATVHSLRALSGEIRGKIPDDWLGTVIRFSR

SEQ ID NO: 195 - DNA, 1257 bp, synthetic construct
  source 1..1257: mol_type = other DNA
atggcgggtgaaaaatgggtgctgatggcgactgcgcagacgccgacgaacatcgcggtgattaagtattggggcaagagggacgaggaattaatcttgcctgtcaatgatagcatcagtgttacccttgatccgagccacctctgcaccatcaccaccgtcgccgttagtcctgccttcgagcaggatcggatgtggctcaatcgcaaggagatatccctttctggagccaggtaccaaaattgtttgagggaaatacgcagccgagctaatgatgttgaggataaagaaaagggtattaagatagcaaaaaaagattgggagaaattgcatgtatatattgattcttacaataatttcccaactgccgctggcttggcttcctctgctgccggtttcgcttgtcttgtctttgctctggcaaatttaatgaatgtcaaggaagatcagagcaagttatctgctattgcaaggcaaggttcaggaagtgcttgccgtagtttatatggtggatttgtgaagtggagcatgggaaaagaaaaggatggaagtgatagtcttgcaattcaacttgctgacgagaagcactgggatgatcttgtaattatcattgctgtggtaagttcacgacaaaaggaaacaagtagcacttcaggaatgcgggaaactgtagaaacaagtttgcttttacaacatagagcaaaggaagttgtaccaaagcgcgtgttagctatggaagaagctatcaaaaatcgcgattttgtatcctttgcacaactgacatgctccgacagcaaccaatttcatgcagtctgtcttgatacgtctcccccaatattttatatgaatgatacatcccacaggataatcagtcttgttgagaagtggaaccgttctgaaggagaacctcaggtggcatatacttttgatgcggggccaaattcggttctaattgcacgtaatagaaaaactgctgtatctttgcttcagaggttgcttttctacttccctcccaatccagaaacggatttaaacagttatgttcttggtgacaagtcaattcttcaagatgctgggattaagggtgttgaggatattgagtccctgctacaacctccagaaattaacagtccattccagaaattccagggggatgtcagttatttcatctgtaccaggccggggaaagggccggttgtgctccccgacagtcaagctctactcgacccaaaaactgggctgccaaagaatctttag

SEQ ID NO: 196 - protein, 418 aa, Siraitia grosvenorii
MAGEKWVLMATAQTPTNIAVIKYWGKRDEELILPVNDSISVTLDPSHLCTITTVAVSPAFEQDRMWLNRKEISLSGARYQNCLREIRSRANDVEDKEKGIKIAKKDWEKLHVYIDSYNNFPTAAGLASSAAGFACLVFALANLMNVKEDQSKLSAIARQGSGSACRSLYGGFVKWSMGKEKDGSDSLAIQLADEKHWDDLVIIIAVVSSRQKETSSTSGMRETVETSLLLQHRAKEVVPKRVLAMEEAIKNRDFVSFAQLTCSDSNQFHAVCLDTSPPIFYMNDTSHRIISLVEKWNRSEGEPQVAYTFDAGPNSVLIARNRKTAVSLLQRLLFYFPPNPETDLNSYVLGDKSILQDAGIKGVEDIESLLQPPEINSPFQKFQGDVSYFICTRPGKGPVVLPDSQALLDPKTGLPKNL

SEQ ID NO: 197 - DNA, 3018 bp, synthetic construct
  source 1..3018: mol_type = other DNA
atgaagctgctgagcgtggccgccgtggccctgctggccgcccaggccgccggcgccagcatcaagcacaggctgaacggcttcaccatcctggagcacccggacccggccaagagggacctgctgcaggacatcgtgacctgggacgacaagagcctgttcatcaacggcgagaggatcatgctgttcagcggcgaggtgcacccgttcaggctgccggtgccgagcctgtggctggacatcttccacaagatcagggccctgggcttcaactgcgtgagcttctacatcgactgggccctgctggagggcaagccgggcgactacagggccgagggcatcttcgccctggagccgttcttcgacgccgccaaggaggccggcatctacctgatcgccaggccgggcagctacatcaacgccgccgtgagcggcggcggcttcccgggctggctgcagagggtgaacggcaccctgaggagcagcgacgagccgttcctgaaggccaccgacaactacatcgccaacgccgccgccgccgtggccaaggcccagatcaccaacggcggcccggtgatcctgtaccagccggagaacgagtacagcggcggcagatgcggcgagaagtacccggacgccgactacatgcagtacgtgatggaccaggccaggaaggccgacatcgtggtgccgttcatcagcaacgacgccagcccgagcggccacaacgccccgggcagcggcaccagcgccgtggacatctacggccacgacagctacccgctgggcttcgactgcgccaacccgagcgtgtggccggagggcaagctgccggacaacttcaggaccctgcacctggagcagagcccgagcaccccgtacagcctgctggagttccaggccggcgccttcgacccgtggggcggcccgggcttcgagaagtgctacgccctggtgaaccacgagttcagcagggtgttctacaggaacgacctgagcttcggcgtgagcaccttcaacctgtacatgaccttcggcggcaccaactggggcaacctgggccacccgggcggctacaccagctacgactacggcagcccgatcaccgaaaccaggaacgtgaccagggagaagtacagcgacatcaagctgctggccaacttcgtgaaggccagcccgagctacctgaccgccaccccgaggaacctgaccaccggcgtgtacaccgacaccagcgacctggccgtgaccccgctgatcggcgacagcccgggcagcttcttcgtggtgaggcacaccgactacagcagccaggagagcaccagctacaagctgaagctgccgaccagcgccggcaacctgaccatcccgcagctggagggcaccctgagcctgaacggcagggacagcaagatccacgtggtggactacaacgtgagcggcaccaacatcatctacagcaccgccgaggtgttcacctggaagaagttcgacggcaacaaggtgctggtgctgtacggcggcccgaaggagcaccacgagctggccatcgccagcaagagcaacgtgaccatcatcgagggcagcgacagcggcatcgtgagcaccaggaagggcagcagcgtgatcatcggctgggacgtgagcagcaccaggaggatcgtgcaagtgggcgacctgagggtgttcctgctggacaggaacagcgcctacaactactgggtgccggagctgccgaccgagggcaccagcccgggcttcagcaccagcaagaccaccgccagcagcatcatcgtgaaggccggctacctgctgaggggcgcccacctggacggcgccgacctgcacctgaccgccgacttcaacgccaccaccccgatcgaggtgatcggcgccccgaccggcgccaagaacctgttcgtgaacggcgagaaggccagccacaccgtggacaagaacggcatctggagcagcgaggtgaagtacgccgccccggagatcaagctgccgggcctgaaggacctggactggaagtacctggacaccctgccggagatcaagagcagctacgacgacagcgcctgggtgagcgccgacctgccgaagaccaagaacacccacaggccgctggacaccccgaccagcctgtacagcagcgactacggcttccacaccggctacctgatctacaggggccacttcgtggccaacggcaaggagagcgagttcttcatcaggacccagggcggcagcgccttcggcagcagcgtgtggctgaacgaaacctacctgggcagctggaccggcgccgactacgccatggacggcaacagcacctacaagctgagccagctggagagcggcaagaactacgtgatcaccgtggtgatcgacaacctgggcctggacgagaactggaccgtgggcgaggaaaccatgaagaacccgaggggcatcctgagctacaagctgagcggccaggacgccagcgccatcacctggaagctgaccggcaacctgggcggcgaggactaccaggacaaggtgaggggcccgctgaacgagggcggcctgtacgccgagaggcagggcttccaccagccgcagccgccgagcgagagctgggagagcggcagcccgctggagggcctgagcaagccgggcatcggcttctacaccgcccagttcgacctggacctgccgaagggctgggacgtgccgctgtacttcaacttcggcaacaacacccaggccgccagggcccagctgtacgtgaacggctaccagtacggcaagttcaccggcaacgtgggcccgcagaccagcttcccggtgccggagggcatcctgaactacaggggcaccaactacgtggccctgagcctgtgggccctggagagcgacggcgccaagctgggcagcttcgagctgagctacaccaccccggtgctgaccggctacggcaacgtggagagcccggagcagccgaagtacgagcagaggaagggcgcctactaa

SEQ ID NO: 198 - protein, 1005 aa, Aspergillus oryzae
MKLLSVAAVALLAAQAAGASIKHRLNGFTILEHPDPAKRDLLQDIVTWDDKSLFINGERIMLFSGEVHPFRLPVPSLWLDIFHKIRALGFNCVSFYIDWALLEGKPGDYRAEGIFALEPFFDAAKEAGIYLIARPGSYINAAVSGGGFPGWLQRVNGTLRSSDEPFLKATDNYIANAAAAVAKAQITNGGPVILYQPENEYSGGRCGEKYPDADYMQYVMDQARKADIVVPFISNDASPSGHNAPGSGTSAVDIYGHDSYPLGFDCANPSVWPEGKLPDNFRTLHLEQSPSTPYSLLEFQAGAFDPWGGPGFEKCYALVNHEFSRVFYRNDLSFGVSTFNLYMTFGGTNWGNLGHPGGYTSYDYGSPITETRNVTREKYSDIKLLANFVKASPSYLTATPRNLTTGVYTDTSDLAVTPLIGDSPGSFFVVRHTDYSSQESTSYKLKLPTSAGNLTIPQLEGTLSLNGRDSKIHVVDYNVSGTNIIYSTAEVFTWKKFDGNKVLVLYGGPKEHHELAIASKSNVTIIEGSDSGIVSTRKGSSVIIGWDVSSTRRIVQVGDLRVFLLDRNSAYNYWVPELPTEGTSPGFSTSKTTASSIIVKAGYLLRGAHLDGADLHLTADFNATTPIEVIGAPTGAKNLFVNGEKASHTVDKNGIWSSEVKYAAPEIKLPGLKDLDWKYLDTLPEIKSSYDDSAWVSADLPKTKNTHRPLDTPTSLYSSDYGFHTGYLIYRGHFVANGKESEFFIRTQGGSAFGSSVWLNETYLGSWTGADYAMDGNSTYKLSQLESGKNYVITVVIDNLGLDENWTVGEETMKNPRGILSYKLSGQDASAITWKLTGNLGGEDYQDKVRGPLNEGGLYAERQGFHQPQPPSESWESGSPLEGLSKPGIGFYTAQFDLDLPKGWDVPLYFNFGNNTQAARAQLYVNGYQYGKFTGNVGPQTSFPVPEGILNYRGTNYVALSLWALESDGAKLGSFELSYTTPVLTGYGNVESPEQPKYEQRKGAY

SEQ ID NO: 199 - DNA, 2289 bp, synthetic construct
  source 1..2289: mol_type = other DNA
atgtggaggttaaaggtgggagcagagagcgttggcgagaatgatgagaaatgggtaaagagcataagcaatcacttgggacggcaagtgtgggagttctgtccagatgccggaactccacaacagctattgcaaatcgagaaagctaagaaagccttccaagataaccgtttccatcgaaagcaaacttccgatctccttgtctccattcagtgcgaaaagggaacaacaaatggtgcaagggtaccgggaaccaagttgaaagagggggaagaggtgaggaaagaggcagtaaagagtacattagaaagggcattaagtttctactctagcatccagacaagcgatgggaactgggcttcggatcttggggggcccatgtttttacttccgggtctggtgattgctctctgcgttacaggcgccctgaattctgttttatccaagcaccacaggcaagagatgtgcagatatctttacaaccaccagaatgaagatggggggtgggctctccacatcgagagcccaagcaccatgtttggttccgcactgaattatgttgcactgaggttgcttggggaagacgcggatggcggagagggcagggcaatgaccaaagcacgtgcttggatcttgggccacggtggcgccaccgcaatcacttcctggggcaaattgtggctttctgtacttggagtctacgaatggagtggcaacaatcctcttccacccgaattttggttacttccttacttcctaccatttcatccaggaagaatgtggtgccattgtcgaatggtttatctaccaatgtcatacttatatgggaagaggtttgttgggcccatcacacccgtagttttgtctcttagaaaagagctctacacagttccatatcatgaaatagactggaacaaatctcgcaatacatgtgcaaaggaggatctgtactatccacattccaagatgcaagatattctgtggggatctatccaccatatgtatgagcccttgttcacacattggcctgccaaacgcctgagggaaaaggctttgaaaactgcaatgcaacatattcactatgaagatgagaacactcgatatatatgcctcgggcctgtcaacaaggtactcaatatgctttgttgttgggttgaagatccctactcagaggccttcaaacttcatcttcaacgagtccatgactatctctgggttgctgaggatggcatgaaaatgcagggttataatggaagccagttgtgggacactgctttctccgtccaagcaatcatatccaccaagcttgtagacaactatggcccaactttaagaaaggcacacgactacgttaaaaattctcagatccaacaggactgtcctggggaacctaatgtttggttccgtcacattcataaaggtgcatggccattttcaactcgagatcatggatggctcatctctgactgtacagcagagggattaaaggcttctttaatgttatccaaacttccatccgaaacggttggggagccattagaaaggaatcgcctttgtgatgctgtaaacgttctcctttctttgcaaaacgacaatggtggctttgcatcatatgagttgacgagatcatatccttggttggagttgatcaaccctgcggaaacgtttggagatattgtcattgattatccgtatgtggagtgcacctcagccacaatggaagcactggcgttgtttaagaaattacatcccggccataggaccaaagaaattgatactgctattgccagggcagccgacttcctcgaaaatatgcaaaggacggatggttcttggtacggatgttggggggtttgcttcacttatgcgggttggtttggcataaagggattagtggctgcaggaagggcatatagtaattgcctcgccattcgcaaggcttgcgattttctactgtctaaagagctgcccggcggtggatggggggagagttacctttcatgtcagaataaggtctacacaaatcttgaaggaaacagaccacacctggttaacacggcttgggttttaatggccctcatcgaagctggccagggtgagagagatccagcaccattgcatcgtgcagcaaggttgttaatcaattcacagttggagaatggcgatttcccacaagaggagatcatgggagtcttcaataaaaattgcatgatcacatatgctgcataccggaacatttttcccatttgggctcttggagagtattgccatagggttttgaccgaataa

SEQ ID NO: 200 - protein, 762 aa, Momordica charantia
MWRLKVGAESVGENDEKWVKSISNHLGRQVWEFCPDAGTPQQLLQIEKAKKAFQDNRFHRKQTSDLLVSIQCEKGTTNGARVPGTKLKEGEEVRKEAVKSTLERALSFYSSIQTSDGNWASDLGGPMFLLPGLVIALCVTGALNSVLSKHHRQEMCRYLYNHQNEDGGWALHIESPSTMFGSALNYVALRLLGEDADGGEGRAMTKARAWILGHGGATAITSWGKLWLSVLGVYEWSGNNPLPPEFWLLPYFLPFHPGRMWCHCRMVYLPMSYLYGKRFVGPITPVVLSLRKELYTVPYHEIDWNKSRNTCAKEDLYYPHSKMQDILWGSIHHMYEPLFTHWPAKRLREKALKTAMQHIHYEDENTRYICLGPVNKVLNMLCCWVEDPYSEAFKLHLQRVHDYLWVAEDGMKMQGYNGSQLWDTAFSVQAIISTKLVDNYGPTLRKAHDYVKNSQIQQDCPGEPNVWFRHIHKGAWPFSTRDHGWLISDCTAEGLKASLMLSKLPSETVGEPLERNRLCDAVNVLLSLQNDNGGFASYELTRSYPWLELINPAETFGDIVIDYPYVECTSATMEALALFKKLHPGHRTKEIDTAIARAADFLENMQRTDGSWYGCWGVCFTYAGWFGIKGLVAAGRAYSNCLAIRKACDFLLSKELPGGGWGESYLSCQNKVYTNLEGNRPHLVNTAWVLMALIEAGQGERDPAPLHRAARLLINSQLENGDFPQEEIMGVFNKNCMITYAAYRNIFPIWALGEYCHRVLTE

SEQ ID NO: 201 - DNA, 2295 bp, synthetic construct
  source 1..2295: mol_type = other DNA
atgtggaggctgaaggtgggagcagagagcgttggggaggaggatgagaaatgggtgaagagcgtaagcaatcacttgggccgccaagtttgggagttctgtgccgacgccgccgccgacactcctcaccagttactacaaattcagaatgctaagaaccacttccatcacaatcgtttccaccggaagcagtcttccgatctctttctggctattcaatatgaaaaggaaatagcaaagggcgcaaaaggtggagcggtgaaagtgaaagaaggggaggaggtggggaaagaggcggtgaagagtacgttagaaagggcactcggtttctactcggccgtgcagacaagagatgggaattgggcctcggatcttggagggcccttgtttttacttccgggcctcgtgattgcccttcatgtcacaggcgtcttgaattcagttttgtccaagcaccaccgcgtagagatgtgcagatatctttacaatcaccagaatgaagatggagggtggggtctacatattgagggcacaagcaccatgtttggttcggcactgaattacgttgcactaaggctgcttggagaagacgccgatggcggagacggtggcgcaatgacaaaagcacgtgcttggatcttggagcgcggcggcgccactgcgatcacttcgtggggaaaattgtggctgtccgtacttggagtgtacgaatggagtggcaacaaccctcttccgcctgagttttggcttctcccttacagcctaccatttcatccaggaagaatgtggtgccattgtcgaatggtttatcttccaatgtcttacttatatgggaagagatttgttgggccaatcactcccaaagttctttctctaaggcaagagctctacacaattccttatcatgaaatagactggaataaatcccgcaatacatgtgcaaaggaggatctgtactatccacatcccaagatgcaagacattctatggggatccatctaccatgtatatgagccattgttcactcgttggcctgggaaacgcctgagggaaaaggctttacaagctgcaatgaaacatattcactatgaagatgaaaatagtcgatatatatgtcttggcccagtcaacaaggtactcaacatgctttgttgttgggttgaagatccctactcagacgccttcaaacttcaccttcaacgcgtccatgactatctctgggttgctgaagatggcatgagaatgcagggctacaatggcagccagttgtgggacactgctttctccatccaagccatcgtagccaccaaacttgtagacagctatgccccaactttaagaaaagcacatgactttgttaaggattctcagatccaggaggactgtcctggggatcctaatgtttggttccgtcatattcataaaggtgcttggccactttcgacacgagatcatggatggctcatctccgactgtacagctgagggattgaaggcttctttgatgttatccaaacttccatccacaatggttggggagccattagaaaagaatcgcctttgtgatgctgttaatgttctcctttctttgcaaaatgataatggtggatttgcatcatacgagttgacgagatcatacccttggttggagttgatcaacccagctgaaacattcggagacattgtcattgactatccgtatgtggagtgcaccgcagcaacaatggaagcactgacgttatttaagaagctacatccaggccataggaccaaagagattgacacagctattggcaaggcagccaacttccttgagaaaatgcagagggcggatggctcttggtacgggtgttggggggtttgttttacgtatgcgggttggtttggcataaagggattggtggctgcaggaagaacatataatagctgccttgccattcgcaaggcttgtgagtttctgctatctaaagagctgcccggcggtggatggggggagagttacctttcatgtcagaataaggtgtacaccaatcttgaggggaacaagccacacttggttaacactgcctgggttttaatggctctcattgaagctggccagggtgagagagatccagcaccattgcaccgtgcagcaaggttgctaatgaattcccaattggagaatggcgatttcgtgcaacaggagatcatgggagtgttcaataagaactgcatgatcacatatgctgcataccgaaacatcttccccatttgggcgcttggagagtattgccatcgggttcttactgaatga

SEQ ID NO: 202 - protein, 764 aa, Cucurbita pepo
MWRLKVGAESVGEEDEKWVKSVSNHLGRQVWEFCADAAADTPHQLLQIQNAKNHFHHNRFHRKQSSDLFLAIQYEKEIAKGAKGGAVKVKEGEEVGKEAVKSTLERALGFYSAVQTRDGNWASDLGGPLFLLPGLVIALHVTGVLNSVLSKHHRVEMCRYLYNHQNEDGGWGLHIEGTSTMFGSALNYVALRLLGEDADGGDGGAMTKARAWILERGGATAITSWGKLWLSVLGVYEWSGNNPLPPEFWLLPYSLPFHPGRMWCHCRMVYLPMSYLYGKRFVGPITPKVLSLRQELYTIPYHEIDWNKSRNTCAKEDLYYPHPKMQDILWGSIYHVYEPLFTRWPGKRLREKALQAAMKHIHYEDENSRYICLGPVNKVLNMLCCWVEDPYSDAFKLHLQRVHDYLWVAEDGMRMQGYNGSQLWDTAFSIQAIVATKLVDSYAPTLRKAHDFVKDSQIQEDCPGDPNVWFRHIHKGAWPLSTRDHGWLISDCTAEGLKASLMLSKLPSTMVGEPLEKNRLCDAVNVLLSLQNDNGGFASYELTRSYPWLELINPAETFGDIVIDYPYVECTAATMEALTLFKKLHPGHRTKEIDTAIGKAANFLEKMQRADGSWYGCWGVCFTYAGWFGIKGLVAAGRTYNSCLAIRKACEFLLSKELPGGGWGESYLSCQNKVYTNLEGNKPHLVNTAWVLMALIEAGQGERDPAPLHRAARLLMNSQLENGDFVQQEIMGVFNKNCMITYAAYRNIFPIWALGEYCHRVLTE

SEQ ID NO: 203 - DNA, 2289 bp, synthetic construct
  source 1..2289: mol_type = other DNA
atgtggaagttaaaagtgggagcagagagcgctggcgagaagaatgagaaatggttgaagagcataagcaatcacttgggccgccaagtgtgggagttctgtcccgacgccgccgctccacaccaccaattgcagctccacaacgctaagaaacacttccatcataaccgtttttcccgcaagcaatcttccgatctccttctcgccattcagtgcgaaaaggaaatagcaaatggtgcaaaagttgcagcaatcaaactgaaagaaggagaaggggtgaggaaagaggcagtaaagattacattagaaagggcattaggtttctactcagccatccagacacctgatggcaactgggcttcggatcttggtgggcccatgtttctcattccgggtctagtaattgctctgtacgtcacaggcgccttgaattctattttatccaagcaccaccgccaggagatgtgcagatatctttacaatcaccagaatgaagatgggggatggggcctccacatcgagggcccaagcaccatgtttggttcaggactgaattatgttgcactcagactgctcggagaagatgctgacggcggagagggcggcgcgatgacaaaagcacgtgcctggatcttggcccgcggtggcgccactgcaatcccttcctggggcaagttgtggctctctgtacttggagtctatgaatggagtggcaataatcctcttccccctgaattttggttacttccttactccctaccatttcatcccggaagaatgtggtgccactgtcgaatggtttatctgccaatgtcatacttatatggaaagagatttgttgggccaatcacccccatagttctatctctcagaaaagagctcttcactattccatatcatgaaatagactggaacaaatctcgcaatacatgtgcaaaggaggatctgttctatccacatccgaagatgcaagatattctgtggggatccctccaccatttgtatgagccattgttcagtcgttggcctggcaaacgcatcagggaaaaggctttgcaaactgcaatgcaacatattcactatgaagatgagaatactcgatatatatgtcttggccctgtcaacaaggttctcaatatgctttgttgttgggttgaagatccctactccgacgccttcaagcttcatcttcaacgagtccatgactatctctggattgctgaagatggcatgagaatgcagggctacaatgggagccaattgtgggacactgctttctccatccaagcaatcctatccaccaaactcgtagacagctatggcccaaccttaagaaaagcacacgacttcgttaaaaattctcagatccagcaggactgtcctggggatcccaatgtttggttccgtcacattcataaaggtgcatggccattttcgactcgagatcatggctggcttatctctgactgtaccgcagagggattaaaggcttctttgatgttatccaaacttccatctgaaatagttggggagccattagagaagaatcgcctttgtgatgctgtaaatgttctcctttctttgcaaaatgataatggtgggtttgcatcgtacgagttgacaagatcatacccttggttggagttaatcaacccttcagaaatgtttggagacatcgtcattgattattcgtatgtagagtgcaccgcagccacaatggaagcactggcgttgtttaagaagttacatccgggccataggaccaaagaaatcgataccactttagccaaggcagccaacttccttgaaaacattcaaaggccggatggctcttggtatggatgttggggggtttgcttcacttatgccggctggtttggcataaagggattggtagctgcaggaaggacatataatagttgcctagccattcgcaaggcttgtgattttctactctctaaagagctgcccggcggtggatggggggagagttacctttcatgtcagaatagggtctacaccaatctcaaaggaaaccgaccacacttggttaacacagcctgggttttattggccctcatcgaagctggccaggctgagagagatccagcaccactgcacagagcagccaggttgttaatcaattcccagttggagaatggtgatttcccccaagaggagatcatgggagtgttcaataaaaattgcatgatcacatatgctgcatacagaaacttttttcccatttgggctcttggagaatattcccatcgggtattgactgaataa

SEQ ID NO: 204 - protein, 762 aa, Trichosanthes cucumerina
MWKLKVGAESAGEKNEKWLKSISNHLGRQVWEFCPDAAAPHHQLQLHNAKKHFHHNRFSRKQSSDLLLAIQCEKEIANGAKVAAIKLKEGEGVRKEAVKITLERALGFYSAIQTPDGNWASDLGGPMFLIPGLVIALYVTGALNSILSKHHRQEMCRYLYNHQNEDGGWGLHIEGPSTMFGSGLNYVALRLLGEDADGGEGGAMTKARAWILARGGATAIPSWGKLWLSVLGVYEWSGNNPLPPEFWLLPYSLPFHPGRMWCHCRMVYLPMSYLYGKRFVGPITPIVLSLRKELFTIPYHEIDWNKSRNTCAKEDLFYPHPKMQDILWGSLHHLYEPLFSRWPGKRIREKALQTAMQHIHYEDENTRYICLGPVNKVLNMLCCWVEDPYSDAFKLHLQRVHDYLWIAEDGMRMQGYNGSQLWDTAFSIQAILSTKLVDSYGPTLRKAHDFVKNSQIQQDCPGDPNVWFRHIHKGAWPFSTRDHGWLISDCTAEGLKASLMLSKLPSEIVGEPLEKNRLCDAVNVLLSLQNDNGGFASYELTRSYPWLELINPSEMFGDIVIDYSYVECTAATMEALALFKKLHPGHRTKEIDTTLAKAANFLENIQRPDGSWYGCWGVCFTYAGWFGIKGLVAAGRTYNSCLAIRKACDFLLSKELPGGGWGESYLSCQNRVYTNLKGNRPHLVNTAWVLLALIEAGQAERDPAPLHRAARLLINSQLENGDFPQEEIMGVFNKNCMITYAAYRNFFPIWALGEYSHRVLTE

SEQ ID NO: 205 - DNA, 2295 bp, synthetic construct
  source 1..2295: mol_type = other DNA
atgtggaggttaaaagttggggcggagagcgttggggagaaagaagagaaatggttgaagagtataagcaatcacttgggacgccaagtgtgggagttttgtgccgaccaacccactgcttctccaaatcacttacaacaaatcgataatgcgaagaagcactttcgtaataatcgcttccaccggaagcaatcttccgatctctttcttgctattcagaatgaaaaggagatagcaaatggtacaaaaggtggaggaatcaaagtgaaagaagaggaagatgtaaggaaagagacagtgaagaatacagtagaaagggcattaagtttctattcagccattcagacaaacgatgggaattgggcttcggatcttggagggcccatgtttttactcccgggtcttgtgattgctctctatgtcactggcgtcttgaattcggttctatccaagcaccatcgacaagaaatgtgtagatatctttataatcaccagaatgaagatggtggatggggcctccacatcgaaggtacgagcacgatgtttggttcggcactgaattatgttgcactgaggttgcttggagaagacgctgatggcggagagggcggtgcaatgacaaaagcacgtggttggatcttggaccgcggcggtgccaccgcaatcacttcttggggaaaattgtggctctctgtacttggagtctatgaatggagtggcaataatcctcttccacctgagttttggttactcccttattgcctaccctttcatccaggaagaatgtggtgccattgtcgaatggtttatctaccaatgtcatacttatatggaaagagatttgttgggccaatcacacccatagttctatctctaagaaaagagctctacacaattccatatcatgaaatagattggaataaatctcgcaatacatgtgcaaaggaggatctgtactatccacatccgaagatgcaagatattctgtggggatcaatataccatttgtatgagcctttatttactcgttggcctggaaaacggctgagggaaaaggctttgcaaatggcaatgaaacatattcactatgaagatgaaaacagtagatatatatgtcttggacctgtcaataaagtacttaatatgctttgttgttgggttgaagatccttattcagatgccttcaaatttcatcttcaacgagtccccgactatctttggattgctgaagatggcatgagaatgcagggttacaatgggagccagttgtgggacactgctttctctgttcaagcaatcatatccaccaaacttatagacagctttggcacaaccttaaaaaaagcacatgattttgtcaaagattctcagatccagcaggactttcctggggatcctaatgtttggttccgtcacattcataaaggtgcttggccattttcaactcgtgatcatggatggctcatctctgactgtacagctgaggggttaaaggcttctttgatgttatccaaacttccatccaaaatagttggggagccattagaaaagagtcgcctttgcgatgctgtaaatgttctcctttctttacaaaatgaaaatggtggatttgcatcatatgagttgacaagatcatacccttggttggagttgatcaaccctgcagaaacattcggagatatcgttatcgattatccctatgtggagtgcacctcagctacaatggaagcattgacactgtttaagaagttacatccaggccataggaccaaagagattgatactgcagttgccaaggcagctaacttcctagaaaatatgcaaaggacggatggctcttggtacgggtgttggggggtttgcttcacgtatgcagggtggtttggcataaagggtttggttgctgcaggaaggacatatagtacttgtgttgccattcgcaaggcttgtgattttttgctatctaaagaattacctggcggtggatggggagagagttacctttcatgtcagaataaggtctataccaatcttgaaggcaacaggccgcatttggttaacactgcttgggttttgatggctctcattgaagctggccaggctgagagagatccagcaccattgcaccgtgcagcaaggttgttaatcaattcccaattggagaatggtgatttcccccaagaggaaatcatgggagtgtttaataaaaattgcatgatcacatatgctgcatatcgaaatatctttcccatttgggctcttggagagtatttccatcgggttcttactgaatga

SEQ ID NO: 206 - protein, 764 aa, Citrullus colocynthis
MWRLKVGAESVGEKEEKWLKSISNHLGRQVWEFCADQPTASPNHLQQIDNAKKHFRNNRFHRKQSSDLFLAIQNEKEIANGTKGGGIKVKEEEDVRKETVKNTVERALSFYSAIQTNDGNWASDLGGPMFLLPGLVIALYVTGVLNSVLSKHHRQEMCRYLYNHQNEDGGWGLHIEGTSTMFGSALNYVALRLLGEDADGGEGGAMTKARGWILDRGGATAITSWGKLWLSVLGVYEWSGNNPLPPEFWLLPYCLPFHPGRMWCHCRMVYLPMSYLYGKRFVGPITPIVLSLRKELYTIPYHEIDWNKSRNTCAKEDLYYPHPKMQDILWGSIYHLYEPLFTRWPGKRLREKALQMAMKHIHYEDENSRYICLGPVNKVLNMLCCWVEDPYSDAFKFHLQRVPDYLWIAEDGMRMQGYNGSQLWDTAFSVQAIISTKLIDSFGTTLKKAHDFVKDSQIQQDFPGDPNVWFRHIHKGAWPFSTRDHGWLISDCTAEGLKASLMLSKLPSKIVGEPLEKSRLCDAVNVLLSLQNENGGFASYELTRSYPWLELINPAETFGDIVIDYPYVECTSATMEALTLFKKLHPGHRTKEIDTAVAKAANFLENMQRTDGSWYGCWGVCFTYAGWFGIKGLVAAGRTYSTCVAIRKACDFLLSKELPGGGWGESYLSCQNKVYTNLEGNRPHLVNTAWVLMALIEAGQAERDPAPLHRAARLLINSQLENGDFPQEEIMGVFNKNCMITYAAYRNIFPIWALGEYFHRVLTE

SEQ ID NO: 207 - DNA, 1365 bp, synthetic construct
  source 1..1365: mol_type = other DNA
atggggagtttaggagcaattttgaaacacccagatgaattttacccacttttgaagttgaagatggctgttaaagaagctgagaaacagatcccatctgaatctcattgggggttttgttactctatgcttcataaagtttccagaagttttgctcttgttattcagcaacttggcactgagcttcgcaatgctgtatgtgtcttttacttggtccttcgagccctcgacactgtagaggatgatacaagcatagctacagatgtcaaattgcctatcctgaaagctttttatcagcacatatatgatcgcgaatggcacttttcttgtggcacaaaacactacaaagttctcatggatgagtttcatcaagtctcaactgcttttttggagcttgaaagaggttaccagctcgcaattgaggatattaccaaaaggatgggtgcgggaatggcaaaattcatctgtcaggaggtagaaacagttagcgactatgacgaatactgccactatgtagcaggactcgtgggtttagggttgtctaagcttttccataatgcaggtttggaggatcttgcatcggatgatctttccaattcgatgggtttatttcttcaggtatttccttttgcttggaacatgaatttgcaaacttctgtgctctgtgagactgtgatactatacctaataagatgttacatagtgtaatcagtaatgtgcctattttttgtgtagaaaaccaacattatccgtgattacctcgaagatataaatgagattccaaaatgtcgcatgttttggcctcgggagatatggagtaaatatgtcaacaagcttgaggacctgaaatatgaggagaactctgtgaaggcagttcaatgtttaaatgacatggtaacaaatgctttattgcatgtggaagattgcctaaagtacatgtcggctctgcgggatcatgctatattccggttctgtgctattccacaaatcatggccattggaactttagctttatgctacaacaacgttgaagtcttcagaggtgtggttaaaatgagacgtggtcttaccgcaagagttattgataagacagactcgatgcctgatgtttatggtgccttctacgattttgcctgcatgataaaaccaaaggtcgacaagaatgatccaaacgccatgaaaacactaagtagaatcgatgctatcgagaagatttgtagggactccggcactctgaacaaaaggaaactgcacatcacaagcaccaagtcggcatacactccaatcatggtgatggtgttattcattgtattggcaatcttcttcaatcgtttatcagaatccaacagaatgattaataactaa

SEQ ID NO: 208 - protein, 415 aa, synthetic construct
MGSLGAILKHPDEFYPLLKLKMAVKEAEKQIPSESHWGFCYSMLHKVSRSFALVIQQLGTELRNAVCVFYLVLRALDTVEDDTSIATDVKLPILKAFYQHIYDREWHFSCGTKHYKVLMDEFHQVSTAFLELERGYQLAIEDITKRMGAGMAKFICQEVETVSDYDEYCHYVAGLVGLGLSKLFHNAGLEDLASDDLSNSMGLFLQKTNIIRDYLEDINEIPKCRMFWPREIWSKYVNKLEDLKYEENSVKAVQCLNDMVTNALLHVEDCLKYMSALRDHAIFRFCAIPQIMAIGTLALCYNNVEVFRGVVKMRRGLTARVIDKTDSMPDVYGAFYDFACMIKPKVDKNDPNAMKTLSRIDAIEKICRDSGTLNKRKLHITSTKSAYTPIMVMVLFIVLAIFFNRLSESNRMINN

SEQ ID NO: 209 - DNA, 1245 bp, synthetic construct
  source 1..1245: mol_type = other DNA
atggggagtttgagggcgattttgaagcaccctgatgatttgtacccgttggtgaaactgaaactggcggcgaggcacgcagagaagcagatcccgcgggagccacactggggattttgctacgctatgctacacaaggtttctaggagttttgctctcgtcattcaacagctcgacaccgatcttcgtgatgccatatgcattttctacttggttcttcgagcgcttgacactgttgaggacgatacaagcatagctacggacatcaaagtacctatcctgatggcttttcatcgtcacatatatgatcgtgaatggcatttttcatgtggtgtaaagcactacaaagttctcatggatgagttccatcacgtttccactgcttttctggagcttggaagaagttatcaggaggcaattgaggatattaccatgaggatgggagcaggaatggcaaaatttatatgcaaggaggtagaaacaattgatgattatgatgaatattgccactatgtggctggacttgttgggttagggttgtcgaagctgttctatgcatctggaaaagaagatttggcttctgatactctctccaactcaatggggttatttcttcagaaaacaaacattatcagagactacctggaggatattaatgagataccaaagtcacgtatgttctggcctcgtcagatatggagtaaatatgtcaacaaacttgaggatttaaaatatgaggaaaactctgtcaaggcagtacagtgcctcaatgacatggttacaaatgctttgatacatgtagaagattgcctgaagtacatgtctgcattgcgagatccagctatctttcggttttgtgctattccacagatcatggcaattggaacactagctttgtgctacaacaatattcaagtcttcaggggtgttgtgaaaatgagacgtggtcttacagctaaagttattgaccgaaccaaaacaatgtcagatgtctatggggatttttatgatttctctcgcatgctgaagtctaaggtggacaacagtgatcctaacgctaataaaacaaaggagaagctagaaacaatcttgaaaatttgcagagactctggatctctaaacaataggaaatcttacatcattaggagcgagcctagatacaattcaactacgatcgttgtcatcttcatcatacttgctatgcttcttgcctacctctctgcgactagaccaaataatatatga

SEQ ID NO: 210 - protein, 414 aa, Olea europaea
MGSLRAILKHPDDLYPLVKLKLAARHAEKQIPREPHWGFCYAMLHKVSRSFALVIQQLDTDLRDAICIFYLVLRALDTVEDDTSIATDIKVPILMAFHRHIYDREWHFSCGVKHYKVLMDEFHHVSTAFLELGRSYQEAIEDITMRMGAGMAKFICKEVETIDDYDEYCHYVAGLVGLGLSKLFYASGKEDLASDTLSNSMGLFLQKTNIIRDYLEDINEIPKSRMFWPRQIWSKYVNKLEDLKYEENSVKAVQCLNDMVTNALIHVEDCLKYMSALRDPAIFRFCAIPQIMAIGTLALCYNNIQVFRGVVKMRRGLTAKVIDRTKTMSDVYGDFYDFSRMLKSKVDNSDPNANKTKEKLETILKICRDSGSLNNRKSYIIRSEPRYNSTTIVVIFIILAMLLAYLSATRPNNI

SEQ ID NO: 211 - DNA, 1386 bp, synthetic construct
  source 1..1386: mol_type = other DNA
atgggaaagttaggggagctactctcacatccaaatgagatcatgccactgctgcgcatgtatcgcgcagctaagcgcgccaccaagcttccaaaggacccggacctggctttctgctacgggatgctgaacgacgtgtctcgaagcttcgccatcgtcattcagcagctgcccaacccgctgcgcgactccatctgcgtgttctacctggtgctgcgcgggctggacaccgtggaggatgacatggcactggacgtggacttcaagctgcccgagctgcgtcgtttccatgagcgcatatacgacaggggcttcacgatggagtgcggctacaaccactacaagcggctgatggcgcagttcggggtggtggtgaacgtgttcctggcgctgccgccgccgcaccaggccgtcatcgccgacatcacgcggcgcatgggctacggcatggcggagttcatcgagaaggaggtggtgacggtggccgactacgacaagtactgccactacgtggcgggcctggtgggcatcggcctcagccagctgttcgccgccagcggcctggaggccaactccatcggcaccgaggaggacctggccaaccacatggggctgttcctgcagaagaccaacatcatccgcgactacctggaggacattctggaggagccggcgccgcgcatgttctggccccgtgacatctggggcaagtacgcgcccgcgctggccgacttcaaggagccggccaacaggggggcggcggtgcggtgcctcaacgacatgatcacggacgcgctgcggcaccttccgtactgcctggcctacatggagcagctgcggcacgtgatggtgttccgcttctgcgccatcccgcagatcatggcggctggcaccctggcagcatgctacaacaacggccgcgtgttcgagggcgtggtgaagatgcggcgcggcgagacggcccgcatctttgagacgtgcaacgacctgggcgacctgtacacctggttcctgcagttcctggcggccatggaggccaaggtgtcaaacgaggtagacccaacggacccctcgctgcccctcacgcggcaacgcatcgcggaggcacgcaaggcctgcgccgccaagctcacagagtggaagcgcacgcacccgccgcccagcgagagcggcgccgcgctgacccgcgacttcctggcgctctctatcaccggcgcctacttctactacgtgtggcagctgggcgggccgggctcggaagcgctgccggagctgctgcgcccctacgcctgggtattggagcagcaggcggggctgcagaaggccgtgagcctggcgctgctggtgctggtgttcctgctggtggcggtactgcggcgtgcgtga

SEQ ID NO: 212 - protein, 461 aa, Chlamydomonas reinhardtii
MGKLGELLSHPNEIMPLLRMYRAAKRATKLPKDPDLAFCYGMLNDVSRSFAIVIQQLPNPLRDSICVFYLVLRGLDTVEDDMALDVDFKLPELRRFHERIYDRGFTMECGYNHYKRLMAQFGVVVNVFLALPPPHQAVIADITRRMGYGMAEFIEKEVVTVADYDKYCHYVAGLVGIGLSQLFAASGLEANSIGTEEDLANHMGLFLQKTNIIRDYLEDILEEPAPRMFWPRDIWGKYAPALADFKEPANRGAAVRCLNDMITDALRHLPYCLAYMEQLRHVMVFRFCAIPQIMAAGTLAACYNNGRVFEGVVKMRRGETARIFETCNDLGDLYTWFLQFLAAMEAKVSNEVDPTDPSLPLTRQRIAEARKACAAKLTEWKRTHPPPSESGAALTRDFLALSITGAYFYYVWQLGGPGSEALPELLRPYAWVLEQQAGLQKAVSLALLVLVFLLVAVLRRA

SEQ ID NO: 213 - DNA, 1575 bp, synthetic construct
  source 1..1575: mol_type = other DNA
atggagttcctatccgcggctcttctcggccttctattggcctccatccttcctctctttttcttcttctctttcagagacggaatcaaacacgacggcgacaacaacaccaccgccgtaatcgattcaaagattccgtcgggaaatgacgtccgtcgcgccgaatcctccgatgattttgacgttatcatcgtcggcgctggagttgccggcgccgcccttgcttatactctcgccaaggatggacgaaaagtgcatgtaattgaaagagacttgtcggagccggagagaatcgtcggagaattgcttcaacccggcggctatttgaagctcatcgagttagggcttgaagacagtctgaaggggatcgatgctcagcaagtgtttggttatgcgctcttcaaagacggaaaccacaccagattggcttatcctttggacaaatttgattccaatatttctgggagaagcttccacaatggccgattcattcagaggttgaggcaaaaggctgctgctctttccaatgtaaaactggaacaagggactgtaacttccatagttgaagaagatgggactgtaaaaggtgtgcaatataaaaccaagaatggacaagagttgacatcttatgctcccctcaccattgtttgtgatggcggcttttcgaatttgcgtcgcaacctctgcaaacctcagattgatattccctcatgctttgttggtttggtcctggagaactgtaagctgccccttgaaaatcatggacatgttgtgcttgctgacccatcgcctatcttgttataccggattagcagcacggagatacgctgcttagtcgatatccccggccaaaaagtgccttctgtggctaacggcgacatggcacactacttgaaaaccgctgtggctcctcagctaccgcccgagttgcagaccgccttcatagcttcagttgataaggggaacataaaaagcacaacaaacagaagcatgccagcagccccacatcccacaccaggagcacttctcttgggtgatgcattcaacatgcgacatcctttaaccggtggaggtatgaccgttgctctcgccgatatcgtcgtgctacgagatcttcttcgacccctttcaaatttgaacgatgccgatgccttgtgcaactatctcgaatccttctacacattgcgaaagccagtggcatctacaataaacacattggccggtgccttataccaggtcttttgtgcatcgcccgacccggccaggaaggaaatgcgcgaagcttgtttcgattatttgagtctaggaggtgtattttcctctggaccagtgtctcttctctctggtctgaacccgcgcccattaagcctgttctgtcacttctttgccgtggccatatacggtgtcactcggttgctaatcccattcccttcgcctaagcgtatatggatcggagttcgattgattacgggagcagcaggcataatttttcccataatcaaggctgaaggagtgagacaaatgttctttcctgcaacaatgcctgcatactacagagcccctcctatgcagtga

SEQ ID NO: 214 - protein, 524 aa, Cucurbita pepo
MEFLSAALLGLLLASILPLFFFFSFRDGIKHDGDNNTTAVIDSKIPSGNDVRRAESSDDFDVIIVGAGVAGAALAYTLAKDGRKVHVIERDLSEPERIVGELLQPGGYLKLIELGLEDSLKGIDAQQVFGYALFKDGNHTRLAYPLDKFDSNISGRSFHNGRFIQRLRQKAAALSNVKLEQGTVTSIVEEDGTVKGVQYKTKNGQELTSYAPLTIVCDGGFSNLRRNLCKPQIDIPSCFVGLVLENCKLPLENHGHVVLADPSPILLYRISSTEIRCLVDIPGQKVPSVANGDMAHYLKTAVAPQLPPELQTAFIASVDKGNIKSTTNRSMPAAPHPTPGALLLGDAFNMRHPLTGGGMTVALADIVVLRDLLRPLSNLNDADALCNYLESFYTLRKPVASTINTLAGALYQVFCASPDPARKEMREACFDYLSLGGVFSSGPVSLLSGLNPRPLSLFCHFFAVAIYGVTRLLIPFPSPKRIWIGVRLITGAAGIIFPIIKAEGVRQMFFPATMPAYYRAPPMQ

SEQ ID NO: 215 - DNA, 1599 bp, synthetic construct
  source 1..1599: mol_type = other DNA
atggagtttcaatctgcggctctctgcggccttctcttggcttccatttccgctctcattttcttcttcgctcacctcagattcaataatggaatatccaacaaaaccaacactgacaacatgatcaagaagaagaagatcttcatcgccggttcccgtgccgccgtcaacccgccgtcggaaaatgtccaatccaacgaacccgatgtcgtcatcgtcggcgccggagtcgctggcgccgccctcgcttatactctcgccaaggatggacgaaaagttcatgtaatcgaaagagacttagcggagccagagagaatcgtgggggaattgcttcaacccggcggctatctgaagctgatcgaattagggcttgaagacagtgtgaatgggatcgatgcgcagcaagtgtttggatatgcgctgtacaaggatgggaagcacaccaaattggcttatcctttgcaaaactttgattcccatgtttctgggagaagcttccacaatggccgtttcattcagcggttgaggcaaatggctgcttctctttccaatgtaagactggaacaggggactgtaacatccatagttgaagaagatgggactgtgaaaggtgtgcagtacaagaccaagaatggccaagagttgacagcttatgctcccctcaccattgtttgcgatggcggattttcgaatttgcgccgcaacctctgcaaacctcagattgatattccctcctgctttgttggattggtcctggagaactgcaagctgccctttgaaaatcatggacatgtcgtgctagcagatccgtcacctatcttgttgtaccctatcagcagcacagagatacgctgcttggtcgatatcccgggccagaaagttccttctgtagctaatggtgaaatggcacactacttgaagaccgttgtggctcctcaggtaccacccgagttgcacgcctccttcattgctgcgatcgagaagggaaacattaaaagcacaacaaacagaaccatgccagcagcaccacgtcccacaccaggagcacttctcttgggggatgcattcaacatgcgacatcctttaacgggcggaggaatgacggtcgctctggctgatatcgttgtgctacgtgaccttcttcgacctcttgcaaatttgaaagatgccgatgcattgtgcaactattttgaatccttctacactttgcgcaagccagtggcatctacaataaacacattggctggtgccttatacaaggtgttctgtgcctcacctgatccagccaggaaggaaatgcgagaagcttgtttcgactatttgagtctaggaggcctgttttcatccggtccagtggctcttctctccggtcttaacccacgcccgttaagcctgttctgtcacttcttcgctgtcgctatatacggtgtcagccgcttgctaatcccattcccttcgcccactcgcatatggatcggagttcggttgattacgggagcagcaggcatcatttttcccataataaaggctgaaggagtgagacaaatgttcttccctgcaacagtgcctgcatattatagagcccctcctatgaactga

SEQ ID NO: 216 - protein, 532 aa, Momordica charantia
MEFQSAALCGLLLASISALIFFFAHLRFNNGISNKTNTDNMIKKKKIFIAGSRAAVNPPSENVQSNEPDVVIVGAGVAGAALAYTLAKDGRKVHVIERDLAEPERIVGELLQPGGYLKLIELGLEDSVNGIDAQQVFGYALYKDGKHTKLAYPLQNFDSHVSGRSFHNGRFIQRLRQMAASLSNVRLEQGTVTSIVEEDGTVKGVQYKTKNGQELTAYAPLTIVCDGGFSNLRRNLCKPQIDIPSCFVGLVLENCKLPFENHGHVVLADPSPILLYPISSTEIRCLVDIPGQKVPSVANGEMAHYLKTVVAPQVPPELHASFIAAIEKGNIKSTTNRTMPAAPRPTPGALLLGDAFNMRHPLTGGGMTVALADIVVLRDLLRPLANLKDADALCNYFESFYTLRKPVASTINTLAGALYKVFCASPDPARKEMREACFDYLSLGGLFSSGPVALLSGLNPRPLSLFCHFFAVAIYGVSRLLIPFPSPTRIWIGVRLITGAAGIIFPIIKAEGVRQMFFPATVPAYYRAPPMN

SEQ ID NO: 217 - DNA, 1611 bp, synthetic construct
  source 1..1611: mol_type = other DNA
atgaattcatcttcttctagtactactgatacgttgcattcttttatggaagctctgctcattgatcaatattttcttggatggatctttgctttcctttttgggttcctgctcttgcttaatttcaaaaggaagagagagaaaaataattccacggagtttggaacagatgatagcaacggttactacacaccggaaaatattgccggaagtacggacgtcatcatcgtcggcgccggggttgctggctctgctcttgcttatacgcttgccaaagatggccggagagttcatgtaattgagagggacttaactgagcaagaccgaattgtaggtgaacttctacaaccaggaggctacttgaaattgattgaattggggttagaggattgtgtgaatgaaatcgatgcgcaacgagtttttggatatgccctatacatggatggtaaaaacaccaggctttcttaccccttggagaaatttcattcggatgtagccggaagaagctttcataacgggcgttttgttcaacgaatgagggagaaagctgcatcacttccaaatgtaagaatggaacaggggactgttacatctctggttgagaaaaaggcaagtgtaaaaggggtgcaatacaaaaccaaggatggccaagaattgtctgcatttgctcctcttacaattgtttgtgatggttgtttttcgaatctgcgtcgctccctctgcaatcccaaggtggaggtgccttcgtgttttgttggtttgattttggaaaatattgatcttccacatgtaaaccatggccatgtcattctagcagatccttctccgatcttgttttataaaattagtagtaccgagattcgctgtttggttgatgtgcctggacaaaaggtgccttgtatttctaatggggaattggctaattatctcaagacagtagtagctcctcaggttccaaaacagctatataactctttcatagcagcagttgacaaaggaaacattagaaccatgccaaacagaagcatgccagccgatcctcatccaactccgggtgcacttctattaggggatgctttcaatatgcgccatcctttaaccggcgggggaatgacagtggctctgtccgatattgtcttgattcgggatcttcttagacctttacgcgatctccatgactcatcaaccctctgcaaatatctcgaatccttttacacccttcgtaagcccgtggcatctactataaatacattggcaggcgccctttataaagttttttgtgcatcacctgataaagcaaggcaagaaatgcgcaatgcgtgttttgattatctgagcctcggaggaatttgttcccaagggccaattgctttactttctggcctaaacccgcgtccaattagcctatttctccacttctttgccgtggctatctatggcgttggccgcttgttgattccttttccttcaccaaaacgaatgtggctcggcgctagattgattttgggtgcatctggaattattttccctattataaagtcagaaggacttcgacaaatgttcttccctgcaacggttcctgcttactacagagctccccccattcactaa

SEQ ID NO: 218 - protein, 536 aa, Panax ginseng
MNSSSSSTTDTLHSFMEALLIDQYFLGWIFAFLFGFLLLLNFKRKREKNNSTEFGTDDSNGYYTPENIAGSTDVIIVGAGVAGSALAYTLAKDGRRVHVIERDLTEQDRIVGELLQPGGYLKLIELGLEDCVNEIDAQRVFGYALYMDGKNTRLSYPLEKFHSDVAGRSFHNGRFVQRMREKAASLPNVRMEQGTVTSLVEKKASVKGVQYKTKDGQELSAFAPLTIVCDGCFSNLRRSLCNPKVEVPSCFVGLILENIDLPHVNHGHVILADPSPILFYKISSTEIRCLVDVPGQKVPCISNGELANYLKTVVAPQVPKQLYNSFIAAVDKGNIRTMPNRSMPADPHPTPGALLLGDAFNMRHPLTGGGMTVALSDIVLIRDLLRPLRDLHDSSTLCKYLESFYTLRKPVASTINTLAGALYKVFCASPDKARQEMRNACFDYLSLGGICSQGPIALLSGLNPRPISLFLHFFAVAIYGVGRLLIPFPSPKRMWLGARLILGASGIIFPIIKSEGLRQMFFPATVPAYYRAPPIH

SEQ ID NO: 219 - DNA, 1254 bp, synthetic construct
  source 1..1254: mol_type = other DNA
atgctggttcgatccgtggtggatggatcaatgccatcgtactctctagaaacccgcctcaaagactgccgtagagcggcgatgatcaggcgtgaggcggtggagatgataactgggaggtcccttgaagggctaccattagaggggtttgattacgagtcgattttgaacaagtgctgtgagatgacagttgggtatgttcagattcctgtaggagttgtgggtccattgttgttggatgggattgagtatattgtaccaatggctactactgaggggtgtttggtagcgagtacaaatcgcgggtgtaagactatatatgcttctgggggtgttactagttttgttcttaaggatccaatgactagagctcctgttgttaggtttccttctgctaaacgagcttctgaattgaagttttttcttgaagatccacttaattttgacaccatggctgttattttcaatgagtcgagcaggttcgcgaggctgcaaagcgtaaaatgtggaatggcagggaagaatttgtacctacgattcagttgtagcacaggggatgcgatgggcatgaacatggcgtccaaagctgttgagcaagtcctcagtttcctccaggatgagtttcctgacatggatgctattggcgtttctgggaacttctgttcagacaaaaaagctgctgcagtgaactggatagaggggcgtggaaagtcggttgtttgtgaagcagtaatcaaggaagaagtggtgacaagggtgttaagaagcagtgcctcggccctagttgagcttaatattctaaaaaatctcgctggttctgccatagctggagctcttggtggttttaatgcctatgccagcaatatagtgtccgcaatatttatagccacaggacaagatccagctcaaaatgttgaaagctctcagtgcatcaccatgattgaagctgtgaatgatggcaaggatcttcatgtctctgtctcaatgccatccattgaggtgggtacagttggaggggggactacacttgcatcgcaagcagcatgtctgaacctgctaggagtgaagggtgcaagtgaaggctcagcaggagcaaactcaaggcttctggctaccatagttgctggatcagtccttgcaggggaactctcattaatgtccgccatagcagaaggtgagctggtgagtagtcacatgaaatacaacaggtcaagcaaggatatggctaaatctatgctcacatcttgctcttaa

SEQ ID NO: 220 - protein, 417 aa, Achyranthes bidentata
MLVRSVVDGSMPSYSLETRLKDCRRAAMIRREAVEMITGRSLEGLPLEGFDYESILNKCCEMTVGYVQIPVGVVGPLLLDGIEYIVPMATTEGCLVASTNRGCKTIYASGGVTSFVLKDPMTRAPVVRFPSAKRASELKFFLEDPLNFDTMAVIFNESSRFARLQSVKCGMAGKNLYLRFSCSTGDAMGMNMASKAVEQVLSFLQDEFPDMDAIGVSGNFCSDKKAAAVNWIEGRGKSVVCEAVIKEEVVTRVLRSSASALVELNILKNLAGSAIAGALGGFNAYASNIVSAIFIATGQDPAQNVESSQCITMIEAVNDGKDLHVSVSMPSIEVGTVGGGTTLASQAACLNLLGVKGASEGSAGANSRLLATIVAGSVLAGELSLMSAIAEGELVSSHMKYNRSSKDMAKSMLTSCS

SEQ ID NO: 221 - DNA, 1275 bp, synthetic construct
  source 1..1275: mol_type = other DNA
atgattgcacccttagtctcggaggaagacgaaatgatcgtcaactccgtcgtggatgggaagataccctcctattctctggagtcgaagctcggggactgcaaacgagcggctgcgattcgacgcgaggctttgcagaggatgacaaggaggtcgctggaaggcttgccagtagaagggttcgattacgagtcgattttaggacaatgctgtgaaatgccagtgggatacgtgcagattccggtggggattgcggggccgttgttgctgaacgggcgggagtactctgttccaatggcgaccacggagggttgtttggtggcgagcactaatagagggtgtaaggcgatttacttgtcaggtggggccaccagcgtcttgttgaaggatggcatgacaagagcgcctgttgtaagattcgcgtcggcgactagagccgcggagttgaagttcttcttggaggatcctgacaattttgataccttggccgtagtttttaacaagtccagtagatttgcgaggctccaaggcattaaatgctcaattgctggtaagaatctttatataagattcagctgcagcactggcgatgcaatggggatgaacatggtttctaaaggggttcaaaacgttcttgaatttcttcaaagtgatttttctgatatggatgtcattggaatctcaggaaatttttgttcggataagaagcctgctgctgtaaattggattgaaggacgtggcaaatcagttgtttgtgaggcaattatcaaggaagaggtggtgaagaaggtgttgaaaaccaatgtggcctccctagtggagcttaacatgctcaagaatcttgctggttctgctgttgctggtgctttgggtggatttaatgcccatgcaggcaacatcgtatctgcaatctttattgccactggccaggatccagcacagaatgttgagagttctcattgcattaccatgatggaagctgtcaatgatggaaaggatctccatatctctgtgaccatgccctccattgaggtgggtacagtcggaggtggaactcaacttgcatctcagtctgcttgtctcaatttgcttggggtgaagggtgcaaacaaagagtcgccaggatcaaactcaaggctccttgctgccatcgtagctggttcagttttggctggtgagctctccttgatgtctgccattgcagctgggcagcttgtcaagagtcacatgaagtacaacagatccagcaaagatatgtctaaagctgcatcttag

SEQ ID NO: 222 - protein, 424 aa, Hevea brasiliensis
MIAPLVSEEDEMIVNSVVDGKIPSYSLESKLGDCKRAAAIRREALQRMTRRSLEGLPVEGFDYESILGQCCEMPVGYVQIPVGIAGPLLLNGREYSVPMATTEGCLVASTNRGCKAIYLSGGATSVLLKDGMTRAPVVRFASATRAAELKFFLEDPDNFDTLAVVFNKSSRFARLQGIKCSIAGKNLYIRFSCSTGDAMGMNMVSKGVQNVLEFLQSDFSDMDVIGISGNFCSDKKPAAVNWIEGRGKSVVCEAIIKEEVVKKVLKTNVASLVELNMLKNLAGSAVAGALGGFNAHAGNIVSAIFIATGQDPAQNVESSHCITMMEAVNDGKDLHISVTMPSIEVGTVGGGTQLASQSACLNLLGVKGANKESPGSNSRLLAAIVAGSVLAGELSLMSAIAAGQLVKSHMKYNRSSKDMSKAAS

SEQ ID NO: 223 - DNA, 1263 bp, synthetic construct
  source 1..1263: mol_type = other DNA
atgtcatcggaggaggacgaagaaatcgtgaaatccgtggtttcggggaaaacgccgtcgtattcgctggaatcaaaactcggggactgttatagagcggcgtcgattcgacgcgaggcggtgcagaggactacggggaggtcactgctggggttgccgttggatggtttcgattatgaatccatattagggcagtgctgtgagatgccgatagggtatgtgcaaattccggtggggattgcgggtccattgctgctcaatggatgcgagtatgtggtgccgatggccacaacggaggggtgtttggttgcgagtactaacagaggctgcaaggccatttatgcgtgcggtggtgccacggggattttgctaaaagatgggatgaccagagcgcctgtggttaggttttctacggcaaaaagggcctcggatttgaagttctttttggaagatcctcttaattttgatacactggccgtcgtttttaataaatcaagcagatttgctaggctgcaaagcattcaatgctcaatggcagggaaaaatctttacatcagattttgctgcagcactggggatgcgatgggaatgaacatggtgtccaagggtgttcagaatgtcttggagtttcttcaaagtgatttccctgacatggatgtgattggtatctctggaaatttttgttctgataagaaaccagctgcagtcaattggattgaagggcgaggtaaatctgttgtttgtgaggcaattataactgatgatgtggtgaagaaggtattgaaaaccacagtgcctgcccttgtagagcttaacatgcttaagaatcttgctggttctgctgttgctggtgctcttggtggctttaatgcccatgctgccaatatcgtctctgcagttttcatagccactgggcaggacccagcacaaaatattgaaagttctcactgcataactatgatggaggccattaacaatggaaaagatcttcacatttctgtcacaatgccatcgattgaggttggtactgtcggaggtggaacacaactggcgtctcaatctgcttgcttgaaccttcttggtgtgaagggcgcaaacaaagaatcccatggatcaaactccaggctcttggccaccatagtagctggctcagttttggctggagagctctccttgatgtctgccattgcagctgggcagcttgtcaggagccacatgaaatataacaggtcaagccgggatatgtccaaaattggatcttaa

SEQ ID NO: 224 - protein, 420 aa, Panax ginseng
MSSEEDEEIVKSVVSGKTPSYSLESKLGDCYRAASIRREAVQRTTGRSLLGLPLDGFDYESILGQCCEMPIGYVQIPVGIAGPLLLNGCEYVVPMATTEGCLVASTNRGCKAIYACGGATGILLKDGMTRAPVVRFSTAKRASDLKFFLEDPLNFDTLAVVFNKSSRFARLQSIQCSMAGKNLYIRFCCSTGDAMGMNMVSKGVQNVLEFLQSDFPDMDVIGISGNFCSDKKPAAVNWIEGRGKSVVCEAIITDDVVKKVLKTTVPALVELNMLKNLAGSAVAGALGGFNAHAANIVSAVFIATGQDPAQNIESSHCITMMEAINNGKDLHISVTMPSIEVGTVGGGTQLASQSACLNLLGVKGANKESHGSNSRLLATIVAGSVLAGELSLMSAIAAGQLVRSHMKYNRSSRDMSKIGS

SEQ ID NO: 225 - DNA, 1872 bp, synthetic construct
  source 1..1872: mol_type = other DNA
atgactgattaccggctacaaccaacgatgaatctttggaccaccgacgacaacgcttctatgatggaagctttcatgagctcttccgatatctcaactttatggcctccggcgtcgacgacaaccacgacggcgacgactgaaacaactccgacgccggcgatggagattccggcacaggcgggatttaatcaagagactcttcagcaacgtttacaagctttgattgaaggaacacacgaaggttggacctacgctatattctggcaaccgtcgtatgatttctccggcgcctccgtgctcggatggggaaacggttattacaaaggtgaagaagataaagcaaacccgagacggagatcgagttcgccgccgttttctactccggcggatcaggagtacaggaaaaaagtgttgagagagcttaactcgttgatctccggtggtgttgctccgtcggatgacgctgttgatgaggaggtgacggatacggaatggtttttcttggtttcgatgacgcagagcttcgcttgcggtgcgggattagctggtaaagcgtttgcaacgggtaacgcggtttgggtttccgggtcagatcaattatccgggtcgggttgtgaacgggctaagcaaggaggagtgtttgggatgcatactattgcgtgtattccttcggcgaacggagttgtggaagtcgggtcaacggagccgatccgacagagttcggaccttattaacaaggttcgaattcttttcaatttcgacggcggagctggagatttatcgggtcttaattggaatcttgacccggatcaaggtgagaacgacccgtctatgtggattaatgacccgattggaacacctggatctaacgaaccgggtaacggagctccaagttctagctcccagcttttttcaaagtctattcagtttgagaacggtagctcaagcacaataaccgaaaacccgaatctggatccgactccgagtccggttcattctcagacccagaatccgaaattcaataacactttctcccgagaacttaatttttcgacgtcaagttctactttagtgaaaccaagatccggcgagatattaaacttcggcgatgaaggtaaacgaagctccggaaacccggatccaagttcttattcgggtcaaacacaattcgaaaacaaaagaaagaggtcgatggttttgaacgaagataaagttctatcattcggagataaaaccgccggagaatcagatcactccgatctagaagcttccgtcgtgaaagaagtagcagtagagaaacgtccaaagaaacgaggaagaaagccagcaaacggtagagaagagccactaaaccacgtcgaagcagagagacaaagacgcgagaaactaaaccaaagattctacgcgttacgagcggttgtaccaaacgtttcaaaaatggataaagcttcgttactcggtgacgcaatcgcttacatcaacgagcttaaatccaaagtagtcaaaacagagtcagagaaactccaaatcaagaaccagctcgaggaagtgaaactcgagctcgccggaagaaaagcgagtgctagtggaggagatatgtcgtcttcgtgttcttcgattaaaccggtggggatggagattgaagtgaagataattggttgggacgcaatgattagagttgaatctagtaagaggaatcatccggcggcgaggttgatgtcggcgttgatggatttggagttggaagtgaatcacgcgagtatgtcggtggttaacgatttgatgattcaacaagcgacggtgaagatgggttttaggatctatacgcaagaacagctcagagcaagtttgatttcaaaaatcggttaa

SEQ ID NO: 226 - protein, 623 aa, Arabidopsis thaliana
MTDYRLQPTMNLWTTDDNASMMEAFMSSSDISTLWPPASTTTTTATTETTPTPAMEIPAQAGFNQETLQQRLQALIEGTHEGWTYAIFWQPSYDFSGASVLGWGNGYYKGEEDKANPRRRSSSPPFSTPADQEYRKKVLRELNSLISGGVAPSDDAVDEEVTDTEWFFLVSMTQSFACGAGLAGKAFATGNAVWVSGSDQLSGSGCERAKQGGVFGMHTIACIPSANGVVEVGSTEPIRQSSDLINKVRILFNFDGGAGDLSGLNWNLDPDQGENDPSMWINDPIGTPGSNEPGNGAPSSSSQLFSKSIQFENGSSSTITENPNLDPTPSPVHSQTQNPKFNNTFSRELNFSTSSSTLVKPRSGEILNFGDEGKRSSGNPDPSSYSGQTQFENKRKRSMVLNEDKVLSFGDKTAGESDHSDLEASVVKEVAVEKRPKKRGRKPANGREEPLNHVEAERQRREKLNQRFYALRAVVPNVSKMDKASLLGDAIAYINELKSKVVKTESEKLQIKNQLEEVKLELAGRKASASGGDMSSSCSSIKPVGMEIEVKIIGWDAMIRVESSKRNHPAARLMSALMDLELEVNHASMSVVNDLMIQQATVKMGFRIYTQEQLRASLISKIG

SEQ ID NO: 227 - DNA, 768 bp, synthetic construct
  source 1..768: mol_type = other DNA
atggacttggattgtatacaatctccattcccttttgatcaagtagatgagttatttccactcccttctctttcccctatacacctttctgttgctgatcaccctccattgatcgcttccatcaacaacaccaacagaaatatctccgaaaagccaaaaaaaggtcgtcggcggaagtcccctaacacttcggccgatattgaggacgagaacccgaacccaaacgaacataaaaagaagaagattatccacagagatgttgaacgccaaagaaggcaagaaatgtccactctttacgccgctcttcgctcacttctcccggtggaatatctcaagggaaagcgatcgatatgtgatcacatgcatgagacagtgaaatacatacaacacatgcaaaccaagattcaaatgctgagaaacaagagagatgagcttaaaaagaatattgaagatggtgaggattccgggaacattaccacaatcgaaacattaaactcctcaaaaagggatagtgtgctagtgatgccaagatcatgtggaggagttcaaattcttctagacacagctacacaccataggcttccactttccaatcttatcaaatttctaattactcaaaatcttcaaatcattagttgtcattctactaggaagaacgacaggttcctccacactattgaaactgaagctgctgtcgatgttgaaacaattgatatgtcggaactacagaataaattgactaacttggaatattttccattagattaa

SEQ ID NO: 228 - protein, 255 aa, Cucumis sativus
MDLDCIQSPFPFDQVDELFPLPSLSPIHLSVADHPPLIASINNTNRNISEKPKKGRRRKSPNTSADIEDENPNPNEHKKKKIIHRDVERQRRQEMSTLYAALRSLLPVEYLKGKRSICDHMHETVKYIQHMQTKIQMLRNKRDELKKNIEDGEDSGNITTIETLNSSKRDSVLVMPRSCGGVQILLDTATHHRLPLSNLIKFLITQNLQIISCHSTRKNDRFLHTIETEAAVDVETIDMSELQNKLTNLEYFPLD

SEQ ID NO: 229 - DNA, 735 bp, synthetic construct
  source 1..735: mol_type = other DNA
atggaatttccattcccatttgatcaagcagatgagttatttccacttcctgctctttcccctatagacctttctgttcctcagtcttcattgataccttccaagaacaagactaacaaaaatatctctgaaaagcccaaaggcagccgtcagcgaaagtctccgaacgcttctgatgatatcgatgacgagaacccgagcgaacacaaaaagaagaagattatccacagagatgttgaacggcaaagaaggcaagaaatgtctactctttatgccactcttcgctcacttctcccggtggaatatctcaaggggaagcgatctatatgtgatcacatgcatgaaacagtgaaatacatagaacatatgcaaagcaagattcagaagctaacaaagaagagagatgagctgaaaaaggacattgaggatgattccaacattagtacaatcgaaacgttaaactcctcgaaaagggacagtgttgtagtgaagccaagacatggaggatttcaaattcttttagacacagccactcaacataggctcccactttcgaaccttatcaaatttctaattactgatcagaggctacaaatcgtcagttgccactccactaagataaacgacaggttccttcacaccattgaatctgaagctctcgttaatattggaaccatcgatgtgtcggaactccaacataaattgaccaacttggaatattttccattagattaa

SEQ ID NO: 230 - protein, 244 aa, Benincasa hispida
MEFPFPFDQADELFPLPALSPIDLSVPQSSLIPSKNKTNKNISEKPKGSRQRKSPNASDDIDDENPSEHKKKKIIHRDVERQRRQEMSTLYATLRSLLPVEYLKGKRSICDHMHETVKYIEHMQSKIQKLTKKRDELKKDIEDDSNISTIETLNSSKRDSVVVKPRHGGFQILLDTATQHRLPLSNLIKFLITDQRLQIVSCHSTKINDRFLHTIESEALVNIGTIDVSELQHKLTNLEYFPLD

SEQ ID NO: 231 - DNA, 756 bp, synthetic construct
  source 1..756: mol_type = other DNA
atggaaatgttcaatgctgagtcccctttttcttttgacgtgggagaagatctggtccctctgccctctcttccatcctcctcagtcatctctccgcccttggacaactacggtaaccgtgtttctcaaaagccaaaaaatggccgccggaaaaagccactgccaaacacttgcaatgatgatggcggcgatgagaaccatgacgaacaaaagaagaagaagataatacatagagatgtggagcgccaaagaagacaagaaatgtcttctctttacaccacacttcgctcgcttctccctcttgaataccttaagggaaaacgatcaatatcagaccacatgcaagagacagttagttacattcaacatatgcaaagaaggatccaacaactgaaagataaaagaggtacgctaagagaactcgccagccaaactacggtcgccatagccggtacgacagaaacgcttaattcctcagaaagggcctcagcagtggtgagggcaaaagatggaataggaattcaagttgttttagacactgccacaaaacaaaggctccctctttccatctttgttcaagctttagtttctgaaggccttgaaatcctcaactgcatttcaaacagactgaatgagaggttcattcacaccattgaatgccaacctcttctaaatgatgatggctgttatccaaccattgatgtctctgtgcttcagcataagttagccaatttggaatattatcctttagattag

SEQ ID NO: 232 - protein, 251 aa, Cucumis sativus
MEMFNAESPFSFDVGEDLVPLPSLPSSSVISPPLDNYGNRVSQKPKNGRRKKPLPNTCNDDGGDENHDEQKKKKIIHRDVERQRRQEMSSLYTTLRSLLPLEYLKGKRSISDHMQETVSYIQHMQRRIQQLKDKRGTLRELASQTTVAIAGTTETLNSSERASAVVRAKDGIGIQVVLDTATKQRLPLSIFVQALVSEGLEILNCISNRLNERFIHTIECQPLLNDDGCYPTIDVSVLQHKLANLEYYPLD

SEQ ID NO: 233 - DNA, 747 bp, synthetic construct
  source 1..747: mol_type = other DNA
atggacttcaatgttgagtctccagtttcatttgatttaggagatgacctacttcctctgccatctcttccatcgtcctccgcaatctctatgccagtggacaaccgtcgtaatggtgtctcccaaaatcaaaaaaatggccgccggaaaaagccattgcccaacacttgcaatgacaatggcaatgagagccccaacgaagaaagaaaaaagaagataatacatagagatgtggagcggcaaagaagacaagaaatgtcttccctttataccactcttcgctcacttctccctcttgaatacctcaagggaaaaaggtcaatatccgaccacatacatgagacagttaactacattcaacatatgcaagaaagaattcaacaactgtctgataaaagagatgagctaagaaaactctccaatggaactacggccaccgtcgatacggcaaaaacacttaattcctcagaaagggactctgtggtggtgaggccaaaagatggaataggaattcaagttgtaatagacactgccacaaaacataggttccctctttccaactttcttcaagctttagctgcggaaggacttgaaatcctcagctgcatttccaacagactgaatgagaggttcattcataccattgaatgccaacctattcttaataatgacggcgtctatccaaacatcgatgtgtttgagcttcaccataagttaacgaatttggaatattttcctttagattag

SEQ ID NO: 234 - protein, 248 aa, Benincasa hispida
MDFNVESPVSFDLGDDLLPLPSLPSSSAISMPVDNRRNGVSQNQKNGRRKKPLPNTCNDNGNESPNEERKKKIIHRDVERQRRQEMSSLYTTLRSLLPLEYLKGKRSISDHIHETVNYIQHMQERIQQLSDKRDELRKLSNGTTATVDTAKTLNSSERDSVVVRPKDGIGIQVVIDTATKHRFPLSNFLQALAAEGLEILSCISNRLNERFIHTIECQPILNNDGVYPNIDVFELHHKLTNLEYFPLD

SEQ ID NO: 235 - DNA, 2418 bp, synthetic construct
  source 1..2418: mol_type = other DNA
atggctgaacgtgctcttactcgtgtccatagcattcgtgaacgtgtagatgagactctcaaggctcatcgcaatgaaattgttgcgttactcacaaggatcgaaggtaagggaaaaggaattctccaacaccaccagattgttgctgagtttgaagcaattcctgaagacaccaggaaaacattggcaggcggtgcttttgctgaagttctcagatccacacaggaagcaattgttgtgcctccatggattgctcttgctctgcgcccgaggcctggtgtctgggagtacattagactgaatgtccaagcacttgttgttgaggagttgcgtgttgctgagtatcttcatttcaaggaggagcttgttgatggaggctgcaacggcaacttcgtgcttgaattggacttcgaaccattcaatgcatctttccctcgcccaactctttcaaagtatattggtaatggtgttgagttcctcaatcgccacctttcggctaagttgttccatgacaaggaaagcctgcatcccctgcttgcatttctcaaagtgcactgtcacaaggggaagaatatgatgctgaatgacagaattcgtaacctagactctctgcaatatgttctgagaaaggctgaggagtttctgtctactctgaaacctgatactccatattctcaatttgaacacaagttccaggagattggcttggagagaggatggggtgatactgctgagcgtgttcttgagatgattcgacttcttttggatcttcttgaggcaccagatccctgcactctcgaaacattcctaggcagaattcctatggtcttcaatgttgtgattatgtcccctcatggatactttgcccaagacaatgttttggggtatcctgatactggaggccaggttgtttacattttggatcaagtccgtgccttggagaatgaaatgcttcagcgtatcaagaagcaaggacttgatattatcccccgaattctcattattactcgactactccctgatgcagtaggaaccacttgtggtcaacgtttggagagagtgtatggatctgagcattgtgatattcttcgagttcccttcagagatggaaagggtatggtccgcaaatggatctctcgctttgaagtgtggccatacctagaaactttcaccgaggacgttgctgctgaaattgctaaggagttgcagggcaagcctgatcttatcattggaaattacagtgatggaaacatcgttgcctccttgttagcacacaaattaggtgttacagagtgcaccattgcgcatgctctagaaaaaacaaagtatccagactcagatatatactggaagaagtttgatgaaaagtaccacttctcatgccagtttacagctgatctttttgcaatgaaccatacagatttcattatcaccagcacattccaagagattgctggaagcaaggatactgttggacagtatgaaagccacactgctttcactctccctggcctctatagagttgttcatggtattgatgtctttgatcccaaattcaacattgtatcccctggcgctgatgagagcatatacttcccctacactgacgagaaacgtaggttgacttctttccatccagaaattgacgagcttctttacagccctgttgagaatgaagagcacttatgtgtgctaaaagaccgaaacaaaccaattctatttaccatggcaaggctggacagagttaagaatttatctggtcttgtagagtggtatggaaagaacaccaagctacgtgaattagttaatcttgttgtagttggtggagatagaagaaaggagtctaaagatctagaagagcaagctgagatgaagaaaatgtacagtcatatagagaaatacaacttgaatggccagttcagatggatttcttcccagatgaaccgtgtgaggaatggagagctttaccgttacatttgtgataccaagggagctttcgtgcagcctgctttgtatgaggcttttggattgacagttgttgaggccatgacttgtggtttgccaacttttgctacctgcaatggtggtcctgctgagatcattgtgaatggaaaatccggattccacatcgatccttatcatggtgagaaggctgctgagctccttgttgacttctttgagaagtgcaaggtcgatcccgctcactgggacaaaatctcccacggaggtctgcaacgaatccaagagaagtatacctggcaaatttactctcaaaggctcctgacgctcacaggagtttatggattctggaagcacgtttcgaaccttgatcgtcttgagagccgtcggtatatggaaatgttctatgcactcaaataccgtaaattggctgagtctgttcctctgactaaggagtaa

SEQ ID NO: 236 - protein, 805 aa, Populus alba
MAERALTRVHSIRERVDETLKAHRNEIVALLTRIEGKGKGILQHHQIVAEFEAIPEDTRKTLAGGAFAEVLRSTQEAIVVPPWIALALRPRPGVWEYIRLNVQALVVEELRVAEYLHFKEELVDGGCNGNFVLELDFEPFNASFPRPTLSKYIGNGVEFLNRHLSAKLFHDKESLHPLLAFLKVHCHKGKNMMLNDRIRNLDSLQYVLRKAEEFLSTLKPDTPYSQFEHKFQEIGLERGWGDTAERVLEMIRLLLDLLEAPDPCTLETFLGRIPMVFNVVIMSPHGYFAQDNVLGYPDTGGQVVYILDQVRALENEMLQRIKKQGLDIIPRILIITRLLPDAVGTTCGQRLERVYGSEHCDILRVPFRDGKGMVRKWISRFEVWPYLETFTEDVAAEIAKELQGKPDLIIGNYSDGNIVASLLAHKLGVTECTIAHALEKTKYPDSDIYWKKFDEKYHFSCQFTADLFAMNHTDFIITSTFQEIAGSKDTVGQYESHTAFTLPGLYRVVHGIDVFDPKFNIVSPGADESIYFPYTDEKRRLTSFHPEIDELLYSPVENEEHLCVLKDRNKPILFTMARLDRVKNLSGLVEWYGKNTKLRELVNLVVVGGDRRKESKDLEEQAEMKKMYSHIEKYNLNGQFRWISSQMNRVRNGELYRYICDTKGAFVQPALYEAFGLTVVEAMTCGLPTFATCNGGPAEIIVNGKSGFHIDPYHGEKAAELLVDFFEKCKVDPAHWDKISHGGLQRIQEKYTWQIYSQRLLTLTGVYGFWKHVSNLDRLESRRYMEMFYALKYRKLAESVPLTKE

SEQ ID NO: 237 - DNA, 1443 bp, synthetic construct
  source 1..1443: mol_type = other DNA
atggctgcagcaccagcagttgcttcacccgcagtcgaaaccgaccggatctttaagcttaaagccgaggtcgcgaagctgaaccagatcagtgttaatgagaaggaaggatttgtccgactagtttctcgctacctaagcggcgaagaagagagaattgaatgggaaaaaatcaagaccccgactgacgaggtcgtagttccctacgatactttggctgatctgggagaagatccatcggaagccaaggaactcctggacaagcttgttgtgctaaagcttaatggcggtttgggaacaaccatgggctgtactggtcccaagtctgtcattgaagtacgaaatggcctaacatttctggacttgattgtgaagcaaatagagtctctgaacaacaaatacgattgcagagtaccattggtgttgatgaattcattcaacacacatgacgatacaattaagattgtagaaaagtacactggctcaaatatcgacatccacatctttaaccagagtcagtatccacgcatggtggcagaagatttgatgccatggccaactaaaggtcgtacagataaaggaggatggtacccacctggccacggagatgttttccctgctcttttgaacagtggaaagcttggtgaactactctcacagggaaaggagtatgtgtttgtagctaactcagacaacttgggagcgatagttgatctcaaaattttgaatcatcttgtcaaaaataaaaacgaatactgcatggaggtcactcccaaaacactcgcagatgtaaaaggtggtactcttatttcttatgaaggcagggttcagcttctggagattgcacaggttcccgaggagcatgttggtgagttcaagtcaattgagaagttcaaaatcttcaataccaataatttgtgggtgaacttgaaggccattaagaggcttgtggagtctgatgctcttaaaatggagataattccaaatccaaaggaggtggatggggtaaaggtgcttcagcttgaaacggctgccggtgctgctataaggttttttgatcgtgcaattggtgtcaatgttccaagatcacgttttctcccagtgaaggcggcttcagatctactacttgtgcagtcggatctttacactgtggaggaaggtttcgtcatccgaaatcctgctagagtcaaccctgcaaatccctccattgagctgggtcctgaattcaagaaggttggcaactttctaaagcgtttcaagtccatacctagtatcattgatcttgatagtctaaaggtgtcaggcgatgtgtggtttggcagtggaatcatcctaaagggaaaggttactattgaagcaaagcagggggttaaacttgagatacctgatgaatctgtgattgaaaacaaggtagtgagcagtcctgatgatattgtgaactag

SEQ ID NO: 238 - protein, 480 aa, Larix gmelinii
MAAAPAVASPAVETDRIFKLKAEVAKLNQISVNEKEGFVRLVSRYLSGEEERIEWEKIKTPTDEVVVPYDTLADLGEDPSEAKELLDKLVVLKLNGGLGTTMGCTGPKSVIEVRNGLTFLDLIVKQIESLNNKYDCRVPLVLMNSFNTHDDTIKIVEKYTGSNIDIHIFNQSQYPRMVAEDLMPWPTKGRTDKGGWYPPGHGDVFPALLNSGKLGELLSQGKEYVFVANSDNLGAIVDLKILNHLVKNKNEYCMEVTPKTLADVKGGTLISYEGRVQLLEIAQVPEEHVGEFKSIEKFKIFNTNNLWVNLKAIKRLVESDALKMEIIPNPKEVDGVKVLQLETAAGAAIRFFDRAIGVNVPRSRFLPVKAASDLLLVQSDLYTVEEGFVIRNPARVNPANPSIELGPEFKKVGNFLKRFKSIPSIIDLDSLKVSGDVWFGSGIILKGKVTIEAKQGVKLEIPDESVIENKVVSSPDDIVN

SEQ ID NO: 239 - DNA, 1971 bp, synthetic construct
  source 1..1971: mol_type = other DNA
atgatcgttaatttctctttcttcctccttctcttcgtctccgtcttcgtttcctctgctgattctaaagcgaccatttcaatttcccctaatgctctcaatcgatctggcgattccgttgtgatacaatggtccggtgtcgattctccgtcagatctcgattggttaggactctactcgccgccggagtctcctaatgatcactttattggttacaaattcctcaatgaatcgtccacttggaaagatggtttcggttcgatttctcttcctttaaccaatctccgatcaaattacacattccggatcttccgttggagcgaatccgagattgatccgaaacataaggatcatgatcagaatcctttaccaggaactaaacatcttctagctgaatcggagcagctgactttcggatccggtgttggtatgccggagcagatccatttgtcgttcacaaatatggttaacacgatgcgtgttatgtttgtagctggagatggtgaagaacgttttgttagatacggtgaatcgaaggatttgttaggtaattccgcggcggcgcgtgggatgaggtacgagagagagcacatgtgtgattcgccggcgaattccactattggttggagagatcctggttggatttttgataccgtcatgaagaatttgaatgatggcgttagatactattatcaggttgggagtgattctaagggatggagtgagatccatagctacattgctcgagatgtgactgcagaagaaaccgtagctttcatgtttggagatatgggttgtgctacaccatacacgacatttatccgcacacaagatgagagcatatctacagtgaagtggatcctccgtgacattgaagctcttggtgataagccagctatgatttcacacattggagatataagttatgctcgtggttactcgtgggtatgggatgagttctttgctcaggttgagcctattgcctcgacagttccttaccatgtttgcattggtaaccatgagtatgatttctctactcagccgtggaaacctgattgggcagcttctatttatggaaacgatggtggtggcgaatgtggtgtgccgtatagcttgaagtttaacatgcctgggaattcttcagagtctacaggaatgaaagctcctccgacaaggaatttatattattcttatgatatgggaacggtccatttcgtttatatctccacagagacgaattttcttaaaggaggtagtcaatatgaattcataaagcgagatctagagtctgtagacaggaagaaaacaccgtttgttgttgtgcaaggacatagaccaatgtacactacgagcaacgaggttagagacactatgattcgacaaaagatggttgagcatctagaacctttgtttgtgaaaaacaatgtcacacttgctctatggggacatgttcatagatacgaaaggttttgtcccataagcaacaacacttgcggcacacagtggcaaggaaatccggttcatcttgtgatcggtatggctggtcaagattggcaaccgatttggcagcctagaccaaaccatccagatcttcctatattccctcagcctgaacaatcaatgtatcgtacaggtgagtttggttacactcgtttagttgcaaacaaagaaaagctcactgtttcttttgtgggtaatcacgatggcgaagttcatgatactgttgagatgttagcatctggggtagtaatcagtgggagcaaagagagtactaaaatcccaaatctgaaaaccgttcctgcttctgctacacttatgggaaaatcagaatctaatgctttgtggtatgccaaaggagcaggcttgatggttgtgggtgtgcttttagggttcattatcgggttttttacccggggaaagaaatcttcgtctggaaaccgttggatcccagtcaagaacgaggagacataa

SEQ ID NO: 240 - protein, 656 aa, Arabidopsis thaliana
MIVNFSFFLLLFVSVFVSSADSKATISISPNALNRSGDSVVIQWSGVDSPSDLDWLGLYSPPESPNDHFIGYKFLNESSTWKDGFGSISLPLTNLRSNYTFRIFRWSESEIDPKHKDHDQNPLPGTKHLLAESEQLTFGSGVGMPEQIHLSFTNMVNTMRVMFVAGDGEERFVRYGESKDLLGNSAAARGMRYEREHMCDSPANSTIGWRDPGWIFDTVMKNLNDGVRYYYQVGSDSKGWSEIHSYIARDVTAEETVAFMFGDMGCATPYTTFIRTQDESISTVKWILRDIEALGDKPAMISHIGDISYARGYSWVWDEFFAQVEPIASTVPYHVCIGNHEYDFSTQPWKPDWAASIYGNDGGGECGVPYSLKFNMPGNSSESTGMKAPPTRNLYYSYDMGTVHFVYISTETNFLKGGSQYEFIKRDLESVDRKKTPFVVVQGHRPMYTTSNEVRDTMIRQKMVEHLEPLFVKNNVTLALWGHVHRYERFCPISNNTCGTQWQGNPVHLVIGMAGQDWQPIWQPRPNHPDLPIFPQPEQSMYRTGEFGYTRLVANKEKLTVSFVGNHDGEVHDTVEMLASGVVISGSKESTKIPNLKTVPASATLMGKSESNALWYAKGAGLMVVGVLLGFIIGFFTRGKKSSSGNRWIPVKNEET

SEQ ID NO: 241 - DNA, 477 bp, synthetic construct
  source 1..477: mol_type = other DNA
atggatttcaggctaatgggtttggattcaccactcttcaacaccctccaccacataatggacctcacagatgacacaacagagaagaatctaaacgcaccaacacgaacctacgttcgcgacgcaaaagcaatggctgcaacaccagcagacgtgaaagagtatccaaattcatacgtgttcgtggtggacatgccagggttgaaatcaggtgacatcaaggttcaggttgaagatgagaatgtgcttctgataagcggtgaaaggaagagagaagaagagaaagaaggtggtaaatatttgaagatggaaagaagggttggtaagtttatgaggaaatttgtgttacctgagaatgctgatgttgaaggtggtgtttctgctgtttgtcaagatggtgttcttactgttactgttaataagttgcctcctcctgaacctaagaagcctaagactattgaggttaaggttgcttga

SEQ ID NO: 242 - protein, 158 aa, Medicago sativa
MDFRLMGLDSPLFNTLHHIMDLTDDTTEKNLNAPTRTYVRDAKAMAATPADVKEYPNSYVFVVDMPGLKSGDIKVQVEDENVLLISGERKREEEKEGGKYLKMERRVGKFMRKFVLPENADVEGGVSAVCQDGVLTVTVNKLPPPEPKKPKTIEVKVA

SEQ ID NO: 243 - DNA, 594 bp, synthetic construct
  source 1..594: mol_type = other DNA
atgagaggtaggaaatacaggaatagcgttcgaccaaaccgagtgaccggttcaggtttctggaaagccactggtattgataaaccggtttactccaatcttgactgtgttggcctcaagaaatctctggtttactatcttggttcagccggtaaaggcaccaaaaccgattggatgatgcatgaattccgcctcccctccaccacgaaaaccgactctccagctcaacaagcagaggtatggacactttgcagaatcttcaaacgagtcacatctcaaagaaacccaaccatcttaccaccaaaccgaaaaccggttatcactttaaccgacacttgttctaagaccagcagcttagattccgaccacacgagccaccgtacagtagattccatgtcccacgagccgccgcttccacagccacagaatccttattggaaccaacatatagttggttttaatcaaccgacatatactggtaatgataataacctcctgatgagtttctggaacggcaacggtggagatttcataggagactcagcaagttgggatgaacttagatctgttatagatggcaacactaaaccctag

SEQ ID NO: 244 - protein, 197 aa, Arabidopsis thaliana
MRGRKYRNSVRPNRVTGSGFWKATGIDKPVYSNLDCVGLKKSLVYYLGSAGKGTKTDWMMHEFRLPSTTKTDSPAQQAEVWTLCRIFKRVTSQRNPTILPPNRKPVITLTDTCSKTSSLDSDHTSHRTVDSMSHEPPLPQPQNPYWNQHIVGFNQPTYTGNDNNLLMSFWNGNGGDFIGDSASWDELRSVIDGNTKP

SEQ ID NO: 245 - DNA, 891 bp, synthetic construct
  source 1..891: mol_type = other DNA
atggcccacggcctcacccatttcaacaccaaatcgggtttattcccatctatcaccaaatcaaaaacaacccgtccatcaacccgacccgtgattctcgccatgacccggacccaaacttaccgggccaccattgaatccgacattgaatcttatctcaagaaggccatcccaatccgggcacctgaatccgtattcgagcccatgcaccacctcacgtttgccgccccaagaaccagcgcatcggctttatgcgtggcagcgtgcgagctcgttggcggcgaccgaagcgatgccatggcggcggcggcggcggtgcatctcatgcacgtggcggcgtacacacacgagaaccttcctcttaccgacgggcccatgtccaaatcggaaatccaacacaagttcgatccgaatatcgaacttttgacaggagacggaatcataccattcgggctggagttgatggcgagatcaatggatccgacccggaacaacccggataggatcctaagagcgataatcgaactcacccgggtcatgggctcagagggaattgtagaagggcaataccatgaacttggtctaaatcaattaaacgatttagaactcatcgagtatgtttgcaagaaaaaagaagggacactacatgcatgtggggccgcttgtggagcaattttaggtggatgtgatgaagataaaatagaaaagttgagaagattcggactttatgtgggtacggttcaagggttgttgggtaaaaatagatccggatttgaaggaagaattaaggaattgaaggaattggctgttaaggaactggagagctttggtggtgagaaaattgagctgattaggggcgtttttgagctagagcatagtcttgctggtgtt

SEQ ID NO: 246 - protein, 297 aa, Antirrhinum majus
MAHGLTHFNTKSGLFPSITKSKTTRPSTRPVILAMTRTQTYRATIESDIESYLKKAIPIRAPESVFEPMHHLTFAAPRTSASALCVAACELVGGDRSDAMAAAAAVHLMHVAAYTHENLPLTDGPMSKSEIQHKFDPNIELLTGDGIIPFGLELMARSMDPTRNNPDRILRAIIELTRVMGSEGIVEGQYHELGLNQLNDLELIEYVCKKKEGTLHACGAACGAILGGCDEDKIEKLRRFGLYVGTVQGLLGKNRSGFEGRIKELKELAVKELESFGGEKIELIRGVFELEHSLAGV

SEQ ID NO: 247 - DNA, 1029 bp, synthetic construct
  source 1..1029: mol_type = other DNA
atgagcgatctgaagacgagatttctggaggtgtactctgttctgaaatccgagctactcaacgaccctgctttcgagttcaccgatgattctcgccaatgggtcgagcggatgctggactataatgtgcctggaggaaagctgaatcgagggctgtctgttattgacagctacaagttgctgaaagaaggaaaagaactaagtgatgatgaaatttttctttcaagtgcacttggttggtgcattgaatggcttcaagcttattttcttgtgctcgatgatattatggatagctctcatacgcgcagaggtcaaccctgttggttcagattacctaaggttggtatgattgccgtaaatgatggcatattacttcgcaaccatatcccaaggattctcaagaagcatttccgacaaaagccttactatgtggatctgttggatctatttaatgaggtagaattccagacagctagtggacagatgatagatttgatcaccacccttgtaggagagaaagacttatcgaaatactcattgcctattcatcgccggattgtgcagtacaaaactgcttactactcattttaccttccagtggcctgtgcacttcttatgtcaggcgaagatctggagaaacatactaatgtaaaggacatacttattgaaatgggaacctattttcaagtgcaggatgattatctggattgctttggtgcaccagaggtgattgggaagattggcacagatattgaagatttcaagtgctcctggttggtagtaaaagcactggaactttctaacgaggaacagaagaagtttttacatgagaactatggaaaggatgatccggcctctgtagcaaaagtgaaagagctttataacactctcaagcttcaggatgtatttgccgagtatgagagcaagagctatgacaagttgatcaaattcattgaagctcatccgagccaagccgtacaagcagtgttaaaatcattcttgggaaagatatataagcggcaaaagtaa

SEQ ID NO: 248 - protein, 342 aa, Panax ginseng
MSDLKTRFLEVYSVLKSELLNDPAFEFTDDSRQWVERMLDYNVPGGKLNRGLSVIDSYKLLKEGKELSDDEIFLSSALGWCIEWLQAYFLVLDDIMDSSHTRRGQPCWFRLPKVGMIAVNDGILLRNHIPRILKKHFRQKPYYVDLLDLFNEVEFQTASGQMIDLITTLVGEKDLSKYSLPIHRRIVQYKTAYYSFYLPVACALLMSGEDLEKHTNVKDILIEMGTYFQVQDDYLDCFGAPEVIGKIGTDIEDFKCSWLVVKALELSNEEQKKFLHENYGKDDPASVAKVKELYNTLKLQDVFAEYESKSYDKLIKFIEAHPSQAVQAVLKSFLGKIYKRQK

SEQ ID NO: 249 - DNA, 1248 bp, synthetic construct
  source 1..1248: mol_type = other DNA
atgggaagtttgggggcaattctgaagcatccggaagatttctatccgttgttgaagcttaaatttgcggctaggcatgcggaaaagcagatccctccggagccacactgggccttctgttactctatgcttcataaagtttctcgaagtttcggcctcgtcattcaacagctcggccctcagctccgcgatgctgtatgcattttttatttggttcttcgagcacttgacactgttgaggatgacacaagtatacctacagaggttaaagtacctatcttgatggcttttcatcgccacatatatgataaggactggcacttttcatgtggtacgaaggaatacaaagttctcatggacgagtttcatcatgtttctaatgcttttcttgagcttggaagcggttaccaggaggcaatagaagatattaccatgagaatgggtgcaggaatggcaaaatttatatgcaaggaggtggagacaataaatgattatgatgaatattgtcactatgtagcaggacttgttggattagggttgtcaaagctcttccatgcctctggggcagaagatttggctacagattctctgtccaattcaatgggtttatttctccagaagacaaacataattcgagattacttggaggacataaatgagataccaaagtcacgcatgttttggcctcgccagatttggagtaaatatgtcgataaacttgaggacttgaaatatgaggaaaactcagccaaggcagtgcagtgcctaaatgacatggtcacagatgctttggttcatgctgaagattgcctaaagtacatgtctgacttgcgaggtcctgctatcttccggttctgtgcaataccacagattatggcaattggaacactagctttatgctttaacaacactcaagtcttcagaggggtagtgaaaatgagacgtggtcttactgctaaagttattgaccaaacaaaaacaatgtcagatgtatatggtgctttcttcgatttttcttgtttgctgaagtccaaggttgacaacaatgatcccaatgctacaaaaactttgagcaggctagaagcaattcagaaaacatgcaaggagtctggaaccctgtccaaaaggaaatcatacataatcgagagcgagtcaggacacaattcagccctgattgctattatcttcattatactagctatcctttatgcatatctatcttcaaacctactactgaacaaacagtga

SEQ ID NO: 250 - protein, 415 aa, Panax ginseng
MGSLGAILKHPEDFYPLLKLKFAARHAEKQIPPEPHWAFCYSMLHKVSRSFGLVIQQLGPQLRDAVCIFYLVLRALDTVEDDTSIPTEVKVPILMAFHRHIYDKDWHFSCGTKEYKVLMDEFHHVSNAFLELGSGYQEAIEDITMRMGAGMAKFICKEVETINDYDEYCHYVAGLVGLGLSKLFHASGAEDLATDSLSNSMGLFLQKTNIIRDYLEDINEIPKSRMFWPRQIWSKYVDKLEDLKYEENSAKAVQCLNDMVTDALVHAEDCLKYMSDLRGPAIFRFCAIPQIMAIGTLALCFNNTQVFRGVVKMRRGLTAKVIDQTKTMSDVYGAFFDFSCLLKSKVDNNDPNATKTLSRLEAIQKTCKESGTLSKRKSYIIESESGHNSALIAIIFIILAILYAYLSSNLLLNKQ

SEQ ID NO: 251 - DNA, 1425 bp, synthetic construct
  source 1..1425: mol_type = other DNA
atggcgtctcatccagaaaatgttggtattttggctatggatatctactttccgagcacatgtgttcagcaggatgccctggaaacctttgatggagtaagtaaagggaaatacacaattggccttggacaagactgcatggcattttgcatggacttggaagatgtgatttccatgagcttgactgtagtatcctcactcttggacaagtatggaatcgatccaaaaagaattggtcgcttggaagttggcagtgaaactgttattgacaagagcaagtccataaagacctggttgatgcagatttttgaggaatgtggcaatagtgagattgaaggtgtggactcaacaaatgcatgctatggaggaactgcagctctgcttaactgtgttaactgggttgaaagcaggtcttgggatgggcgatatggactcgttgtttctacagacagtgcagtctatgctgaaggtccagcccgacctacagggggagcagctgctgttgctatgctgatagggcccaatgcacctatagtgtttgaaagcaaatacaggggaacacacatgtctcatgcgtatgacttttataagcccaatttagccagcgagtacccagttgtggatgggaagctttcacaaacttgctatctcatgggactggactcatgctacaaacggttttgtaatcggtttgaggagggagaaggaagacaattttctctttcagatgcagattatgtagcatttcactctccatacaataagcttgtgcaaaagagctttgctcgactgttgttcaatgatttctcaagacatgccagttctgttgggaaggatgcgcaagaaaagctagagccgtatgcaggcttgtctgatgaagatagctacagtagccgtgaactagaaaaggtttctcagcaggttgctaggccattgtatgatgtaaaagttcaaccatcaaccttattgccaaagcaagtaggcaacatgtacacagcatctctttatgcagcgtttgcctctattatacataacaagcatgataccctggaggggcagagggtgttgatgttttcttatgggagtggtttggcttccacaatgttttctttgaaaattcgggagggtcagcatcctttcaccttaacaaatattgcagatgttatggatgtctcgagcaagcttgattctcgacatgtgttgtcccctgaagattttatggtcaacatgaagctgatggagactctatatggagcaaaagatttcatttcatgctctgatcacaacttgctgcgatctggaacattttatttgactcaagtagattcaatgtaccgacgtttctatgcccagaaggttgcaagtccagatgacactcatgagaaggtcaaacttgcaaatggtatcatccatgatcaattgtaa

SEQ ID NO: 252 - protein, 474 aa, Ginkgo biloba
MASHPENVGILAMDIYFPSTCVQQDALETFDGVSKGKYTIGLGQDCMAFCMDLEDVISMSLTVVSSLLDKYGIDPKRIGRLEVGSETVIDKSKSIKTWLMQIFEECGNSEIEGVDSTNACYGGTAALLNCVNWVESRSWDGRYGLVVSTDSAVYAEGPARPTGGAAAVAMLIGPNAPIVFESKYRGTHMSHAYDFYKPNLASEYPVVDGKLSQTCYLMGLDSCYKRFCNRFEEGEGRQFSLSDADYVAFHSPYNKLVQKSFARLLFNDFSRHASSVGKDAQEKLEPYAGLSDEDSYSSRELEKVSQQVARPLYDVKVQPSTLLPKQVGNMYTASLYAAFASIIHNKHDTLEGQRVLMFSYGSGLASTMFSLKIREGQHPFTLTNIADVMDVSSKLDSRHVLSPEDFMVNMKLMETLYGAKDFISCSDHNLLRSGTFYLTQVDSMYRRFYAQKVASPDDTHEKVKLANGIIHDQL

SEQ ID NO: 253 - DNA, 1488 bp, synthetic construct
  source 1..1488: mol_type = other DNA
atggattctcacacccatggaacacctcattttcttctcttccctttcatggcacaaggccatatgatccccatgatcgacctagccaagtttctagctcgccgtggagccattgtcaccatcgtcaccactcctctcaactctgctcgcttccactctgttctcactcgcgccattgattccggccatcaaatccatgtccgtgaactccaattccctagccaccaagaaaccggcctcccagaaggctgcgagaatgtcgacttgctaccttcacttgcttccatatcccaattctacagagctatcagtctccttcaccaaccatcggaaaagttgttcgaacaactcacccctcgtcccaattgcatcatctccgacatgtgcatcccttggactttcgatatttctcagaaattccacgtccccagactcgttttctacagtttaagctgtttcttcctgctctgcatgcggagtttaaccactaactatgaatttctgaactccaaccctgattccgaattcctgactttaccaggcctcccttctcaagttgagtttcgcaggtcccagatctttacatccaccgatgattacttaatccaatatagtttcagaatgtgggaggttgatcggcaatcgtatggcgtaattgtaaatgtgtttgaagaaatggagccagagcatgtaacagagtatataaaaggaagagaatcccctgaaaaagtatggtgcgttggtcctctttcactttccaacgacaacgagcttgacaaagccgaaagaggaaacaaagcgataatcgacggccatgaatgcatcaaatggatggacgaacagaagccgtcgtcggtggtttacgtgtcattaggaagtctctgcaatctgtgtacagagcaaatcaaagagctgggattgggattggtggcatcgaacaaaccattcatttgggtgataagaaaagcaaatctgaccgaagctctagtcaaatggatggatgaatatgaattcgaggagaaaacaaaagggcgtgggttagtgatacgtggatgggctccacaagttctgatactctcgcactctgcaattgggtgcttcttgacacattgtggatggaattcaagcgttgaagggatatccgcaggggtgccgatgatcacatggccattgtttgcagatcaactgtacaaccacaagttcatcgtggagatactgaaagtgggtgtgagtgttggagagggaacagttggagatttgggaggtgtgcaaaaggtagtggtgaagagggagaaagtgaaggaagccattgaaatggtgatggatggagatggaagtgaagaaaggagaaagagatgtaaagagtacggtgagaaggcgaagaaggcggcagaagaaggaggttcatctcatcggaatctgaatcggctagttgaagatattactgctcatgcctttgcctgtggtaatggaaatggaagctgctga

SEQ ID NO: 254 - protein, 495 aa, Cucumis sativus
MDSHTHGTPHFLLFPFMAQGHMIPMIDLAKFLARRGAIVTIVTTPLNSARFHSVLTRAIDSGHQIHVRELQFPSHQETGLPEGCENVDLLPSLASISQFYRAISLLHQPSEKLFEQLTPRPNCIISDMCIPWTFDISQKFHVPRLVFYSLSCFFLLCMRSLTTNYEFLNSNPDSEFLTLPGLPSQVEFRRSQIFTSTDDYLIQYSFRMWEVDRQSYGVIVNVFEEMEPEHVTEYIKGRESPEKVWCVGPLSLSNDNELDKAERGNKAIIDGHECIKWMDEQKPSSVVYVSLGSLCNLCTEQIKELGLGLVASNKPFIWVIRKANLTEALVKWMDEYEFEEKTKGRGLVIRGWAPQVLILSHSAIGCFLTHCGWNSSVEGISAGVPMITWPLFADQLYNHKFIVEILKVGVSVGEGTVGDLGGVQKVVVKREKVKEAIEMVMDGDGSEERRKRCKEYGEKAKKAAEEGGSSHRNLNRLVEDITAHAFACGNGNGSC

SEQ ID NO: 255 - DNA, 1428 bp, synthetic construct
  source 1..1428: mol_type = other DNA
atgaagatagagcggaagcagagtgtcgtactggttccccatccctaccaggggcacctcacgccaatgcttcagctcggcagcattctccactcgcaaggcttctccgtcatcgtcgcgcacacccagtacaatacgcccaactactccaatcacccgcagttcgtcttccattccatggacgatggcctccagggcatcgacatgagctttccgtccctcgagaacatctacgacatgaacgagaactgcaaggcccccctccgcaactatctggtttccatgatggaggaggaaggggaccagctggcgtgcatcgtctacgacaacgtcatgttcttcgtcgacgacgttgcgacgcagttgaagcttccgtcgatcgtgctgcggaccttctcggccgcctacctgcactctatgatcaccatcctgcagcagccggagatctacctgcccttcgaggactcgcaactgctggacccgctgcccgagctccaccccctccgcttcaaggacgtgcccttccccatcatcaacaacaccgtgccggagccgatcctcgacttctgccgcgcgatgtccgacatcgggtcctccgtcgcgaccatctggaacaccatgcaggacctcgagagctccatgctcctgcgcctgcaggagcactacaaggtgcccttcttcccgatcggcccggtgcacaagatggcctcgctcgtgtcctcaacgtcgatcctcgaggaggacaacagctgcatcgagtggctcgaccgtcaggccccgaactccgtcctgtacgtcagcctggggagcttggtccggattgaccacaaggagctcatcgagacagcatgggggctggccaactcggaccagccgttcctctgggtcatccggccgggctcggtcagtggcttccagtgcgcggaagcactgcccgacggcttcgagaagatggtcggcgagcggggacgaatcgtgaagtgggcgccccagaagcaggtcctggcccatcccgcggtcgcggggttcttcacgcattgcgggtggaactccacgctcgagtccatctgcgaagaggtcccgatggtgtgccgccccttcctggcggaccagctggtgaacgcgcggtacctgagccagatctacaaagtgggcttcgagctggaggtcatcgagcgcacggtgatcgagaagaccattcgcaagctgatgctgtccgaggaggggaaggacgtcaagaagcgggtcgcggacatgaagcagaagatcgtggccggcatgcagatcgactgcacgtcgcacaagaacctgaacgacctcgtggactttatctcggcgctgccgtcgcggctcgccccgcccacccccgtcttcggagccatcatgtcctcgaaccacatagcaagcaagtgtatcatagagtcttga

SEQ ID NO: 256 - protein, 475 aa, Solanum lycopersicum; identical to SEQ ID NO: 120

SEQ ID NO: 257 - DNA, 1488 bp, synthetic construct
  source 1..1488: mol_type = other DNA
atggactcccacacacatggcacgcctcacttcctgttgttccccttcatggcccagggccacatgatccccatgatcgacctggccaagttcctggcccgccgcggggccatcgtcacgatcgtcaccacccccctcaacagcgcgcggttccacagcgtcctcacccgcgcgattgactccggccaccaaatccatgtccgagagctccagttcccgagccaccaggagacgggcctccccgaggggtgcgagaacgtcgacctcctgccgagcctcgcaagcatctcccagttctaccgggcgatctcgctactccaccagccttcggagaagctgttcgagcagctcacgccccggcccaactgcatcatctcggacatgtgcatcccgtggaccttcgacatcagccagaagttccacgtcccccgcctcgtgttctactcgctgtcgtgcttcttcctgctgtgcatgcggtcgctcaccaccaactacgagttcctgaactccaacccggacagcgagttcctgacgctgcccggcctgccctcccaggtcgagttccggaggagccagatcttcacgtcgaccgacgactacctgatccagtacagtttccggatgtgggaggtcgaccggcagagctacggggtgatcgtcaacgtgttcgaggagatggagcccgagcacgtgacggagtacatcaagggccgggagtcgccggagaaggtgtggtgcgtggggcccctttcgcttagcaacgacaatgagcttgacaaggccgagcgggggaacaaggcgatcatcgacgggcacgagtgcatcaagtggatggacgagcagaagccgagcagcgtcgtctacgtgagcctcggcagcctctgcaacctctgcacggagcagatcaaggagctggggctaggcctcgtggcgtcgaacaagccgttcatctgggtgatccggaaggccaacctgaccgaggccctcgtcaagtggatggatgagtacgagttcgaggagaagacgaaggggcgcgggctggtcatccgcgggtgggccccgcaggtcctaatcctctcgcactctgcgatcgggtgcttcctgacgcactgcggctggaacagctcggtcgaggggatctccgccggcgttccgatgatcacgtggccgctgttcgccgaccagctctacaaccacaagttcatcgtggagatcctgaaggtcggggtgtcggtgggggaggggaccgtgggggacctcgggggggtgcagaaggttgtcgtcaagcgcgagaaggtgaaggaggccatcgagatggtgatggacggcgacggcagcgaggagcggcggaagcgctgcaaggagtacggcgagaaggccaagaaggcggcagaggagggcggctcgtcgcaccggaacctgaaccgcctcgtggaggacatcacggcccacgccttcgcgtgcggtaacgggaatgggagttgctga

SEQ ID NO: 258 - protein, 495 aa, Cucumis sativus; identical to SEQ ID NO: 254

SEQ ID NO: 259 - DNA, 1327 bp, Arabidopsis thaliana
  source 1..1327: mol_type = genomic DNA
cgcttggcgcgccgtcaacggatcaggatatccttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaagacgattttctgggtttgatcgttagatatcatcttaattctcgattagggtttcataaatatcatccgatttgttcaaataatttgagttttgtcgaataattactcttcgatttgtgatttctatctagatctggtgttagtttctagtttgtgcgatcgaatttgtcgattaatctgagtttttctgattaacag

SEQ ID NO: 260 - DNA, 242 bp, Peanut chlorotic streak virus
  source 1..242: mol_type = genomic DNA
tgagccaatcaaagaggagtgatgtagacctaaagcaataatggagccatgacgtaagggcttacgccattacgaaataattaaaggctgatgtgacctgtcggtctatcagaacctttactttttatatttggcgtgtatttttaaatttccacggcaatgacgatgtgacctgtggatccgctttgcctataaataagttttagtttgtattgatcgacacgatcgagaagacacggcca

SEQ ID NO: 261 - DNA, 1504 bp, Citrullus lanatus
  source 1..1504: mol_type = genomic DNA
ggagtactccttaccctaaatacttctttaatctcaaaatttcattaaatacgtactttaaatttttcgggttaatcaatttaaaataactatgaagcgatattttaaatttaatagttataaaaaatagaaaattttaattaattataatctttatctttttaaataaaacttatttcccaaatttttaatatttctctcaacataatgtttcatgaatagtaatcacaagtcaaattaaacctattatgatagtgaaattagaagtaaaacggttaaaagtgaatttaatttagtgataattgacctatattatttattttggagatggagattccatttcaatttcgataattgtcatagaaaaaatagtgaaattagaaaatttcttaacatatattagcagaataaattaaaaaaaaaaaagtgttggagcataatttatttattgaaacaaaaactgatggagcattcattcatttttttaaagcatcgtccaagtcaatttaaacattacccaacattgaaattataaaatttctgtctaaaaaaagtcttgacggtatatgtataataaataaatattaaaatatctgttagaaagaataaattgaaaatgattaaataagtttttaacgcaaatataaagttgaaatttaagatttagagaataaaattatacaactgtgttaagtttattattattattatttttttaatttttatattagtttactttaaatattttggccaacttatatgcaattcaattaatctcaagtgaaaattcgtctaatcctacaacaattttaatataaagattgattattgctgtcaaaattcattctctcttattggagactttttcaaatataagaaaattaactaaaatatttataaatatagcaaaatttcaacatctctttgaaatagatcgtaatagactactatctatgttatcatacacatcagtagtctatcgcaatttataaaaaataaattgtgattttttattattattagtaaatattttaaaaaaatttgttttttaaactaattttcattattttccttattgttattattattattaatactaaccaggatggagcattagtaacttaggaagggaaaaggttgatttactcgtcaaacgccaatcacaatgattacggattcccaacccatgagaatccacagagatcaccgcactccaaaaaccactttttctttctttctttattttcttttcaaaaccgcttttatttattattattactaccattattttaaattcttataatttaaaattatgttaaatggcttcttctctaaatttctcttaccacttggccacccactgccactcccacgccactccctctgtttatatataaacatcttcatttcctctccatttcatcactcaaaaaccaccattctctgattccccaaaaaccacttttctgtcttccaaaaccctcattgaatcactcctcattca

SEQ ID NO: 262 - DNA, 1216 bp, Arabidopsis thaliana
  source 1..1216: mol_type = genomic DNA
caactatttttatgtatgcaagagtcagcatatgtataattgattcagaatcgttttgacgagttcggatgtagtagtagccattatttaatgtacatactaatcgtgaatagtgatatgatgaaacattgtatcttattgtataaatatccataaacacatcatgaaagacactttctttcacggtctgaattaattatgatacaattctaatagaaaacgaattaaattacgttgaattgtatgaaatctaattgaacaagccaaccacgacgacgactaacgttgcctggattgactcggtttaagttaaccactaaaaaaacggagctgtcatgtaacacgcggatcgagcaggtcacagtcatgaagccatcaaagcaaaagaactaatccaagggctgagatgattaattagtttaaaaattagttaacacgagggaaaaggctgtctgacagccaggtcacgttatctttacctgtggtcgaaatgattcgtgtctgtcgattttaattatttttttgaaaggccgaaaataaagttgtaagagataaacccgcctatataaattcatatattttcctctccgctttgaattgtctcgttgtcctcctcactttcatcagccgttttgaatctccggcgacttgacagagaagaacaaggaagaagactaagagagaaagtaagagataatccaggagattcattctccgttttgaatcttcctcaatctcatcttcttccgctctttctttccaaggtaataggaactttctggatctactttatttgctggatctcgatcttgttttctcaatttccttgagatctggaattcgtttaatttggatctgtgaacctccactaaatcttttggttttactagaatcgatctaagttgaccgatcagttagctcgattatagctaccagaatttggcttgaccttgatggagagatccatgttcatgttacctgggaaatgatttgtatatgtgaattgaaatctgaactgttgaagttagattgaatctgaacactgtcaatgttagattgaatctgaacactgtttaagttagatgaagtttgtgtatagattcttcgaaactttaggatttgtagtgtcgtacgttgaacagaaagctatttctgattcaatcagggtttatttgactgtattgaactctttttgtgtgtttgcagctcataaaaa

SEQ ID NO: 263 - DNA, 1425 bp, synthetic construct
  source 1..1425: mol_type = other DNA
tgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgaagatctgattaaacatggaagtttctctcttgagggaggttgctcgtggaatgggacacatatggttgttataataaaccattccattgtcatgagattttgaggttaatatatactttacttgttcattattttatttggtgtttgaataaatgatataaatggctcttgataatctgcattcattgagatatcaaatatttactctagagaagagtgtcatatagattgatggtccacaatcaatgaaatttttgggagacgaacatgtataaccatttgcttgaataaccttaattaaaaggtgtgattaaatgatgtttgtaacatgtagtactaaacattcataaaacacaaccaacccaagaggtattgagtattcacggctaaacaggggcataatggtaatttaaagaatgatattattttatgttaaaccctaacattggtttcggattcaacgctataaataaaaccactctcgttgctgattccatttatcgttcttattgaccctagccgctacacacttttctgcgatatctctgaggtaagcgttaacgtacccttagatcgttctttttctttttcgtctgctgatcgttgctcatattatttcgatgattgttggattcgatgctctttgttgattgatcgttctgaaaattctgatctgttgtttagattttatcgattgttaatatcaacgtttcactgcttctaaacgataatttattcatgaaactattttcccattctgatcgatcttgttttgagattttaatttgttcgattgattgttggttggtggatctatatacgagtgaacttgttgatttgcgtatttaagatgtatgtcgatttgaattgtgattgggtaattctggagtagcataacaaatccagtgttccctttttctaagggtaattctcggattgtttgctttatatctcttgaaattgccgatttgattgaatttagcgcgcttagctcagatgatagagcaccacaatttttgtggtgagaaatcggtttgactccgatagcggctttttactatgattgttttgtgttaaagatgattttcataatggttatatatgtctactgttttttattgattcaatatttgattgttcttttttttgcagatttgttgacagtctctaaca

SEQ ID NO: 264 - DNA, 385 bp, Figwort mosaic virus
  source 1..385: mol_type = genomic DNA; note = Figwort mosaic virus hybrid P
cagctggcttgtggggaccagacaaaaaaggaatggtgcagaattgttaggcgcacctaccaaaagcatctttgcctttattgcaaagataaagcagattcctctagtacaagtggggaacaaaataacgtggaaaagagctgtcctgacagcccactcactaatgcgtatgacgaacgcagtgacgaccacaaaagcccgactcgaacatcttgaaggtgtacaaaacgttttagcagattgcctcacgagagattttaatgcttaaaacgtaagcgctgacgtatgatttcaaaaaacgcagctataaaagaagccctccagcttcaaagttttcatcaacacaaattctaaaaacaaaattttttagagagggggagtgaca

SEQ ID NO: 265 - DNA, 230 bp, Arabidopsis thaliana
  source 1..230: mol_type = genomic DNA
ggatgatggtgaagaaattgtcgacctttctcttgtctgtttgtcttttgttaaagaagctatgcttcgttttaataatcttattgtccattttgttgtgttatgacattttggctgctcattatgttatgtgggaagttagtgttcaaatgttttgtgtcggtattgttcttctcatcgctgttttgttgggatcgtagaaatgtgaccttcggacagtaaaactcttg

SEQ ID NO: 266 - DNA, 220 bp, Arabidopsis thaliana
  source 1..220: mol_type = genomic DNA
gaatcacaagttcttgtcatgttttgatttgtatctcataataaagcaaaatgttcatttttgaatgagctttactctctccatctcttgtttgttgtcatcccatttatttcctctcagatgctttcgtagtgagttccaaagacaactaaatgactcagttttattgttcgatggttcactaatcagcacagaatggaacaattgtttttatctgctt

SEQ ID NO: 267 - DNA, 309 bp, Cucumis sativus
  source 1..309: mol_type = genomic DNA
agttgaagaatttgatgaagaatgctttgtttgggttgatttggttaataaaggacttcaaatgtattttgtgtaacttttgagtgtcgaaacagttcgatcgtttactttttgtattatattatcgtgggaatgaatacaataaaataaaatttcatggtataccacactgctacattgaaaatctaatcatttatcaattataagatacacatgttacttcaattcgagagcccatgaagtctgggtgtaagcgatcttgaagaaaaaccccgaagccaattatgttttcttttatatattatattc

SEQ ID NO: 268 - DNA, 299 bp, Cucumis sativus
  source 1..299: mol_type = genomic DNA
aaatgaagatgaaggttttgcaataaaagtgttctcttttttttgttttggtaattacctattaatgtagagaagttgaagaaggatgttaactagtggggttttgtcttctgtttgatatataataaacgtaagctttgcatataattcaatggatatctttttctgttttcttcttctctctctctatctcaaagccatactttggtgaagaaatgggttttttgtaataaaatgaagccattctcaagcaacgaatatgccacgcgcgccttattagcgggcgcgggcgtggacac

SEQ ID NO: 269 - DNA, 858 bp, Citrullus lanatus
  source 1..858: mol_type = genomic DNA
gaccaaatttatttatggttgaaatcttaaaggcctatttgattggtaatttagattctgttatgttttgaaattcactgggttaagcaaaatatacgcttgacagacaatttaaattatatttactgtatagttagattttgaatttgatttataaataaagttttttatgttataaactcaaactttttttccttgagaaaatattgtaaatttaattcactatttaaaaaattaaggatttttttcaaatatagccaaagcaatcaaaatatttataaatatagcaaaattttattatttatctacaatagaccacgataaatcgtgatagatggtgtattttactattaatccatatatttttaagcagttttcacatttaagatgatttctgattttattatgtttttcctcaaaaagtaattaatgatataaattgatttcctttttatcttcctttacgagtcccatgacttatctctttggactcctctccaacggcggattacaatgatctactcctacacacattgttgtcacttttctgccaatcataggcatccacgtaactcgagatcacataaaccttagcgcagtggatcgaaattagcctctataaataaagcaaacgccattgatggattcaccggagctagggtttctccccaattctctgcctataaaaaaaacttctcctccaattcggctcgtattctctcaatcaacaaattcaaaaaaacccatcgatttcttttgttttgattggatttgctaaacccgtcgatttcctttgtttcgatttgatttgctttacccagaaacaaagaagaacaaaaaagcaacaatcaatcggcc

SEQ ID NO: 270 - DNA, 1229 bp, Cucumis melo
  source 1..1229: mol_type = genomic DNA; note = Cucumis melo L.
gatcttactttaccataatggtgaaaaggatagtgacccacatggtttttacttcgttatagagacaagatgaaaacaaatctaaaatttaatattatagatggatagatgatggacaacaaaaagagaaaagaagatactggtcattggtccaaaacagccacccgaatcaatatatgaccgaaaaacaaaagctacagaatcatatctgtgcaacggtgccacagtgctataggatagcacaaccacactgtcacataaaaaagaggattttgcactcgttttagatggagtttcgtaattttcgggtctttcaagcttaaatatatacttcattaaagcttcgaattttgtaatgttcaattctacctctttgatgttcgatacctataaaataattaaataaacgtatagacgtaggaacaattaagcggagttagatagtgcatttatgattctacctgtgagtgcaatggtaaaatggacattataaaagagtaggggcaaagagggaagtgaaaaattctccccacttagccatgtttaatatagtagggataggaatatgtaataagtagtgttttttctatttaattttctgtatacttcttccatctcctttaattattaaaaggttttcctctctttactctttctctctaaattactattctgaagtatattttcttttataaaaagagtaataaactttatttccattaaaagaacaaacaacaagaaatgataatcaaatacacattcatatttttaaaaaaaaagttaaacaagatatagaaatagttatcaaatatatttatgttgtcattccttgtatacaatggcattcctttagctttgtttatgtatttcctgagcttctcttagtgtactatatcctttaatattaatgcatctttcgatcttgctaagatatgataaaaatagacgacacgtgtcacaacctaattgagatatttcgatgtactttctatccgtcttagcttgtaattaattattgttaaaaaagaatactcaattaactagaaacaagaaataagaaacgaaaacattacaaaacggagttgaagcgtgcaaatttgtggaaatgattgttatcatgaaccagaaaacattaaataactcttcctataaaaggcccttattcttcactttctcaaatcacgtcctaaagatatcaaagatttcaactgatagcaaaaagcactact

SEQ ID NO: 271 - DNA, 2001 bp, Siraitia grosvenorii
  source 1..2001: mol_type = genomic DNA
attagtattttcaaaagtcatcaaacttactaaaagaaaattttatatttttacattcctctgtacttaattgcatttattcaataaactttgacaataattataatttgatttgatattatcatatgaatttactacgaaaatagttttgcatgcatgttaatcatgaaatgcacaatgattttcgtatataaaattcttttgctgaatggtattatttgtcgccataaccattattttatactaaataataaattgataaaattcctagacacgaacgctttgttgacttggatagaacgtaaacacgtgaatcatgagtattgcgaagtaatggtcagaaaggtttcttttgatttttttttttaattccatgtaagtttaatttaatatgtctatctaattttattaaattaaactaaaaaataagtggtagaaattaatatcttctattagttgagttttttttttttttttttttaaattatggctatttattagctctattgtgactggaaaaatctattttgtatttagactagattatatgctttttttttgtttgttaatttggagattttagtagtaagtttggacaattttacctaattaatcttgtggctatgttagatactagcatttctggtggcaagagacaatttatccctctaatacttgaccttaattgtggtcatatgaccaacatgaacatagctaatggttaaggtattactacttcttctaagaagtcgtaggttcgaatcctcaccccacatttgtgatatgatatctcaaaataaaaattgtggttatatgtattttctttttctgaaacaagccttctcgttaaaaagataataaattataattcgctacaaatagttgaggtatatgttttgaaattaataattaattaaaaaaatagtaagtcatgcattattattattattttggtttaaatattatgttagtccttgtactttgaacttttgtttattttagtccttgtactttcaaaatgttcatttttggtccctgtactttgagcttttattcattttgatcattgtactttcaaaatgttcatttttggactctgtactatcattttttgtccactttagtccctatactttcaagaaatttaccattttggtcttttgattttcctttttatctcatttttttaatcacattttcagcacaataatacactcagtaaatttcttgaaatattatcatatttttgtattaaaaggttttcgttgagtatagaatttgtattagaatttatcaaatatgaaccaaaatgattattttttaaaagtacagggactaaaatggatgttttaaaagtataaggactaaaatggataaaaactgaaagtatagggactaaaaatgaacattttgaaagtatatggaccaaaatgaataaaaactcaaagtatagagactaaaataatatttaaacctattatttattattttgaaaatatagcattaattacaatagtaggagtgggaatttgaagcttggatctataggttgaaccatttttacagtacaggtatagaataattttgaagagagataatttgagaaaaatagacaccattggcttgatagaaacattttattatatatatttatgagaatatgaagagatacataaaaatgcattactttacttggtagcaggcgcagatatataagtgtcgttggtgtgtggaaagctgaaagcagaggcgtccaagtgttaatgtagattggttttatgtggaagaaaaagttgggcgttgggatgagttgaagttgccaaacgtcccagagagtctgagctgtgaagagagaggtgaggcagtatgggtatatagttagcgttaatataatttagccagcttgtgtcccatttaatgaatatataatttgtgtatttgtatgcattggtggtatacaaagtagttaagaggtagaagaagcagaaattatattaaagg

SEQ ID NO: 272 - DNA, 1275 bp, synthetic construct; identical to SEQ ID NO: 16
  source 1..1275: mol_type = other DNA

SEQ ID NO: 273 - protein, 424 aa, Citrullus lanatus; identical to SEQ ID NO: 17

SEQ ID NO: 274 - DNA, 1272 bp, synthetic construct
  source 1..1272: mol_type = other DNA
atgaacacgaccccgctgccggaggaggacgaggaggtggtgaagatggtggtggacgggtccgtgccgtcctactcgctcgagtccaagctcggcgacccgaagcgcgcggcgtccatccggcgggaggcgctgcagcgcatgacggggcggagcatccacgggctgccgttcgagggcttcgactacgagagcatcctgggccagtgctgcgagatgccggtggggtacgtccagatcccggtgggcatcgcggggccgctgctgctcgacgggttcgagtacaccgtgccgatggccaccacggagggctgcctggtggcctccaccaacaggggctgcaaggccatctacgcctccggcggggcgacctcaatgctcctcaaagacggcatgacgagggccccggtggtcaggttcggcagcgcgaagagggcgagcgagctgaagttcttcctggaggacccgaacaacttcgacaccctggccgtggtgttcaaccgctccagccgcttcgcccgcctgcagagcatcaggtgcagcatcgcgggcaagaacctgtacgtccgcttctgctgctccaccggcgacgccatgggcatgaacatggtgtccaagggcgtgcagaacgtgctggagttcctgcagcacgacttctccgacatggaggtgatcggcatctccggcaacttctgcgccgacaagaagccggccgccgtgaactggatcgagggcaggggcaagtccgtggtgtgcgaggccattatcaaggacgaggtggtgcgcaaggtgctcaagaccagcgtggcgagcctggtggagctgaacatgctgaagaacctgaccggctccgccatggccggggccctcggcggcttcaacgcgcacagcagcaacatcgtgtccgccatcttcctggccaccggccaggacccggcccagaacgtggagtcctcccactgcatcacgatgatggagccggtcaacaacggcaaggacctccacatctccgtgaccatgccgtccatcgaggtcgggaccgtggggggcggcacccagctggcgtcccagagcgcgtgcctgaacctgctgggggtcaagggcgcgtccaaggagagcccgggggccaacagcaggctgctggcgaccatcgtggccggctccgtgctggccggcgagctctccctgatgtccgccatcgcggcgggccagctggtgaggtcccacatgaagtacaaccgctcctccagggatgtgtccaaggtggagagc

SEQ ID NO: 275 - protein, 424 aa, Citrullus lanatus; identical to SEQ ID NO: 17

SEQ ID NO: 276 - DNA, 1275 bp, synthetic construct
  source 1..1275: mol_type = other DNA
atgaacacgacccctttgccggaggaggatgaagaagttgtgaaaatggtggttgatggcagcgtgccatcgtactcgctcgagtcaaagctcggcgacccaaaacgggcagcctcaatccggcgggaggcattgcagcggatgacaggtcggagcatccacgggctgccttttgaaggctttgactatgaatctatacttgggcaatgctgcgaaatgcccgtcggttatgtccagattccggttggcatcgctggacctctgttgctcgatgggtttgaatatacggtgcccatggcaactacggagggttgccttgtggcttctactaatcgcggttgcaaagctatctacgcctcaggaggggcgacctcaatgctcctcaaagacggtatgacgagggcacctgttgtcagattcggtagcgcgaagagagcgtcagagttgaaattctttcttgaagacccgaataatttcgacaccttggctgtcgtgttcaatcggtcttcgagatttgcgagattgcaatcgatcaggtgcagcatcgcgggcaaaaatctctatgttagattctgttgctcaacaggagatgcaatgggtatgaacatggtttccaaaggggtccaaaacgtgcttgaatttctgcaacacgattttagcgatatggaagttataggcatttcaggcaatttctgcgccgataagaagcccgctgccgttaattggatcgagggtcgcggaaaatctgttgtctgtgaggcaattatcaaggatgaggttgtgcgcaaagttctcaagactagcgtcgcaagcttggtcgaattgaacatgctgaagaacctgactggctccgcgatggcaggggccttgggtggtttcaacgctcacagcagcaacatagtttctgccatatttttggcgaccggacaggacccggcacaaaatgttgagtcttctcactgcatcacgatgatggaaccagtcaacaatggaaaagacctccatatttctgtcacgatgccttcaatcgaagtcgggacagttggtggaggaacacaattggcttcacagagcgcgtgccttaatttgctgggtgtcaagggtgcctccaaggaatcgcctggggcgaattctaggcttttggcgaccatcgttgcaggaagcgttttggcgggggagctctcgctgatgtccgccatcgcggcgggtcagttggtgagatcgcacatgaaatataacagaagctccagagatgtgtcaaaagttgaaagctaa

SEQ ID NO: 277 - protein, 424 aa, Citrullus lanatus; identical to SEQ ID NO: 17

SEQ ID NO: 278 - DNA, 1557 bp, synthetic construct; identical to SEQ ID NO: 1
  source 1..1557: mol_type = other DNA

SEQ ID NO: 279 - protein, 518 aa, Siraitia grosvenorii; identical to SEQ ID NO: 2

SEQ ID NO: 280 - DNA, 1425 bp, synthetic construct
  source 1..1425: mol_type = other DNA
atggtgcaacctcgggtactgctgtttcctttcccggcactgggccacgtgaagcccttcttatcactggcggagctgctttccgacgccggcatagacgtcgtcttcctcagcaccgagtataaccaccgtcggatctccaacactgaagccctagcctcccgcttcccgacgcttcatttcgaaactataccggatggcctgccgcctaatgagtcgcgcgctcttgccgacggcccactgtatttctccatgcgtgagggaactaaaccgagattccggcaactgattcaatctcttaacgacggtcgttggcccatcacctgtattatcactgacatcatgttatcttctccgattgaagtagcggaagaatttgggattccagtaattgccttctgcccatgcagtgctcgctacttatcgattcacttttttataccgaagctcgttgaggaaggtcaaattccatacgcagatgacgatccgattggagagatccagggggtgcccttgttcgaaggtcttttgcgacggaatcatttgcctggttcttggtctgataaatccgcagatatatctttctcgcatggcttgattaatcagacccttgcagctggtcgagcctcggctcttatactcaacaccttcgacgaactcgaagctccatttctgacccatctctcttccattttcaacaaaatctacaccattggacccctccatgctctgtccaaatcaaggctcggcgactcctcctcctccgcttctgccctctccggattctggaaagaggatagagcctgcatgtcctggctcgactgtcagccgccgaggtctgtggttttcgtcagtttcgggagtacgatgaagatgaaagccgatgaattgagagagttctggtatgggttggtgagcagcgggaaaccgttcctctgcgtgttgagatccgacgttgtttccggcggagaagcggcggaattgatcgaacagatggcggaggaggagggagctggagggaagctgggaatggtagtggagtgggcagcgcaagagaaggtcctgagccaccctgccgtcggtgggtttttgacgcactgcgggtggaactcaacggtggaaagcattgccgcgggagttccgatgatgtgctggccgattctcggcgaccaacccagcaacgccacttggatcgacagagtgtggaaaattggggttgaaaggaacaatcgtgaatgggacaggttgacggtggagaagatggtgagagcattgatggaaggccaaaagagagtggagattcagagatcaatggagaagctgtcaaagttggcaaatgagaaggttgtcaggggtgggttgtcttttgataacttggaagttctcgttgaagacatcaaaaaattgaaaccatataaattttaa

SEQ ID NO: 281 - protein, 474 aa, Siraitia grosvenorii; identical to SEQ ID NO: 9

SEQ ID NO: 282 - DNA, 1362 bp, synthetic construct; identical to SEQ ID NO: 10
  source 1..1362: mol_type = other DNA

SEQ ID NO: 283 - protein, 453 aa, Siraitia grosvenorii; identical to SEQ ID NO: 11

SEQ ID NO: 284 - DNA, 2280 bp, synthetic construct
  source 1..2280: mol_type = other DNA
atgtggcgccttaaggttggggccgagtctgtcggcgagaacgatgaaaagtggctcaaatcaatcagcaatcatcttggcaggcaggtctgggaattttgtcccgatgccggaactcaacaacagttgttgcaagtccacaaagcgagaaaggcgttccatgacgatagatttcatcggaaacagtcgtcggatcttttcattactatacaatacgggaaggaagttgagaacggtgggaagacggctggcgtgaaacttaaagagggcgaagaggtgagaaaggaagccgttgagagctccttggagcgggccttgtcgttttattcaagcatccagacgtctgacggtaattgggcatcggacctcgggggccccatgttcctcctgcccgggctggtcatcgcgctctacgttaccggtgttttgaattcggtcctgtcaaaacatcacaggcaggagatgtgtcggtatgtctataatcatcagaatgaggatggcggttggggcctgcacatagaagggccgtctactatgttcggttcagcattgaactacgtggctctgcggcttctcggtgaggacgccaatgcgggtgcaatgccaaaagcgcgcgcttggattctggaccacggcggggccaccggaattacttcttggggaaagctctggctctcagttcttggcgtgtacgagtggtctggcaacaacccactccccccagaattttggctctttccgtatttccttcccttccatccgggcagaatgtggtgtcattgtcggatggtgtatttgccgatgtcctatttgtacgggaaaaggttcgttgggccgataacgccgatcgttcttagccttagaaaagagctctatgcggtcccttatcacgagatcgactggaacaaatctcggaacacatgtgcaaaagaggacttgtactacccccaccccaagatgcaggacatcttgtggggatcattgcaccatgtttacgagcccctgtttactcggtggccagcgaaaaggcttagggaaaaggcgctgcagacggctatgcaacatattcactatgaagatgaaaacactcgctatatttgcctgggcccagttaataaagtgctgaatctgctttgttgttgggtcgaagatccatactctgatgcttttaaactgcatcttcaacgcgtgcatgattacctttgggtggcagaggatggcatgaaaatgcagggatataacggttcccaactttgggataccgctttcagcattcaagcaatagtctcaacgaagttggtcgataactacggtcccactttgcggaaggcgcacgatttcgtcaaatcatcgcaaatacaacaggactgccctggcgatcccaacgtgtggtacaggcatatccacaaaggagcatggccattttctacgagggaccatgggtggctgatatcggattgtaccgccgaaggactcaaggcagcccttatgctcagcaagttgccgtccgagacagtgggtgagtcgcttgaacgcaatcggctgtgcgacgctgttaacgtcttgctgtctttgcagaacgacaacggcgggttcgcatcgtatgagcttactagatcgtatccgtggcttgaactcattaatcccgccgaaacattcggagatatcgtgattgactatccctatgtggaatgcacttctgcaacaatggaagcgcttacacttttcaaaaaacttcaccctgggcacaggactaaggaaatcgacacagcgatagtgcgcgccgcgaacttcctggagaatatgcagagaactgacggctcttggtatggctgctggggcgtttgcttcacatatgcaggctggtttggaattaaaggcctcgttgccgccgggcgcacgtacaataactgtcttgctatcaggaaagcttgtgattttctgctttccaaggagcttccgggcggtgggtggggcgagtcgtatttgagctgccaaaataaagtctatacaaacctcgagggtaaccggcctcaccttgtcaacacagcttgggttttgatggcgttgatagaggctggtcaagctgagcgggaccccacaccacttcatagggccgcaaggcttttgataaattcacagctcgaaaatggcgatttcccgcaacaggaaatcatgggagtttttaacaaaaactgcatgataacctacgccgcttataggaatatattcccgatctgggccctgggtgaatactgccatcgggtgttgaccgagtaa

SEQ ID NO: 285 - protein, 759 aa, Siraitia grosvenorii
MWRLKVGAESVGENDEKWLKSISNHLGRQVWEFCPDAGTQQQLLQVHKARKAFHDDRFHRKQSSDLFITIQYGKEVENGGKTAGVKLKEGEEVRKEAVESSLERALSFYSSIQTSDGNWASDLGGPMFLLPGLVIALYVTGVLNSVLSKHHRQEMCRYVYNHQNEDGGWGLHIEGPSTMFGSALNYVALRLLGEDANAGAMPKARAWILDHGGATGITSWGKLWLSVLGVYEWSGNNPLPPEFWLFPYFLPFHPGRMWCHCRMVYLPMSYLYGKRFVGPITPIVLSLRKELYAVPYHEIDWNKSRNTCAKEDLYYPHPKMQDILWGSLHHVYEPLFTRWPAKRLREKALQTAMQHIHYEDENTRYICLGPVNKVLNLLCCWVEDPYSDAFKLHLQRVHDYLWVAEDGMKMQGYNGSQLWDTAFSIQAIVSTKLVDNYGPTLRKAHDFVKSSQIQQDCPGDPNVWYRHIHKGAWPFSTRDHGWLISDCTAEGLKAALMLSKLPSETVGESLERNRLCDAVNVLLSLQNDNGGFASYELTRSYPWLELINPAETFGDIVIDYPYVECTSATMEALTLFKKLHPGHRTKEIDTAIVRAANFLENMQRTDGSWYGCWGVCFTYAGWFGIKGLVAAGRTYNNCLAIRKACDFLLSKELPGGGWGESYLSCQNKVYTNLEGNRPHLVNTAWVLMALIEAGQAERDPTPLHRAARLLINSQLENGDFPQQEIMGVFNKNCMITYAAYRNIFPIWALGEYCHRVLTE

SEQ ID NO: 286 - DNA, 1425 bp, synthetic construct; identical to SEQ ID NO: 8
  source 1..1425: mol_type = other DNA

SEQ ID NO: 287 - protein, 474 aa, Siraitia grosvenorii; identical to SEQ ID NO: 9

SEQ ID NO: 288 - DNA, 1422 bp, synthetic construct; identical to SEQ ID NO: 6
  source 1..1422: mol_type = other DNA

SEQ ID NO: 289 - protein, 473 aa, Siraitia grosvenorii; identical to SEQ ID NO: 7

SEQ ID NO: 290 - DNA, 1587 bp, synthetic construct; identical to SEQ ID NO: 12
  source 1..1587: mol_type = other DNA

SEQ ID NO: 291 - protein, 528 aa, Siraitia grosvenorii; identical to SEQ ID NO: 13

SEQ ID NO: 292 - DNA, 951 bp, synthetic construct; identical to SEQ ID NO: 14
  source 1..951: mol_type = other DNA

SEQ ID NO: 293 - protein, 316 aa, Siraitia grosvenorii; identical to SEQ ID NO: 15

SEQ ID NO: 294 - DNA, 1422 bp, synthetic construct
  source 1..1422: mol_type = other DNA
atggtgcagccccgggtgctgctgttccctttccccgcgctgggccacgtgaagcccttcctgtcgctggcggagctcctgtccgacgccggcatcgacgtggtcttcctctcgacggagtataaccaccgccgcatctccaacaccgaggccctggcctcccgcttccccaccctgcacttcgaaacgataccggacggcctgccccccaacgagtcgcgcgccctcgcggacggccccctgtacttcagcatgagggagggcaccaagccgcgcttccggcagctgatccagagccttaacgacggccgttggcccatcacctgtatcatcacggacatcatgctcagttcgcccatcgaggtggcggaggagttcgggatcccggtgatcgcgttctgcccgtgctccgctcgctacctctcgatccacttcttcatcccgaagctggtggaggaggggcagatcccgtacgcggacgacgaccccattggggagatccagggggtgcccctcttcgaggggcttctccgacggaaccacctcccgggatcttggtccgacaagtccgcggacatttccttctcgcacggcctcatcaaccagacgctcgcggccgggcgggcctcggccctgatcctcaacaccttcgacgagctcgaggcccctttcctcacccacctgagctccatcttcaacaagatctacaccatcggccccctccacgccctgtccaagagccggctcggggactcctcctcgtcggccagcgcgctctcggggttctggaaggaggaccgcgcgtgcatgtcctggctcgactgccagccgccgcgctcggtcgtgttcgtctccttcgggtcgaccatgaagatgaaggcggacgagcttcgggagttctggtacgggctggtgagcagcgggaagccgttcctctgcgtgttgcggtccgacgtcgtcagcggcggcgaggcggcggagctcatcgagcagatggcggaggaggagggggctgggggcaagctgggcatggtcgtcgagtgggcggcgcaggagaaggtcctctcccaccctgccgtggggggcttcctgacgcactgcggctggaactcgacggtggagagcatcgccgccggcgtccccatgatgtgctggccgatcctcggggaccagccctccaacgccacttggatcgaccgggtctggaagatcggggtcgagcgaaacaaccgggagtgggaccggttgacggtggagaagatggtccgcgcattgatggagggccagaagcgggtggagatccagcggtcgatggagaagctgtcgaagctggcgaacgagaaggtcgtccggggggggctgtctttcgacaacctcgaggtcctcgtggaggacatcaagaagctgaagccgtacaagttc

SEQ ID NO: 295 - protein, 474 aa, Siraitia grosvenorii; identical to SEQ ID NO: 9

SEQ ID NO: 296 - DNA, 1557 bp, synthetic construct
  source 1..1557: mol_type = other DNA
atgtccagctcagtcgcggctaccataagcatatggatggtcgttgtctgtatagtgggtgtcgggtggcgggtggtcaactgggtttggcttcgccccaaaaaacttgagaagcggctccgggaacaaggtctggctggaaattcgtatcggctccttttcggggaccttaaagaaagagccgcgatggctgaacaggcaaattcaaagccaattaacttttcgcatgatatagggccacgggtttttccgtcgatgtataaaactattcagaactatggtaagaactcttacatgtggttgggaccttatccgagggttcatatcatggatcctcagcagctgaagactgtgtttacgcttgtgtacgacatacagaagcccaatcttaatcccttgattaaatttttgttggatggaattgttacgcatgaaggggaaaagtgggctaaacaccgcaaaatcattaaccccgcattccatatggcaaagttgaaagatatgattccagcttttttccaatcctgtaatgaaattgtcagcgaatgggagaggatgatcccgaaggaggggagcttcgaattggatgtgatgccctatatgcagaatctggccgctgacgctattagccggactgccttcggaagcagctataaggaaggtcaaatgatttttcagttgctcaaagagttgaccgatcttgtggtgaaagcagccttcggcatttatatccccggatggagattcctccccacgaaaagcaacaataaaatgaaggagataaaccggaagattaaaactcttttgcttggtattatagataagaggcagaaggcaatgaaagaaggcgaggtcgtccagaatgaccttcttgggattttggtcgagtcgaaccttaacgagataagagggcatgagaataataaagacgcggggatgtccatcgaagatgtgatagaagagtgtaaggtgttctacataggtggccaggagacaactgcaggcctgttgatatggaccatgatattgctcagcttccatacggaatggcaggaacgcgctagaaccgaagttctcgaagtcgttgggaacaaaaaacctgatttcgacggattgaatcggctgaagatagttacaatgattctctatgaggtgctccggttgtatcctcccgcctctatgttgacccgcattgtccaaaaggaaacgagaatagggaagcttactcttccggctggagtcacactgataatcccaacggtgttgatacaccgcgatcacgaattgtggggcgaggacgcgaatgaattcaagcccgaacgcttcagcgagggagttagcaaggccgctaaggtgcagcctgcgttcttcccgttcggatgggggcccagactttgtctcgggcaaaacttcgcaatgattgaggccaaaatggcgttttctctgattctgcaaagattctctttccacctctcatcatcatatgcacacgcgcctaccgtcgttatgacaactcagcctcagcatggcgctcatatagttctcagaaagctctaa

SEQ ID NO: 297 - protein, 518 aa, Siraitia grosvenorii; identical to SEQ ID NO: 2

SEQ ID NO: 298 - DNA, 1359 bp, synthetic construct
  source 1..1359: mol_type = other DNA
atggacgcgcagcggggccacacgacgacgatcctgatgttcccctggctcggctacgggcacctcagcgccttcctcgagctggcgaagagcctgtcccggcgcaacttccacatctacttctgctcgaccagcgtcaacctggacgcgatcaagcccaagctgccctccagcagctcctccgacagcatccagctggtggagctgtgcctgccctccagccccgaccagctccccccgcacctccacaccaccaacgcgctcccgccccacctcatgcccaccctccaccaggccttcagcatggcggcgcagcacttcgcggccatcctgcacaccctcgcgccccacctcctgatctacgactcgttccagccgtgggcaccccagctggcctcctcgctcaacatcccggccatcaacttcaacacgaccggggcgtccgtcctgacccggatgctgcacgcgacccactacccgagcagcaagttccccatcagcgagttcgtgctccacgactactggaaggccatgtacagcgcggccgggggcgcggtgacgaagaaggaccacaagatcggcgagacgctggcgaactgcctccacgccagctgctcggtcatcctgatcaactccttccgcgagctggaggagaagtacatggactacctgtccgtgctcctgaacaagaaggtggtgccggtggggccgctggtgtacgagccgaaccaggatggagaggacgaggggtactccagcataaagaactggctcgacaagaaggagccgagctccacggtgttcgtgtccttcggcagcgagtacttcccgagcaaggaggagatggaggagatcgcccacgggctcgaggcctctgaagttcatttcatctgggttgtgcggttcccgcagggggacaacacctccgcgatcgaggacgccctgccgaagggcttcctcgagagggtgggcgagaggggcatggtggtgaagggctgggccccgcaggcgaagatcctgaagcactggtccaccgggggcttcgtgtcccactgcggctggaacagcgtgatggagagcatgatgttcggggtgccgatcatcggcgtgccgatgcacctggaccagccgttcaacgccggcctggccgaggaggccggcgtgggggtggaggcgaagcgcgaccccgacgggaagatccagcgcgacgaggtcgccaagctgatcaaggaggtggtggtggagaagacccgggaggacgtgaggaagaaggcgcgcgagatgtccgagatcctccgcagcaagggggaggagaagatggacgagatggtggccgcgatctccctcttcctcaagatc

SEQ ID NO: 299 - protein, 453 aa, Siraitia grosvenorii; identical to SEQ ID NO: 11

SEQ ID NO: 300 - DNA, 1425 bp, synthetic construct
  source 1..1425: mol_type = other DNA
atggtccaaccccgggtcctgctctttccgtttccggctcttggccatgttaaaccattcctttcccttgccgagctcctgtcggacgcaggaatagatgtcgtctttttgtcaacggagtataatcatcggaggatctcaaatacagaggcacttgcaagccggtttcccactcttcacttcgaaaccattccagatgggctgccgccaaatgagagcagggctttggccgacggccctctttacttctcgatgagggaggggacaaaaccgaggtttcgccagcttatacagagcctgaatgatggtcggtggcccattacttgtataattacggatataatgctttccagccccatcgaagttgcggaggagtttgggataccggtgatagcattttgtccttgctcggctcgctacttgtctatacacttctttatcccgaagctcgtcgaagaaggccagataccatacgcggatgacgaccccattggagaaatacaaggagtgcctctttttgaggggctcctgcgcagaaatcatctgcctggttcttggagcgataaatccgctgacatctcattcagccatgggctgataaatcagacgcttgcggccggtcgggcatcggcactgattcttaacacctttgatgaacttgaggccccatttcttactcacctctcgtcgatttttaacaagatttacacaatcgggccacttcatgctctttccaaatcacggcttggagattcgagcagcagcgcatccgctctgtcgggattctggaaagaggacagagcgtgcatgtcttggcttgattgccaaccaccccggtcggttgtttttgtttcctttgggtcgacaatgaagatgaaggcggatgagttgcgggagttttggtacgggcttgtctcttccggaaagcccttcctctgtgtgctgagatcagatgttgtctcgggcggtgaggcagccgagctcatagagcaaatggcggaagaggagggggccgggggtaaactcggtatggtggtcgagtgggcggctcaggagaaagtgttgtcacatcctgctgtcgggggattcctgacacattgcggatggaattctactgtcgagtcaattgcggcgggcgtgcctatgatgtgctggcccatactgggcgaccagccctctaatgcgacttggatagaccgggtctggaaaattggcgttgaacggaacaaccgcgaatgggaccgcctgacagttgagaagatggttagagcactcatggaagggcaaaagagagtcgaaattcaacgctctatggaaaaattgagcaaacttgcaaatgaaaaggtcgtgagaggtgggctgtcgtttgataatctggaggttctggttgaggacataaaaaagcttaagccctacaaattttga

SEQ ID NO: 301 - protein, 474 aa, Siraitia grosvenorii; identical to SEQ ID NO: 9

SEQ ID NO: 302 - DNA, 1425 bp, synthetic construct; identical to SEQ ID NO: 280
  source 1..1425: mol_type = other DNA

SEQ ID NO: 303 - protein, 474 aa, Siraitia grosvenorii; identical to SEQ ID NO: 9

SEQ ID NO: 304 - DNA, 1362 bp, synthetic construct; identical to SEQ ID NO: 10
  source 1..1362: mol_type = other DNA

SEQ ID NO: 305 - protein, 453 aa, Siraitia grosvenorii; identical to SEQ ID NO: 11